(19) United States
(12) Patent Application Publication
(10) Pub. No.: US 2026/0155228 A1
(43) Pub. Date: Jun. 4, 2026

(54) SYSTEMS, METHODS, COMPUTING PLATFORMS, AND STORAGE MEDIA FOR MEDICINE RECOMMENDATIONS, PHARMACIST-PROVIDER REAL-TIME COMMUNICATIONS, DISPLAYING A VISUALIZATION OF PROGRAMMING INSTRUCTIONS FOR A MEDICAL DEVICE

(71) Applicant: Caleb Hernandez, New York, NY (US)

(72) Inventor: Caleb Hernandez, New York, NY (US)

(21) Appl. No.: 19/444,814

(22) Filed: Jan. 9, 2026

Related U.S. Application Data
(63) Continuation-in-part of application No. 17/526,244, filed on Nov. 15, 2021.
(60) Provisional application No. 63/113,809, filed on Nov. 13, 2020.
(60) Provisional application No. 63/144,415, filed on Feb. 1, 2021.

(51) Int. Cl. G16H 20/10; G16H 10/40; G16H 10/60; G16H 15/00; G16H 50/30; G16H 70/40; G16H 80/00

(52) U.S. Cl. CPC G16H 20/10; G16H 10/40; G16H 10/60; G16H 15/00; G16H 50/30; G16H 70/40; G16H 80/00

(57) ABSTRACT

A device may include a data acquisition layer comprising a patient information collector and a drug information collector, each operable to ingest patient-specific clinical data and drug-specific data, respectively. A device may include an analysis engine comprising a multi-criteria ranking engine configured to compute composite suitability score for candidate medications by weighting one or more of medication efficacy, medication safety, medication resistance, medication cost, or patient-specific coverage factors. A device may include a presentation layer comprising a summary dashboard configured to display a ranked medication recommendation set produced by the analysis engine.

### CLAIM OF PRIORITY UNDER 35 U.S.C. § 119

The present Application is a continuation-in-part of U.S. application Ser. No. 17/526,244 filed Nov. 15, 2021, which claims priority to U.S. Provisional Application No. 63/113,809 filed Nov. 13, 2020 and U.S. Provisional Application No. 63/144,415, filed Feb. 1, 2021, all of which are hereby incorporated by reference herein in their entireties and for all purposes.

### FIELD OF THE DISCLOSURE

The present disclosure relates to systems, methods, computing platforms, and storage media for displaying and ranking customized candidate medications, enabling real-time communication between pharmacists and providers, and displaying a visualization of programming instructions for a medical device.

### BACKGROUND

Solutions and suspensions are difficult to comprehend and work with. Anyone who has taken first level chemistry lab has had to convert units to make solutions and suspensions. A first step in creating these solutions and suspensions is often to take a particular mass unit of a powdered chemical that is mixed into a liquid diluent. The amount (i.e., mass) of the active ingredient in the liquid determines the concentration of the solution or suspension. This ratio of mass unit can change in relation to the liquid. For example, 10 mg of calcium can be diluted in 1 mL, 10 mL or 100 mL of sterile water. This would make a 10 mg/mL, 1 mg/mL and 0.1 mg/mL concentration, respectively.

What also complicates things for those making a solution or suspension is that there are different ways to represent the same information. For example the 10 mg/mL solution can be described as a “1% Calcium Solution” or a “1:100 concentration.” There are also issues where different units of measure can be used, which can also create confusion. For example, Moles, Millimoles, mEq (milliequivalents), IU (international units), and grams are all units of measure that can be used interchangeably when mixing or dispensing various solutions/suspensions. This can create confusion, loss of reference, and lead to errors. As long as humans must mix a mass of any substance, e.g., medications, into a volume of liquid, this challenge/problem will be constant.

This task is further complicated by the various measuring containers on the market. For example, in chemistry there are various sized graduated cylinders and flasks, and in medicine, there are various sized and shaped syringes, which are used for both pulling and measuring substances, e.g., medication, as well as mixing all pulled substances together, which requires a certain volume of distribution (VD). An operator could make a mistake by choosing an incorrectly sized measuring device for a task and then set themselves up for a mistake translating the calculated number or volume they want to use or administer on to the syringe. For example, a user can misplace the decimal point on a syringe even if they have calculated the decimal point correctly on paper. Studies have showed with the mean error for such mistakes is substantial—in some studies, as high as 808%. In another example, since some containers, such as syringes, are not uniform in shape, additional mistakes can occur. Some medications are distributed into the blood volume of a person (5 liters for the average adult male) and require precise concentration; however, some medications displace into the tissue volume of a person which is significantly larger volume (42 liters for the average adult male). Therefore, medications with a low VD can't be treated in the same way as medications with a high VD.

Those who are working with solutions and suspension of chemicals are typically doing important work in their field, whether it be industry, academia or medical practice. A mistake within calculation could lead to injury, harm, loss or valuable resources or even loss of life. In some fields, such as medicine, there are high error rates for dosing liquid medicines up to 39% of the time (https://pediatrics.aappublications.org/content/pediatrics/141/3/e20174066.full.pdf).

There is a disconnect—a form of functional illiteracy—that makes it difficult to connect the various abstract tasks (e.g., sequence math conversions/measurements) required to convert values for a specific practical task (drawing a syringe of medicine for a specific sized patient, or mixing the appropriate amount of concrete for a specific job). It is nearly impossible to ensure that the people doing the practical jobs have the academic training to understand many of these tasks, and even when there is academic training (medical) the success rate is not 100% . . . . This functional illiteracy results in human error. This disconnect can happen for multiple reasons. In medicine there are thousands of medications, which can have different dosing instructions that are specific. It is challenging for a prescriber, pharmacist, nurse on any part of the healthcare team to retain this information and stay abreast with new and changing information. To further complicate prescribing and administering, general rules that can be applied to all medications or even groups of medication do not exist, since each medication has specific requirements. For example, some medications have a low volume of distribution in the body (e.g., 5 liters for the average adult male) while other drugs have large volume of distribution (tissue volume 42 liters for the average adult male), applying the same rules to a large VD medication to a Low VD medication would result in a nearly 10-fold overdose. Similarly, the total blood volume of a newborn infant can be as little as 85 mL or approximately 1.5% of the volume of an adult. Also, some medications require specific routes of administration for different use cases. For example, in the case of Epinephrine, the volume administered for an adult having an anaphylactic reaction is 0.3 mL intramuscularly, whereas the volume given for cardiac arrest is 10 mL intravenous. A mistake related to either of these volumes or routes, e.g., if they were to be mistakenly interchanged, the patient would be injured and likely die. Further, many medications are metabolized by the liver, or cleared by the kidneys at different rates, which must be factored in as well. For example, giving one medication 3 times a day may be appropriate for one medication; however, it may be lethally toxic for another. In other words, timing of medication is essential, but this information is different for depending on the medication. Healthcare providers cannot memorize or retain this type of information for every medication and use case, and typically cannot reference this information during an emergency where time is a limiting factor. Other case specific variables must be considered. In an example scenario, two patients may require the antibiotic cephalexin for a skin infection. However if one of the patients is concurrently taking the medication cimetidine, that patient will require 2-3 times the dose of the other patient. This is the case even if everything else is equal since cimetidine affects the metabolic rate of cephalexin, e.g., the cimetidine and cephalexin have a drug-drug interaction.

In summary, the specific nuances of proper medication selection and dosing depend on multiple variables, including drug interactions, patient characteristic, and the like, which are too numerous for healthcare providers to properly factor in, especially in emergency situations with time constraints. Unfortunately, the consequence of a miscalculation during any part of the drug administration process can be very serious, and can easily result in death to the patient. Moreover, if a member of the healthcare team makes a mistake during the process, that error can travel downstream and reach the patient with the undesired negative consequence of ineffectiveness, harm or even death. For example, once a medication is administered it cannot be taken back and it is too late to prevent or reduce harm. The medicine recommendation platform can assist the prescriber, pharmacist, nurse, and the like to work together in their various roles to increase the likelihood that the appropriate medication in the correct amount of medication is administered to the patient.

There is the need for a reliable way to confirm the correct amount of a solution or suspension to use for particular tasks across a variety of fields and industries.

### SUMMARY

In some aspects, the techniques described herein relate to a medicine recommendation platform including: a data acquisition layer including a patient information collector and a drug information collector, each operable to ingest patient-specific clinical data and drug-specific data, respectively; an analysis engine including a multi-criteria ranking engine configured to compute composite suitability score for candidate medications by weighting one or more of medication efficacy, medication safety, medication resistance, medication cost, or patient-specific coverage factors; and a presentation layer including a summary dashboard configured to display a ranked medication recommendation set produced by the analysis engine.

In some aspects, the techniques described herein relate to a medicine recommendation platform, further including: a real-time sensitivity and resistance data connector communicatively linked to the drug information collector and configured to supply continuously updated drug efficacy information; wherein the analysis engine further includes an efficacy and resistance evaluator configured to incorporate the drug efficacy information.

In some aspects, the techniques described herein relate to a medicine recommendation platform, wherein the analysis engine further includes: an interaction and contraindication checker configured to screen the candidate medications against one or more of patient allergies, comorbidities, or concomitant therapies.

In some aspects, the techniques described herein relate to a medicine recommendation platform, wherein the interaction and contraindication checker is configured to assign severity grades to identified risks and to annotate the ranked medication recommendation set with risk indicators.

In some aspects, the techniques described herein relate to a medicine recommendation platform, wherein the analysis engine further includes: a cost and coverage analyzer configured to retrieve patient-specific cost and formulary information.

In some aspects, the techniques described herein relate to a medicine recommendation platform, further including: a data-refresh subsystem including: a monitoring module configured to monitor real-time data feeds; and a recalculation trigger module configured to trigger score recalculation upon detecting parameter changes surpassing a configurable threshold.

In some aspects, the techniques described herein relate to a medicine recommendation platform, wherein the data acquisition layer further includes an EMR interface configured to retrieve structured patient data.

In some aspects, the techniques described herein relate to a medicine recommendation platform, wherein the analysis engine further includes a dose calculation engine configured to generate drug-specific organ-adjusted dose guidance based on organ function.

In some aspects, the techniques described herein relate to a medicine recommendation platform, further including: a pictogram generator and a packaging display module configured to render visual identifiers of at least one candidate medication in the ranked medication recommendation set.

In some aspects, the techniques described herein relate to a patient-specific medication recommendation method including: acquiring, via a data acquisition step, data including patient-specific clinical parameters and contemporaneous drug information; calculating patient-specific dosage ranges for at least one candidate medication; evaluating contraindications and interaction risks for each candidate medication relative to a patient profile; computing, with a multi-criteria ranking routine, a composite suitability score for each candidate medication that integrates one or more of medication efficacy, medication resistance, medication safety, medication cost, or patient-specific coverage factors; and presenting a ranked medication recommendation set on a summary dashboard.

In some aspects, the techniques described herein relate to a patient-specific medication recommendation method, further including: harmonizing the acquired data to generate a harmonized clinical and drug dataset.

In some aspects, the techniques described herein relate to a patient-specific medication recommendation method, wherein acquiring the patient-specific clinical parameters further includes extracting: patient insurance information.

In some aspects, the techniques described herein relate to a patient-specific medication recommendation method, wherein acquiring the patient-specific clinical parameters further includes extracting: laboratory results including one or more of serum creatinine, liver enzymes, or a blood test.

In some aspects, the techniques described herein relate to a patient-specific medication recommendation method, wherein calculating the patient-specific dosage ranges scale doses according to respective FDA-labels or manufacturer label prescribing information.

In some aspects, the techniques described herein relate to a patient-specific medication recommendation method, wherein computing the composite suitability score applies user-configurable weighting coefficients to one or more of the medication efficacy, medication resistance, medication safety, medication cost, or patient-specific coverage factors.

In some aspects, the techniques described herein relate to a patient-specific medication recommendation method, further including: monitoring real-time clinical or pharmaceutical data feeds and, in response to detecting a threshold change in a monitored parameter, automatically recomputing the composite suitability scores and updating the presented ranked medication recommendation set.

In some aspects, the techniques described herein relate to a patient-specific medication recommendation method, further including: generating a visual summary interface view that displays, for each candidate medication, an efficacy percentage, a recommended dose, contraindication alerts, and an estimated patient out-of-pocket cost or institutional cost.

In some aspects, the techniques described herein relate to a non-transitory computer-readable storage medium storing instructions that, when executed by one or more processors, cause a system to perform the method.

In some aspects, the techniques described herein relate to a non-transitory computer-readable storage medium, wherein the instructions further cause the system to monitor real-time data feeds.

In some aspects, the techniques described herein relate to a non-transitory computer-readable storage medium, wherein the instructions further cause the system to trigger automatic score recomputation in response to a susceptibility rate changing by at least a predefined percentage.

Accordingly, there is provided a medicine recommendation platform and method as detailed in the respective independent claims. A medicine recommendation platform and method per the independent claim is also provided. Advantageous features are provided in the dependent claims.

In some aspects, the techniques described herein relate to a pharmacy-provider communication platform including: a data management module that maintains patient data for concurrent access by pharmacist-side and provider-side users; a synchronization engine that propagates any modification to the patient data to all users in real time; and a user interface module having a pharmacist interface and a provider interface each configured to display one or more of a modification parameter or an acknowledgment control in response to receipt of a structured modification entry.

In some aspects, the techniques described herein relate to a pharmacy-provider communication platform, wherein the structured modification entry is a structured prescription modification entry, the platform further including: a notification engine that, responsive to receipt of the structured prescription modification entry, generates a prescription change notification package including one or more of pre-modification prescription parameters, post-modification prescription parameters, or a clinical rationale field.

In some aspects, the techniques described herein relate to a pharmacy-provider communication platform, wherein the notification engine includes: a change alert module configured to prioritize and throttle alerts based on predefined clinical criteria and acknowledgment status.

In some aspects, the techniques described herein relate to a pharmacy-provider communication platform, wherein the user interface module further includes: one or more of a shared journal interface or a messaging system configured to present to the pharmacist-side and provider-side users a message thread linked to the prescription change notification package, wherein the message thread include a summary including one or more of: who made the prescription change, what the prescription change was, where the prescription change was made, when the prescription change was made, or why the prescription change was made.

In some aspects, the techniques described herein relate to a pharmacy-provider communication platform, further including: a mobile application interface configured to employ adaptive data compression to deliver the prescription change notification package over variable cellular networks.

In some aspects, the techniques described herein relate to a pharmacy-provider communication platform, wherein the user interface module further includes: one or more of a shared journal interface or a messaging system configured to receive input from either the pharmacist-side or provider-side users and present the input to the other user in real time.

In some aspects, the techniques described herein relate to a pharmacy-provider communication platform, further including: an acknowledgment tracking module that captures an acknowledgment signal produced via the acknowledgment control and updates a status log linked to the structured modification entry.

In some aspects, the techniques described herein relate to a pharmacy-provider communication platform, further including: an audit and tracking module configured to record an auditable change and acknowledgment record for each prescription change event and an associated acknowledgment.

In some aspects, the techniques described herein relate to a pharmacy-provider communication platform, wherein the audit and tracking module is configured to produce a chronologically timestamped event record for each prescription change, acknowledgment, or message.

In some aspects, the techniques described herein relate to a pharmacy-provider communication platform, wherein the structured modification entry is a structured prescription modification entry, the platform further including: a Medication Recommendation Platform configured to analyze laboratory data contained in unified patient record data and to output a prescription adjustment recommendation pre-populated into the structured prescription modification entry.

In some aspects, the techniques described herein relate to a computer-implemented method for real-time change notification in a pharmacy-provider communication platform, including: initiating a prescription modification by generating, within a pharmacist interface or a provider interface, a structured prescription modification entry; generating, by a notification engine, a prescription change notification package from the structured prescription modification entry; delivering the prescription change notification package to the pharmacist interface or the provider interface; and presenting, via the pharmacist interface or the provider interface, a change-summary interface that displays modified prescription parameters associated with the structured prescription modification entry.

In some aspects, the techniques described herein relate to a method, further including: synchronizing patient data by retrieving an external clinical data payload and merging the external clinical data payload into a set of unified patient record data.

In some aspects, the techniques described herein relate to a method, wherein: the structured prescription modification entry includes a clinical rationale; and the change-summary interface further displays the clinical rationale.

In some aspects, the techniques described herein relate to a method, wherein presenting the change-summary interface includes: visually highlighting each modified prescription parameter using color coding and providing tooltips showing pre-modification and post-modification values.

In some aspects, the techniques described herein relate to a method, further including: providing, within the change-summary interface, direct access to at least one retrieved supporting record selected from laboratory results, medication histories, or clinical notes.

In some aspects, the techniques described herein relate to a method, further including: capturing an acknowledgment signal issued by the pharmacist interface or the provider interface via an acknowledgment control in response to receiving the prescription change notification package.

In some aspects, the techniques described herein relate to a method, wherein capturing the acknowledgment signal includes: detecting that the change-summary interface has remained open on a pharmacist or provider device for at least a threshold duration.

In some aspects, the techniques described herein relate to a method, further including: facilitating bi-directional clarification messaging by creating a clarification message thread linked to the prescription change notification package and logging follow-up query message or follow-up response journal entries in the clarification message thread.

In some aspects, the techniques described herein relate to a method, further including: timestamping each event related to the prescription change, the prescription change notification package, or an acknowledgment signal to produce a chronological timestamped event record.

In some aspects, the techniques described herein relate to a non-transitory computer-readable storage medium storing instructions that, when executed by one or more processors, cause a system to perform the method.

Accordingly, there is provided a pharmacy-provider communication platform and method as detailed in the respective independent claims. A pharmacy-provider communication platform and method per the independent claim is also provided. Advantageous features are provided in the dependent claims.

In some aspects, the techniques described herein relate to a programming instruction display platform for IV pumps, including: a processor configured to: receive input regarding pump specifications including at least one of make, model, brand, series, or type; receive patient information including at least one of patient-specific demographic information, patient-specific measurements, patient-specific disease information, or patient-specific prescription; calculate or retrieve a treatment plan based on the patient information and the pump specifications; and a display configured to provide step-by-step visual instructions for programming an IV pump corresponding to the pump specifications, wherein the visual instructions include images of a display screen of the IV pump at one or more programming steps.

In some aspects, the techniques described herein relate to a programming instruction display platform, wherein the processor is configured to receive the input regarding the pump specifications through a user selection interface that presents at least one of pump manufacturers, models, brands, or series information.

In some aspects, the techniques described herein relate to a programming instruction display platform, wherein the processor is configured to receive the input regarding the pump specifications through an image submission and identify the IV pump from at least one submitted photograph of the IV pump.

In some aspects, the techniques described herein relate to a programming instruction display platform, wherein the processor is further configured to analyze the at least one submitted photograph of the IV pump and extract identifying information including one or more of manufacturer logos, model numbers, or display configurations.

In some aspects, the techniques described herein relate to a programming instruction display platform, wherein: the patient-specific demographic information includes at least one of patient age, patient weight, patient height, patient gender, or patient ethnicity; the patient-specific measurements include at least one of patient body surface area, patient vital signs, patient values from laboratory test, or patient organ function parameters; the patient-specific disease information includes at least one of patient condition severity, treatment protocols, clinical guidelines, or contraindications; and the patient-specific prescription includes at least one of medication name, concentration, dosing requirements, administration routes, treatment duration, or physician-specified parameters.

In some aspects, the techniques described herein relate to a programming instruction display platform, wherein the processor is configured to determine an administration method based on the treatment plan, wherein the administration method includes liquid to be administered as a bolus dose or liquid to be metered out over time.

In some aspects, the techniques described herein relate to a programming instruction display platform, wherein the determination of the administration method is based on one or more of medication properties, patient factors, or clinical requirements.

In some aspects, the techniques described herein relate to a programming instruction display platform, wherein the display is further configured to show a final verification screen displaying a completed pump face display that matches the pump specifications for the IV pump in accordance with the treatment plan.

In some aspects, the techniques described herein relate to a programming instruction display platform, wherein the final verification screen enables comparison to the IV pump programmed by a provider before patient treatment begins.

In some aspects, the techniques described herein relate to a programming instruction display platform, wherein the verification screen displays appropriate drip rate, liquid to be dispensed, treatment duration, and programming parameters specific to the treatment plan and pump specifications.

In some aspects, the techniques described herein relate to a method for providing programming instructions for IV pumps, including: receiving input regarding pump specifications including at least one of make, model, brand, series, or type; receiving patient information including at least one of patient-specific demographic information, patient-specific measurements, patient-specific disease information, or patient-specific prescription; calculating or retrieving a treatment plan based on the patient information and the pump specifications; and displaying step-by-step visual instructions for programming an IV pump corresponding to the pump specifications, wherein the visual instructions include images of a display screen of the IV pump at one or more programming steps.

In some aspects, the techniques described herein relate to a method, wherein receiving input regarding pump specifications includes presenting a user selection interface with at least one of pump manufacturers, models, brands, or series information.

In some aspects, the techniques described herein relate to a method, wherein receiving input regarding pump specifications includes: receiving submitted photographs of the IV pump; analyzing the photographs; and determining appropriate pump specifications and programming requirements.

In some aspects, the techniques described herein relate to a method, wherein analyzing the photographs includes: extracting identifying information from the submitted photographs, wherein the identifying information includes one or more of manufacturer logos, model numbers, or display configurations; and comparing the submitted photographs against pump images in a database.

In some aspects, the techniques described herein relate to a method, further including: displaying a final verification screen showing a completed pump face display that matches the pump specifications for the IV pump in accordance with the treatment plan, wherein the final verification screen enables comparison to the IV pump programmed by a provider before patient treatment begins.

In some aspects, the techniques described herein relate to a universal IV pump programming system, including: an input interface configured to receive pump identification information for IV pumps from multiple different manufacturers; a patient data interface configured to receive patient-specific treatment parameters; a calculation engine configured to determine a treatment plan based on the pump identification information and the patient-specific treatment parameters; and a visual instruction generator configured to provide step-by-step programming guidance including simulated pump display images that correspond to the pump identification information.

In some aspects, the techniques described herein relate to an universal IV pump programming system, wherein the input interface is configured to receive pump identification information through at least one of a user selection interface that presents at least one of pump manufacturers, models, brands, or series information, or through image submission, wherein the input interface is configured to analyze submitted images of IV pumps.

In some aspects, the techniques described herein relate to an universal IV pump programming system, wherein the input interface is configured to compare the submitted images against pump images in a database and extract identifying information including one or more of manufacturer logos, model numbers, or display configurations.

In some aspects, the techniques described herein relate to an universal IV pump programming system, wherein the calculation engine is configured to determine an administration method based on the treatment plan, wherein the administration method includes liquid to be administered as a bolus dose or liquid to be metered out over time.

In some aspects, the techniques described herein relate to an universal IV pump programming system, wherein the visual instruction generator is further configured to provide a final verification screen displaying a completed pump face display that matches pump specifications for an IV pump in accordance with the treatment plan.

Accordingly, there is provided a medical device programming instruction display platform and method as detailed in the respective independent claims. A medical device programming instruction display platform and method per the independent claim is also provided. Advantageous features are provided in the dependent claims.

One aspect of the present disclosure relates to a system configured for automatically displaying a visualization of a desired volume of material. The system may include one or more hardware processors configured by machine-readable instructions. The processor(s) may be configured to receive a first measurement of a subject for which a desired material is to be applied. The processor(s) may be configured to receive a name of the desired material receive a desired concentration of the desired material. The processor(s) may be configured to receive a use case scenario for an application of the desired material to the subject. The processor(s) may be configured to calculate, based on the first measurement of the subject, the name of the desired material, the desired concentration, and the use case scenario, a correct volume of the desired material for the application. The processor(s) may be configured to retrieve, from a database, at least one image associated with the correct volume of the desired material for the application. The processor(s) may be configured to display, on an interface, the at least one image associated with the correct volume of the desired material.

Another aspect of the present disclosure relates to a method for automatically displaying a visualization of a desired volume of material. The method may include receiving a first measurement of a subject for which a desired material is to be applied. The method may include receiving a name of the desired material receive a desired concentration of the desired material. The method may include receiving a use case scenario for an application of the desired material to the subject. The method may include calculating, based on the first measurement of the subject, the name of the desired material, the desired concentration, and the use case scenario, a correct volume of the desired material for the application. The method may include retrieving, from a database, at least one image associated with the correct volume of the desired material for the application. The method may include displaying, on an interface, the at least one image associated with the correct volume of the desired material.

Yet another aspect of the present disclosure relates to a computing platform configured for automatically displaying a visualization of a desired volume of material. The computing platform may include a non-transient computer-readable storage medium having executable instructions embodied thereon. The computing platform may include one or more hardware processors configured to execute the instructions. The processor(s) may execute the instructions to receive a first measurement of a subject for which a desired material is to be applied. The processor(s) may execute the instructions to receive a name of the desired material receive a desired concentration of the desired material. The processor(s) may execute the instructions to receive a use case scenario for an application of the desired material to the subject. The processor(s) may execute the instructions to calculate, based on the first measurement of the subject, the name of the desired material, the desired concentration, and the use case scenario, a correct volume of the desired material for the application. The processor(s) may execute the instructions to retrieve, from a database, at least one image associated with the correct volume of the desired material for the application. The processor(s) may execute the instructions to display, on an interface, the at least one image associated with the correct volume of the desired material.

Still another aspect of the present disclosure relates to a system configured for automatically displaying a visualization of a desired volume of material. The system may include means for receiving a first measurement of a subject for which a desired material is to be applied. The system may include means for receiving a name of the desired material receive a desired concentration of the desired material. The system may include means for receiving a use case scenario for an application of the desired material to the subject. The system may include means for calculating, based on the first measurement of the subject, the name of the desired material, the desired concentration, and the use case scenario, a correct volume of the desired material for the application. The system may include means for retrieving, from a database, at least one image associated with the correct volume of the desired material for the application. The system may include means for displaying, on an interface, the at least one image associated with the correct volume of the desired material.

Even another aspect of the present disclosure relates to a non-transient computer-readable storage medium having instructions embodied thereon, the instructions being executable by one or more processors to perform a method for automatically displaying a visualization of a desired volume of material. The method may include receiving a first measurement of a subject for which a desired material is to be applied. The method may include receiving a name of the desired material receive a desired concentration of the desired material. The method may include receiving a use case scenario for an application of the desired material to the subject. The method may include calculating, based on the first measurement of the subject, the name of the desired material, the desired concentration, and the use case scenario, a correct volume of the desired material for the application. The method may include retrieving, from a database, at least one image associated with the correct volume of the desired material for the application. The method may include displaying, on an interface, the at least one image associated with the correct volume of the desired material.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1 illustrates a system configured for automatically displaying a visualization of a desired volume of material, in accordance with one or more implementations.

FIG. 2 illustrates a method for automatically displaying a visualization of a desired volume of material, in accordance with one or more implementations.

FIGS. 3A, 3B, 3C, and 3D show exemplary interfaces of an application that guides a medical provider through a process of inputting variables related to medication dosing and shows a visual display of a correct volume of medication, in accordance with one or more implementations.

FIGS. 4A and 4B show another set of exemplary interfaces of an application that guide a medical provider through a process of inputting variables related to medication dosing and show a visual display of a correct volume of medication, in accordance with one or more implementations.

FIGS. 5A and 5B show exemplary images that may be displayed to indicate a particular volume and type of medication to be given to a patient, in accordance with one or more implementations.

FIGS. 6-11 depict process-flow diagrams associated with aspects of the present disclosure.

FIG. 12 illustrates a block diagram depicting an exemplary machine that includes a computer system within which a set of instructions can execute for causing a device to perform or execute any one or more of the aspects and/or methodologies of the present disclosure, in accordance with one or more implementations.

FIGS. 13A and 13B illustrate examples of a computing device comprising an application for receiving patient measurements, according to various aspects of the disclosure.

FIG. 14 illustrates an example of a computing device displaying a particular volume and type of medication to be taken by a patient, according to various aspects of the disclosure.

FIG. 15 illustrates a system configured for recommending a solution for a specific patient, in accordance with one or more implementations.

FIGS. 16-17 illustrate sub-systems of the system configured for automatically recommending a solution for a specific patient, in accordance with one or more implementations.

FIG. 18 illustrates a method for recommending a solution for a specific patient, in accordance with one or more implementations.

FIGS. 19-22 illustrate sub-method steps of the method for recommending a solution for a specific patient, in accordance with one or more implementations.

FIG. 23 illustrates a system configured for enabling communication across a healthcare team, in accordance with one or more implementations.

FIG. 24 illustrates a method for enabling communication across a healthcare team, in accordance with one or more implementations.

FIGS. 25-41B illustrate examples of graphical user interfaces, in accordance with one or more implementations.

### DETAILED DESCRIPTION

Disclosed herein is a system to confirm the correct amount of a solution or suspension to use for particular tasks across a variety of fields and industries. This system can match the desired concentration of substance (or medication) in the volume of distribution (blood volume or tissue volume in a patient) for a specific required period of time, factoring in all of the specific variables for each component, such as medication (route, metabolism, volume of distribution type, frequency of dosing, and the like) and patient characteristics. Additionally, the system described herein can factor in overall cost and/or particular patient costs associated with different medications. The increasing rising cost of healthcare is a barrier for many patients to access the medication prescribed my a health care professional. For example, a provider may calculate a solution that is adequate and safe for the patient, which is the first priority but many times the only priority for providers given the structure of the healthcare system (e.g., which can reduce or eliminate time available for providers to consider one or more alternative medications or solutions). However, since providers do not consider, have access to, or know how to factor in financial factors for the prescribed medication, a medication that is safe and adequate for a patient can be unaffordable for the patient or the medication may not be covered by the insurance, and thus the patient may be unable to fill the prescription and thus never receive treatment or the prescribed medication. The system described herein can factor in these factors as well as use cases to provide a specific treatment (e.g., an antibiotic) with a specific chemistry/pharmacology (e.g., kidney cleared 3 times per day) for a specific patient (e.g., a newborn child) for a specific medical condition (e.g., pneumonia) at the specific cost requirements (e.g., insured by Medicaid). In this way, the system can reduce errors during the prescription process and assist a provider in prescribing an effective, yet affordable medication for a patient.

Disclosed herein is a system to enable consistent and repeatable interaction with mass and volume units. Once such system provides a confirmation check system, enabling a user to confirm the user is measuring an accurate volume of material and, in fact, doing their job correctly. Since errors in some industries are seen as “never events” (i.e., events that should never happen because such an error would be catastrophic), such a confirmation check system may prevent or reduce such errors. It has been found that system-based interventions like simplification and standardizations are more effective than education at reducing human error.

The present disclosure addresses the need for a high-reliability tool that can be used in industries where measuring volumes of solutions and suspensions performed frequently by many people and where a mistake can have serious consequences. The present disclosure provides a system that is easy to use in association with various volume measuring devices that are commonly available in the market. The system disclosed herein helps users visualize and have a frame of reference for the volume being measured and how to apply the measured volume of material to their specific tasks. The system further provides relevant information a user may need for the to complete an application of the desired material to a subject. It is contemplated that a combination of the first measurement of a subject, a desired material identifier (e.g., a name of the desired material), the concentration of the desired material, and the use case scenario comprises one such application. It is further contemplated that a use case scenario may identify an application (and/or a reason) for associating the desired material with the subject. In one such association, the subject may receive the desired material. In another such association, the subject may ingest the desired material. Providing a visual to users as a frame of reference may enable users to maintain an understanding of the measurement scales involved, potentially reducing mistakes and improving safety. For example, mcg and mg are two very similar-looking units of measure that differ by several orders of magnitude. Many users are not experts in dealing with or understanding such differing scales, allowing many measurement-related mistakes to occur. In a medical setting these measurement-related mistakes can be particularly dangerous and potentially fatal to patients. The present disclosure may comprise one or more key components of a safety system to help users avoid such measurement-related mistakes.

The systems, methods, and apparatuses of the present disclosure have the flexibility and ease of use to take a very technical and complex thought process and simplify the work based on the desired task. They also allow the user to input the specific variables of their current task so that there is no “trial and error,” as elimination of errors is the goal, and “trial and error,” by definition, makes allowance for errors.

In embodiments, a user may use the system of the present disclosure to mix the appropriate amount of cement for a specific job (e.g., paving) associated with a subject (e.g., a driveway). In embodiments, the system may be implemented as a software application on a smartphone. The user could input a first measurement of the subject. Here, a first measurement may comprise more than one value like the length and the width, or the driveway. The user may input the material to be used or mixed into the software application, and input the job type (i.e., “use case,”), which, in this example, may comprise “driveway cement”. The software application may calculate the correct volume of cement that would be needed to pave a driveway of that particular size. It may then guide the user through a process step-by-step how much cement (kg), water (liters), and hardening agent (oz) they should mix for that task, including the size of the container it should be mixed in (25 gallon) and the type of mixing (electric stirring) and length of time mixing should occur. Several of these measurements and variables (in particular, the solution concentrations and resulting volumes) are items that an inexperienced person may not know. For example, a person may not know how much space all the ingredients, when mixed together, would take up. Such a lack of knowledge leads to the possibility of trial and error instead of a perfect result on the first try.

The system of the present disclosure may also automatically account for the fact that both metric and imperial units are used as measures of various components of the solution. Often times, one manufacturer of a particular component will only list instructions in metric units, while another essential component is made by another manufacturer using imperial units. The present system may automatically convert different units and systems of measurement. The ability to convert unlike units, such as, but not limited to, imperial and metric units, into like units may enable the prevention of costly mistakes, save time, and save money by potentially eliminating the need for additional equipment associated with multiple systems of measurement.

In the cement-related example, the system of the present disclosure may display a visualization of the volume of the total amount of cement to be used for the job having a driveway of a particular size. For example, it may show that eight 25-gallon containers should be used to complete the job. The display may be a picture of eight buckets on a screen of a smartphone. In embodiments, text labels, such as the words “25-gallon” may accompany the actual images of the buckets. Such a visualization may reduce waste of materials.

Other embodiments contemplate the administrations of medicine. The use of medication in patients may require the use of several variables related to patient size and drug solution concentration. Variables related to other items may also be included in the administration of medicine. Additionally, the term: variable” may comprise at least one measurement. Patients are various sizes (e.g., small adult (55 kg), child (12 kg), obese adult (140 kg) and certain medications require a specific ratio of milligrams of medicine per each kilogram of body weight, while taking into account the medicant concentrations (10 mg/mL, 50 mg/mL, or 100 mg/mL). When a particular medical condition requires multiple medications or when there are more than one patient who needs to be treated at one time, the amount of variables, medications, patients, and factors can result in a higher likelihood of human error and could cause harm or death.

The embodiments of the present disclosure may aid users in selecting the appropriate tools for a given task. For example, in medical applications of the present invention, a particular syringe size or volume may be suggested to users based on a calculation involving a medication dose, concentration, and volume. Providing aid to users in tool selection may enable a potential reduction in errors and time wasted and may increase success rates, such as by avoiding the use of an improper syringe size that may lead to an error in dosage measurement.

The presently disclosed systems, methods, and apparatuses for displaying appropriate volumes can assist nurses, doctors, pharmacists, and other medical providers in their practice environments, and are highly advantageous, both for the speed of selecting the correct volume, but also to ensure accuracy.

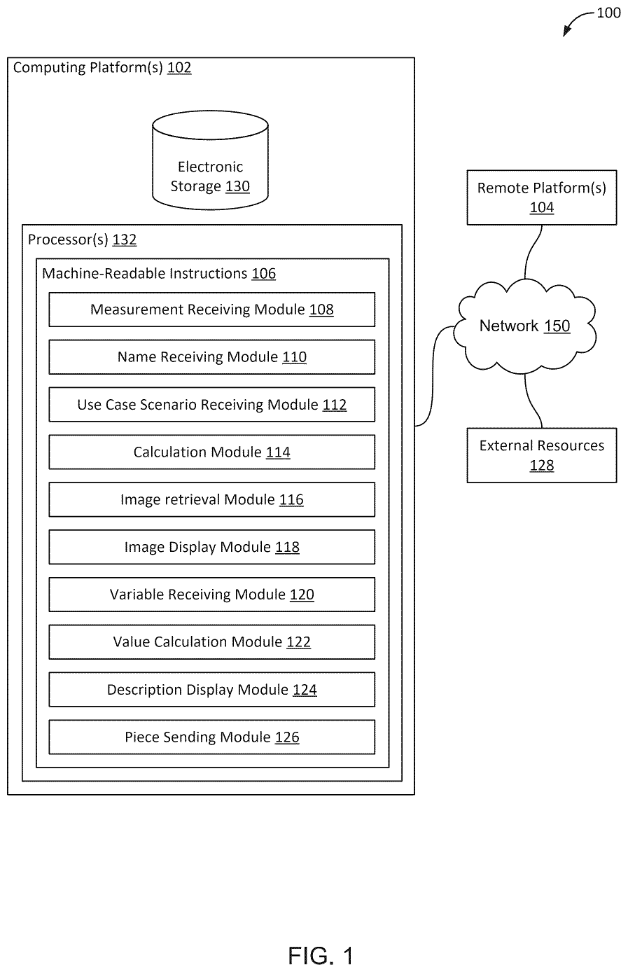

FIG. 1 illustrates a system 100 configured for automatically displaying a visualization of a desired volume of material, in accordance with one or more implementations. In some implementations, system 100 may include one or more servers 102. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Users may access system 100 via client computing platform(s) 104.

Server(s) 102 may be configured by machine-readable instructions 106. Machine-readable instructions 106 may include one or more instruction modules. The instruction modules may include computer program modules. The instruction modules may include one or more of measurement receiving module 108, name receiving module 110, use case scenario receiving module 112, calculation module 114, image retrieval module 116, image display module 118, variable receiving module 120, value calculation module 122, description display module 124, piece sending module 126, and/or other instruction modules.

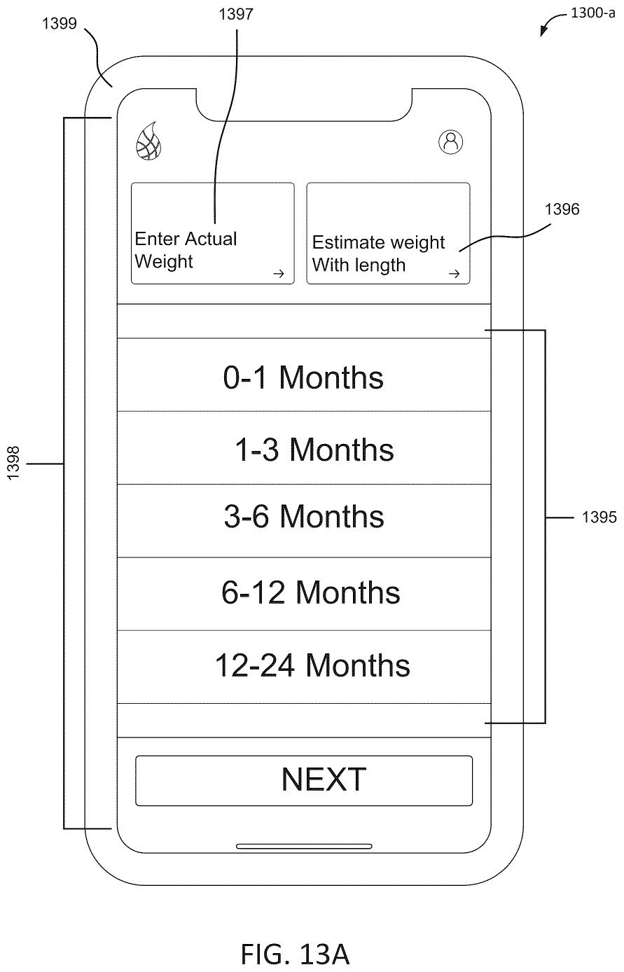

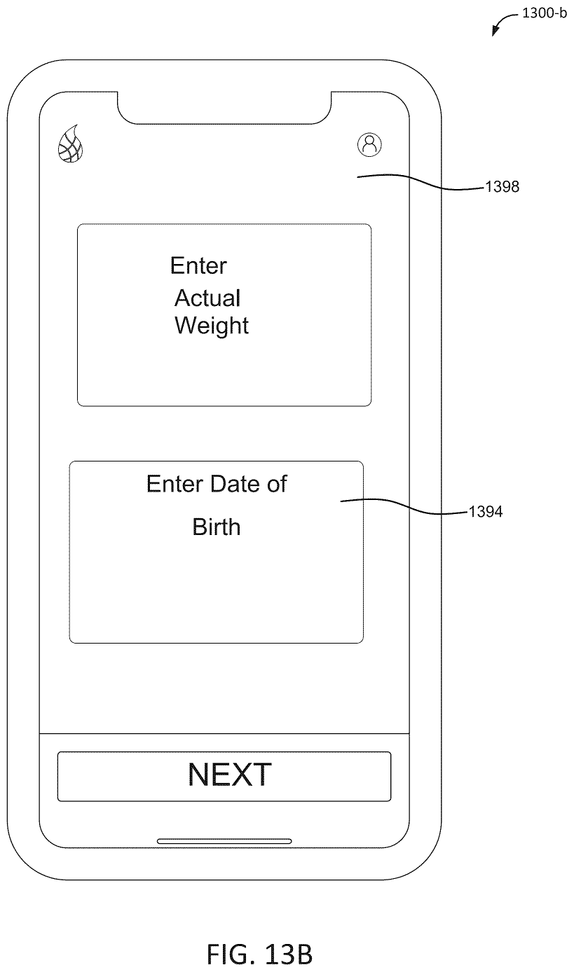

Measurement receiving module 108 may be configured to receive a first measurement of a subject. The subject may comprise any item that can be measured in any way. For example, the driveway in the earlier cement example may comprise a subject. A human medical patient may be another non-limiting example of a subject. Subjects may be associated with a desired material. For example, the driveway subject is associated with the desired material comprising cement in the earlier example and a human patient may be associated with a desired material comprising medicine in a medical example. As seen, the desired material may comprise a desired amount of a material for application to, or association with, the identified subject. Desired materials may comprise a liquid or solid medication, a construction material, a chemical solution, a cooking material, or any other type of material that may be associated with a concentration and a volume. The first measurement of the subject may be a measurement of a patient. However, the term “subject” may refer to any entity (animal, molecular) or item (device, system, area, etc.) associated with the receipt, or application, of the desired material. The measurement of a patient may be a measurement of one of patient length (e.g., inches, centimeters), a patient weight (e.g., pounds, kilograms), and/or patient surface area (e.g., square feet, square meters). Other measurements known in the art are contemplated. The desired material may comprise a medication such as, but not limited to, a liquid oral solution, a liquid injectable solution, a tablet, a pill, or a capsule. Seen in FIG. 13A is one example of a mobile computing device 1399 comprising a software application 1398 which enables a user (e.g. a healthcare provider) to enter a subject (e.g., patient) weight 1397 or a weight estimate 1396. The application 1398 further enables a user to identify the age 1395 of the subject. Alternatively, and as seen in FIG. 13B, the application 1398 may request a date of birth 1394 and the application 1398 may determine the age of the subject.

Measurement receiving module 108 may be configured to receive one or more additional measurements besides the first measurement(s) of a subject. For example, if a first measurement comprise a measurement of a patient's weight, another measurement may comprise the patient's length, height, or surface area. If a first measurement is a length of a physical space, another measurement may be its width, height, or weight.

It is contemplated that the system 100 may comprise an error notification feature. Such a feature may alert a user that a subject (e.g., a patient) should not receive the identified desired material (e.g., a medication). For example, if a child patient is identified as the subject and the desired material is identified as an adult medication, the system may notify the user the medication should not be provided to the patient. In one such embodiment, the information associated with the desired material and subject may be compared to a database to ensure accuracy. For example, a database such as, but not limited to a database associated with eh National Library of Medicine, may be referenced to determine whether an identified medication amount is associated with a patient age and weight. Such a database may comprise a locally-stored database on the device providing the interface or the database may comprise a cloud-based database. Portions of the database may be cloud-based and/or locally-stored. Such alerts and notifications may comprise an audible alarm, a visual notice, and/or may require additional steps to display the correct volume. For example, one such additional step may require an additional device associated with an additional user and/or user account, to approve or otherwise authorize the medication for the identified subject.

Measurement receiving module 108 may receive the first measurement of the subject through a measurement device. Once such measurement device may comprise a hardware measurement device. A hardware measurement device may comprise a camera, laser, scale, accelerometer, or other device, and may be internal or external to the system of the present disclosure. It is contemplated that a hardware measurement device may comprise software and/or firmware. For example, an image of a patient may be taken with a hardware measurement device comprising a camera-enabled mobile computing device (i.e., a smartphone camera), with the image uploaded to the system 100 and the system providing the patient's height upon analyzing the image.

Measurement receiving module 108 may be configured to receive one or more measurements from a device or database that stores subject information, such as subject measurements. For example, an electronic medical record of a patient may be used to provide the measurement receiving module 108 with at least one of a patient length, weight, and surface area. Other modules may also receive information from a device or database that stores subject information. Alternatively, a user may enter the measurement information manually.

Name receiving module 110 may be configured to receive a name of the desired material. It is contemplated that the name of the desired material may comprise an identifier for the material. One such identifier may be an abbreviation associated with the material or may comprise the material name. Such an identifier and/or name may be manually provided. It is further contemplated that the name receiving module 110 may be configured to receive a concentration (e.g., mg/ml) of the desired material. Such a concentration may be referred to herein as a “desired concentration.” In one such example, a user may manually input the desired material name and the concentration of the desired material into the application. It is also contemplated that the computing platform may include optical scanning equipment (e.g., a camera and software) to scan encoded information (e.g., a barcode and/or quick response (QR) code) to obtain the desired information. For example, upon scanning a QR code of a medicine, the application may automatically receive the name of the medicine/desired material and the concentration of the desired material/medicine from a communicatively coupled database.

In another example, machine learning and/or artificial intelligence techniques may be used to interpret an image of a label on a desired material container, and provide information from the label to the name receiving module 110. For example, an imaging device such as, but not limited, to, a camera and software may capture an image and utilize machine learning and/or artificial intelligence techniques to obtain the name, concentration, volume, etc. of the desired material within the container, Such information from the label of the desired material container may also be used in other modules, such as, but not limited to, the calculation module 114. Those of ordinary skill in the art in artificial neural network (ANN) technology will readily appreciate that an ANN model may be trained to interpret the label on a bottle of medication and take information (such as mg/ml, volume, etc.) and incorporate that information into the decision logic described herein.

Use case scenario receiving module 112 may be configured to receive a use case scenario. The use case scenario may be related to the application of the desired material to the subject. For example, the use case scenario may comprise one of a disease state and a treatment protocol. Disease states may also be referred to as “medical conditions” in the present disclosure and disease states and treatment protocols may be referred to together as a “medical use”.

Calculation module 114 (which may be referred to herein as name calculation module) may be configured to calculate, based on (a) the first measurement of the subject, (b) the name of the desired material, (c) the concentration, and (d) the use case scenario, a correct volume (i.e., the proper dose or proper amount of a solution or mixture) of the desired material for the application.

The image retrieval module 116 may be configured to retrieve, from a database, at least one image associated with the correct volume of the desired material for the application. Such an image may comprise a pictogram. The database may be internal or external to the system of the present disclosure. In embodiments, the database may be a library of images that are publicly available, or alternatively, created specifically for the system of the present disclosure. In some implementations, by way of non-limiting example, the at least one image may be of one of a syringe which displays the correct volume of the desired material. Alternatively, the image may display a tablet or a pill having the correct volume of the desired material. The correct volume of tablets, pills, and/or capsules may comprise portions (e.g., half) of such items. In some implementations, the desired material in the at least one image may comprise a color and the color may comprise a first color with the first color comprising a different color as compared to the remainder of the image.

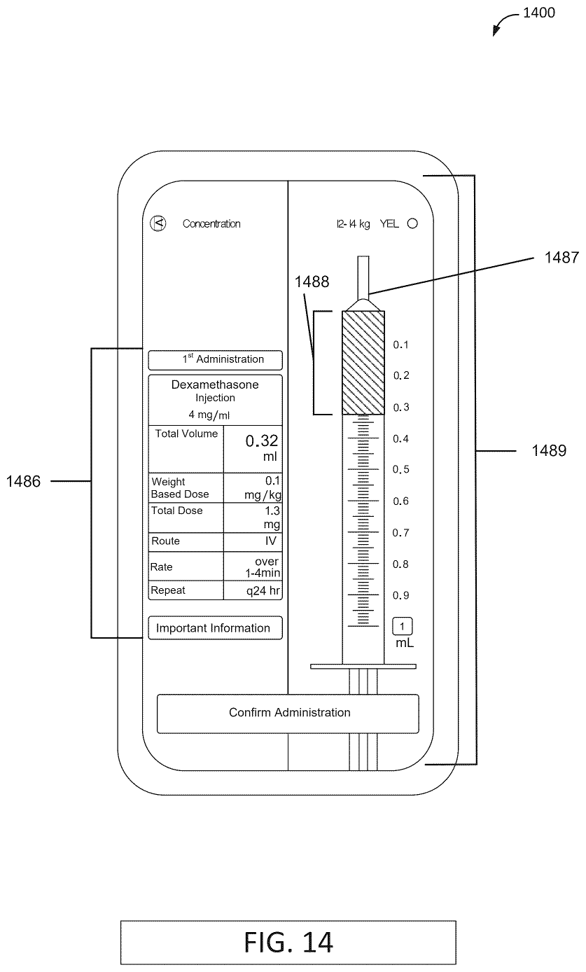

Image display module 118 may be configured to display, on an interface, the at least one image associated with the correct volume of the desired material. A size of the at least one image displayed on the interface may be an actual size of the correct volume. Seen in FIG. 14 is one example of an interface 1400 comprising a mobile computing device/smartphone screen 1489 displaying the at least one image. The at least one image in FIG. 14 comprises the correct volume 1488 of desired material. The desired material in the FIG. 14 example comprises a liquid medicine, or drug, located within a syringe 1487 displayed on the screen. The size of the syringe 1487 and the correct volume 1488 displayed on the screen 1489 may comprise an actual size, enabling a provider to place the actual syringe, used by the provider to give the drug to a patient, next to the syringe 1487 on the screen 1489 to verify the size of the syringe the provider is using is the correct size of syringe and to also verity that the correct volume is the same size as the volume 1488 displayed on the screen 1489. It is contemplated that the correct volume 1488 may comprise a pictogram having feature such as, but not limited to, an identifiable color or pattern (dotted area, slashed/slanted lines, etc.), associated with a subject's age, size, or other subject characteristic. One such pictogram, for example, may comprise a correct volume 1488 having the color pink where pink is associated with a patient having an age less than 3 months or having a weight less than 15 pounds. Characteristics known in the art other than age and size are contemplated. Subject characteristics may comprise any feature associated with a measurement.

The interface 1400 in FIG. 14 further displays a written description 1486 of the correct volume 1488 adjacent to the at least one image of the correct volume 1488 within the syringe 1487. In the FIG. 14 example, the written description 1486 comprises the administration number (i.e., this may comprise the “1^{st} administration” for a particular day or other time period, total administrations of a particular drug, etc.), the drug identifier/name and concentration, current volume, a weight-based dose, a total dose, an administration route for the current volume 1487, a rate of dosage (here, the 3.2 ml will be provided over 1-4 minutes), and a repeat rate, shown as q24 in this example. The above list of items in the written description identify the items shown in FIG. 14 from the top to the bottom of the written description, respectively. In the FIG. 14 display, the correct volume 1488 may comprise an identifiable color. One such identifiable color may comprise a color different from the other colors in the display. Additionally, the color of the correct volume may comprise a color associated with the size of the syringe/pipette and/or a size of the correct volume. For example, a correct volume under 1 ml may comprise a pink color, a correct volume from 1-10 ml may comprise a blue volume, etc.

Variable receiving module 120 may be configured to receive one or more variables related to the subject. Variables may include any number of factors impacting the use case, such as weather, temperature, atmospheric pressure, or physical or chemical requirements associated with a solution or suspension. They may include, in the case of medications, information associated with the administration of the medication and any medical condition associated with the subject. Calculating the correct volume of the desired material may be further based on the one or more variables.

Value calculation module 122 may be configured to calculate one or more estimated values for the subject based on one or more formulas. At least one of the one or more formulas may be used to estimate an ideal body weight of the patient. The calculating of the correct volume may be further based on the one or more estimated values.

Description display module 124 may be configured to display a written description of the correct volume adjacent to the at least one image. The written description of the correct volume may be shown in a plurality of formats.

Piece sending module 126 may be configured to send one or more pieces of information associated with the modules (108, 11, 112, 114, 116, 118, 120, 122, and 124) to an electronic medical records system. For example, the subject measurements from the measurement receiving module and the displayed image from the image display module may be sent to an electronic medical records system.

In some implementations, server(s) 102, client computing platform(s) 104, and/or external resources 128 may be operatively linked via one or more electronic communication links. For example, such electronic communication links may be established, at least in part, via a network such as the Internet and/or other computing networks. It will be appreciated that this is not intended to be limiting, and that the scope of this disclosure includes implementations in which server(s) 102, client computing platform(s) 104, and/or external resources 128 may be operatively linked via some other communication media.

A given client computing platform 104 may include one or more processors configured to execute computer program modules. The computer program modules may be configured to enable a user associated with the given client computing platform 104 to interface with system 100 and/or computing platforms 102 and external resources 128, and/or provide other functionality attributed herein to client computing platform(s) 104. By way of non-limiting example, the given client computing platform 104 may include one or more of a desktop computer, a laptop computer, a handheld computer, a tablet computing platform, a NetBook, a Smartphone, a gaming console, and/or other computing platforms.

External resources 128 may include sources of information outside of system 100, external entities participating with system 100, and/or other resources. In some implementations, some or all of the functionality attributed herein to external resources 128 may be provided by resources included in system 100, and vice versa.

Server(s) 102 may include electronic storage 130, one or more processors 132, and/or other components. Server(s) 102 may include communication lines, or ports to enable the exchange of information with a network and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 130 may comprise non-transitory storage media that electronically stores information. The electronic storage media of electronic storage 130 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 130 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 130 may include one or more virtual storage resources (e.g., cloud storage, a virtual private network, and/or other virtual storage resources). Electronic storage 130 may store software algorithms, information determined by processor(s) 132, information received from server(s) 102, information received from client computing platform(s) 104, and/or other information that enables server(s) 102 to function as described herein.

Processor(s) 132 may be configured to provide information processing capabilities in server(s) 102. As such, processor(s) 132 may include one or more of a digital processor, an analog processor, a digital circuit designed to process information, an analog circuit designed to process information, a state machine, and/or other mechanisms for electronically processing information. Although processor(s) 132 is shown in FIG. 1 as a single entity, this is for illustrative purposes only. In some implementations, processor(s) 132 may include a plurality of processing units. These processing units may be physically located within the same device, or processor(s) 132 may represent processing functionality of a plurality of devices operating in coordination. Processor(s) 132 may be configured to execute modules 108, 110, 112, 114, 116, 118, 120, 122, 124, and/or 126, and/or other modules. Processor(s) 132 may be configured to execute modules 108, 110, 112, 114, 116, 118, 120, 122, 124, and/or 126, and/or other modules by software; hardware; firmware; some combination of software, hardware, and/or firmware; and/or other mechanisms for configuring processing capabilities on processor(s) 132. As used herein, the term “module” may refer to any component or set of components that perform the functionality attributed to the module. This may include one or more physical processors during execution of processor readable instructions, the processor readable instructions, circuitry, hardware, storage media, or any other components.

It should be appreciated that although modules 108, 110, 112, 114, 116, 118, 120, 122, 124, and/or 126 are illustrated in FIG. 1 as being implemented within a single processing unit, in implementations in which processor(s) 132 includes multiple processing units, one or more of modules 108, 110, 112, 114, 116, 118, 120, 122, 124, and/or 126 may be implemented remotely from the other modules. The description of the functionality provided by the different modules 108, 110, 112, 114, 116, 118, 120, 122, 124, and/or 126 described herein is for illustrative purposes, and is not intended to be limiting, as any of modules 108, 110, 112, 114, 116, 118, 120, 122, 124, and/or 126 may provide more or less functionality than is described. For example, one or more of modules 108, 110, 112, 114, 116, 118, 120, 122, 124, and/or 126 may be eliminated, and some or all of its functionality may be provided by other ones of modules 108, 110, 112, 114, 116, 118, 120, 122, 124, and/or 126. As another example, processor(s) 132 may be configured to execute one or more additional modules that may perform some or all of the functionality attributed below to one of modules 108, 110, 112, 114, 116, 118, 120, 122, 124, and/or 126.

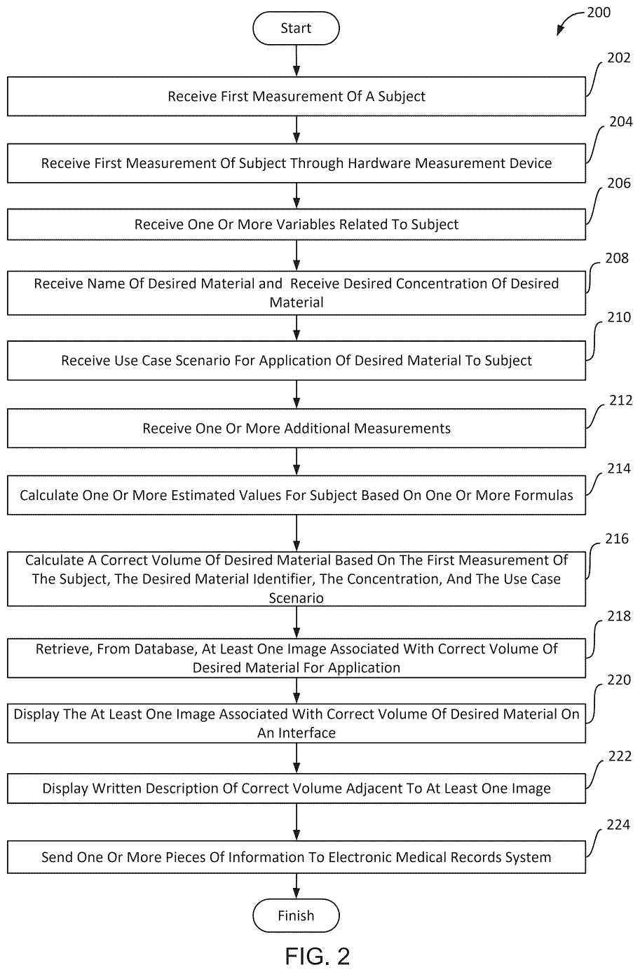

FIG. 2 illustrates a method 200 for automatically displaying a visualization of a desired volume of material, in accordance with one or more implementations. The operations of method 200 presented below are intended to be illustrative. In some implementations, method 200 may be accomplished with one or more additional operations not described, and/or without one or more of the operations discussed. In particular, a subset of the method 200 operations may be executed in some implementations. Additionally, the order in which the operations of method 200 are illustrated in FIG. 2 and described below is not intended to be limiting.

In some implementations, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

Operation 202 of method 200 may include receiving a first measurement of a subject. As disclosed here, one such subject is associated with an application (i.e. receiving) of a desired material. Operation 202 may be performed by one or more hardware processors configured by machine-readable instructions, including a module that is the same as or similar to measurement receiving module 108, in accordance with one or more implementations.

An operation 204 may comprise receiving the first measurement of the subject through a hardware measurement device. Operation 204 may also be performed by one or more hardware processors configured by machine-readable instructions, including a module that is the same as or similar to measurement receiving module 108, in accordance with one or more implementations.

Operation 206 may comprise receiving one or more variables related to the subject. The calculating of a correct volume of the desired material may be further based on the one or more variables. Operation 206 may be performed by one or more hardware processors configured by machine-readable instructions, including a module that is the same as or similar to variable receiving module 120, in accordance with one or more implementations.

An operation 208 may comprise receiving an identifier associated with the desired material and further receiving a concentration of the desired material. Operation 208 may be performed by one or more hardware processors configured by machine-readable instructions including a module that is the same as, or similar to, name receiving module 110, in accordance with one or more implementations.

An operation 210 may comprise receiving a use case scenario. One such use case scenario comprises a reason the desired material will be provided to the subject. Operation 210 may be performed by one or more hardware processors configured by machine-readable instructions including a module that is the same as or similar to use case scenario receiving module 112, in accordance with one or more implementations.

An operation 212 may include receiving one or more additional measurements. Operation 212 may be performed by one or more hardware processors configured by machine-readable instructions including a module that is the same as or similar to measurement receiving module 108, in accordance with one or more implementations.

An operation 214 may include calculating one or more estimated values for the subject based on one or more formulas. The calculating of the correct volume may be further based on the one or more estimated values. Operation 214 may be performed by one or more hardware processors configured by machine-readable instructions including a module that is the same as or similar to value calculation module 122, in accordance with one or more implementations.

An operation 216 may comprise calculating a correct volume of the desired material, where such calculation is based on (a) the first measurement of the subject, (b) the desired material identifier, (c) the concentration of the desired material, and (d) the use case scenario. Operation 216 may be performed by one or more hardware processors configured by machine-readable instructions including a module that is the same as or similar to Calculation module 114, in accordance with one or more implementations.

An operation 218 may include retrieving, from a database, at least one image associated with the correct volume of the desired material. It is contemplated that the correct volume of the desired material displayed in the image is for the specific application associated with measurements, the subject, use case scenario, and any other variables or information. Operation 218 may be performed by one or more hardware processors configured by machine-readable instructions including a module that is the same as or similar to image retrieval module 116, in accordance with one or more implementations.

An operation 220 may include displaying, on an interface, the at least one image associated with the correct volume of the desired material. Operation 220 may be performed by one or more hardware processors configured by machine-readable instructions including a module that is the same as or similar to image display module 118, in accordance with one or more implementations.

An operation 222 may include displaying a written description of the correct volume adjacent to the at least one image on the interface. One such interface may comprise a smartphone display screen. Other user interfaces known in the art are also contemplated. Operation 222 may be performed by one or more hardware processors configured by machine-readable instructions including a module that is the same as or similar to description display module 124, in accordance with one or more implementations.

An operation 224 may include sending one or more pieces of information to an electronic medical records system. Operation 224 may be performed by one or more hardware processors configured by machine-readable instructions including a module that is the same as or similar to piece sending module 126, in accordance with one or more implementations. In such an embodiment, the mobile computing device screens (e.g., FIGS. 13a-14, etc.) may be integrated in such a medical records system. For example, these screen may be displayed by the system prior to the provider giving the dose to a patient for verification of dosage. Furthermore, although the figures shown herein such, but not limited to FIG. 14, display the correct volume 1487 associated with a syringe, it is contemplated that other dosing mechanisms may be provided. For example, an oral dosing mechanism such a, but not limited, to a cup, may be utilized. Cups in such a system may be color-coded to identify the correct volume. It is further contemplated that the pipette/syringe may be color coded to identify the correct dosing amount. Pipette/syringe sizes may range from 1 ml with a .25 ml dosing size, 3 ml with a. 1 ml dosing size, and 5 ml with a 2 dosing size. Other sizes known in the art are also contemplated. The timing of such doses may vary as well—e.g., 1×/day, 2×/day, and 4×, and the dose size may vary across one or more doses/day.

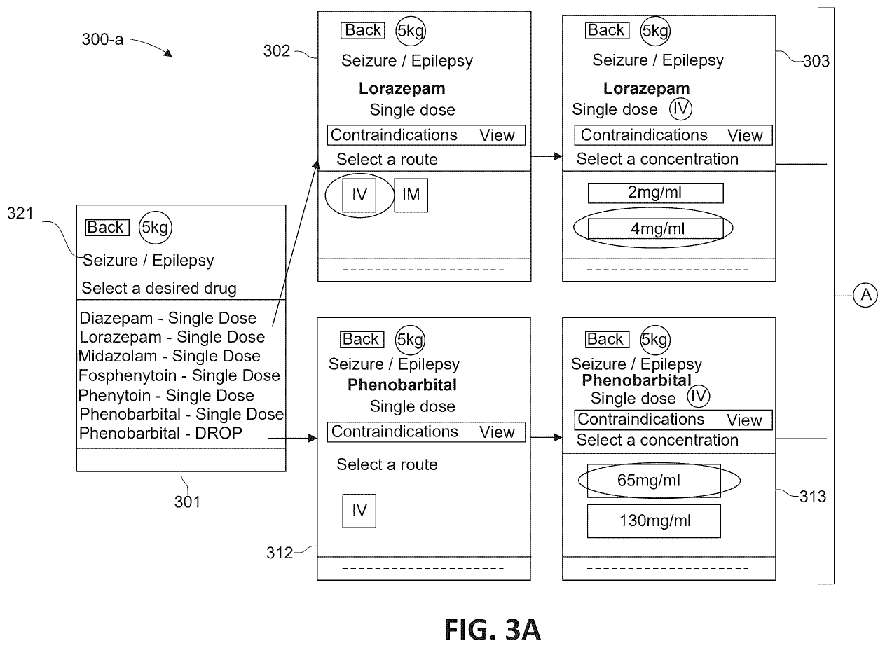

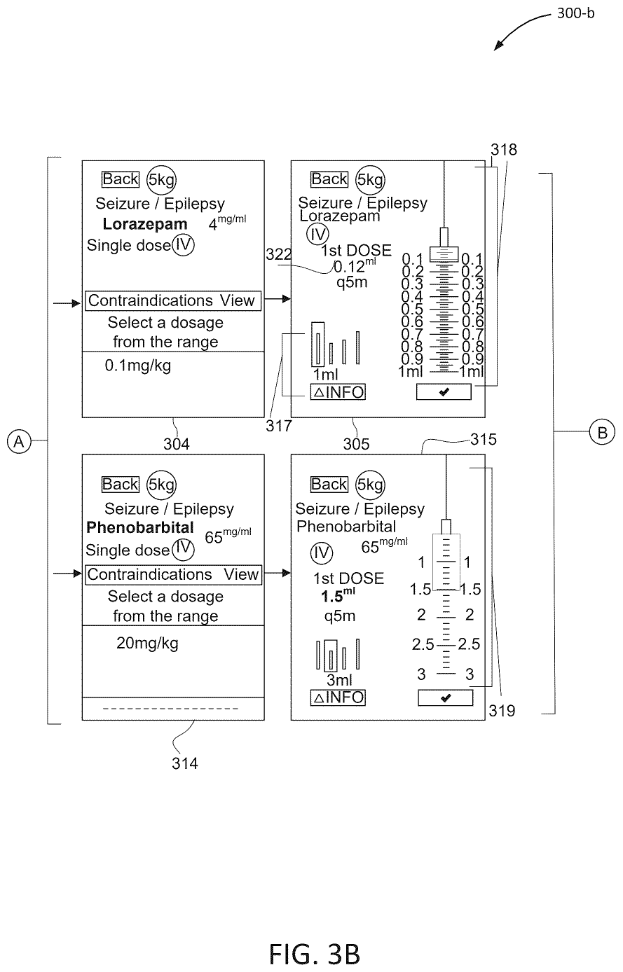

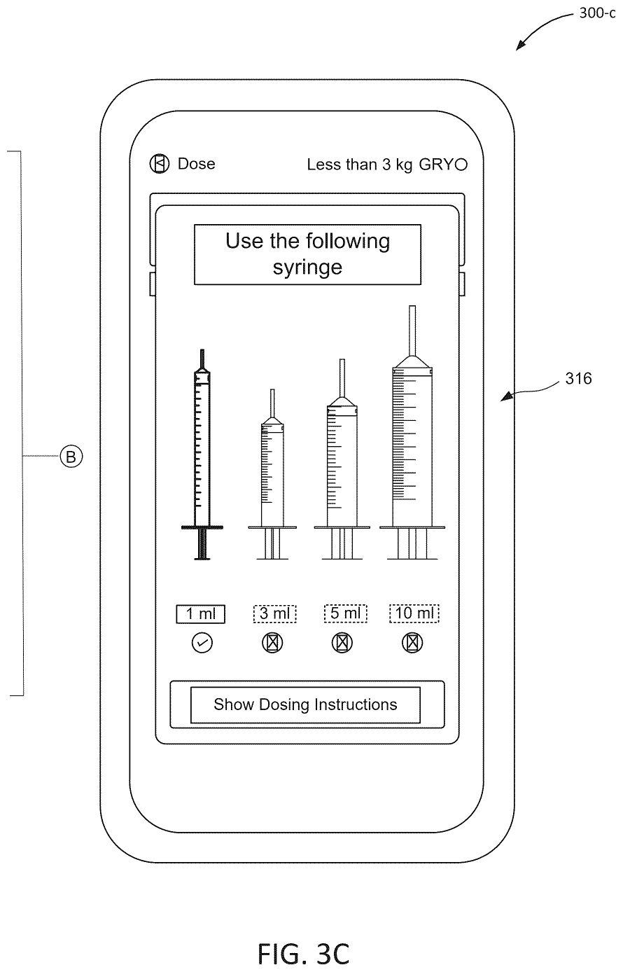

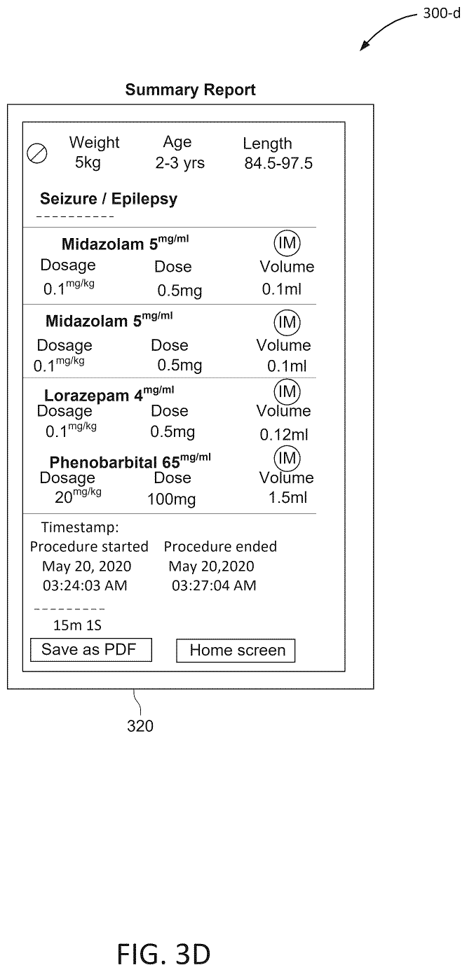

FIGS. 3A-3C illustrate various interface screens 300-a-c of a smartphone application for in accordance with the present disclosure. Process diagram in FIG. 3a begins with a first screen 301. First screen 301 shows a plurality of different types of a desired material comprising a medication (Diazepam, Lorazepam, etc.) for use in association with an already-identified use case 321 (which may have been selected in a prior screen (not shown). Moving now to second screen 302, shown is an example display after a provider selects a particular medication from first screen 301. In second screen 302, Lorazepam was selected from Screen 301. Upon selecting the medication in Screen 301, screen 302 is automatically displayed, which may show options for inputting additional variables. For example, in second screen 302, two routes of administration of the medication are provided: IV (intravenous) and IM (intramuscular). It is contemplated that each of these administration routes or methods have different dosages of the medication. Prior to administering the medication, a medical provider, in one such example, would select the appropriate “route” after third screen 303 is automatically displayed after the administration route variable has been selected (as seen, the IV administration route was selected in this example). In screen 303, the medical provider may be further prompted to select a concentration of the medication, which may also be referred to herein as a drug. As shown in third screen 303, concentrations for the IV route comprise a 2 mg/ml and a 4 mg/ml concentration in this example, which may comprise common manufacturer-provided concentrations. Each of the variables and/or prompts may be stored in a memory or database either internal or external to the system. Upon selecting the concentration in third screen 303, fourth screen 304, as seen in FIG. 3b, may be displayed. In fourth screen 304, one or more dosages of the medicine for the selected concentration may be presented. Here, only one dosage is shown: 0.1 mg/kg. Upon selecting the appropriate dosage, fifth screen 305 may be automatically displayed. Fifth screen 305 may comprise a plurality of different-sized commercially available syringes 317 (with additional information regarding displayed information available in the “INFO” drop-down box). Upon selecting the syringe variable on the screen 305 to align with the syringe size they are using (here, 1 ml), the screen 305 may display a visual indication of the correct volume of the dose, based on each of the received inputs and concentrations. One such visual indication comprises the visual indication 318.

Particular medical conditions or occurrences may require the administration of more than one drug in succession. For example, seizures may require several drugs. FIGS. 3a-3c also display a workflow for a second drug, phenobarbital, which may be required after the implementation of an initial desired material/medicine/drug. Returning now to FIG. 3a, seen is additional second screen 312, which may be automatically displayed after selection of the Phenobarbital menu selection in first screen 301. Additional second screen 312 shows only one option, IV, for the administration route variable. Additionally, third screen 313 shows two possible concentrations: 65 mg/ml and 130 mg/ml of the desired material for the selected IV administration route. Turning now to FIG. 3b, additional fourth screen 314 shows one possible dosage of the identified medicine: 20 mg/kg. Then, on additional fifth screen 315, the provider may select the appropriate size syringe (in this example, 3 ml), and the screen may display a visualization 319 of the correct volume of phenobarbital as it should appear in the syringe.

In some variations, after the fifth screens 305, 315, and as seen in FIG. 3c, a sixth screen 316 may be automatically displayed, where the sixth screen shows photographs of multiple syringe options for the identified desired material, concentration, use case scenario, and variables. A recommended syringe for the dosage displayed in FIG. 3b is with the correct volume is highlighted or otherwise identified as the correct syringe to use in the FIG. 3c display. The system identifies the 1 ml syringe in screen 316 (for a 1 ml dose) as the appropriate syringe to use in for the Lorazepam example above. As seen in screen 305 on FIG. 3b, the 1st dose Lorazepam comprises a .12 ml dose. With a .12 ml dose, in a 1 ml syringe the dosage will displace the plunger of the syringe along the axial dimension to a greater extent than other syringes. A great axial displacement makes it easier for a user to accurately measure the dose amount and validate the proper dose has been drawn. Screen 316 is exemplary of the types of measurement-tool-size recommendations that may be made according to the embodiments disclosed herein. These calculations and visualizations may be systematized for a specific industry, company, hospital, insurance, etc., and automated, allowing a user of the application to calculate and acquire a proper dosage of drugs quickly and accurately, even in high-pressure situations. Turing now to FIG. 3D, which illustrates an example of an interface screen 300-d, according to an embodiment of the disclosure. As seen, the interface screen 300-d displays an application summary screen 320, where the summary screen 320 may be displayed after the screen 316 shown in FIG. 3C. The summary screen 320 may display all drugs (or other desired material) that may have been dosed (or otherwise provided) for a particular patient/subject during treatment (or for the use case).

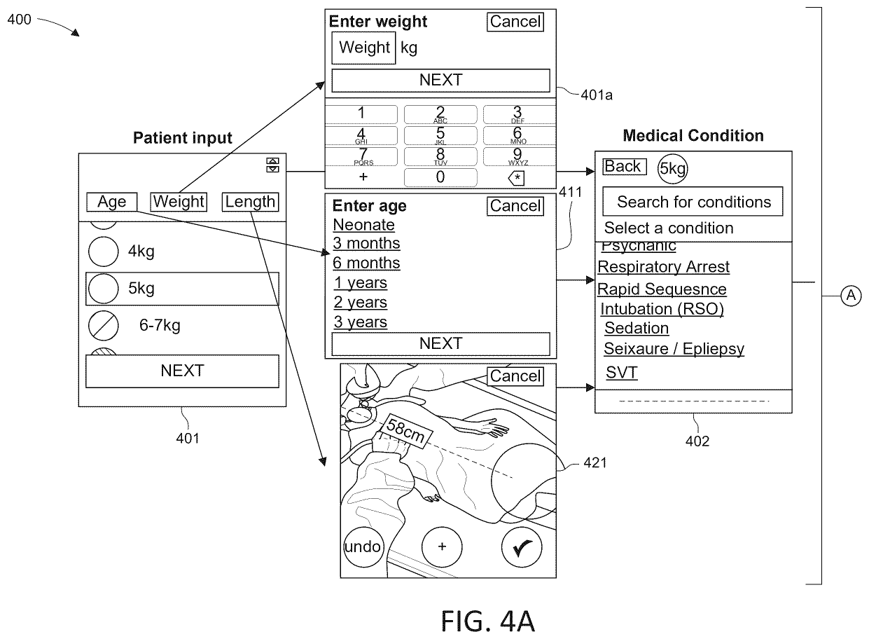

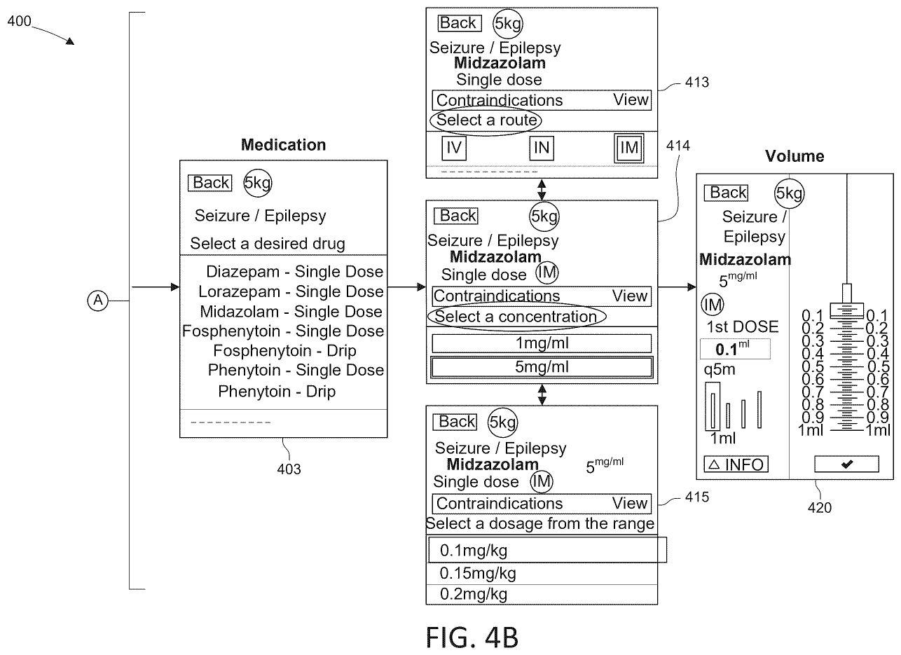

FIGS. 4a and 4b display another workflow 400 of the disclosed system via software application screens. One such workflow starts at screen 401. Screen 401 displays with examples of first measurement(s) for a subject, where the subject in FIG. 4a comprises a patient. There are several measurement examples shown. If the user of the application, which may comprise a medical provider, selects weight as the measurement, the user may enter the weight in screen 401a. If the user selected age as the first measurement, the user may enter or select the age on screen 411. And, if the user selects length as the measurement, they may enter it or use a hardware and/or software measuring device (e.g., a camera configured to measure length) to input length on screen 421. Other ways of entering measurements of a patient and/or any other subject may be used. It is contemplated, and as described herein, the system comprises an age-appropriate weight-based system to help reduce dosing errors.

After entering the proper first measurement, the next screen 402 may be displayed, enabling a user to enter another variable and/or the use case scenario. In the FIG. 4a example, the another variable comprises a medical condition. After selecting the variable, screen 403, as seen in FIG. 4b, may be displayed. Screen 403 may then allow the user (which may comprise a medical provider) to select a medication or other desired material to treat that medical condition/use case scenario. After selecting the medication/desired material in Screen 403, the application may present one or more screens as appropriate for the particular drug (i.e., desired material) and medical condition (i.e., use case). For example, and as shown in screen 414, if Midazolam is selected for the drug associated with a Seizure/Epilepsy use case (or in other cases/drug combinations), the provider may have to identify further variables. The further variables in FIG. 4b comprise: a drug concentration, which, as seen in screen 414, may have the provider select between two concentrations; a drug dose, as seen in screen 415; and a drug administration type, which, as seen in screen 413 may have user select between three different administration routes. Not all drugs may have this exact number of variables, so fewer or more screens may be shown to a user. After the appropriate number of variables screens is displayed, as determined by the user case, desired material, etc., screen 420 may automatically be displayed. The provider may select the correct syringe in screen 420 and the screen may then display the correct volume based on each of the patient measurement, the variables (including medical condition), the drug concentration, and the syringe size.

In embodiments, the system may utilize additional formulas to calculate estimates of other variables. For example, tidal volume (Dose) setting for a ventilator machine is based off length of a patient/subject. A patient length helps to estimate ideal body weight, and known formulas for calculating ideal body weight, and therefore appropriate tidal volume, may be implemented in this disclosure. Thus, knowing ideal body weight can determine tidal volume (how much air goes in or out with each breath) as a ventilator setting, which may also be displayed in a visualization for volume.

In embodiments, the system may alter information or instructions provided to users based on at least one of a use case scenario and a variable associated with a subject, such as a variable received by the variable receiving module 120 of FIG. 1. For example, the suggested medication doses provided to users may differ when treating cardiac bradycardia instead of cardiac arrest, even though the same medication type and administration method is used in treating both conditions.

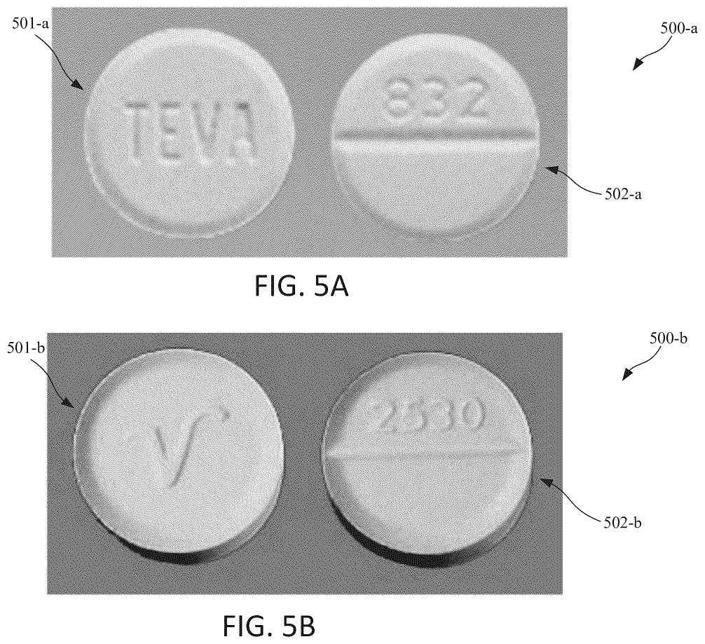

It is contemplated that a correct volume of dosages for medication desired material may be displayed as solid medications such as, but not limited to, tablets and pills. FIGS. 5A and 5B show images of tablets and/or pills 500-a and 500-b, respectively, that may be used as displays of a correct volume of desired material. As see, a front image 501 (e.g., front image 501-a, front image 501-b) and a rear or back image 502 (e.g., rear image 502-a, rear image 502-b) may be displayed for each pill 500. These types of images can be helpful to show not only the number of pills (e.g., 2 pills vs 1 or 1½ pills), but can also show the color and the specific markings (e.g., imprint, such as manufacturer name, a logo, a serial code or another identifier, etc.) the pills have to enable a visual validation by the provider is giving the correct dosage of the correct medication.

In embodiments, the system may further provide timing information to users regarding the application of the desired material to the subject. Timing information may include at least one of drug administration timing, such as IV drip timing, setting or curing timing, such as cement set time, and cooking-related timing, such as proofing time after the application of yeast to a dough in a recipe use case. For example, IV drips may be ordered in mcg/min units, but users may administer IV drips in mL/hour units, which produces a high error rate. The present disclosure may enable a reduction in such error rates by automatically calculating conversions between such units and providing users with a more user-friendly IV drip rate having the appropriate units and a duration of IV drip application. Such timing information may be displayed to users in the application. For example, screens 305 and 315 in FIG. 3b display not only a .12 ml and 1.5 ml dosage, abut also display the dosage as “q5m”. Such a term comprises a dosage timing that informs the provider to apply the dosage amount every five minutes.

As discussed herein, part of the system 100 (e.g., a mobile device such as a smart phone or other similar device) may be utilized to interpret an image of a label on a desired material container, with the image obtained using an imaging device (e.g., a camera and software), and the system 100 may obtain information from the label, such as the name, concentration, volume, etc. of the desired material within the container, and provide the information to the name receiving module 110. Such information from the label of the desired material container may also be used in other modules, such as the calculation module 114. And the calculation module 114 may be configured to calculate a correct concentration. The ability to interpret the label to obtain the correct concentration facilitates several functional aspects.

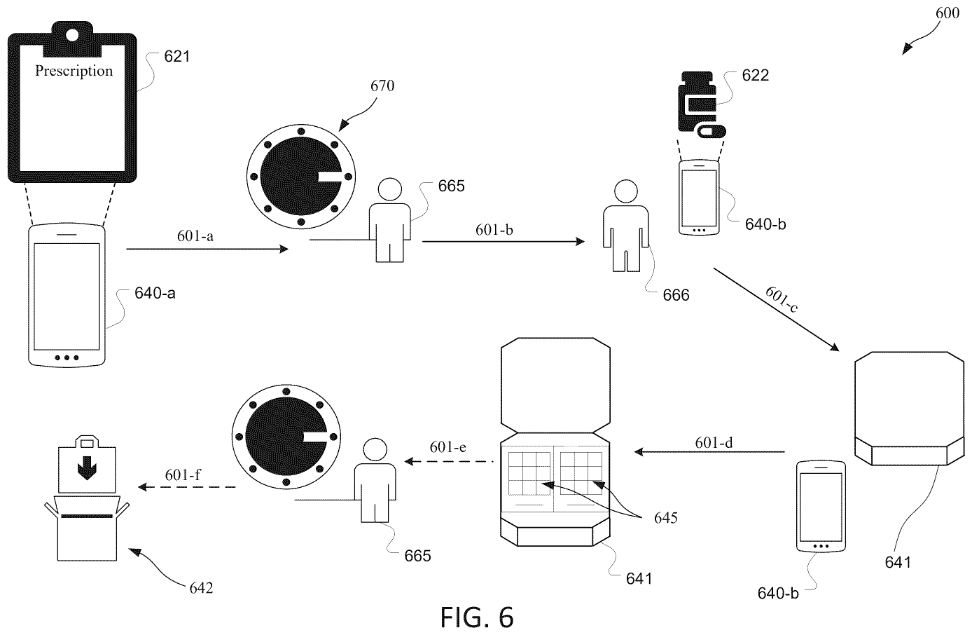

Referring to FIG. 6 for example, shown is a process-flow diagram 600 depicting one or more mobile device(s) 640 (e.g., mobile device 640-a, mobile device 640-b) used in connection with pharmacy ordering; patient pill minding and adherence (e.g., the mobile device 640-b may remind a patient what pills to take); and securing controlled substances in a smart medication security system 641 to prevent or reduce the controlled substances from being diverted away from the patient and/or taken by children; thus, preventing misappropriation and accidents.

As seen, a prescription 621 written for a patient (e.g., patient 666) may be scanned using a first mobile device 640-a. In some cases, the prescription 621 may be scanned by the doctor or another medical professional, such as a nurse practitioner. Alternatively, the patient may scan the prescription 621. In either case, at step 601-a, information pertaining to the prescription 621, such as a list of medications, recommended dosage, etc., may be transmitted to a pharmacist 655. Pharmacies typically store medicines in a secure location 670, such as a vault or safe, or an area requiring keycard access. After the pharmacist 655 has filled the prescription, at step 601-b, the patient 666 may collect medicines 622 and scan the container (e.g., bottle, or another storage container) containing the medicines using the mobile device 640-b. In some cases, the mobile device 640-b may be similar or substantially similar to the mobile device 640-a. In other cases, the mobile device 640-b and the mobile device 640-a may be the same mobile device. One or more of the mobile device 640-a and 640-b may comprise a software application, described herein and elsewhere throughout the disclosure. The software application may interface with one or more hardware components (e.g., barcode scanner, camera or imaging device) of the mobile device 640 to scan and/or image a label on the container containing the medicines 622. In some examples, the application may obtain information from the label, such as, but not limited to, name, concentration, volume, etc., of the medicine 622.

At step 601-c, the user or patient 622 may store the container containing the medicine 622 in the smart medication security system 641. The smart medication security system 641 may enable the patient 622 to store the medicines 622 safely and securely. For instance, the smart medication security system 641 may comprising locking features, and may be unlocked using a security PIN or passcode, biometrics (e.g., fingerprint scan, voice recognition, facial ID), a one-time password (OTP) transmitted to the mobile device 640-b, or through any other applicable means.

Although not necessary, in some examples, the smart medication security system 641 may be unlocked (e.g., shown as step 601-d) using the software application stored on the mobile device 640-b. As seen, the smart medication security system 641 comprises a plurality of compartments 645 for storing the user or patient's medication. In some examples, the compartments 645 may be labeled (e.g., with letters or numbers), one for each type of medicine. Further, the application on the mobile device 640-b may designate different compartments for different medicines (e.g., compartment 1 for medicine A, compartment 2 for medicine B, and so on) and instruct the patient 666 to place the container(s) containing the medicines into the assigned compartments.

In some cases, the smart medication security system 641 may transmit a notification to the pharmacist 665, for instance, for an automatic refill, when it determines the patient's medication is running low, to name two non-limiting examples. In such cases, the pharmacist 665 may fill the prescription (e.g., based on the information in prescription 621, previously scanned by mobile device 640-a) and mail it to the patient, at step 601-f. It should be noted that, steps 601-e and/or 601-f may be optional (shown as optional by the dashed lines).

In some other cases, steps 601-b through 601-f may be performed by the pharmacist 665. In this case, the user 666 may be the pharmacist, not the patient. For instance, after the pharmacist fills the prescription 621 at step 601-b, the pharmacist may scan the container containing the medicines 622 using the mobile device 640-b. Information pertaining to the medicine, including the name, dosage, color and/or shape of pills, etc., may be retrieved by the application stored on the mobile device 640-b and linked to the patient and prescription 621. At steps 601-c and 601-d, the pharmacist (i.e., user 666 in this case) may store the medicines 622 in the smart medication security system 641, for instance, in the compartments 645 assigned by the application on the mobile device 640-b. In this way, the smart medication security system 641 may be used to store a plurality of medicines, each in a different compartment 645. At step 601-e, the pharmacist 665 may lock the smart medication security system 641 (e.g., via the application on the mobile device 640-b, a physical keycard or fob, a PIN or passcode, etc.) so that it can be delivered to the patient. As seen, at step 601-f, a package 642 containing the smart medication security system 641 may be delivered to the patient's home address. In some cases, the patient or another user (e.g., caregiver) may receive the passcode for unlocking the smart medication security system 641 via an application stored on their mobile device, as a text message, an email, etc. In some cases, this passcode may be a temporary passcode (e.g., only valid for 24 hours after package delivery) and a user/patient may need to update the passcode, set up biometrics authentication (e.g., on the smart medication security system 641 or their user device), and/or set up two-factor authentication to unlock the smart medication security system 641 in the future.

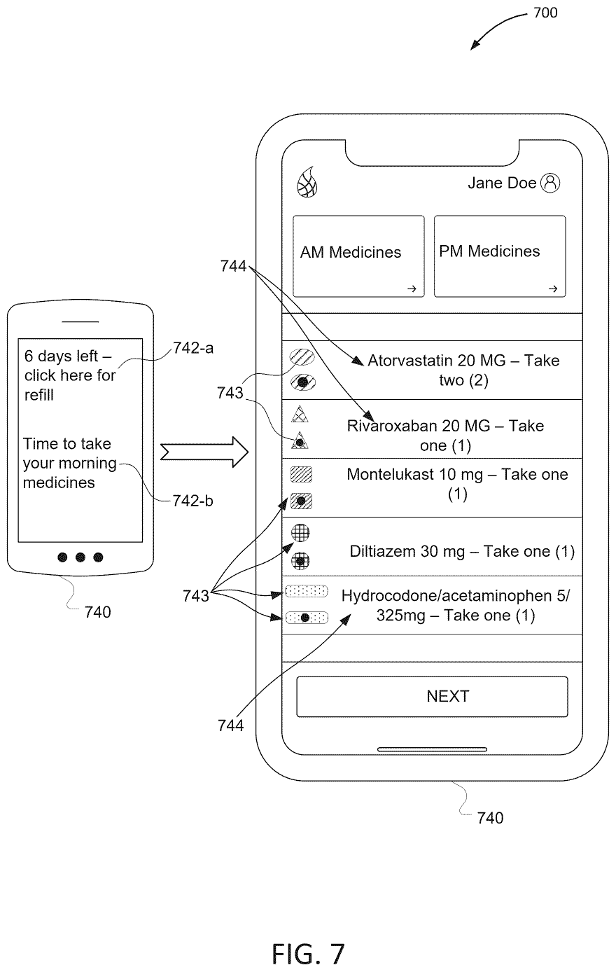

FIG. 7 illustrates an example of a process flow 700, according to various aspects of the disclosure. As shown in FIG. 7, a patient may receive one or more reminder notifications 742 on their mobile device 740. In some cases, when a reminder notification 742 (e.g., notification 742-a, notification 742-b) is selected, the mobile device 740 may display one or more images 743 of the actual pills along with an indication 744 of the name and/or number of pills (e.g., Atorvastatin 20 MG—take two, Rivaroxaban 20 MG—take one, etc.) that the patient is reminded to take. In some cases, the images 743 may comprise an image or photo of the front and back of the pills. For instance, in the example shown, the images 743 for each pill includes a visual depiction (e.g., a shape, such as ellipse for Atorvastatin, triangle for Rivaroxaban, rectangle with rounded edges for Singular, circle for Diltiazem, and an elongated rounded rectangle for Hydrocodone/acetaminophen) of the pill. Further, the images 743 with the black dot/circle represent images of the back of the pills. In some cases, the images 743 may also depict an imprint or label (if any) on the front and/or back sides of each pill, the color of the pill, etc. In some aspects, the visual depiction of the correct pill and dosage provides a simple and easily interpreted indication to help ensure proper dosing.

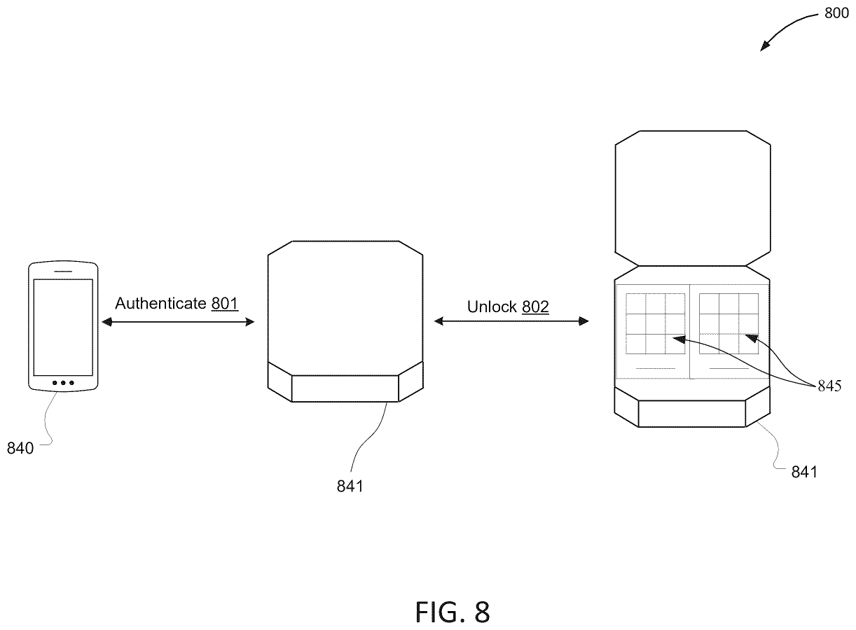

Turning now to FIG. 8, which illustrates a process flow 800 for authenticating and unlocking a smart medication security system 841, according to various aspects of the disclosure. In some cases, the smart medication security system 841 implements or more aspects of the smart medication security system 641, previously described in relation to FIG. 6. In some cases, process flow 800 may be implemented using a mobile device 840, where the mobile device 840 is in connection (e.g., using wireless means, such as Bluetooth, Wi-Fi, Near Field Communication or NFC) with the smart medication security system 841. The smart medication security system 841 may include a locking mechanism (e.g., a lid that is attached by hinges and secured by known electromechanical locking mechanisms to a base) that may be unlocked by the mobile device 840 (in response to an authentication process) to ensure that only the patient (or authorized user, e.g., a caregiver) has access to the pharmaceuticals.

In the example shown, the mobile device 840 may be used to authenticate 801 with the smart medication security system 841. In some cases, the authentication process may utilize two-factor authentication and/or biometric authentication such as finger, retinal, and/or facial recognition. For instance, a user or patient may need to open an application on their mobile device 840, where the application is associated with the smart medication security system 841, and input a PIN or passcode, provide a fingerprint scan or face ID, etc., to the application in order to unlock the security system 841. In some examples, the authentication process may be performed by the mobile device 840, the smart medication security system 841, or a combination thereof. For example, the mobile device 840 may transmit the information (e.g., PIN, passcode, etc.) input by the user to the smart medication security system 841, which then decides to authenticate or deny access to the user based on evaluating the received information. In other cases, the authentication 801 is performed at the mobile device, for instance, by the application stored on the mobile device 840. The mobile device 840 then transmits the authentication results (e.g., successful or unsuccessful) to the smart medication security system 841. If the authentication 801 is successful, the smart medication security system 841 unlocks 802 to allow the user/patient to retrieve the required medications.

In this way, the smart medication security system 841 helps to prevent or reduce narcotics and toxic medications from getting into children's hands while not being burdensome to patients. Medicines can be just as dangerous to children as a loaded gun, and they should be kept secure. Narcotics and addictive chemicals have the possibility of being diverted and stollen. These overdoses cause numerous deaths a year. The smart medication security system 841 helps to make sure medicine gets only to the people who need it, and only in the amounts needed. The smart medication security system 841 also helps ensure the wrong people don't get the dangerous medications.

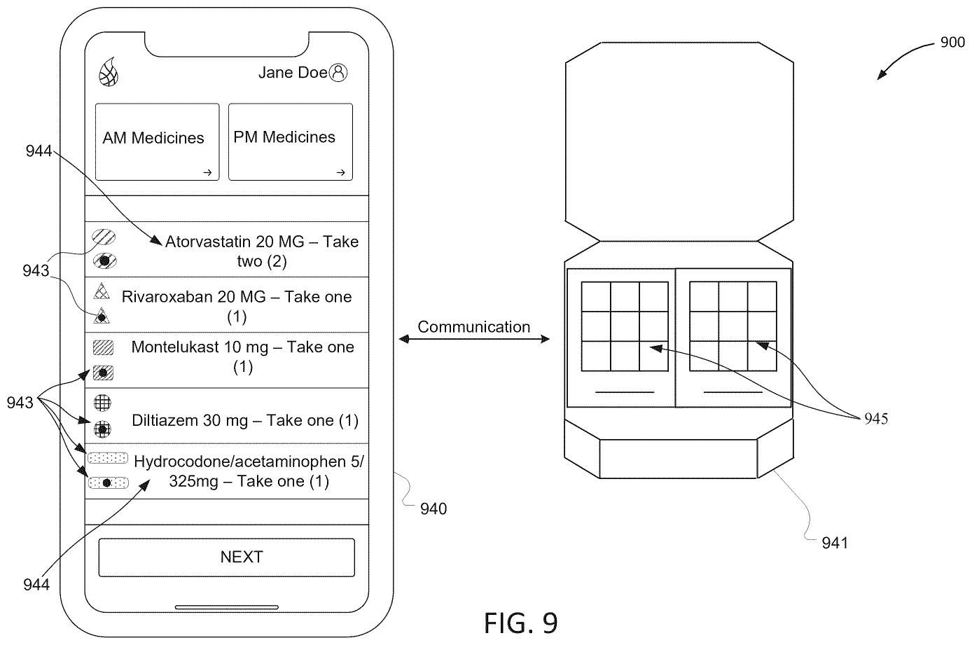

FIG. 9 illustrates a process flow 900, according to various aspects of the disclosure. As shown in FIG. 9, a locking smart medication security system 941 may include individual compartments 945 for each dose of a particular medication, and when the patient opens the security system 941, the compartment(s) 945 containing the medication(s) the patient should be taking (at that time) may be illuminated or otherwise identified to help prevent or reduce confusion about the medication that should be taken next. Additionally, or alternatively, a mobile device 940 in communication with the smart medication system 941 may display one or more images 943 of the actual pills along with an indication 944 of the name and/or number of pills (e.g., Atorvastatin 20 MG—take two, Rivaroxaban 20 MG—take one, etc.) that the patient is reminded to take. In some cases, the images 943 may comprise an image or photo of the front and back of the pills. For instance, in the example shown, the images 943 for each pill includes a visual depiction (e.g., a shape, such as ellipse for Atorvastatin, triangle for Rivaroxaban, rectangle with rounded edges for Montelukast, circle for Diltiazem, and an elongated rounded rectangle for Hydrocodone/acetaminophen) of the pill. Further, the images 943 with the black dot/circle represent images of the back of the pills. In some cases, the images 943 may also depict an imprint or label (if any) on the front and/or back sides of each pill, the color of the pill, etc. In some aspects, the visual depiction of the correct pill and dosage provides a simple and easily interpreted indication to help ensure proper dosing. In one non-limiting example, a user/patient may click or tap on the pill/medicine on the user device 940 and the smart medication system 941 may illuminate the compartment 945 containing the medicine, which may further enhance user experience. In some cases, once the user/patient has retrieved the medicine from the correct compartment, the application on the mobile device 940 may automatically update the list of medicines displayed on the device 940. For instance, the application may remove the medicine from the list or display a strikethrough over the text to prevent or reduce the user from accidentally taking more than the required dosage.

The smart medication security system 941 may be communicatively coupled to both a patient's mobile device (e.g., mobile device 940) and identified remote people/entities/devices via Wi-Fi connection and/or Bluetooth connection.

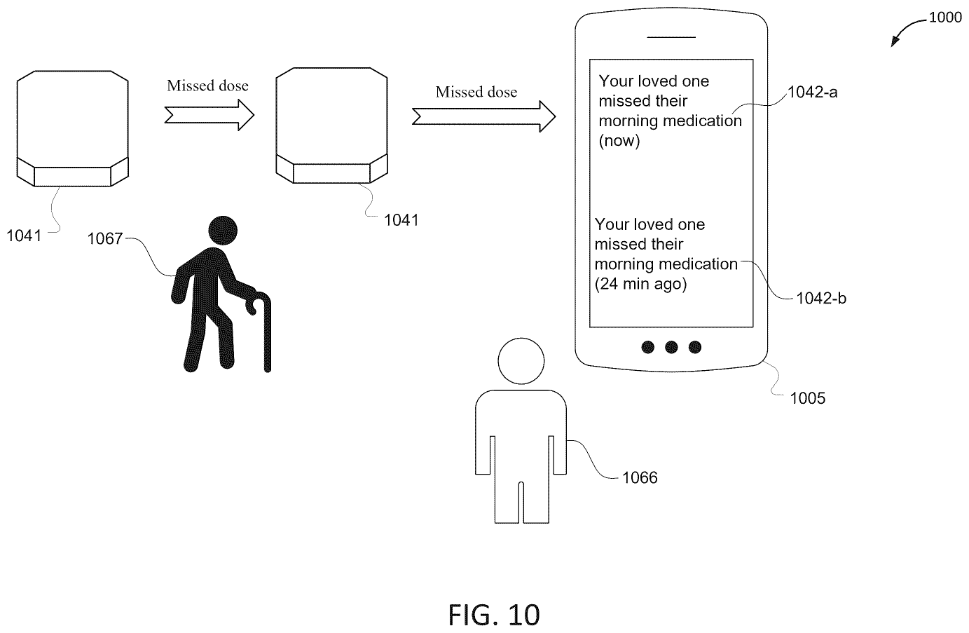

FIG. 10 illustrates a process flow 1000 to notify an individual 1066 (e.g., doctor, caregiver) when a patient 1067 has missed a dose, according to various aspects of the disclosure. Beneficially, as shown in FIG. 10, the network connection enables family members and/or doctors to be notified if a dose is missed. For example, the smart medication security system 1041 may initiate one or more notifications 1042 (e.g., notification 1042-a, notification 1042-b) on mobile device 1005 if the locking lid is not opened by the patient 1067 for a threshold period of time. In some cases, the smart medication security system 1041 may automatically trigger an alert (i.e., notification 1042) that may be transmitted and displayed on the user device 1040 associated with the user 1066.

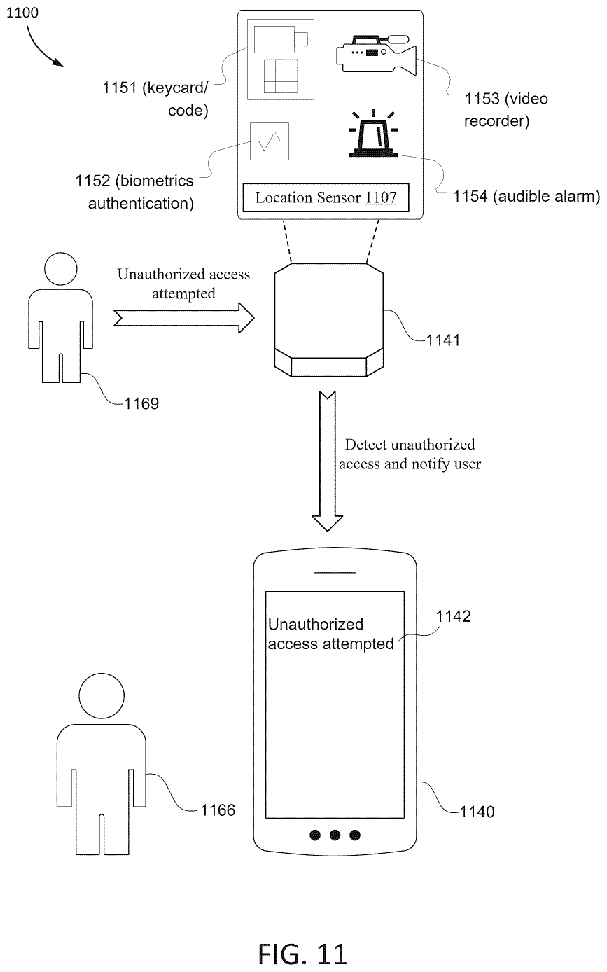

FIG. 11 illustrates a process flow 1100 for identifying and notifying a user 1166 when unauthorized access is attempted, according to an embodiment of the disclosure. In some cases, a notification and alarm may be triggered when unauthorized access is attempted. For example, incorrect entry of credentials (e.g., by a rogue or unauthorized user 1169) may trigger the alarm. It is also contemplated that the smart medication security system 1141 may have location sensing capabilities (Wi-Fi and/or GPS) that trigger an alarm (e.g., audible alarm 1154) if the smart medication security system 1141 is relocated. For instance, the smart medication security system 1141 may comprise one or more components, such as a keypad 1151 for entering a PIN or passcode, an imaging device 1153 capable of capturing images and/or video, a biometrics authentication system 1153 (e.g., for fingerprint recognition, voice recognition, retina or iris scan, facial recognition, etc.), an audible alarm 1154, and/or a location sensor 1107. In some embodiments, the smart medication security system may also include shock-detection capabilities (e.g., accelerometers and associated logic) to trigger the audible alarm 1154 if attempts are made to break into the smart medication security system 1141. As shown in process flow 1100, the smart medication security system 1141 may trigger a notification or alert 1142 for display on the user device 1140 (i.e., associated with the user/patient 1166) when it detects unauthorized access.

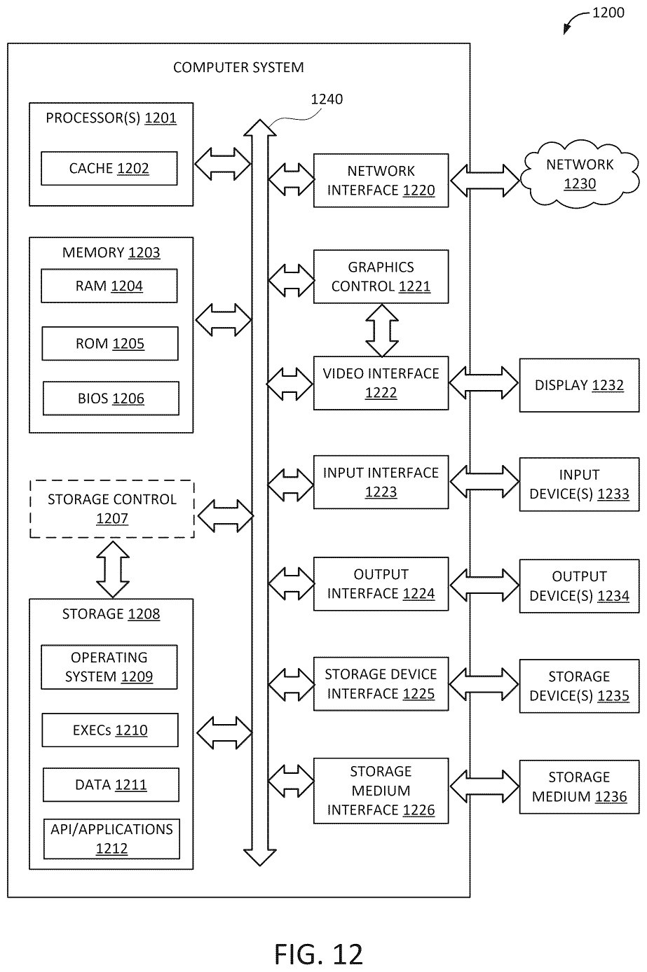

Referring to FIG. 12, it is a block diagram depicting an exemplary machine that includes a computer system 1200 within which a set of instructions can execute for causing a device to perform or execute any one or more of the aspects and/or methodologies of the present disclosure. For example, the exemplary machine may be utilized to realize aspects of the mobile devices described herein and aspects of the smart medication security system. The components in FIG. 12 are examples only and do not limit the scope of use or functionality of any hardware, software, embedded logic component, or a combination of two or more such components implementing particular embodiments.

Computer system 1200 may include a processor(s) 1201, a memory 1203, and a storage 1208 that communicate with each other, and with other components, via a bus 1240. The bus 1240 may also link a display 1232, one or more input devices 1233 (which may, for example, include a keypad, a keyboard, a mouse, a stylus, etc.), one or more output devices 1234, one or more storage devices 1235, and various tangible storage media 1236. All of these elements may interface directly or via one or more interfaces or adaptors to the bus 1240. For instance, the various tangible storage media 1236 can interface with the bus 1240 via storage medium interface 1226. Computer system 1200 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Processor(s) 1201 (or central processing unit(s) (CPU(s))) optionally contains a cache memory unit 1202 for temporary local storage of instructions, data, or computer addresses. Processor(s) 1201 are configured to assist in execution of computer readable instructions. Computer system 1200 may provide functionality for the components depicted in FIGS. 1-5 as a result of the processor(s) 1201 executing non-transitory, processor-executable instructions embodied in one or more tangible computer-readable storage media, such as memory 1203, storage 1208, storage devices 1235, and/or storage medium 1236. The computer-readable media may store software that implements particular embodiments, and processor(s) 1201 may execute the software. Memory 1203 may read the software from one or more other computer-readable media (such as mass storage device(s) 1235, 1236) or from one or more other sources through a suitable interface, such as network interface 1220. The software may cause processor(s) 1201 to carry out one or more processes or one or more steps of one or more processes described or illustrated herein. Carrying out such processes or steps may include defining data structures stored in memory 1203 and modifying the data structures as directed by the software.

The memory 1203 may include various components (e.g., machine readable media) including, but not limited to, a random-access memory component (e.g., RAM 1204) (e.g., a static RAM “SRAM”, a dynamic RAM “DRAM, etc.), a read-only component (e.g., ROM 1205), and any combinations thereof. ROM 1205 may act to communicate data and instructions unidirectionally to processor(s) 1201, and RAM 1204 may act to communicate data and instructions bidirectionally with processor(s) 1201. ROM 1205 and RAM 1204 may include any suitable tangible computer-readable media described below. In one example, a basic input/output system 1206 (BIOS), including basic routines that help to transfer information between elements within computer system 1200, such as during start-up, may be stored in the memory 1203.

Fixed storage 1208 is connected bidirectionally to processor(s) 1201, optionally through storage control unit 1207. Fixed storage 1208 provides additional data storage capacity and may also include any suitable tangible computer-readable media described herein. Storage 1208 may be used to store operating system 1209, EXECs 1210 (executables), data 1211, API applications 1212 (application programs), and the like. Often, although not always, storage 1208 is a secondary storage medium (such as a hard disk) that is slower than primary storage (e.g., memory 1203). Storage 1208 can also include an optical disk drive, a solid-state memory device (e.g., flash-based systems), or a combination of any of the above. Information in storage 1208 may, in appropriate cases, be incorporated as virtual memory in memory 1203.

In one example, storage device(s) 1235 may be removably interfaced with computer system 1200 (e.g., via an external port connector (not shown)) via a storage device interface 1225. Particularly, storage device(s) 1235 and an associated machine-readable medium may provide nonvolatile and/or volatile storage of machine-readable instructions, data structures, program modules, and/or other data for the computer system 1200. In one example, software may reside, completely or partially, within a machine-readable medium on storage device(s) 1235. In another example, software may reside, completely or partially, within processor(s) 1201.

Bus 1240 connects a wide variety of subsystems. Herein, reference to a bus may encompass one or more digital signal lines serving a common function, where appropriate. Bus 1240 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 1200 may also include an input device 1233. In one example, a user of computer system 1200 may enter commands and/or other information into computer system 1200 via input device(s) 1233. Examples of an input device(s) 1233 include, but are not limited to, an alpha-numeric input device (e.g., a keyboard), a pointing device (e.g., a mouse or touchpad), a touchpad, a joystick, a gamepad, an audio input device (e.g., a microphone, a voice response system, etc.), an optical scanner, a video or still image capture device (e.g., a camera), and any combinations thereof. Input device(s) 1233 may be interfaced to bus 1240 via any of a variety of input interfaces 1223 (e.g., input interface 1223) including, but not limited to, serial, parallel, game port, USB, FIREWIRE, THUNDERBOLT, or any combination of the above.

In particular embodiments, when computer system 1200 is connected to network 1230, computer system 1200 may communicate with other devices, specifically mobile devices and enterprise systems, connected to network 1230. Communications to and from computer system 1200 may be sent through network interface 1220. For example, network interface 1220 may receive incoming communications (such as requests or responses from other devices) in the form of one or more packets (such as Internet Protocol (IP) packets) from network 1230, and computer system 1200 may store the incoming communications in memory 1203 for processing. Computer system 1200 may similarly store outgoing communications (such as requests or responses to other devices) in the form of one or more packets in memory 1203 and communicated to network 1230 from network interface 1220. Processor(s) 1201 may access these communication packets stored in memory 1203 for processing.

Examples of the network interface 1220 include, but are not limited to, a network interface card, a modem, and any combination thereof. Examples of a network 1230 or network segment 1230 include, but are not limited to, a wide area network (WAN) (e.g., the Internet, an enterprise network), a local area network (LAN) (e.g., a network associated with an office, a building, a campus or other relatively small geographic space), a telephone network, a direct connection between two computing devices, and any combinations thereof. A network, such as network 1230, may employ a wired and/or a wireless mode of communication. In general, any network topology may be used.

Information and data can be displayed through a display 1232. Examples of a display 1232 include, but are not limited to, a liquid crystal display (LCD), an organic liquid crystal display (OLED), a cathode ray tube (CRT), a plasma display, and any combinations thereof. The display 1232 can interface to the processor(s) 1201, memory 1203, and fixed storage 1208, as well as other devices, such as input device(s) 1233, via the bus 1240. The display 1232 is linked to the bus 1240 via a video interface 1222, and transport of data between the display 1232 and the bus 1240 can be controlled via the graphics control 1221.

In addition to a display 1232, computer system 1200 may include one or more other peripheral output devices 1234 including, but not limited to, an audio speaker, a printer, and any combinations thereof. Such peripheral output devices may be connected to the bus 1240 via an output interface 1224. Examples of an output interface 1224 include, but are not limited to, a serial port, a parallel connection, a USB port, a FIREWIRE port, a THUNDERBOLT port, and any combinations thereof.

In addition, or as an alternative, computer system 1200 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

### Customized Medication Recommendation Platform

Doses of medication are dependent on multiple variables. For example, a patient who has normal liver or kidney function and ideal body weight is the primary determinant of dose. However, humans have medical problems as they age and kidney and liver function change, and other organ systems can have alterations as well such but not limited to coagulation function. For example, the human body is dynamic, and organs can be compromised by various factors, such as age, physiology (shock), chemistry, or other medications. In these cases, the metabolism of medications can be altered and the dose of the medication must be adjusted. A dose that is appropriate for a person with normal liver, kidney, or other organ function could be a dangerous overdose in a person with compromised liver, kidney, or other organ function. So, the dose must be adjusted to account for the limitation in these organs. For example, a the organ function can be measured and the dose can be modified or adjusted accordingly, so as to not overwhelm that organ and reduce the likelihood of a disproportionate rise or fall in the desired blood or tissue concentration of the medication (e.g., based on the volume of distribution of that drug). Additionally, providers such as physicians do not have time or access to review current data for medicine and drugs every time they need to prescribe a respective medicine or drug. For example, antibiotics can become less effective overtime, e.g., if the bacteria the antibiotic is intended to treat develops a resistance to the antibiotic. However, physicians do not review the most current data on sensitivity to antibiotics or current bacteria resistivity every time before prescribing it. Since bacteria develops resistance to antibiotics, it's a constantly moving target. Thus, certain antibiotics a physician may have prescribed a year or month previous may be outdated and ineffective at a later date. Thus, physicians tend to prescribe medicine based on their perceived effectiveness of the drug, as opposed to actual real-time data. Further, most providers do not compare effectiveness of medicines, such as antibiotics, to price of the medicine due to time constraints. Mainly because insurance information and other financial information, such as cost of medicine out of network, is not readily available, siloed or cumbersome to access. Physicians are typically content with prescribing an effective medicine, but do not have the resources or time to factor price into the prescription or recommendation.

The system described herein can assists a provider in determining the best solution for a specific patient diagnosed with a medical problem out of vast possible solutions, while also avoiding creating a new problem (e.g., giving the patient a medicine that negatively interacts with a medicine the patient is already taking, giving the patient a medicine he/she is allergic to, and the like). The system retrieves or receives all patient information available for the specific patient at a current state (e.g., current disease or illness to be treated, height, weight, liver function, kidney function, current medicine regimen, current blood tests, and the like) from a vast number of sources (e.g., electronic medical record, patient/provider input, and the like) to have a holistic understanding of the patient's current state. The system retrieves or receives all drug information available for each drug that could be used to treat the patient's current disease or illness, which may be referred to as candidate drugs, (e.g., contraindications, doses for certain kidney/liver function levels, drug availability, drug cost) from a vast number of sources (e.g., Food and Drug Administration (FDA) databases and labels (e.g., which include specific legal instructions and directions for usage for a respective substance that is controlled by prescription, and which can include hundreds of pages) for each drug, hospital or pharmacy database, and the like) to have a holistic understanding of the candidate drugs available. The system analyzes patient information with drug information and creates a summary of potential drugs, e.g., candidate drugs, ranked for the specific patient. In this way, the provider can prescribe an optimal drug, at the optimal dose, and the optimal time, while also factoring in the cost for the specific patient. As used herein, the term “optimal” can refer to an improved recommendation, as compared to a recommendation produced by traditional means and systems.

The system utilizes reports and lab tests to help determine the (1) correct or appropriate medicine(s)/medication(s) and (2) correct volume based on one or more measurements, e.g., from the patient's electronic medical record (“EMR”); a doctor inputting lab tests into the system, such as blood tests; or the patient inputting the measurement(s) into the system. For example, medicine type and medicine dose are based on reports or lab tests of specific people or general information, such as studies/reports on certain medications. A person's liver function, kidney function and other organs' functions are typically determined through blood tests. Likewise, for directing antibiotic use tests of a person's blood, urine, sputum or other body fluids can show what type of organism they are infected with. For example, general information can include culture and sensitivity data for antibiotics or antibiograms, which is a report that summarizes the antimicrobial susceptibility testing results of specific microorganisms to various antimicrobial agents—it provides information on the percentage of organisms that are susceptible to the tested antibiotics, helping to guide empirical therapy and monitor resistance patterns over time. Reports on medicines, such as antibiotics, can include real-time and current effectiveness, side effects, contraindications of antibiotic, availability information, and cost information. Retrieving and using real-time data of the medicine is beneficial since efficacy is constantly changing. Reports for specific patients can include personal details, such as measurements (age, body weight, height, metabolism rate, and the like), which can include values in addition to anthropometric measurements, such as blood test measurements as well as physiologic functions tests (such as an cardiac ultrasound ECHO test to determine the pumping strength of the heart); insurance carrier or patient-specific coverage; vitamin intake; medications; diseases; allergies; and the like.

The system can retrieve or calculate and display real-time “efficacy” (e.g., based on the most current data) as a variable of one or more medicines/medications. The system can also display a treatment protocol for the medical condition of the subject or for each medicine of the one or more medicines. Additional variables associated with each medicine of the one or more medicines can comprise a contraindication, e.g., a condition or circumstance that suggests or indicates that a particular technique or drug should not be used in the case in question; an estimated cost of the medicine; insurance coverage for the medicine; availability information of the medicine. Additionally, the system can retrieve and analyze information related to the problem being treated. For example, can retrieve data regarding the environment, e.g., if treating pneumonia, system will look at actionable data to determine which microbiologic organisms are mostly likely the cause of the pneumonia. The system factors this into which medication will be recommended and how the medicines will be ranked. The system can also adjust the dose for each available medicine, customized to the specific patient. For example, if the patient has kidneys that do not function properly, the system can calculate and recommend a dose based on patient specific lab tests, such as the most current renal or kidney function tests, reducing the likelihood that the recommended dose and/or medication will negatively affect the patient because of the kidney issues, e.g., a recommendation in accordance with the respective FDA label for that drug and indicated kidney function.

The system can then rank the candidate drugs for the specific patient, e.g., from a best to a worst match and present a display to the provider. For example, the provider can access a summary screen that includes a table that displays all this information in a holistic way for all possible medications for a specific patient. It will be tailored to that specific patient since the system considers the specific patient's medical history, conditions/diseases, and measurements, as well as the patient's insurance information. For example, insurance, blood tests, other patient labs make the output customized for that specific patient. The summary screen can further comprise a pictogram associated with each medicine of the one or more medicines. The summary can also display the packaging of the recommended medications and/or medication selected by provider. This reduces the likelihood of mistakes involved around giving the patient the wrong medication or incorrect amount of medication.

The system (1) evaluates the drug/potential drugs (e.g., efficacy, availability, and the like), (2) evaluates the problem being treated, (3) evaluates the patient (e.g., disease state, organ function, lab test measurements, age, weight, vitamins, medications/diseases, allergies) and the patient's situation (e.g., insurance coverage and cost for each potential drug for the specific patient). Then, the system can weigh all these factors and variables and rank all available and appropriate medications in an order that is customized for the specific patient. So, the system can recommend the best drug for a particular patient, factoring in patient specific factors, such as allergies and other medications so the recommended medication will not adversely mix with other medications the patient is taking. The system can process diseases and respective medications for illnesses, heart issues, diabetes, daily medicines, daily vitamins and the like, e.g., any use-case a patient would need medicine for. For example, the system can evaluate a patient's most current blood test (which is how organ function is determined, which can be done by the system or provided to the system) and the specific drug calculation equation for that specific drug at that specific level of organ function. These plethora of drug dose adjustment equations are specific for each drug, impossible to memorize, voluminous and complicated. Any mistake in this calculation would lead to an incorrect dose, which can result in harm or death to the patient. The system can match the correct equation to the desired drug and input the relevant blood test variables from the patients record to determine the correct dose, which can prevent human error while in the calculation of dose. The ability of the system to do this in reduced time, as compared to a provider, reduces the risk of miscalculations and dosing errors, especially with respect to ICU patients whom can have multiple organs compromised and can need multiple medications at once, each of which can increasingly complicate their management and metabolism of the medications due to the required adjustments and variables affecting those adjustments.

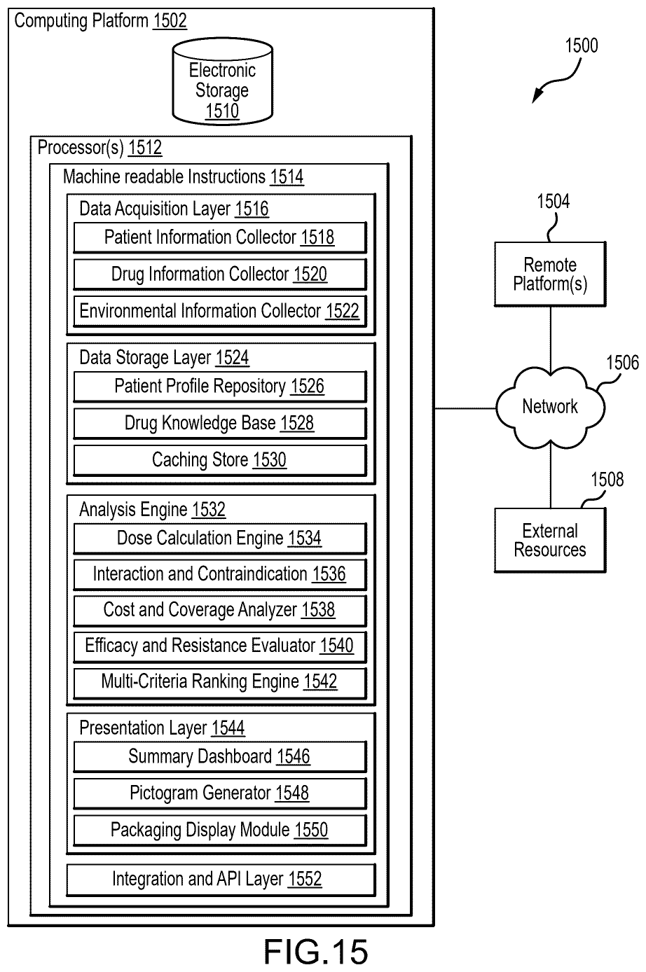

FIG. 15 illustrates a platform or system 1500 configured for automatically recommending a solution, e.g., a medicine/drug, a treatment plan, or the like, for a specific patient diagnosed with a medical problem, condition, disease, or the like, in accordance with one or more implementations. The system 1500 can be referred to herein as a medicine recommendation platform 1500. In some implementations, the system 1500 can include one or more servers or computing platform(s) 1502. Server(s) 1502 may be configured to communicate with one or more remote, e.g., client, computing platforms 1504 according to a client/server architecture and/or other architectures. Computing or remote platform(s) 1504 may be configured to communicate with other computing platforms via server(s) 1502 and/or according to a peer-to-peer architecture and/or other architectures. Users may access system 1500 via client computing platform(s) 1504. In some examples, the medicine recommendation platform 1500 can be an agent, e.g., a system that can interact with various other system items. For example, the medicine recommendation platform 1500 can be accessible on an application and/or website.

Server(s) 1502 can include electronic storage 1510, one or more processors 1512, and/or other components. Server(s) 1502 can include communication lines, or ports to enable the exchange of information with a network and/or other computing platforms. Illustration of server(s) 1502 in FIG. 15 is not intended to be limiting. Server(s) 1502 can include a plurality of hardware, software, and/or firmware components operating together to provide the functionality attributed herein to server(s) 1502. For example, server(s) 1502 may be implemented by a cloud of computing platforms operating together as server(s) 1502.

Electronic storage 1510 may comprise non-transitory storage media that electronically stores information. The electronic storage media of electronic storage 1510 can include one or both of system storage that is provided integrally (i.e., substantially non-removable) with server(s) 1502 and/or removable storage that is removably connectable to server(s) 1502 via, for example, a port (e.g., a USB port, a firewire port, etc.) or a drive (e.g., a disk drive, etc.). Electronic storage 1510 can include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 1510 can include one or more virtual storage resources (e.g., cloud storage, a virtual private network, and/or other virtual storage resources). Electronic storage 1510 may store software algorithms, information determined by processor(s) 1512, information received from server(s) 1502, information received from client computing platform(s) 1502, and/or other information that enables server(s) 1502 to function as described herein.

In some examples, server(s) 1502, client computing platform(s) 1504, and/or external resources 1508 may be operatively linked via one or more electronic communication links. For example, such electronic communication links may be established, at least in part, via a network such as the Internet and/or other computing networks. It will be appreciated that this is not intended to be limiting, and that the scope of this disclosure includes implementations in which server(s) 1502, client computing platform(s) 1504, and/or external resources 1508 may be operatively linked via some other communication media.

External resources 1508 can include sources of information outside of system 1500, external entities participating with system 1500, and/or other resources. In some implementations, some or all of the functionality attributed herein to external resources 1508 may be provided by resources included in or otherwise in communication with the system 1500, and vice versa.

Server(s) 1502 may be configured by machine-readable instructions 1514. Machine-readable instructions 1514 can include one or more instruction modules. The instruction modules can include computer program modules. The instruction modules can include one or more of a data acquisition layer 1516, which can include one or more of a patient information collector 1518, a drug information collector 1520, or an environmental information collector 1522; a data storage layer 1524, which can include one or more of a patient profile repository 1526, a drug knowledge base 1528, or a caching store 1530; an analysis engine 1532, which can include one or more of a dose calculation engine 1534, an interaction and contraindication 1536 analyzer, a cost and coverage analyzer 1538, an efficacy and resistance evaluator 1540, or a multi-criteria ranking engine 1542; a presentation layer 1544, which can include one or more of a summary dashboard 1546, a pictogram generator 1548, or a packaging display module 1550; or an integration and API layer 1552.

Processor(s) 1512 may be configured to provide information processing capabilities in server(s) 1502. As such, processor(s) 1512 can include one or more of a digital processor, an analog processor, a digital circuit designed to process information, an analog circuit designed to process information, a state machine, and/or other mechanisms for electronically processing information. Although processor(s) 1512 is shown in FIG. 15 as a single entity, this is for illustrative purposes only. In some implementations, processor(s) 1512 can include a plurality of processing units. These processing units may be physically located within the same device, or processor(s) 1512 may represent processing functionality of a plurality of devices operating in coordination. Processor(s) 1512 may be configured to execute modules 1516, 1524, 1532, 1544, and/or 1552, and/or other modules, such as the sub-modules, e.g., 1518, 1520, 1522, 1526, 1528, 1530, 1534, 1536, 1538, 1540, 1542, 1546, 1548, and/or 1550. Processor(s) 1512 may be configured to execute modules 1516, 1524, 1532, 1544, and/or 1552, and/or other modules, such as the sub-modules, by software; hardware; firmware; some combination of software, hardware, and/or firmware; and/or other mechanisms for configuring processing capabilities on processor(s) 1512. As used herein, the term “module” may refer to any component or set of components that perform the functionality attributed to the module. This can include one or more physical processors during execution of processor readable instructions, the processor readable instructions, circuitry, hardware, storage media, or any other components.

Although modules 1516, 1524, 1532, 1544, and/or 1552, and/or other modules, such as the sub-modules, are illustrated in FIG. 15 as being implemented within a single processing unit, in implementations in which processor(s) 1512 includes multiple processing units, one or more of modules 1516, 1524, 1532, 1544, and/or 1552, and/or other modules, such as the sub-modules, may be implemented remotely from the other modules. The description of the functionality provided by the different modules 1516, 1524, 1532, 1544, and/or 1552, and/or other modules, such as the sub-modules, described herein is for illustrative purposes, and is not intended to be limiting, as any of modules 1516, 1524, 1532, 1544, and/or 1552, and/or other modules, such as the sub-modules, may provide more or less functionality than is described. For example, one or more of modules 1516, 1524, 1532, 1544, and/or 1552, and/or other modules, such as the sub-modules, may be eliminated, and some or all of its functionality may be provided by other ones of modules 1516, 1524, 1532, 1544, and/or 1552, and/or other modules, such as the sub-modules. As another example, processor(s) 1512 may be configured to execute one or more additional modules that may perform some or all of the functionality attributed below to one of modules 1516, 1524, 1532, 1544, and/or 1552, and/or other modules, such as the sub-modules.

### Medicine Recommendation Platform 1500

The medicine recommendation platform 1500 can include and/or orchestrate a data acquisition layer 1516, a data storage layer 1524, an analysis engine 1532, a presentation layer 1544, and an integration and Application Programming Interface (API) layer 1552 (and the sub-modules) to compute and deliver patient-specific medication guidance. In one example, the medicine recommendation platform 1500 can interface with EMR sources, external drug repositories, and epidemiological feeds to assemble a current clinical context and candidate drug set, and the medicine recommendation platform 1500 can expose outputs as ranked lists with dosing guidance and reasoning. Therefore, the medicine recommendation platform 1500 reduces clinician effort while mitigating interaction risk and incorporating real-time efficacy, availability, and cost information at the point of prescribing.

The medicine recommendation platform 1500 can include a configurable weighting framework that persists per-user or per-facility weight vectors for efficacy, safety, cost, and availability and applies semantics to modify scores without service interruption. Additionally, the medicine recommendation platform 1500 can implement a modular microservices architecture that containerizes ingestion, analytics, and presentation services behind well-defined contracts to enable independent scaling of compute-intensive modules, and the medicine recommendation platform 1500 can maintain a unified health-data schema that normalizes EMR, drug, and environmental entities with controlled vocabularies. Therefore, the medicine recommendation platform 1500 adapts quickly to local guideline nuances, preserves sub-second response under load, and enables semantic consistency needed to combine heterogeneous clinical and financial data.

The analysis engine 1532 can perform aggregate multi-criteria medication scoring by normalizing efficacy, resistance, interaction risk, organ-adjusted dosing suitability, availability, and cost metrics to a common scale and combining the metrics according to adjustable weight vectors. In one example, the analysis engine 1532 can apply rule-based logic and/or machine learning outputs as inputs to the aggregate computation to produce a quantitative suitability score per candidate drug. Therefore, the analysis engine 1532 enables direct comparison of therapeutics across clinical benefit, safety, and affordability dimensions required for comprehensive decision support.

The multi-criteria ranking engine 1542 can generate a patient-specific ranked medication list by sorting candidates on composite suitability and attaching dose ranges adjusted for renal and hepatic function, interaction flags, drug availability indicators, and insurance-adjusted cost estimates. In one example, the presentation layer 1544 can render the ranked list with visual summaries and packaging depictions to enable rapid clinician review and selection. Therefore, the multi-criteria ranking engine 1542 and the presentation layer 1544 deliver actionable choices that incorporate dose adjustment complexity and coverage constraints within clinician time limits as well as assist providers in confirming the correct medicine. This can reduce the likelihood of errors, such as confusing similar sounding medications since the system can use unique drug identifiers (e.g. NDC codes, and the like) to reduce the likelihood that the information for the drug is incorrect or inconsistent. The presentation layer 1544 can display the ranked choices with the information provided by the respective packaging and labels and can display visual images of the medications, e.g., a tablet, such as a red pill with a unique identifier “45TP” code on it, to enable healthcare providers to visually confirm the drug and dose they are going to administer matches the prescribed and/or recommended drug and dose, e.g., the selected drug and dose.

The data acquisition layer 1516 can ingest EMR, pharmacy, regulatory, formulary, pricing, and resistance feeds, e.g., from external resources 1508, normalize records into the unified schema, and timestamp events for freshness assessment. Additionally, the data storage layer 1524 can persist normalized datasets and caches for low-latency retrieval, the analysis engine 1532 can consume the persisted datasets to compute scores and rankings, the presentation layer 1544 can present recommendations and justifications to end users, and the integration and API layer 1552 can expose messaging interfaces for bidirectional exchange, e.g., see Pharmacist-Provider Communication Platform discussed herein. Therefore, the data acquisition layer 1516 and associated hierarchical components maintain continuous, compliant data flow required to surface real-time susceptibility, prevent or reduce contraindications, and align recommendations with cost and coverage realities.

### Data Acquisition Layer 1516

The data acquisition layer 1516 can orchestrate ingestion from a plurality of connectors, including a patient information collector 1518, a drug information collector 1520, and an environmental information collector 1522, via interfaces that support Health Level Seven (HL7), Fast Healthcare Interoperability Resources (FHIR), custom APIs, and the like. For example, external resources 1508, e.g., databases, that the data acquisition layer 1516, modules of the data acquisition layer 1516, and/or other modules described here can access can include CMS Blue Button 2.0 API, CMS Medicaid NADAC API, Medicare Part D Formulary Data, DaVinci PDex Formulary, openFDA/DailyMed, GoodRx API, FDB MedKnowledge, Medi-Span, NAVLIN, and the like. The data acquisition layer 1516 can be or can include a natural language processing model that can collect, receive, or retrieve data that is in text (e.g., in various formats, such as formats not including API or FHIR), process the text data, and convert the text data into a usable input for the system. For example, the data acquisition layer 1516 can analyze websites including authoritative data (such as a Stanford antibiogram), extract information, such as the data, and provide it to the system to guide therapy and/or prescriptions.

In one example, the data acquisition layer 1516 aggregates EMR data, laboratory results, pharmacy and insurance feeds, regulatory notifications, and epidemiological streams into a unified intake pipeline with configurable source adapters and back-pressure control. Additionally, the data acquisition layer 1516 can timestamp records at observation time and ingestion time and can expose a streaming and/or batch pathway to the data storage layer 1524 to maintain freshness guarantees for downstream analytics. Thus, the data acquisition layer 1516 supplies current clinical variables, availability and cost signals, and real-time resistance inputs, reducing manual review burden and enabling timely prescribing under physician time constraints.

An entity resolution subsystem can reconcile duplicate and conflicting representations of patients, practitioners, and drugs by applying deterministic identifier matches and probabilistic similarity scores and by assigning a surrogate key with linkage tables to preserve source traceability. More specifically, inline data validation and anomaly flagging can execute syntactic checks for presence and datatype conformance and semantic checks for physiologic plausibility, routing nonconforming records to an exception queue with machine-readable error codes. In one example, the entity resolution subsystem can emit resolved, validated entities to downstream normalization only when confidence thresholds exceed a configurable score range. Thus, the entity resolution subsystem and inline data validation and anomaly flagging reduce misattribution of medication histories and interactions, lowering adverse interaction risk in the recommendation workflow.

A schema mapping and normalization engine can transform heterogeneous source schemas (e.g., Logical Observation Identifiers Names and Codes (LOINC), Systemized Nomenclature of Medicine-Clinical Terms (SNOMED CT), RxNorm, a standardized naming system for generic and branded drugs, National Drug Code (NDC), proprietary codes, and the like) into a unified data model using table-driven mappings with unit harmonization and post-mapping type validation. In one example, real-time normalization and deduplication can execute during ingestion so that downstream components receive semantically consistent, non-redundant records aligned to a single patient or drug identity. Additionally, a temporal tagging mechanism can attach dual timestamps with millisecond precision for observation time and ingestion time to enable longitudinal reconstruction and freshness-aware models. Thus, the schema mapping and normalization engine, real-time normalization and deduplication, and the temporal tagging mechanism provide accurate organ function parameters and time-aligned labs for dosage calculations while preventing analytic skew from duplicates.

The patient information collector 1518 can ingest structured and unstructured patient data via an EMR interface 1602, a provider input interface 1604, a patient portal input, and the like, e.g., in FHIR or other available formats, and can align demographics, vitals, laboratory values, allergy histories, active medications, comorbidities, and payer data to a unified patient context. More specifically, the patient information collector 1518 can interoperate with the drug information collector 1520 to reconcile medication identifiers (e.g., RxNorm, NDC, and the like), to associate formulary inclusion and availability with active and historical regimens, and to request refreshes when coverage or pricing changes are detected. Additionally, the patient information collector 1518 can exchange location and encounter metadata with the environmental information collector 1522 so that epidemiological prevalence and resistance signals are bound to patient encounters and used to annotate suspected syndromes. Thus, the patient information collector 1518, in cooperation with the drug information collector 1520 and the environmental information collector 1522, supplies organ-function parameters for dose adjustment, integrates coverage and cost with medication history, incorporates locality-specific resistance data at prescribing time, and reduces clinician review workload while lowering interaction and contraindication risk.

### Data Acquisition Layer 1516—Patient Information Collector 1518

The patient information collector 1518 can acquire, aggregate, and structure patient-specific clinical, demographic, medication, and payer data (e.g., patient insurance information, such as patient insurance tier, payor, or formulary information) for use by the data acquisition layer 1516. In one example, the patient information collector 1518 executes extraction of patient-specific clinical parameters and extraction of patient financial and medication history to generate a patient-specific data package. Additionally, the patient information collector 1518 can ingest structured and unstructured records via standardized healthcare APIs (e.g., HL7, FHIR, and the like) and/or direct user inputs to support near-real-time synchronization. Alternatively, in an API-centric implementation, the patient information collector 1518 can expose Representational State Transfer (REST) endpoints that stream incremental changes with event metadata and timestamps to enable temporal alignment downstream.

The patient information collector 1518 can include a repository that stores machine-readable rules, which can be referred to herein as the machine readable instructions 1514, expressing physiologic plausibility and regulatory constraints in an interpretable language. In one example, the patient information collector 1518 normalizes and maps heterogeneous inputs into a unified schema by applying unit conversions (e.g., mg/dL to μmol/L), coding translation (e.g., local codes to LOINC/SNOMED/RxNorm), and text parsing for semi-structured notes. Additionally, the patient information collector 1518 can evaluate every element against the stored rules and divert failed elements into an isolated quarantine queue with machine-readable error codes and provenance. In one example, the patient information collector 1518 hot-reloads updated rule artifacts and then reprocesses quarantined elements to clear recoverable failures without halting ingestion.

The patient information collector 1518 can include an EMR interface 1602 that retrieves diagnoses, laboratory results, vital signs, allergies, and medication lists via HL7, FHIR, and the like, e.g., from external resources 1508. In one example, the patient information collector 1518 can include a provider input interface 1604 that accepts structured fields and natural-language entries with real-time prompts to capture missing renal or hepatic parameters or other organ functions tests that impact drug function (e.g., coagulation studies or labs) or heart function studies. Additionally, the patient information collector 1518 can include a patient portal input that receives patient-reported medications, adherence updates, and device-captured metrics, and then synchronizes mapped entries to the unified schema. Alternatively, the patient information collector 1518 can configure each interface to operate in a bidirectional mode that returns validated updates to the originating system and records audit trails for traceability.

### Patient Information Collector 1518—EMR Interface 1602

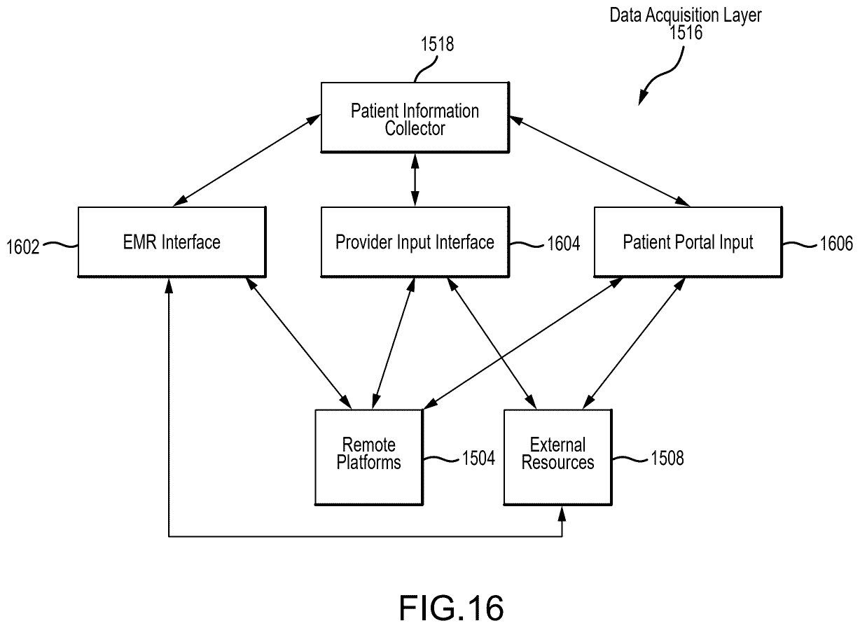

As depicted in FIG. 16 among others, the EMR interface 1602 can establish bidirectional data exchange between the patient information collector 1518 and remote platforms 1504 and/or external electronic medical record systems (e.g., the external resources 1508) via standardized interoperability protocols. More specifically, the EMR interface 1602 can consume HL7 messages and/or FHIR APIs, authenticate (e.g., via OAuth 2.0 and/or mutual Transport Layer Security (TLS)), and map incoming codes such as LOINC, SNOMED CT, and RxNorm into an internal data model for demographics, diagnoses, medication history, laboratory results, vital signs, and documented allergies. In one example, the EMR interface 1602 can apply configurable filters to prioritize elements relevant to medication selection, including organ function measures (e.g. renal, hepatic, coagulation or any other relevant organ function test/lab), recent microbiology results, and current medication regimens, and can operate as a microservice and/or middleware within the data acquisition layer 1516. Therefore, the EMR interface 1602 enables comprehensive and structured patient data ingestion that supports organ-adjusted dosing and interaction screening, thereby reducing risks associated with incomplete data during medication selection.

The medicine recommendation platform 1500, e.g., the EMR interface 1602, can access other EMRs from different vendors, which do not typically communicate with each other and silo information, and for numerous patients. For example, the EMR interface 1602 can scour through the EMR of or included in other platforms and acquire data from multiple EMR. In this way, the data from all EMR can be accessible to a user, e.g., a physicican, to use, e.g., for physician order entry (POE). The EMR interface 1602 (or other modules described herein) can time stamp changes, summarize the changes, and present the summary to a user. In some cases, this information is incomplete, however, with the vast amount of information pulled and summarized, patterns can be detected or otherwise observed. For example, the EMR interface 1602 can be referred to herein as an auditor or audit analyzer. The audit analyzer can audit the history of medication usage and changes. By auditing the EMR or patient record from all available EMR and sources (e.g., and each hospital may have a different vendor) this reduces the likelihood of losing information, e.g., when a patient travels to another state. The audit analyzer can then enable the provider to more quickly (as compared to having to attempt to access each separate EMR) ascertain the changes and cross reference the changes with relevant data in the medical record from similar dates.

The EMR interface 1602 can execute automated retrieval of patient-specific data by subscribing to EMR event streams and/or invoking scheduled queries keyed to a patient identifier. More specifically, the EMR interface 1602 can detect freshness thresholds for parameters such as serum creatinine, bilirubin, and platelet count, and can trigger re-queries or differential fetches using versioned timestamps and/or ETags to maintain continuous data freshness assurance without duplicating records. In one example, the EMR interface 1602 can write refreshed elements to the patient profile repository 1526, invalidate entries in a caching store 1530, and signal the analysis engine 1532 to trigger recalculation on threshold change for downstream dose calculation and interaction evaluation. Thus, the automated retrieval and freshness mechanisms reduce physician workload, maintain up-to-date clinical context for dosing and contraindication checks, and address the need for timely data at the point of prescribing.

### Patient Information Collector 1518—Provider Input Interface 1604

As depicted in FIG. 16 among others, the provider input interface 1604 can establish bidirectional data exchange between the patient information collector 1518 and remote platforms 1504 and/or external resources 1508. For example, the provider input interface 1604 can present clinician-facing modules that receive patient-specific observations and orders at the point of care, e.g., via the remote platforms 1504 and/or the external resources 1508. More specifically, the provider input interface 1604 can render graphical forms with structured fields, accept natural-language dictation via voice-to-text, and parse free-text into coded elements relevant to renal function, hepatic function, allergy status, and current regimens, e.g., via or to the remote platforms 1504. Additionally, the provider input interface 1604 can enforce user authentication, attach time-stamps and audit trails to each entry, and transmit validated entries to the patient information collector 1518 for inclusion in a patient-specific data package. In one example, the provider input interface 1604 integrates with an EMR container and/or external laboratory information systems, e.g., the external resources 1508, to auto-populate context-sensitive fields and reduce manual transcription, while supporting web and mobile delivery variants.

The provider input interface 1604 can include a multi-modal data entry pane that hosts graphical controls, a free-text zone, and an audio capture channel linked to a speech-to-text engine under a common metadata schema. More specifically, the multi-modal data entry pane can route each selection, narrative, and dictation through a shared data bus that assigns synchronized provenance tags and standardized codes for downstream harmonization. Additionally or alternatively, a validation layer of the multi-modal data entry pane can enforce data completeness and consistency by scanning entries in real time, prompting for missing renal (or other organ function) metrics or medication histories, and guiding reconciliation of conflicting allergy statements. Thus, the multi-modal data entry pane can reduce data quality alerts and reduce downstream overrides while preserving clinician workflow continuity.

### Patient Information Collector 1518—Patient Portal Input 1606

As depicted in FIG. 16 among others, the patient portal input 1606 can establish bidirectional data exchange between the patient information collector 1518 and remote platforms 1504 and/or external resources 1508. For example, the patient portal input 1606 can provide a mobile and web interface configured to receive structured entries for medications, supplements, allergies, adherence updates, and self-monitored metrics, e.g., via the remote platforms 1504 and/or the external resources 1508. In one example, the patient portal input 1606 can transmit patient-entered updates in real time, e.g., received from the remote platform(s) 1504, to the data acquisition layer 1516 and synchronize records via the patient information collector 1518 and the EMR interface 1602. Additionally, the patient portal input 1606 can present guided questionnaires and pictorial aids to improve accuracy for names, strengths, and schedules, and can issue clarification prompts aligned to detect missing/conflicting data and request update. In one embodiment, the patient portal input 1606 can authenticate users through the security and compliance module to protect regulated data during capture and transmission.

The patient portal input 1606 can include an adaptive questionnaire generator that assembles context-specific forms from declarative decision trees (e.g., JavaScript Object Notation (JSON) based rules) driven by problem lists, demographics, and prior submissions. More specifically, the adaptive questionnaire generator can update questions and branching logic client-side without page reloads to reduce burden while collecting clinically relevant variables. Additionally, the patient portal input 1606 can embed an inline data validation engine that enforces syntactic, cross-field, and EMR-referenced consistency checks. In particular, the inline data validation engine can perform real-time data quality assurance by producing visual cues and blocking erroneous submissions before the data acquisition layer 1516 consumes the records. The patient portal input 1606 (or other modules described herein) can differentiate or filter out subjective data that can be unreliable, e.g., a patient may enter inaccurate data related to symptoms or characteristics (e.g., a patient may enter a weight significantly less than a weight entered by a healthcare provider during an exam). The patient portal input 1606 (or other modules described herein) can determine objective data (e.g. factual blood tests values). In some examples, the patient portal input 1606 can assign data determined to be objective a higher weight than data determined to be subjective. For example, the patient portal input 1606 (or other modules described herein) can prioritize the objective data over subjective data.

The patient portal input 1606 can capture patient-reported medication regimen entries for prescription drugs, over-the-counter agents, and supplements with strength, dose, route, and administration schedule, e.g., via the external resources 1508. In one example, the patient portal input 1606 can normalize captured products to RxNorm identifiers and apply timestamps to enable longitudinal comparison. Additionally, the patient portal input 1606 can flag duplications and allergy conflicts and can request corrections prior to packaging the data for the patient-specific data package. Thus, the patient portal input 1606 can supply complete and standardized regimen data to downstream contraindication checking, dose calculation, and ranking operations.

### Data Acquisition Layer 1516—Drug Information Collector 1520

The drug information collector 1520 can retrieve candidate drug information from external drug data repositories, e.g., external resources 1508 such as the FDA, the respective drug manufacturer, and the like, and can standardize drug attributes for downstream consumption. More specifically, the drug information collector 1520 can aggregate therapeutic indications, dosing and organ-adjustment tables, contraindications and interactions, availability signals, and dynamic pricing and coverage variables into a current drug knowledge dataset. In one example, the drug information collector 1520 can publish updates to the drug knowledge base 1528 and the caching store 1530 to supply low-latency reads to the analysis engine 1532, and then display the results to a provider or user via the presentation layer 1544. Further, the drug information collector 1520 can retrieve medications to recommend by patient indication, such as hypertension, sinus infection, and the like, based on approved FDA indications. Thus, the drug information collector 1520 addresses physician time constraints and the inability to integrate up-to-date efficacy, availability, and cost information into prescribing decisions.

A conflict resolution engine of the drug information collector 1520 can compute canonical values using weighted provenance, recency checks, and consensus rules. Additionally or alternatively, an event-driven refresh scheduler can trigger ingestion on intervals and/or webhooks and can adapt polling rates based on observed volatility. In one example, an integrity assurance and alerting function can generate structured warnings and confidence scores for records that exceed conflict thresholds and can propagate alerts to the integration and API layer 1552. Thus, the conflict resolution engine improves data reliability and prevents or reduces propagation of erroneous guidance, reducing risk of adverse interactions and supporting safe, timely recommendations.

A schema-agnostic normalization pipeline of the drug information collector 1520 can parse heterogeneous payloads, apply semantic tagging, harmonize units, and map entities to standardized identifiers such as RxNorm. In one example, the pipeline can emit columnar buffers for high-throughput processing and can accept rule updates without redeployment to accommodate evolving FHIR, HL7, and unstructured label formats. Thus, the schema-agnostic normalization pipeline enables reliable cross-source comparison required for organ-adjusted dosing, interaction evaluation, and multi-criteria ranking without manual clinician reconciliation.

The drug information collector 1520 can aggregate heterogeneous drug data by orchestrating concurrent adapters that merge regulatory, formulary, inventory, clinical efficacy, and public health feeds into a single query store. For example, in the case of antibiotics, efficacy can be collected or determined by analyzing National Institutes of Health (NIH), CDC, WHO, and hospital databases such as antibiograms, FHIR data, EMR data or any data related to the patient, e.g., cultures and sensitivities. For example, in the case of other drugs or medications, efficacy can be collected or determined by analyzing trends from other patients with the same condition. For example, this can be collected by accessing and analyzing data available in other EMR and comparing the most up-to-date data being entered into the EMR (e.g., blood pressure trends for medicine A versus medicine B). In other examples, this can be collected by accessing the scientific studies and summarizing them for the prescriber, pharmacist, or healthcare provider.

More specifically, a dynamic pricing consolidation function can normalize currencies, dosage frequencies, and administration overhead to compute per-course cost vectors, and a real-time resistance trend injection function can attach temporally scoped susceptibility metrics to relevant antibiotics. Thus, aggregating heterogeneous drug data enables comparison of effectiveness against patient insurance coverage and local prices while incorporating current resistance patterns for improved antibiotic selection.

The data acquisition layer 1516 can host the drug information collector 1520 and can coordinate retrieve candidate drug information and standardize drug attributes alongside patient and environmental collection pipelines, e.g., via remote platforms 1504 and/or external resources 1508. In one example, the data acquisition layer 1516 can manage secure connectivity, batching or streaming modes, and timestamping to preserve freshness guarantees for downstream scoring. Thus, the data acquisition layer 1516 provides scalable, continuously refreshed pipelines that alleviate physician time constraints by automating data collection and preparation.

The drug information collector 1520 can include a regulatory data interface that acquires approved indications, warnings, and dosing guidance from regulatory repositories and maps the guidance to standardized codes. Additionally or alternatively, a hospital/pharmacy database connector 1704 can retrieve formulary status, stock levels, and contract pricing, and a real-time resistance data connector 1706 can ingest antibiogram and culture-derived susceptibility updates with minute-level latency. In one example, the drug information collector 1520 can merge these feeds to reflect actionable dose adjustments, availability constraints, and local resistance dynamics in the current drug knowledge dataset. Thus, the regulatory data interface and associated connectors address dose adjustment complexity, availability and cost integration, and the lack of real-time resistance data at the point of prescribing.

### Drug Information Collector 1520—Regulatory Data Interface 1702

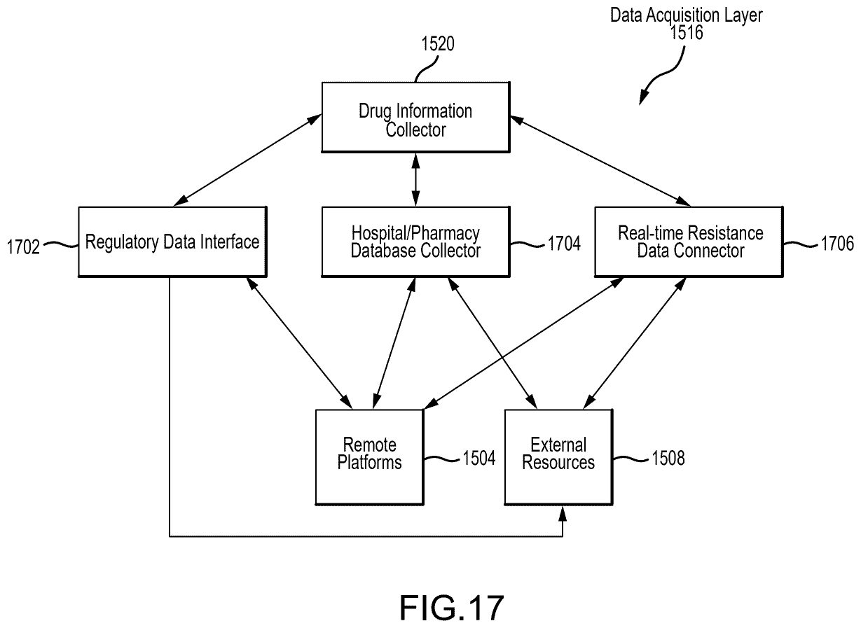

As depicted in FIG. 17 among others, the regulatory data interface 1702 can establish bidirectional data exchange between the drug information collector 1520 and remote platforms 1504 and/or external resources 1508. For example, the regulatory data interface 1702 can acquire, parse, and structure regulatory drug information from external resources 1508, such as authoritative repositories, such as FDA labeling databases, manufacturer instructions for use (which can often include hundreds of pages of information), structured approval documents, and electronic drug labels. In one example, the regulatory data interface 1702 retrieves safety statements, approved indications, dosing guidance, contraindications, boxed warnings, drug specific dose adjustment equations and instructions, and pharmacokinetic summaries via APIs and/or controlled scraping, and encodes the results into a machine-readable interchange format (e.g., JSON or Extensible Markup Language (XML)). Additionally, the regulatory data interface 1702 can persist versioned records and propagate updates to the drug knowledge base 1528 for immediate availability to the dose calculation engine 1534 and the interaction and contraindication checker 1536. Therefore, the regulatory data interface 1702 supplies current labeling constraints that reduce physician time burden and lower the risk of contraindicated therapy due to outdated guidance.

The regulatory data interface 1702 can include an identifier cross-reference table that links NDC, RxNorm Concept Unique Identifier (RXCUI), Unique Ingredient Identifier (UNII), marketing authorization numbers, the like, and the equivalents in other countries or regions of the world, to an internal master drug key. In one example, an incremental update scheduler retrieves delta records and synchronises real-time updates when source repositories publish new label files, while checksum validation protects data integrity before commit. More specifically, a schema normalisation engine can transform heterogeneous payloads into a versioned intermediate representation with canonical field mappings and controlled vocabulary resolution, and a regulatory discrepancy flagger can annotate conflicts with provenance, severity, and timestamps for governance review. Thus, the identifier cross-reference table and associated update, normalisation, and flagging features enable unambiguous data fusion and prevent or reduce unvetted label changes from propagating into recommendations.

The regulatory data interface 1702 can acquire authoritative regulatory data by establishing secure connections to governmental and quasi-governmental repositories and by ingesting structured and unstructured records according to source capabilities. In one example, the regulatory data interface 1702 can generate compliance alerts when newly acquired information diverges from stored guidance, and can route alert payloads to remote platforms 1504, the security and compliance module, or the presentation layer 1544, e.g., for acknowledgement and audit by a provider or a user. Additionally, the regulatory data interface 1702 can log acquisition provenance and effective dates to support policy checks within the analysis engine 1532. Therefore, the acquisition and alert generation workflow improves visibility into label amendments and supports safe prescribing without manual surveillance by clinicians.

The regulatory data interface 1702 can map data to unified drug identifiers by reconciling source-specific product codes to the platform master drug key for each pharmaceutical entity. In one example, the regulatory data interface 1702 can parse and canonicalise heterogeneous records (e.g., XML, JSON, Portable Document Format (PDF), and text) to extract dosing tables, boxed warnings, and pharmacokinetic parameters into an ontology-aligned schema consumed by the drug knowledge base 1528. Additionally, the regulatory data interface 1702 can preserve bidirectional mappings so downstream components can reference original source citations during traceability. Thus, the mapping and canonicalization pipeline enables consistent linkage between regulatory constraints and patient-specific analytics, enabling accurate dose adjustment and interaction evaluation.

### Drug Information Collector 1520—Hospital/Pharmacy Database Connector 1704

As depicted in FIG. 17 among others, the hospital/pharmacy database connector 1704 can establish bidirectional data exchange between the drug information collector 1520 and remote platforms 1504 and/or external resources 1508. For example, the hospital/pharmacy database connector 1704 can establish secure, automated links to formulary systems, inventory modules, and supply-chain databases via FHIR, HL7, and/or site-specific APIs, and the like, and can support bidirectional polling and event subscriptions. In one example, the hospital/pharmacy database connector 1704 aggregates stock levels, formulary status, contract pricing, and procurement constraints from multiple facilities and group-purchasing networks, and streams normalized payloads to the drug information collector 1520 and the drug knowledge base 1528. Additionally, the hospital/pharmacy database connector 1704 can publish update events to the integration and API layer 1552 for downstream consumers in the analysis engine 1532. Thus, the hospital/pharmacy database connector 1704 enables the medicine recommendation platform 1500 to factor real-time availability and cost into recommendations, addressing the inability of providers to integrate dynamic availability and price data under time constraints.

A schema mapping and normalization engine within the hospital/pharmacy database connector 1704 can reconcile identifiers such as NDC, RxNorm, formulary codes, and internal Stock Keeping Unit (SKU) values into a canonical schema aligned with the drug knowledge base 1528. More specifically, the schema mapping and normalization engine can execute rule-based mappings with site-level overrides, convert units across packaging and dose forms, append lineage metadata, and publish a normalized stream for concurrent microservice consumption as data normalization and publishing. Thus, the schema mapping and normalization engine eliminates semantic mismatches and unit inconsistencies that would otherwise corrupt downstream dose, availability, and cost computations, enabling reliable automated analysis without manual reconciliation.

An availability-driven recommendation updating function of the hospital/pharmacy database connector 1704 can emit events on stockouts, backorder changes, and price shifts to trigger re-ranking within the multi-criteria ranking engine 1542. In one example, a contract pricing acquisition process retrieves contract-specific costs, discount tiers, and fees, computes a standardized cost-per-dose, and forwards the result to the cost and coverage analyzer 1538. Additionally, a real-time inventory retrieval process can capture on-hand quantities, reorder thresholds, and restock dates, and can stream the results to the drug knowledge base 1528 within seconds. Thus, the availability-driven recommendation updating function keeps recommendations synchronized with real-world supply and economics, reducing canceled orders and enabling timely, cost-aware selections.

### Drug Information Collector 1520—Real-Time Resistance Data Connector 1706

The real-time resistance data connector 1706 can be referred to herein as a real-time sensitivity and resistance data connector. As depicted in FIG. 17 among others, the real-time resistance data connector 1706 can establish bidirectional data exchange between the drug information collector 1520 and remote platforms 1504 and/or external resources 1508. For example, the real-time resistance data connector 1706 can establish and maintain electronic connections to external antibiogram feeds, machine-readable microbiology culture report streams, and the like, e.g., using subscription callbacks and/or periodic polling to retrieve updates within minutes of publication. More specifically, the real-time resistance data connector 1706 can parse HL7, FHIR, and XML/JSON messages to extract minimum inhibitory concentrations, percent susceptibility, and organism-antibiotic resistance trends. In one example, the real-time resistance data connector 1706 feeds normalized real-time data update to the analysis engine 1532 to support trigger recalculation on threshold change for affected recommendations. Thus, the real-time resistance data connector 1706 addresses the lack of real-time antibiotic susceptibility data at the point of prescribing by making current resistance evidence readily available to downstream evaluators. Thus, the real-time resistance data connector 1706 can collect drug efficacy information from various sources including Center for Disease Control data, World Health Organization data, Hospital microbiology lab data, FHIR, patient specific data (e.g., a particular patient's cultures revealing personal susceptibilities), and the like.

A configurable alert generation module can monitor aggregated susceptibility metrics for rule-defined threshold changes (e.g., a 5-20% drop in susceptibility for a pathogen-drug pair or the emergence of pan-resistant isolates) and emit structured alert objects over a messaging bus and/or annotate entries in the summary dashboard 1546. More specifically, an integrity validation and quarantine subsystem can execute checksum verification, pharmacological range checks for Minimum Inhibitory Concentration (MIC) values, and cross-field consistency logic, diverting failed records to a quarantine queue with audit metadata for remediation. In one example, an administrative console can allow institutions to edit organism scope, antibiotic lists, change magnitudes, and notification channels. Therefore, the configurable alert generation module reduces physician time burden while ensuring trustworthy inputs by filtering corrupted data and proactively signaling clinically meaningful shifts.

A resistance profile aggregation store can maintain rolling aggregates such as 7-90 day percent susceptibility, MIC distributions, and trend slopes, keyed by geographic region, institution, and specimen source for microsecond-range query performance. More specifically, the acquisition of external susceptibility data can occur through live sessions with antibiogram services and laboratory information systems that deliver newly published culture results via subscription or schedule. In one example, event-based stewardship notification can publish standardized events to stewardship dashboards and optional mobile endpoints when statistical tests detect significant regime changes. Thus, the resistance profile aggregation store enables rapid retrieval of context-appropriate resistance profiles, supporting integration of up-to-date efficacy evidence without manual reconciliation.

A schema mapping and normalization engine can map organism names, antibiotic codes, and susceptibility metrics from heterogeneous vocabularies such as LOINC, SNOMED CT, and local taxonomies into a canonical schema aligned with the drug knowledge base 1528. More specifically, the engine can perform real-time normalization and aggregation of metrics by applying regex-based field cleaning and computing moving-average MIC values and percent-susceptible statistics as records arrive. In one example, the engine can provide a single authoritative resistance profile to the efficacy and resistance evaluator 1540 and the interaction and contraindication checker 1536. Therefore, the schema mapping and normalization engine mitigates errors from heterogeneous feeds and supports timely recalculation with structurally consistent inputs.

### Data Acquisition Layer 1516—Environmental Information Collector 1522

The environmental information collector 1522 can acquire, aggregate, and process epidemiological and environmental datasets relevant to a specific patient, e.g., the patient's general or habitual location or a patient-specific encounter. In one example, the environmental information collector 1522 retrieves local and regional pathogen prevalence, outbreak alerts, seasonal trend indicators, and antimicrobial resistance statistics from external resources 1508, such as public-health repositories and/or hospital antibiograms. More specifically, the environmental information collector 1522 can operate in real-time or near-real-time with a refresh cadence (e.g., between 5 and 60 minutes) to provide updated inputs for retrieve contextual epidemiological data within the data acquisition workflow. Thus, the environmental information collector 1522 generates structured records that downstream modules can join with patient and drug datasets to influence or assist in medication selection.

A geolocation context engine can resolve patient and/or facility coordinates into standardized geographic identifiers and constrain data retrieval to relevant catchment areas. In one example, the geolocation context engine executes acquire multisource epidemiological data by orchestrating asynchronous connectors to public-health feeds, laboratory surveillance systems, and third-party networks while reconciling heterogeneous update cadences. More specifically, the geolocation context engine can contextualize data to patient location by filtering, weighting, and/or discarding records based on postal code, county code, or facility ward to align resistance and prevalence statistics with the point of care. Thus, the geolocation context engine reduces irrelevant signals and enhances clinical relevance for subsequent efficacy calculations.

The on-device machine-learning inference core can execute pre-trained statistical or machine-learning models to transform environmental features into actionable predictions. In one example, the on-device machine-learning inference core can predict likely causative organisms for a specified clinical syndrome using feature vectors that include temporal prevalence trends, seasonal factors, and demographic covariates. More specifically, the on-device machine-learning inference core can provide near-real-time refresh and alerting, e.g. the remote platforms 1504, by monitoring data freshness thresholds and emitting notifications through the integration layer when staleness exceeds a limit (e.g., between 15 and 180 minutes), while continuing local inference using cached parameters. Thus, the on-device machine-learning inference core supplies probability distributions that the efficacy and resistance evaluator 1540 can consume to privilege, e.g., rank higher, medications with higher predicted coverage.

The environmental information collector 1522 can normalize and standardize data schema by mapping raw feed fields to controlled vocabularies, attaching canonical timestamps, and resolving coding systems used for organisms and locations. In one example, the environmental information collector 1522 converts heterogeneous inputs into a shared JSON structure with consistent units, code systems, and provenance metadata, such as JSON and the like. More specifically, the environmental information collector 1522 can validate record completeness and generate transformation logs to support auditing and traceability for downstream analytics. Thus, the environmental information collector 1522 delivers uniform datasets that reduce schema mismatches during joins with patient and drug information.

The data acquisition layer 1516 can host the environmental information collector 1522 as a peer component to the patient information collector 1518 and the drug information collector 1520. In one example, the data acquisition layer 1516 routes retrieve contextual epidemiological data requests to the environmental information collector 1522 and merges the returned datasets with concurrent patient and drug inputs. More specifically, the data acquisition layer 1516 can apply checkpointing and idempotent writes so the environmental information collector 1522 achieves reliable ingestion across network interruptions. Thus, the data acquisition layer 1516 provides coordinated intake and prepares comprehensive inputs for downstream suitability scoring and ranking.

### Data Storage Layer 1524

The data storage layer 1524 can persist patient-specific and drug-related datasets in secure, query-optimized repositories and/or high-speed caches. In one example, the data storage layer 1524 can maintain normalized structures for demographics, laboratory results, medication histories, coverage data, efficacy metrics, cost data, and real-time antibiogram information, while enforcing versioning and audit trails for longitudinal analysis. Additionally, the data storage layer 1524 can employ encryption in transit and at rest, access control, and data partitioning, and can expose retrieval interfaces that support batch and real-time query patterns for upstream analytics. Alternatively, the data storage layer 1524 can utilize relational, non-relational (NoSQL), and/or hybrid models or databases to optimize diverse access patterns and can generate data access logs to support traceability.

The data storage layer 1524 can guarantee atomic consistency for concurrent access by wrapping write operations within a distributed commit protocol and by applying a transaction isolation policy that prevents or reduces observation of partial updates. In one example, the data storage layer 1524 can apply deadlock detection and lock-free read strategies to sustain high-throughput clinical workloads while preserving integrity of concurrent recommendation queries. Additionally, the data storage layer 1524 can persist heterogeneous clinical and drug data by validating schema conformance, assigning global identifiers, and triggering encryption during ingestion of normalized payloads from HL7 laboratory messages, insurance Electronic Data Interchange (EDI) files, and antibiogram feeds. Consequently, the data storage layer 1524 can abstract storage heterogeneity and present a unified, query-ready surface to analytic components.

The patient profile repository 1526 can store longitudinal patient records with time-stamped demographics, laboratory values, allergies, comorbidities, medication histories, and insurance coverage, and can expose low-latency queries that supply variables for dosing, interaction screening, and coverage verification. In one example, the data storage layer 1524 can further include a drug knowledge base 1528 that aggregates regulatory labeling, formulary and pharmacy data, clinical studies, interaction and contraindication relationships, efficacy and resistance metrics, and price and availability feeds for real-time and scheduled updates. Additionally or alternatively, the data storage layer 1524 can include a caching store 1530 that retains frequently accessed profiles, resistance snapshots, and cost and coverage fragments with configurable eviction (e.g., time-to-live ranges between minutes and hours) to achieve sub-second read latency. Thus, the data storage layer 1524 can organize the patient profile repository 1526, the drug knowledge base 1528, and the caching store 1530 in a hierarchical arrangement that supports rapid retrieval for the analysis engine 1532 and presentation workflows.

### Data Storage Layer 1524—Patient Profile Repository 1526

The patient profile repository 1526 can maintain longitudinal patient records within the data storage layer 1524, using a relational schema and/or a document model to support rapid retrieval and update. In one example, the patient profile repository 1526 aggregates demographics, laboratory results, medication histories, allergies, comorbidities, coverage details, and clinical measurements received through the EMR interface 1602, the provider input interface 1604, and the patient portal input 1606 using standardized exchange protocols such as HL7, FHIR, and the like. More specifically, the patient profile repository 1526 can persist versioned entries with time stamps and provenance tags to enable retrospective analysis across a time horizon (e.g., across weeks to years). Additionally, the patient profile repository 1526 can enforce access control, audit logging, and encryption in coordination with the security and compliance module to support regulated operation.

The patient profile repository 1526 can aggregate patient data streams by ingesting heterogeneous records and normalizing coding systems such as LOINC, SNOMED, and/or RxNorm into internal reference tables. In one example, the patient profile repository 1526 consolidates multi-source inputs into a canonical patient record that the analysis engine 1532 and the dose calculation engine 1534 can query with low-latency access paths. More specifically, the patient profile repository 1526 can return most-recent validated values with associated provenance-derived confidence intervals and measurement timestamps to support dose calculation requests. Thus, the patient profile repository 1526 can reduce reconciliation errors and provide consistent inputs for downstream computations.

The patient profile repository 1526 can expose structured allergy entries, comorbidity codes, and concurrent medication lists through parameterized queries and/or stored procedures. In one example, the interaction and contraindication checker 1536 invokes the patient profile repository 1526 to retrieve patient-specific risk factors and to cross-reference candidate drugs defined by the drug knowledge base 1528. More specifically, the patient profile repository 1526 can supply temporally scoped lists and problem codes aligned to standardized vocabularies to enable deterministic screening. Thus, the patient profile repository 1526 can provide pre-order flags that support safe recommendation generation by the medicine recommendation platform 1500.

### Data Storage Layer 1524—Drug Knowledge Base 1528

The drug knowledge base 1528 can store a graph-structured repository of drug entities with relationships encoding mechanisms of action, pharmacokinetics, pharmacodynamics, contraindications, interactions, and adverse effects. In one example, the data acquisition layer 1516 can populate the drug knowledge base 1528 using the regulatory data interface 1702, the hospital/pharmacy database connector 1704, and the real-time resistance data connector 1706 to generate a current drug knowledge dataset. More specifically, the data harmonization and preprocessing pipeline can write a standardized drug attribute set and versioned edge properties into the drug knowledge base 1528, e.g., a dataset connected to or accessible by the drug knowledge base 1528, for consumption by the analysis engine 1532. In one embodiment, the drug knowledge base 1528 can expose query endpoints that retrieve organ- and age-stratified dosing guidance and/or availability and coverage attributes to support downstream suitability scoring and ranking.

The drug knowledge base 1528 can execute a drug interaction graph traversal that returns a subgraph of contraindication, synergistic, and adverse-reaction paths with severity and mechanism annotations. In one example, the interaction and contraindication checker 1536 can invoke the traversal and consume the returned subgraph without additional parsing. Additionally or alternatively, the drug knowledge base 1528 can perform patient-contextual query resolution by evaluating constraints such as Estimated Glomerular Filtration Rate (eGFR) ranges, Crockcroft-Gault equation, hepatic scores, allergy lists, and formulary tiers inside the graph engine. In one embodiment, the drug knowledge base 1528 can return a pre-screened candidate set in millisecond-scale latency, thereby reducing computation in the analysis engine 1532.

The drug knowledge base 1528 can co-locate, e.g., via the external resources 1508, pricing, coverage tiers, prior-authorization flags, availability, and administration resource attributes with pharmacologic properties to support economic impact computation support. In one example, the cost and coverage analyzer 1538 can query the drug knowledge base 1528 to join therapeutic equivalence with patient-specific cost and coverage data and institutional formulary rules. More specifically, the drug knowledge base 1528 can return per-drug out-of-pocket estimates and projected institutional cost ranges (e.g., per course or per day) to enable side-by-side comparisons. Alternatively, the drug knowledge base 1528 can surface clinically comparable substitutes that reduce total cost while satisfying safety and efficacy constraints.

The drug knowledge base 1528 can ingest antibiogram updates and epidemiological data feeds to support real-time resistance trend provisioning. In one example, the drug knowledge base 1528 can compute rolling susceptibility aggregates and temporal slopes on drug-pathogen edges upon receipt of a normalized real-time data update from the real-time resistance data connector 1706. More specifically, the efficacy and resistance evaluator 1540 can request current susceptibility percentages and trend directions to adjust efficacy contributions during suitability scoring. Then, the analysis engine 1532 can trigger recalculation on threshold change to propagate an updated harmonized dataset for subsequent ranking and presentation.

### Data Storage Layer 1524—Caching Store 1530

The caching store 1530 can retain frequently accessed datasets relevant to medication recommendation, including real-time resistance profiles, drug cost data, and insurance coverage mappings, in volatile memory for rapid access and/or non-volatile storage for persistence. More specifically, the caching store 1530 can ingest asynchronous updates from the data acquisition layer 1516 and the integration and API layer 1552, apply versioning based on source timestamps, and maintain configurable eviction policies such as least recently used and time-to-live with exemplary durations (e.g., 30 to 3600 seconds). In one example, the caching store 1530 can distribute shards across multiple nodes with an exemplary replication factor (e.g., 2 to 5) to provide high availability and horizontal scalability in large healthcare environments. Additionally, the caching store 1530 can expose an API that the analysis engine 1532, the presentation layer 1544, and the integration and API layer 1552 can query and/or update within the medication recommendation workflow.

The caching store 1530 can reduce data retrieval latency by serving the first lookup for efficacy, cost, and coverage datasets directly from Random Access Memory (RAM), thereby avoiding network calls to external repositories and lowering average end-to-end response time to an exemplary sub-second range (e.g., 100 to 800 ms). More specifically, the caching store 1530 can pre-warm entries based on active patient context and recent query patterns, and can invalidate or refresh entries upon signals from continuous data refresh and/or trigger recalculation on threshold change. In one example, the caching store 1530 can return partial results while background refresh completes, thereby enabling the analysis engine 1532 to commence scoring without waiting on remote systems.

### Analysis Engine 1532

The analysis engine 1532 can process heterogeneous clinical, epidemiological, and financial datasets to generate patient-specific medication recommendations. More specifically, the analysis engine 1532 can receive EMR-derived parameters, laboratory values, demographics, medication history, organ function metrics, real-time resistance and availability data, and insurance coverage, then execute rule-based logic and/or machine-learning models to compute discrete metrics per candidate drug. Additionally, the analysis engine 1532 can aggregate the metrics into composite scores and can expose outputs via an API and/or a clinician-facing interface with real-time refresh. Therefore, the analysis engine 1532 addresses dose individualization, interaction risk mitigation, incorporation of up-to-date efficacy and cost data, and reduction of clinician review time.

The analysis engine 1532 can implement a modular algorithm pipeline architecture in which independently deployed modules execute efficacy scoring, interaction screening, cost analysis, and dose adjustment. More specifically, containerized modules can expose a common remote procedure interface and can exchange normalized payloads, allowing insertion, removal, or reordering without recompilation. In particular, an aggregation stage can aggregate multi-domain data by performing entity reconciliation and temporal alignment into a unified patient-context object for downstream modules. Therefore, the modular execution and coherent aggregation reduce inconsistency-driven errors and enable scalable throughput under clinical time constraints.

The analysis engine 1532 can load a pluggable scoring weight matrix from a version-controlled configuration to map evaluative metrics to numeric coefficients. More specifically, an administrative interface can hot-swap the matrix at runtime to support institution- or disease-specific tuning without altering executable code. In particular, the multi-criteria kernel can generate cost-adjusted utility scores by incorporating wholesale acquisition cost, coverage effects, copay estimates, administration effort, and monitoring expense into a normalized economic score. Therefore, configurable weighting and economic normalization enable comparison of therapeutic effectiveness against patient insurance coverage and out-of-network prices.

The analysis engine 1532 can calculate patient-specific efficacy scores by combining regional susceptibility data, pharmacodynamic targets, and patient organ function into a bounded probability-of-success value. More specifically, the analysis engine 1532 can detect drug interaction and contraindication risk by traversing a knowledge graph against the active medication list, comorbidities, and allergy profile, and can assign standardized severity grades that translate to inverse safety scores. Additionally, the analysis engine 1532 can feed the efficacy and safety scores into downstream ranking with traceable rationale. Therefore, data-driven efficacy estimation and automated risk detection address the lack of real-time resistance awareness and the risk of adverse interactions.

The analysis engine 1532 can produce a ranked medication list by applying the active weight matrix to per-drug metric vectors and by sorting on the resulting utilities. More specifically, the analysis engine 1532 can attach explanatory metadata for transparency and can meet interactive latency targets for point-of-care use. In particular, a live recommendation refresh can monitor laboratory updates, formulary changes, and user inputs, then can selectively recompute affected metrics and re-invoke the ranking module to publish updated results. Therefore, rapid ranking and continuous refresh alleviate physician time constraints while maintaining alignment with evolving clinical and market conditions.

The analysis engine 1532 can orchestrate a hierarchy that includes a dose calculation engine 1534, an interaction and contraindication checker 1536, a cost and coverage analyzer 1538, an efficacy and resistance evaluator 1540, and a multi-criteria ranking engine 1542. More specifically, the dose calculation engine 1534 can generate organ-adjusted dosing ranges that flow to composite scoring, the interaction and contraindication checker 1536 can produce risk profiles, the cost and coverage analyzer 1538 can compute patient-specific economic metrics, and the efficacy and resistance evaluator 1540 can output success probabilities. Additionally, the multi-criteria ranking engine 1542 can integrate all upstream outputs to compute utilities and to select a patient-specific recommendation set. Therefore, coordinated execution across the hierarchy provides dose adjustments and holistic trade-offs required for safe, effective, and economically appropriate prescribing.

### Analysis Engine 1532—Dose Calculation Engine 1534

The dose calculation engine 1534 can operate within the analysis engine 1532 to execute calculate organ-adjusted dosage range against the harmonized clinical and drug dataset and the drug knowledge base 1528. The dose calculation engine 1534 can ingest laboratory values, demographics, and clinical measurements from the patient profile repository 1526 and apply dosing protocols sourced from labels and/or institutional formularies. In one example, the dose calculation engine 1534 can compute medication-specific dose and interval outputs that reflect protocol constraints and patient parameters and can return drug-specific organ-adjusted dose guidance to downstream scoring. Therefore, the dose calculation engine 1534 reduces manual arithmetic and enables dose adjustment across multiple variables, addressing difficulty with patient-specific dosing and physician time constraints. Moreover, some patients lack certain enzymes to process certain medications, and these medications entering their body can be dangerous since instead of producing the expected beneficial results, the lack of the enzyme can create toxic metabolites. Furthermore, these enzyme or genetic abnormalities are not zero-sum or all or nothing, many times they are partially present or graded. People with these enzyme abnormalities can be identified through genetic testing, and the dose calculation enzyme can determine if a dose should be adjusted based on the genetic testing of the individual or a different medication prescribed altogether.

In one example, such as for medication dosing adjustments, a commonly used lab value can be an estimated creatinine clearance (eCrCl) value, which can be calculated with the Cockcroft-Gault equation. The variables of this equation can include serum creatinine (sCr), age, weight, and sex of the patient. The dose calculation engine 1534 can receive these variables as well as the equation from other modules described herein, calculate the estimated creatinine clearance, and recommend a dosage of medication based on the calculated estimated creatinine clearance.

The dose calculation engine 1534 can implement contraindication-aware dose limiting by querying the interaction and contraindication checker 1536 for concurrent medication and comorbidity constraints. More specifically, the dose calculation engine 1534 can revise a preliminary dose, extend an interval, or nullify a recommendation when interaction severity or contraindication rules exceed configured thresholds. In one example, the dose calculation engine 1534 can propagate limitation metadata to the compute composite suitability score step for traceable penalty application. Therefore, the dose calculation engine 1534 mitigates adverse interaction risk during dosing, addressing the risk of adverse drug interactions when holistic patient data is required.

The dose calculation engine 1534 can perform individualized dose computation by transforming baseline protocols using patient-specific renal and hepatic adjustment, body size metrics, and age. More specifically, the dose calculation engine 1534 can apply pharmacogenomic modifier application when Cytochrome P450 (CYP450) or Human Leukocyte Antigen (HLA) genotypes from the patient profile repository 1526 indicate ultra-rapid or poor metabolism, scaling dose and/or interval accordingly. In particular, the dose calculation engine 1534 can reference estimated glomerular filtration rate and Child-Pugh or Model for End-Stage Liver Disease (MELD scores) to proportionally reduce dose or elongate dosing intervals within exemplary bounds (e.g., reductions between 10% and 75%). Therefore, the dose calculation engine 1534 addresses difficulty adjusting doses based on multiple patient variables and supports precision dosing without a provider or team member being required to recalculate, which can be especially helpful in emergency situations. In other examples, other values can be used to adjust or determine a dose. For example, genetics and laboratory tests can identity unique differences in humans, and the dose calculation engine 1534 can use results from these tests to identify a difference in the physiologic and pharmacologic function of the patient, and recommend an adjusted dose or a first dose in response to the identified difference. Thus, the dose calculation engine 1534 can use any measurement, such as blood test results, genetic test results, imaging studies, function studies (e.g., Cardiac echocardiogram), heart rate measurements (e.g., medicines that lower heart rate will not be recommended if the heart rate measurement is below a threshold number, anthropometric, and the like.

The dose calculation engine 1534 can subscribe to HL7, FHIR, and the like events from the EMR interface 1602 and can trigger real-time recalculation triggering when a normalized real-time data update modifies renal, hepatic, weight, or drug level parameters. More specifically, the dose calculation engine 1534 can perform therapeutic drug level simulation using one- or two-compartment pharmacokinetic models to predict peak/trough ranges and loading strategies over exemplary horizons (e.g., between 6 and 72 hours). In one example, a pluggable algorithm architecture can load rule-based, regression-based, or machine-learning plug-ins that conform to a published interface for inputs, metadata, and versioning. Therefore, the dose calculation engine 1534 prevents or reduces outdated dosing during volatile clinical states and reduces clinician workload by updating recommendations automatically.

The dose calculation engine 1534 can perform structured output generation by emitting drug-specific organ-adjusted dose guidance as a machine-readable payload containing dose, interval, route, and justification codes and a human-readable rationale. In one example, the dose calculation engine 1534 can package traceable parameter contributions and limitation flags for presentation by the summary dashboard 1546 and for order entry via the integration and API layer 1552. Additionally, the dose calculation engine 1534 can version outputs to align with compute composite suitability score provenance requirements. Therefore, the dose calculation engine 1534 enables automated EMR handoff and transparent clinician review, addressing workflow efficiency and enabling integration of up-to-date dosing information into prescribing decisions.

### Analysis Engine 1532—Interaction and Contraindication Checker 1536

The interaction and contraindication checker 1536 can also be referred to herein as an interaction and contraindication identifier. The interaction and contraindication checker 1536 can access information acquired by other modules described herein, such as patient information obtained by the patient information collector 1518 (e.g., allergies, comorbidities, concomitant therapies, diagnosis, and the like) as well as drug information obtained by the drug information collector 1520 (e.g., dosing information, drug-drug interaction information, efficacy information, which can be published by sources including the FDA, the respective drug manufacturer, and the like) and compare the information. For example, the interaction and contraindication checker 1536 can compare drug information to a particular patient's unique and specific characteristics and situation, e.g., by cross checking the drug information with the patient's diagnosed disease, surgical history, medication list, allergies, and the like. The interaction and contraindication checker 1536 executes the evaluate drug-patient contraindications and interactions step using the harmonized clinical and drug dataset and the candidate medication set from the analysis engine 1532. In one example, the interaction and contraindication checker 1536 traverses a drug knowledge base 1528 to compute a drug-patient interaction risk profile and emits structured findings consumable by the compute composite suitability score step. Additionally, the interaction and contraindication checker 1536 references organ function metrics and allergy data to contextualize rules and to suppress non-actionable signals. Thus, the interaction and contraindication checker 1536 automates safety screening to reduce the risk of adverse interactions while conserving provider time.

A real-time knowledge-base synchronizer maintains REST and/or WebSocket sessions to external drug data repositories, regulatory feeds, and institutional formularies, and the real-time knowledge-base synchronizer merges validated deltas into a local knowledge graph with timestamping and retry logic. Additionally or alternatively, a user-configurable alert threshold registry persists versioned weights and trigger levels that the interaction and contraindication checker 1536 references at runtime to determine blocking alerts versus passive notifications, with live propagation to active sessions. Thus, the real-time knowledge-base synchronizer and the user-configurable alert threshold registry enable up-to-date rules and site-specific alerting behavior, addressing the lack of current information at the point of prescribing.

The interaction and contraindication checker 1536 applies an embedded severity matrix to classify interaction severity into categorical levels based on pharmacodynamic overlap, therapeutic index, and available organ reserve. More specifically, the interaction and contraindication checker 1536 uses the user-configurable alert threshold registry to generate real-time alerts that match local sensitivity settings and to route blocking versus informational messages. Thus, the classify interaction severity function reduces alert fatigue while prioritizing clinically meaningful risks within provider workflow constraints. For example, the interaction and contraindication checker 1536 can present interaction and/or contraindication information to the provider e.g., via the presentation layer 1544, to enable the provider in reviewing and appreciating the relation to other factors while making the final determination.

The interaction and contraindication checker 1536 detects drug-drug interactions. For example, the interaction and contraindication checker 1536 can traverse knowledge graph edges representing interaction pairs for the candidate medication set and by evaluating associated rules. In one example, the interaction and contraindication checker 1536 annotates each detected conflict with a severity grade and supporting rationale before emitting entries into the drug-patient interaction risk profile. Thus, the detect drug-drug interactions function prevents or inhibits high-risk combinations from reaching order finalization and reduces adverse event risk. The medicine recommendation platform 1500 permits or enables the prescriber to access data, that otherwise would require numerous logins and access to platforms and tools, in one location efficiently. Thus, a prescriber can access a vast amount of relevant information without logging in or otherwise accessing numerous separate platforms, or switching or toggling between multiple platforms. Instead, the medicine recommendation platform 1500 can obtain data from various databases, e.g., in some cases hundreds of databases, each which can have a login, and present it to a provider on one screen, e.g., make the data accessible in one place.

The interaction and contraindication checker 1536 identifies drug-disease contraindications. For example, the interaction and contraindication checker 1536 can cross-reference active diagnoses, organ function measures, and comorbidities against contraindication tables within the drug knowledge base 1528. In one example, the interaction and contraindication checker 1536 outputs structured contraindication objects that downstream modules use to filter or de-prioritize candidates during ranking and optimization. Thus, the identify drug-disease contraindications function aligns selections with patient conditions and reduces contraindicated prescribing.

The interaction and contraindication checker 1536 recommends safer alternatives. For example, the interaction and contraindication checker 1536 can query the drug knowledge base 1528 for pharmacologic substitutes that avoid detected risks and by returning a ranked substitution list with dose guidance. In one example, the interaction and contraindication checker 1536 refines risk prediction via machine learning that augments rule outputs with probabilistic scores derived from historical outcomes. Additionally, the interaction and contraindication checker 1536 synchronizes the clinical knowledge base via the real-time knowledge-base synchronizer so that alternative sets reflect current approvals and warnings. Thus, the recommend safer alternatives function accelerates safe choice selection and addresses provider time constraints while improving safety.

### Analysis Engine 1532—Cost and Coverage Analyzer 1538

The analysis engine 1532 can include a cost and coverage analyzer 1538 that receives an initial ranked medication list and patient-specific cost and coverage data, e.g., via the integration and API layer 1552 and/or the hospital/pharmacy database connector 1704. Factoring in cost information is particularly challenging for prescribers since they often do not have access to or time to access medication costs, let alone specific costs for particular patients, since this information can be siloed on various platforms and in numerous locations. Feasibly, it is impractical for a prescriber to access each of these various databases and cross reference them to determine the best cost for the patient, while also considering drug efficacy. This information is often patient specific and can be influenced by the patient's specific economic condition, e.g., whether the patient is insured or not, or which particular insurance brand a patient has (e.g., Medicare, Medicaid, Veterans, TriCare, or the like) and which specific plan they are on. This is further complicated by what is available at various hospital formularies and other supply and demand factors. Due to the incredible complexity of these economic factors that are scattered and siloed in various platforms, the medicine recommendation platform 1500 can save a prescriber vast amounts of time compiling and cross checking the vast information from numerous locations. This tool brings vast amounts of data from the various platforms to the prescriber in one easily accessible and viewable location, while having already cross referenced the data, e.g., evaluating medications based on expected efficacy for the particular patient and also factoring in and eliminating, or ranking lower, choices that would be incompatible, or less compatible, with the patient due to factors such as drug-drug interaction incompatibility, allergic reaction incompatibility, or medical condition incompatibility (e.g. liver or kidney disease with a medication that causes further liver or kidney damage). Thus, the medicine recommendation platform 1500 enables the prescriber to view and consider all the relevant information related to safety, efficacy, and cost in one place.

In one example, the cost and coverage analyzer 1538 executes apply cost and insurance adjustment to generate a cost-adjusted ranked medication list annotated with payer, formulary tier, and pricing descriptors for delivery to the multi-criteria ranking engine 1542 and the presentation layer 1544. The cost and coverage analyzer 1538 can interface with the patient profile repository 1526 and the drug knowledge base 1528 to validate identifiers and normalize pricing units. Therefore, the cost and coverage analyzer 1538 addresses the challenges of integrating up-to-date cost and insurance information into prescribing without requiring manual review by a clinician.

The cost and coverage analyzer 1538 can include a cost-factor weight matrix persisted in a configuration partition to modulate contributions from acquisition price, administration labor, laboratory monitoring, and treatment duration. In one embodiment, the analyzer multiplies each cost vector element by a configurable weight to pivot between patient-focused and institution-focused cost perspectives without modifying computational kernels. Therefore, the cost-factor weight matrix reduces recalculation burden and aligns economic scoring with local policy, mitigating clinician time constraints.

In one example, the cost and coverage analyzer 1538 can include a coverage rule vault that stores encrypted, versioned mappings from drug codes to formulary tiers, prior-authorization triggers, step-therapy sequences, and coinsurance breakpoints. The analyzer evaluates coverage locally against the vault to avoid network latency and to produce deterministic coverage determinations during bedside use. Therefore, the coverage rule vault enables rapid comparison of therapy options against plan rules, addressing the absence of reliable coverage comparison at the point of prescribing.

The cost and coverage analyzer 1538 can include a patient-specific co-pay calculator that consumes deductible status, accumulator balances, and plan tier parameters imported from the integration and API layer 1552. In one embodiment, the calculator derives individualized co-pay or coinsurance estimates for each candidate medication and returns structured values for display and downstream ranking. Therefore, the patient-specific co-pay calculator surfaces wallet impact alongside clinical fit, improving economic personalization for the patient.

In one example, the cost and coverage analyzer 1538 can aggregate economic inputs by retrieving wholesale, contract, and out-of-network prices, formulary assignments, and ancillary cost factors from multi-source feeds. More specifically, the analyzer can compute total cost of therapy by combining normalized acquisition price with administration overhead, laboratory monitoring expense, expected duration, and dose guidance from the dose calculation engine 1534. Therefore, the aggregation routine produces a consistent economic dataset, enabling automated use of current market data in medication selection.

The cost and coverage analyzer 1538 can estimate patient out-of-pocket expense by applying coverage rules, deductible phase, coinsurance percentages, and out-of-pocket ceilings to the aggregated price data. In one example, the analyzer can flag coverage barriers by attaching structured indicators for formulary exclusion, prior authorization, and step therapy to each medication. Therefore, the out-of-pocket estimation and barrier flags expose affordability and access risks early, reducing downstream prescription delays.

In one embodiment, the cost and coverage analyzer 1538 can generate real-time cost updates by subscribing to pricing and coverage delta events, incrementally recalculating affected medications, and publishing refreshed metrics to the presentation layer 1544. The analyzer can also trigger recalculation on threshold change events produced by the analysis engine 1532 to maintain internal consistency. Therefore, the real-time updates keep cost data synchronized with external feeds, alleviating the need for manual refresh by the clinician.

The cost and coverage analyzer 1538 can rank medications by economic impact by weighting cost vectors using the cost-factor weight matrix and summing to produce a composite economic impact score per drug. In one example, the analyzer sorts candidates by this score and sends the ordered list to the multi-criteria ranking engine 1542 for fusion with efficacy and safety metrics. Therefore, the economic ranking supplies a quantified, comparable cost dimension, enabling final recommendations that balance affordability with clinical suitability.

Additionally, the cost and coverage analyzer 1538 can generate preapprovals for insurance as well as supporting documents needed, e.g., based on data retrieved and stored in the data storage layer 1524. This data is the typically fractionated data in the medical record and other databases. Therefore, by generating patient-specific preapprovals for insurance as well as supporting documents needed, the system enables a more efficient approval process, which can result in the patient receiving care faster than traditional methods.

### Analysis Engine 1532—Efficacy and Resistance Evaluator 1540

The efficacy and resistance evaluator 1540 can compute a predicted clinical success probability for each candidate medication given a pathogen or disease label and a normalized patient parameter set. In one example, the evaluator can fuse institution-specific susceptibility rates from antibiograms with literature-derived effectiveness curves to map susceptibility percentages to expected cure probability across medication-pathogen pairings. Additionally, the evaluator can incorporate real-time updates from a real-time resistance data connector 1706 and adjust scores according to most recent observations to reflect current microbiological conditions. Therefore, the evaluator reduces physician time spent synthesizing dynamic evidence and supplies point-of-prescribing efficacy estimates that address the lack of real-time resistance awareness.

A configurable threshold registry can store version-controlled parameters, such as a minimum acceptable probability of success (e.g., 0.6-0.9) and/or a maximum tolerated resistance rate (e.g., 0.1-0.4), as signed configuration records. In one embodiment, the evaluator can reference the registry during scoring to filter medications below thresholds, to flag borderline agents for review, and to route excluded candidates with rationale codes to downstream modules. Additionally, the evaluator can subscribe to registry change events and trigger recalculation when a threshold update crosses a configured delta. Thus, the threshold registry enables rapid stewardship policy alignment without manual rework, addressing physician time constraints at the moment of decision-making.

The evaluator can aggregate multi-source resistance data by retrieving susceptibility observations from internal microbiology systems, regional public health feeds, and the real-time resistance data connector 1706, and by harmonizing codes (e.g., Anatomical Therapeutic Chemical (ATC) classification system and/or RxNorm) and deduplicating records into a susceptibility matrix cache. More specifically, the evaluator can apply temporal resistance trend weighting by decaying older observations according to an age-based kernel (e.g., exponential half-life between 30 and 180 days) to preferentially weight recent measurements. In one embodiment, the evaluator can recalculate source confidence weights based on sample size and recency before recomposing composite susceptibility estimates per drug-pathogen-region tuple. Therefore, the aggregation and temporal weighting provide a current and comprehensive resistance landscape at the point of prescribing, addressing the lack of real-time susceptibility information.

The evaluator can deliver a ranked medication list with explanatory metadata by ordering candidates by predicted success probability and publishing a structured payload to an analysis engine 1532 shared memory bus. In one example, the evaluator can flag medications with emerging resistance when a monitored resistance rate increases beyond a configurable delta (e.g., 5-20 percentage points over 14-90 days) and append the flag to the metadata bundle to enable downstream deprioritization. Additionally, the evaluator can stratify efficacy by infection site and comorbidity by filtering the susceptibility matrix to strata-aligned subsets and recomputing probabilities for bloodstream, pulmonary, urinary, and/or other sites with comorbidity modifiers. Thus, the ranked and annotated output provides an actionable list tailored to the clinical scenario, addressing physician time constraints and enabling integration of up-to-date efficacy signals into prescribing decisions.

### Analysis Engine 1532—Multi-Criteria Ranking Engine 1542

The multi-criteria ranking engine 1542 can execute ranking and optimization using inputs from the medication suitability score list and auxiliary metadata from the analysis engine 1532. In one example, the multi-criteria ranking engine 1542 can rank drugs by composite score and select a top medication set according to configurable constraints and/or provider preferences. Further, the multi-criteria ranking engine 1542 or other modules described herein (such as the data acquisition layer 1516) can monitor real-time clinical or pharmaceutical data feeds and, in response to detecting a threshold change in a monitored parameter, recompute the composite suitability score and update the presented ranked medication recommendation set. For example, the medicine recommendation platform 1500 can scout the EMR and compare data from millions of patients for two or more different medications for a given condition and then analyze all the data available in the EMR to evaluate and determine which medication has a more effective real-world effect in a de-identified way, e.g., such that the patient privacy is not impacted. For example, by comparing actual blood pressure results for two or more antihypertensives. The medicine recommendation platform 1500 can prevent or reduce reliance on studies that are conducted by the drug manufacturer, and thus inherently biased (e.g., in studies in which a smaller number of participants have been particularly chosen, in some cases for particular characteristics or attributes, to obtain FDA approval). The medicine recommendation platform 1500 can also impact pharmacovigilance since it can identify trends and correlations in certain medications. For example, if a certain blood pressure medication is also causing kidney function issues in the patients who take it, the trends would be captured and could be appreciated.

Additionally, the multi-criteria ranking engine 1542 can support multiple decision techniques, such as weighted sum models and/or pairwise priority schemes, exposed through an API of the analysis engine 1532. Thus, the multi-criteria ranking engine 1542 consolidates multifactor evidence into an actionable ordering, reducing physician time burden while enabling integrated clinical and economic decision making.

A configurable weight matrix repository can store weighting coefficients indexed by indication, protocol, and user role, with version tags and effective dates for audit. In one example, a criteria normalization pipeline can transform heterogeneous criterion outputs into a common scale using linear and/or logistic and/or piece-wise mappings with clamped bounds and reversible transformations, optionally vectorized for SIMD acceleration. Additionally, a thread-safe API can permit governance tools and/or authorized clinicians to update active matrices without redeploying the multi-criteria ranking engine 1542. Thus, the configurable weight matrix repository and the criteria normalization pipeline prevent or reduce scale bias and enable policy-driven customization, improving fairness and clinical validity of rankings across diverse prescribing contexts.

The multi-criteria ranking engine 1542 can compute composite medication scores by aggregating normalized criterion vectors with coefficients retrieved from the configurable weight matrix repository. In one example, the multi-criteria ranking engine 1542 can apply penalties and/or hard exclusions based on a drug-patient interaction risk profile and can incorporate organ-adjusted dose guidance as modifiers to efficacy and safety criteria. Additionally, the multi-criteria ranking engine 1542 can parallelize computations across candidate drugs to achieve sub-second latency on commodity hardware. Thus, the composite scoring function aligns dose adjustments, interaction risks, and efficacy with patient-specific data, addressing dosing complexity and adverse-interaction concerns.

The multi-criteria ranking engine 1542 can generate a ranked medication list by sorting composite scores and resolving ties using secondary keys such as contraindication severity and/or availability. In one example, the multi-criteria ranking engine 1542 can request a cost and insurance adjustment from the cost and coverage analyzer 1538 to produce a cost-adjusted ranked medication list prior to selecting a top medication set. Additionally, the multi-criteria ranking engine 1542 can attach references to dosing, safety, and cost metadata to support downstream presentation. Thus, the ranking and selection workflow incorporates up-to-date cost and coverage signals into prescribing, reducing manual effort and enabling comparison across clinically viable options.

The multi-criteria ranking engine 1542 can perform dynamic weight recalibration by reloading updated matrices upon governance changes and/or provider overrides while preserving historical version references for audit. In one example, the multi-criteria ranking engine 1542 can execute real-time re-ranking on data change by listening for trigger recalculation events and recomputing only impacted criteria before regenerating the ranked list. Additionally, the multi-criteria ranking engine 1542 can respond to normalized real-time data update sourced from resistance, availability, and epidemiological feeds. Thus, the recalibration and re-ranking capabilities maintain recommendation fidelity under changing clinical signals, addressing real-time resistance updates and shifting availability without requiring manual refresh.

Thus, the multi-criteria ranking engine 1542 can retrieve, compile, and evaluate data to rank medications in a particular order for a particular patient (e.g., most effective and affordable). With the medications ranked, the provider can view effectiveness, safety, and cost all in one place (e.g., the summary dashboard 1546 described herein). This presentation and ranking system can enable the provider to access a vast amount of information they would otherwise not have access to, in order to make a decision regarding a prescription that is appropriate for each individual patient. The medicine recommendation platform 1500 can use the provider's decision to calculate a dose that accounts for patient specific variables, such as other medications being taken, other illnesses, and the like.

### Presentation Layer 1544

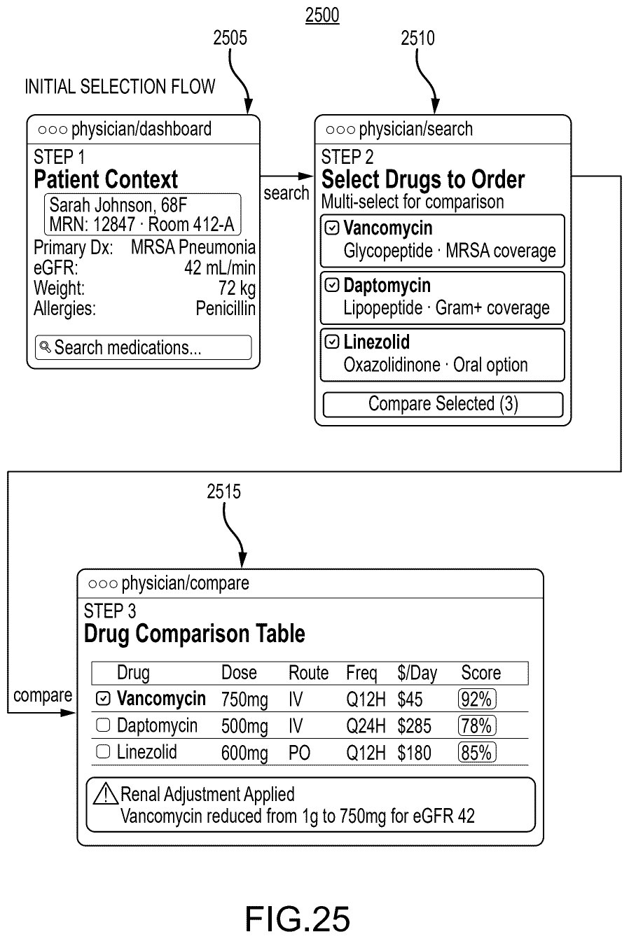

The presentation layer 1544 can deliver recommendation delivery outputs, justification data, and interaction controls through graphical interfaces and programmatic endpoints. For example, as depicted in FIG. 25 among others, a graphical user interface (GUI) 2500 presenting an example initial selection flow is depicted. In this example, the presentation layer 1544 presents patient context data in a first box or screen 2505, multiple drugs, which can also be referred to herein as candidate drugs, for comparison in a second box or screen 2510, and a drug comparison table in a third box or screen 2515. In this example, the patient context data of the first screen 2505 can be collected from the data acquisition layer 1516, e.g., the patient information collector 1518. As depicted on the first screen 2505, the presentation layer 1544 can present a patient name; age; gender; hospital ID number; room number; diagnosis or indication (e.g., pneumonia); estimated Glomerular Filtration Rate (“GFR”), an indicator of kidney function that can assist a provider in assessing kidney function; weight; allergies; and the like.

In this example, the multiple drugs for comparison of the second screen 2510 can be retrieved from the data storage layer 1524, e.g., the drug knowledge base 1528. In some examples, the analysis engine 1532 can use the data from the data storage layer 1524 to identify the appropriate candidate drugs, e.g., defined by the drug knowledge base 1528. As depicted on the second screen 2510, the presentation layer 1544 can present a list of candidate drugs by generic or brand names; the class of drug, e.g., the class of antibiotic as shown in FIG. 25; types of coverage of the drug, e.g., MRSA coverage refers to the ability of antibiotics to treat Methicillin-resistant Staphylococcus aureus, a type of bacteria that is resistant to many common antibiotics, Gram-positive coverage refers to the classification of bacteria based on their ability to retain the crystal violet dye during the Gram staining process and Gram-positive bacteria are more susceptible to certain antibiotics due to their cell wall composition; the type of administrations, such as oral or intravenous; and the like. On the second screen 2510, a provider can select which drugs to compare in more detail.

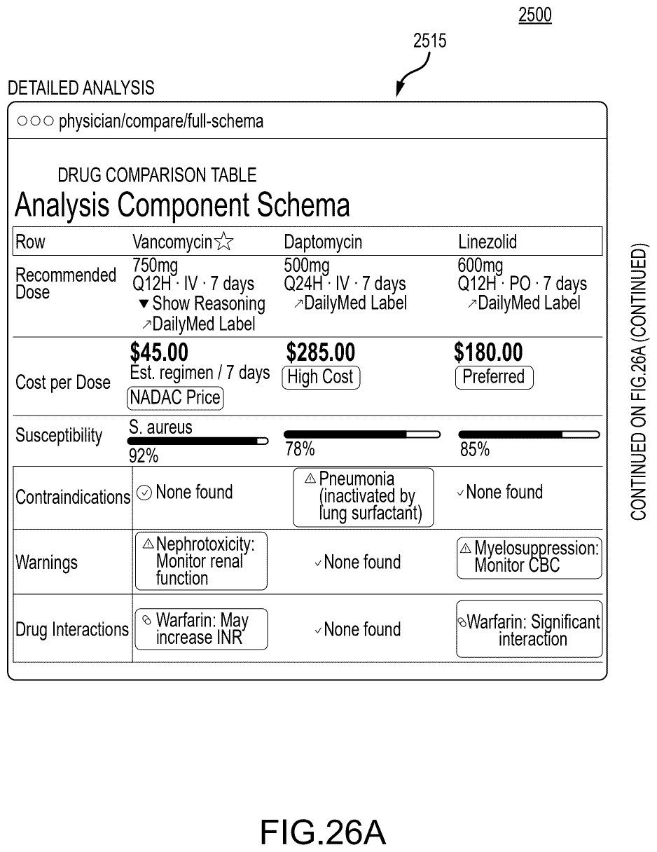

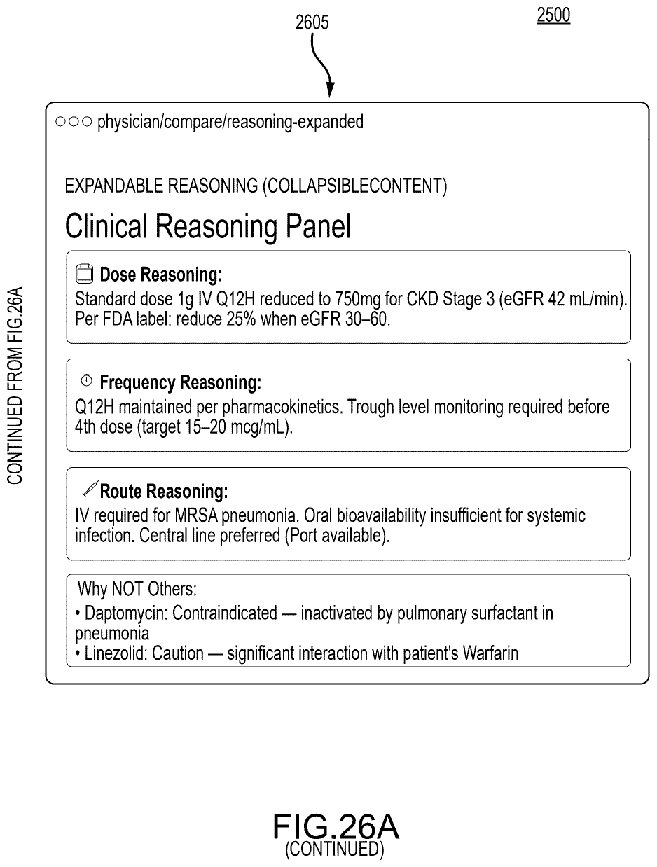

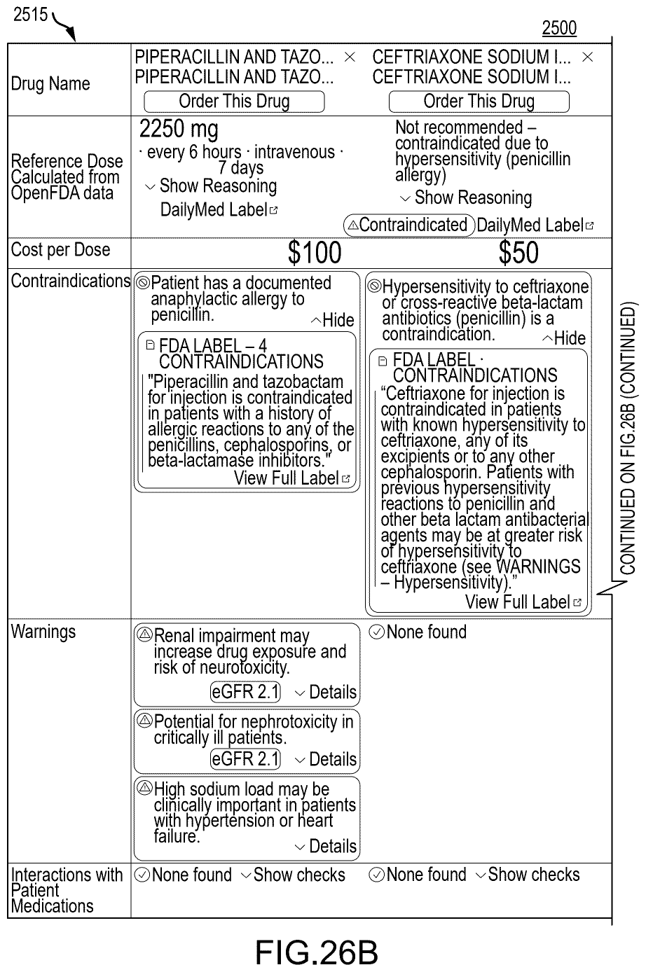

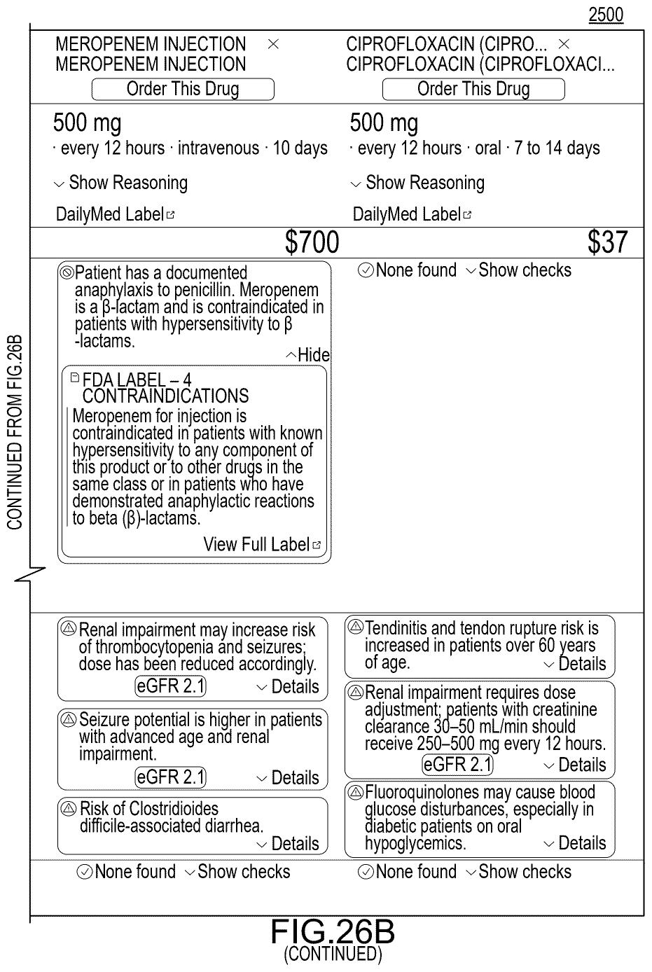

In this example, the drug comparison table of the third screen 2515 can be generated by the analysis engine 1532. As depicted on the third screen 2515, the presentation layer 1544 can present a comparison, e.g., a tabular comparison chart, of the candidate drugs selected by the provider on the second screen 2510. For example, the drug name; recommended dose; administration route; administration frequency; cost per day, which can be specific to the patient accounting for insurance coverage; a ranking or score, e.g., a susceptibility score; notes, such as dose adjustments applied for patient specific attributes, e.g., renal adjustments based on kidney function; and the like. FIGS. 26A and 26B depict additional examples of the third screen 2515. In these examples, additional details or information can be presented by the presentation layer 1544. For example, as depicted in FIG. 26A, the third screen 2515 can also include duration or estimated regimen of recommended dose, links to the drug label, different icons for presenting information, such as the bar for presenting the score; contraindications, warnings, and drug interactions, including if none are found; and the like. Additionally, the third screen 2515 can include collapsible content or expandable reasoning 2605. For example, a provider can click or select the “Show Reasoning” link and the presentation layer 1544 can present additional content or clinical reasoning. The expandable reasoning 2605 can include dose reasoning; frequency reasoning; route reasoning; notes, including contraindications, drug interactions, warnings, and the like; and the like. For example, as depicted in FIG. 26B, the third screen 2515 can include the recommended drug name with links to order the drug or add it to an order cart; a calculated reference dose including the amount, regimen, administration route, and duration, and the like, which can each be calculated by the analysis engine 1532, e.g., the dose calculation engine 1534 using FDA data, patient data, and the like, e.g., acquired by the data acquisition layer 1516; in cases where a contraindication, drug interaction, allergy, or the like exists, a not regarding why the drug is not recommended can be included, e.g., instead of the calculated reference dose (in either case, links to additional reasoning 2605 and/or the drug label can be included, similar to or the same as described above with respect to FIG. 26A); a cost per dose or regime, which can be patient-specific; contraindications, warnings, and drug interactions (including if none are found), which can include additional expandable menus with additional information, e.g., sourced from the FDA label, links to patient files, such as kidney function test results, and the like; and the like.

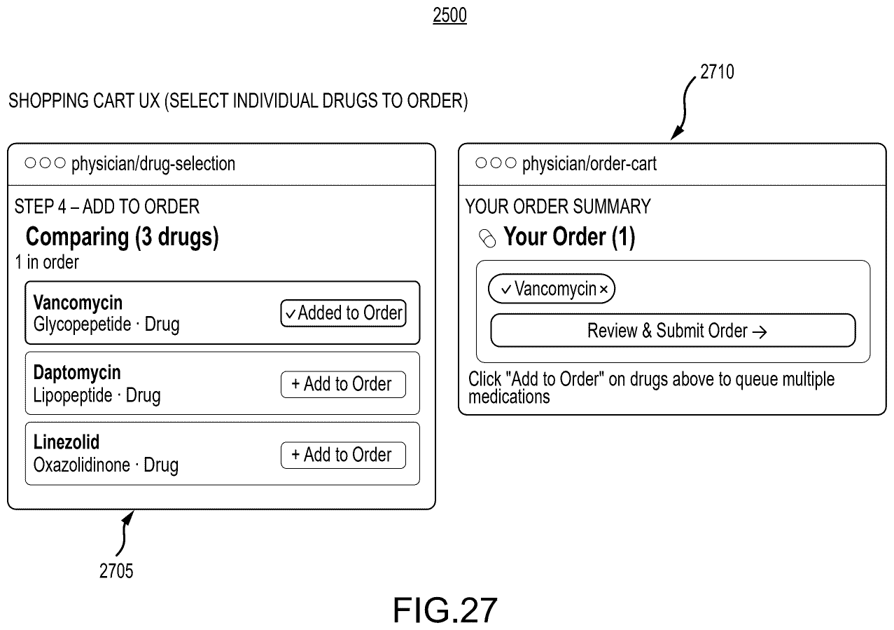

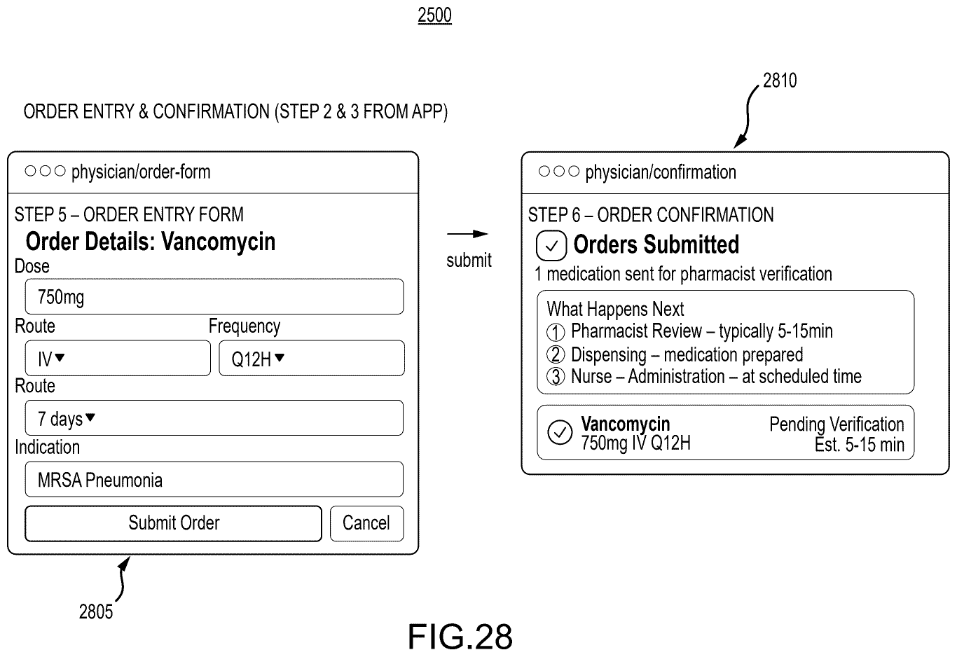

As depicted in FIG. 27 among others, in some examples the presentation layer 1544 can present a shopping cart in a fourth box or screen 2705, where the provider can add any of the candidate drugs to an order. Further, the presentation layer 1544 can present an order summary in a fifth box or screen 2710, in which the selected candidate drug(s) are listed and a provider can choose to add more candidate drugs to the order or continue to review and submit the order. As depicted in FIG. 28 among others, in some examples the presentation layer 1544 can present an order entry form for the selected candidate drug on a sixth box or screen 2805, which can include the recommended dose, route, frequency, duration, indication, and the like. The provider can adjust and of the order details, submit the order, and/or cancel the order. Further, the presentation layer 1544 can present an order confirmation for the submitted order on a seventh box or screen 2810, which can include a confirmation that the selected medication has been sent for pharmacist verification; a summary of the status and next steps, including an estimated time frame for each step; the drug and the administration information; the order status; and the like. The presentation layer 1544 can present one or more of the screens 2505, 2510, 2515, 2605, 2705, 2710, 2805, 2810, and in some examples may not present one or more of the screens 2505, 2510, 2515, 2605, 2705, 2710, 2805, 2810, e.g., which can be dependent on user preference selections.

In one example, the presentation layer 1544 formats ranked medication recommendation set data, contraindication alerts, cost and coverage insights, and real-time efficacy indicators into GUIs and/or APIs, and the presentation layer 1544 can accept clinician overrides and feedback for downstream processing. Additionally, the presentation layer 1544 can orchestrate the summary dashboard 1546, the pictogram generator 1548, and the packaging display module 1550 to reduce cognitive load and to streamline selection. Thus, the presentation layer 1544 addresses physician time constraints and the inability to combine efficacy, safety, and cost information by presenting consolidated, actionable content at the point of prescribing.

The presentation layer 1544 can render a context-sensitive alert zone that reserves a fixed container for high-severity outputs of the interaction and contraindication checker 1536. More specifically, the presentation layer 1544 can display contraindication alerts with adaptive coloration and prioritized ordering, and a user can expand an alert to view pharmacological rationale, affected laboratory values, and system-suggested alternatives. In one example, the presentation layer 1544 maintains a subscription to real-time alert streams and updates the alert zone without a page refresh. Therefore, the context-sensitive alert zone mitigates the risk of adverse interactions by surfacing safety issues directly within the decision context.

The presentation layer 1544 can employ a modular widget framework that assembles self-contained components such as ranked-list tables, dosage calculators, cost ribbons, alert badges, and packaging viewers. In one example, each widget declares a manifest describing data contracts, style tokens, and event hooks, and the framework can hot-swap widget versions while preserving bindings and analytics callbacks. Additionally, the framework can enable or reorder widgets to align with local workflows without recompilation. Thus, the modular composition reduces clinician effort and supports rapid adaptation to institutional needs, addressing time constraints while maintaining complete views of efficacy, safety, and cost.

The presentation layer 1544 can render ranked medication list outputs from the multi-criteria ranking engine 1542 as a sortable, real-time updating table. In one example, the presentation layer 1544 can render ranked medication list columns that include dose guidance, efficacy probability, interaction severity, and cost and coverage, and the presentation layer 1544 can visualize packaging and pictograms by invoking the packaging display module 1550 and the pictogram generator 1548 adjacent to each row. Additionally, the presentation layer 1544 can update rows in place on receipt of new analytics payloads to reflect shifting resistance or coverage conditions. Therefore, the rendered list enables providers to integrate up-to-date efficacy, interaction, and cost information rapidly, addressing data overload and selection errors.

The presentation layer 1544 can synchronize with EMR views by launching within a Substitutable Medical Applications and Reusable Technologies (SMART)-on-FHIR session and exchanging OAuth scopes and patient context tokens. In one example, the presentation layer 1544 can synchronize with EMR views by mirroring current medications and laboratory values in real time and by posting edits back using FHIR PATCH operations to preserve a single source of truth. Additionally, the presentation layer 1544 can align timestamps and provenance with EMR records to enable traceability of clinician overrides. Thus, EMR synchronization reduces reconciliation effort and lowers interaction risk by ensuring recommendations reflect authoritative, current patient data.

The summary dashboard 1546 can present the comprehensive view of candidate drugs with sortable attributes for dose recommendations, expected efficacy, interaction risk indicators, and cost estimates. In one example, the summary dashboard 1546 integrates hierarchical components by invoking the summary dashboard 1546 for tabular views, the pictogram generator 1548 for standardized iconography and color cues, and the packaging display module 1550 for packaging thumbnails and barcodes. Additionally, the summary dashboard 1546 can refresh in near real time when laboratory, resistance, availability, or coverage data change. Therefore, the summary dashboard 1546 enables holistic, rapid comparison across clinical and economic factors, addressing multi-variable dose adjustment, resistance awareness, and coverage comparison within a single interface.

### Presentation Layer 1544—Summary Dashboard 1546

The summary dashboard 1546 can render a tabular view in which each row represents a candidate medication and columns enumerate patient-adjusted dose guidance, expected efficacy, interaction and contraindication risk, and cost and coverage metrics. In one example, the summary dashboard 1546 receives the ranked medication recommendation set and the traceability metadata bundle from the presentation layer 1544 and generates a visual summary interface that also embeds availability and packaging information with optional pictograms. Additionally, the summary dashboard 1546 can subscribe to normalized real-time data update and trigger recalculation outputs surfaced by the analysis engine 1532 to refresh displayed attributes without user intervention. Therefore, the summary dashboard 1546 enables rapid, holistic comparison that addresses physician time constraints, integrates cost and coverage alongside effectiveness, and surfaces dosing tailored to renal and hepatic function.

The summary dashboard 1546 can display colour-coded risk indicator cells that apply an indexed palette to encode interaction severity, contraindication category, and allergy conflict generated by the interaction and contraindication checker 1536. In one example, the summary dashboard 1546 can also render a pictogram and packaging embed pane that consumes assets from the pictogram generator 1548 and the packaging display module 1550 to provide brand and form-factor recognition. Additionally, the summary dashboard 1546 can map categorical risk values to vector or CSS styles so a clinician perceives high-risk options without parsing text. Therefore, the colour encoding and embedded visuals mitigate adverse interaction risk and reduce cognitive load during time-pressured prescribing.

The summary dashboard 1546 can expose interactive sort and filter controls bound to a multi-attribute tabular grid so a user can reorder by efficacy percentage, out-of-pocket cost, coverage tier, or interaction severity. In one example, the summary dashboard 1546 executes client-side predicate evaluation to re-render sorted or filtered grids within an exemplary sub-100 ms latency, thereby enabling rapid candidate ranking aligned with outputs of the multi-criteria ranking engine 1542. Additionally, the summary dashboard 1546 can maintain column header toggles for ascending/descending order and persist control state for session continuity. Therefore, the interactive controls operationalize complex quantitative results into an intuitive workflow that addresses limited clinician time and the need to integrate heterogeneous decision variables.

The summary dashboard 1546 can facilitate attribute-based filtering through sliders, toggles, and check boxes that exclude medications above a configurable cost threshold, below an efficacy cutoff, or with major contraindications. In one example, the summary dashboard 1546 maps each control to underlying fields such as formulary status, coverage tier, renal adjustment requirement, and interaction category to construct filters without query syntax. Therefore, the filtering operations focus attention on feasible options and address cost-coverage constraints and safety concerns.

The summary dashboard 1546 can present a patient-specific medication overview by aggregating outputs from the dose calculation engine 1534, the efficacy and resistance evaluator 1540, the interaction and contraindication checker 1536, and the cost and coverage analyzer 1538. In one example, the summary dashboard 1546 listens for updated harmonized datasets triggered by threshold changes so resistance shifts or new labs readily update efficacy and dosing columns. Additionally, the summary dashboard 1546 can interoperate within an EMR pane or a web module via the integration and API layer 1552 while maintaining identical data bindings. Therefore, the unified presentation resolves fragmentation across data sources and addresses the lack of real-time resistance and dose personalization.

The summary dashboard 1546 can support clinical documentation by exporting a time-stamped visual summary interface view with embedded rationale derived from the traceability metadata bundle. In one example, the summary dashboard 1546 can also serialize clinician actions from the allow clinician override and feedback capture flow into a clinician override and feedback record that accompanies the final selected medication order. Additionally, the summary dashboard 1546 can generate printable PDFs and/or structured files for EMR attachment or stewardship reporting. Therefore, the documentation features streamline transparent justification and audit readiness while reducing manual effort during clinical encounters.

### Presentation Layer 1544—Pictogram Generator 1548

The pictogram generator 1548 can render icons, color-coded icons, graphical symbols, and packaging thumbnails adjacent to each entry of a ranked medication recommendation set within the presentation layer 1544. More specifically, the pictogram generator 1548 can map drug class, route of administration, dosage form, contraindication indicators, interaction risks, and insurance coverage status to standardized visual cues such as overlays, badges, and border treatments, with palettes selected from accessibility-safe schemes (e.g., color-blind safe ranges). Additionally, the pictogram generator 1548 can retrieve packaging imagery from pharmaceutical and/or regulatory repositories via the drug information collector 1520 and then composite the imagery with generated iconography according to institutional style rules and user preferences. In one example, the pictogram generator 1548 can update rendered pictograms in real time when the analysis engine 1532 adjusts recommendations or dosage guidance, thereby maintaining alignment with current traceability metadata and reducing selection errors in the summary dashboard 1546. The pictogram generator 1548 can generate and display the correct, recommended, or prescribed drug as well as the correct, recommended, or prescribed dose for that drug.

### Presentation Layer 1544—Packaging Display Module 1550

The packaging display module 1550 can retrieve, generate, and render visual representations of medication packaging keyed by identifiers from the ranked medication recommendation set, such as National Drug Code ranges and manufacturer references, and can source assets from the drug knowledge base 1528 and/or hospital/pharmacy database connector 1704 via the integration and API layer 1552. More specifically, the packaging display module 1550 can render raster and/or vector images and optional 3D models, can expose interactive zoom, rotation, and side-by-side comparison, and can vary the presentation by manufacturer, dosage form, strength, and regional labeling. Additionally, the packaging display module 1550 can overlay scannable barcodes or QR codes for EMR and pharmacy system verification and can annotate packaging with availability status and unit-of-use information retrieved at query time, while optionally caching frequently accessed assets in the caching store 1530. Therefore, the packaging display module 1550 enables rapid visual and barcode-based confirmation of the recommended medication, which reduces selection errors and shortens verification time under physician time constraints, while supporting availability awareness to address dynamic prescribing context.

By presenting a visualization of the package of a drug ordered by a physician (e.g., identified by NDC or another unique identifier) to the team members, the team member who is tasked with administering the drug to the patient can confirm that the real-time package and drug is the correct, prescribed medication. Likewise, the medication, e.g., the tablet, capsule, pill, and the like can include unique identifiers, e.g., shape, color, alphanumeric markings, and the like, that are specific to a medication and/or dose of that medication. The packaging display module 1550 (or other modules described herein) can also present an image of the medication and/or the unique identifier, which can provide the medication administrator with the opportunity to confirm the correct or prescribed medication is going to be administered, which can help in cases where the medication is loose and/or being sent from the pharmacy without the standard packaging.

### Integration and API Layer 1552

The integration and API layer 1552 exposes versioned RESTful and FHIR endpoints and/or secure messaging interfaces (e.g., HL7 v2.x over Minimal Lower Layer Protocol (MLLP), Advanced Message Queuing Protocol (AMQP), Message Queuing Telemetry Transport (MQTT), and the like) to exchange patient data, medication orders, laboratory results, insurance data, and drug availability or cost data with EMR, pharmacy, and analytics systems. More specifically, the integration and API layer 1552 can authenticate calls using OAuth2 and/or JSON Web Token (JWT), enforce authorization scopes and rate limits, and record request and response metadata for auditability under the security and compliance module. In one example, the integration and API layer 1552 transforms and maps payloads between external standards and internal schemas, and can validate, filter, and enrich messages according to configurable clinical and business rules before writing to the data storage layer 1524 and notifying the analysis engine 1532. In another example, the integration and API layer 1552 operates as an API-centric cloud-native microservice, supports batch ingestion and real-time streaming, and applies retries and circuit breakers while maintaining backward-compatible versioning to preserve interoperability during upgrades.

The integration and API layer 1552 can facilitate real-time bidirectional synchronization by subscribing to change-data-capture feeds and/or FHIR Subscriptions, transforming delta messages, and committing updates to the data storage layer 1524 with exemplary end-to-end latency (e.g., 100 to 900 ms). More specifically, the integration and API layer 1552 can propagate provider actions from the presentation layer 1544—such as a finalized medication order-back to EMR and pharmacy systems through the same endpoints or queues to maintain cross-system consistency. Additionally, the integration and API layer 1552 can publish domain events to the analysis engine 1532 to trigger recalculation on threshold change and can throttle or coalesce bursts to avoid redundant computations. In one embodiment, the integration and API layer 1552 manages conflict resolution using versioning metadata (e.g., timestamps and sequence numbers) and configurable precedence rules, and can schedule backfill jobs when upstream feeds are delayed to prevent or reduce divergence between clinical documentation and decision support outputs.

### Patient-Specific Medication Recommendation or Ranking Method 1800

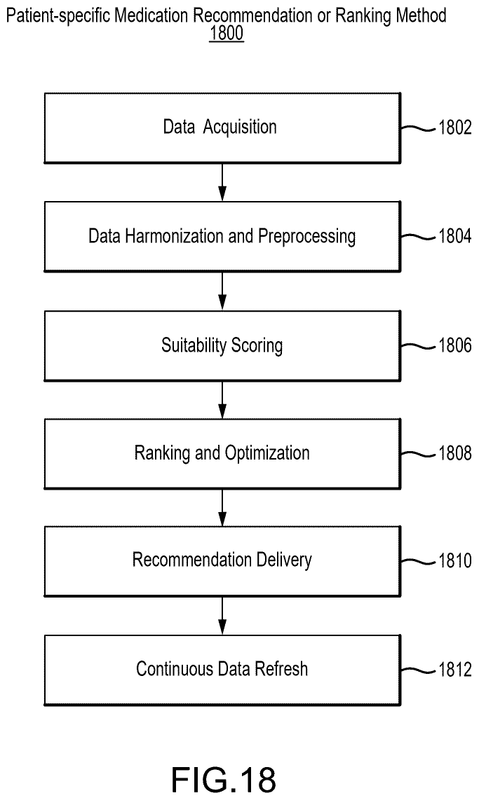

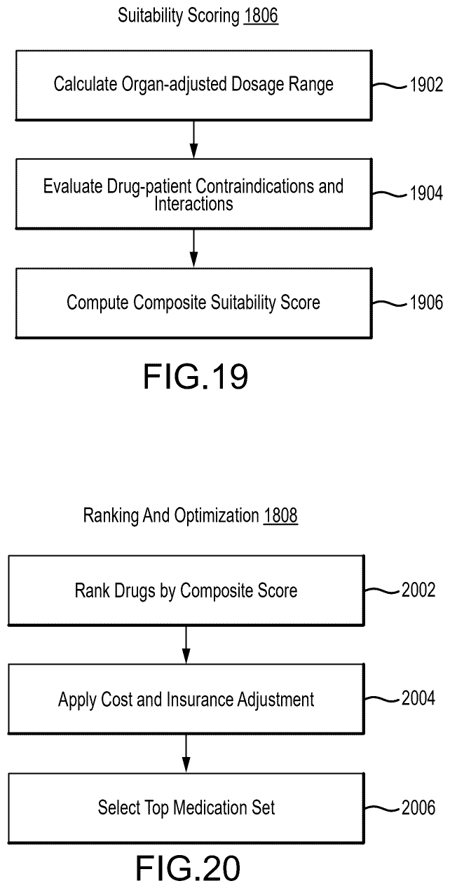

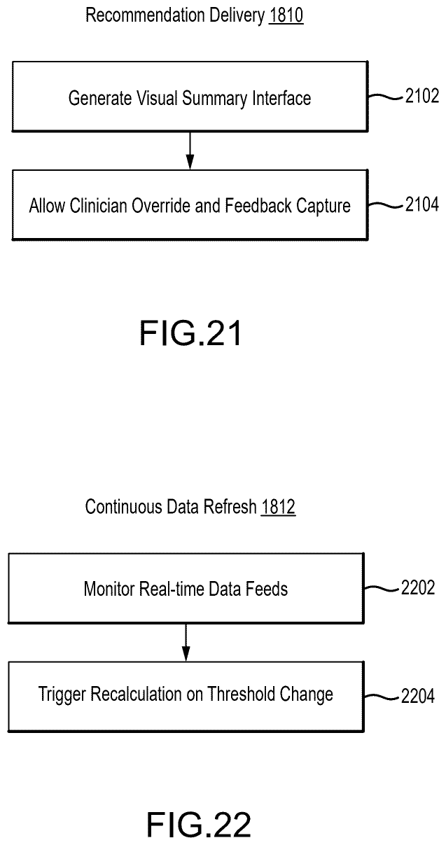

FIGS. 18-22 depict a method 1800 and sub-methods, e.g., FIG. 19 depicts a sub-method of the suitability scoring step 1806, FIG. 20 depicts a sub-method of the ranking and optimization step 1808, FIG. 21 depicts a sub-method of the recommendation delivery step 1840, and FIG. 22 depicts a sub-method of the continuous data refresh step 1812. The patient-specific medication recommendation method 1800, which can also be referred to herein as a patient-specific medication ranking method, can orchestrate data acquisition, data harmonization and preprocessing, suitability scoring, ranking and optimization, recommendation delivery, and continuous data refresh to generate individualized therapy guidance. In one example, the method 1800 can execute the patient-specific medication recommendation method 1800 step iteratively so that each downstream operation consumes updated inputs and produces traceable outputs. More specifically, the method 1800 can align patient parameters, drug knowledge, and epidemiological context to produce scores, ranks, and justifications with clinician feedback capture. Thus, the method 1800 addresses dose adjustment complexity, lack of real-time resistance visibility, fragmented efficacy-cost information, and clinician time constraints by automating end-to-end computation and presentation.

The presentation layer 1544 can choose presentation template prior to generating a visual summary so that rendering matches a clinician workflow without modifying analytical results. In one example, an administrative interface can configure factor weighting matrix for efficacy, safety, organ feasibility, resistance, cost, availability, and insurance coverage, with versioned persistence for traceability. Additionally, administrators can define recalculation thresholds for susceptibility shifts, price changes, and regulatory updates to drive automated re-scoring. Thus, template selection improves usability while configurable weights and thresholds align outputs with local policy and solve the challenge of integrating up-to-date efficacy, availability, and cost information into decisions.

The analysis engine 1532 can individualize medication selection by combining patient physiology, comorbidities, laboratory values, and medication history into a patient-specific ranking process. In one example, the multi-criteria ranking engine 1542 can optimize cost efficiency by adjusting positions based on insurance coverage and projected out-of-pocket expense while preserving clinical suitability. In particular, the cost and coverage analyzer 1538 can apply payer and formulary data to differentiate clinically comparable agents. Thus, the configuration improves individualized therapy and addresses absence of effectiveness-coverage comparison in routine prescribing. This prevents or reduces the problem of patients being sent to a pharmacy with a prescription they do not have the means to fill and then having to go back to the physician for a change in the prescription. This could also increase the speed at which prior authorizations are submitted by providing the medical data supporting a decision to prescribe that particular medication, e.g., as opposed to a lower cost medication.

The interaction and contraindication checker 1536 can reduce adverse events by screening candidate medications against allergies, concurrent therapies, and disease-based contraindications. In one example, the dose calculation engine 1534 can constrain organ-adjusted dosage ranges using renal and hepatic metrics, age, and weight to avoid toxicity. Additionally, the analysis engine 1532 can down-weight or exclude options that exceed risk thresholds derived from the drug-patient interaction risk profile. Thus, the workflow reduces interaction and contraindication risk during selection. For example, some interactions are not “Zero-sum” or “all-or-nothing”, and the medical prescriber should determine the risk to benefit ratio with the patient, e.g., especially in the case when there are no other options available. The medicine recommendation platform 1500 and/or the method 1800 can help providers stratify the risk to benefit in one place and determine if the interactions are minor or more serious, which helps the prescriber make a determination efficiently during the workflow.

The data acquisition layer 1516 can maintain decision currency by ingesting real-time resistance updates, formulary availability, and pricing signals from external clinical and pharmaceutical data streams. In one example, the platform can evaluate defined thresholds and trigger recalculation on threshold change to refresh rankings without manual intervention. Additionally, the caching store 1530 can propagate normalized real-time data update into the harmonized dataset for immediate scoring. Thus, the system prevents or reduces reliance on outdated information and addresses missing real-time susceptibility at the point of prescribing.

The method 1800 can accept as inputs the current drug knowledge dataset and the patient-specific data package to build a harmonized clinical and drug dataset. In one example, the data acquisition layer 1516 can retrieve dosing guidance, contraindications, safety metrics, real-time resistance, availability, pricing, and regulatory labeling while collecting demographics, organ function, allergies, and insurance data or information (e.g., patient insurance tier, payor, or formulary information) for the patient. Additionally, the preprocessing pipeline can normalize formats and units across both inputs to enable deterministic scoring and ranking. Thus, the structured inputs enable accurate dosing, safety evaluation, and cost integration needed to resolve multi-variable dose adjustment and financial comparison challenges.

The medicine recommendation platform 1500 and/or the method 1800 can output a ranked medication recommendation set containing ordered options with individualized dosing, efficacy estimates, safety annotations, cost and coverage parameters, and rationale. In one example, the multi-criteria ranking engine 1542 can produce the set from the medication suitability score list and cost adjustments, and the presentation layer 1544 can render the set for clinician review. Additionally, the platform can attach traceability metadata and capture clinician override to finalize a selected medication order. Thus, the delivered set streamlines decision-making under time constraints while preserving transparency and alignment with current clinical and financial data.

### Method Step—Data Acquisition 1802

The method 1800 can include a data acquisition step 1802. For example, the data acquisition layer 1516 executes data acquisition by concurrently querying patient-facing systems, drug repositories, and epidemiological feeds to reduce end-to-end latency. In one example, the data acquisition layer 1516 schedules and parallelizes source-specific connectors, applies source authentication, and timestamps each record for freshness assessment (e.g., within 1 to 24 hours). More specifically, the data acquisition layer 1516 normalizes heterogeneous payloads into a unified schema suitable for downstream preprocessing and triggers substeps according to the clinical context and/or medication class under consideration. Thus, the data acquisition layer 1516 prepares complete inputs for subsequent harmonization by orchestrating the subordinate extraction and retrieval steps.

The patient information collector 1518 executes extract patient financial and medication history by querying electronic medical records, pharmacy benefit managers, and payer APIs to assemble active medication lists, allergies, formulary tiers, coverage restrictions, and the like. In one example, the patient information collector 1518 retrieves real-time copay ranges (e.g., within USD 1 to 500), out-of-network pricing, prior authorization requirements, and claims-based adherence indicators, and then reconciles duplicate entries. More specifically, the patient information collector 1518 aligns timestamps and source priorities to prefer the most recent fill history and merges results into the patient-specific data package. Alternatively, the patient information collector 1518 can schedule periodic refreshes or event-driven updates when payer coverage changes.

The patient information collector 1518 executes extract patient-specific clinical parameters by acquiring demographics, vitals, laboratory results (e.g., serum creatinine, liver enzymes, or blood tests relevant to the specific patient or medication being considered, such as INR for a blood thinner or platelet count for a platelet deactivator), comorbidities, and current medications from EMR systems and/or provider and patient inputs. In one example, the patient information collector 1518 maps laboratory codes to a unified ontology, converts measurement units, and selects recent values within a configurable lookback window (e.g., 1 hour to 90 days). More specifically, the patient information collector 1518 validates outliers, flags missing renal or hepatic metrics, and prompts for supplemental entry when required. Thus, the patient information collector 1518 inserts standardized clinical parameters into the patient-specific data package.

The drug information collector 1520 executes retrieve candidate drug information by aggregating regulatory labels, pharmacokinetic and pharmacodynamic profiles, dosing guidance parameterized by organ function, lab tests, drug specific calculations, contraindications, side-effect frequencies, and availability. In one example, the drug information collector 1520 accesses regulatory data via a regulatory data interface 1702 and queries inventory and acquisition cost via a hospital/pharmacy database connector 1704, while normalizing pricing across sources. More specifically, the drug information collector 1520 structures attributes for each candidate medication and stores the results for downstream use. Thus, the drug information collector 1520 contributes structured attributes to the current drug knowledge dataset.

The environmental information collector 1522 executes retrieve contextual epidemiological data by obtaining local and regional antibiograms, resistance trends, and outbreak alerts from laboratory information systems and public health feeds. In one example, the environmental information collector 1522 filters feeds by facility, pathogen, and geography, and updates at intervals ranging from near real-time to daily. More specifically, the environmental information collector 1522 aligns susceptibility statistics with candidate antimicrobials and time-stamps the trends for recency weighting. Thus, the environmental information collector 1522 appends epidemiological context to the current drug knowledge dataset.

The data acquisition layer 1516 emits the current drug knowledge dataset and the patient-specific data package as machine-readable outputs formatted for downstream analysis. In one example, the data acquisition layer 1516 serializes outputs as HL7 FHIR bundles and/or JSON documents, embeds versioned schemas, and records source provenance and acquisition timestamps. More specifically, the data acquisition layer 1516 includes for each medication dosing guidance parameterized by patient variables, contraindications, side-effect profiles, availability, pricing, and resistance context, while the patient-specific data package aggregates standardized clinical, medication history, allergy, and coverage data. Thus, the data acquisition layer 1516 provides stable, queryable artifacts that feed subsequent harmonization and scoring.

### Method Step—Data Harmonization and Preprocessing 1804

The method 1800 can include a data harmonization and preprocessing step 1804. For example, the data acquisition layer 1516 can execute a data harmonization and preprocessing step that converts heterogeneous patient and drug records into a standardized, machine-readable schema. In one example, the harmonizer can map ICD-10, SNOMED CT, LOINC, and RxNorm codes to a unified ontology, align timestamps, and validate integrity against reference standards. Additionally, the harmonizer can clean erroneous entries and reconcile duplicate entities to prepare data for suitability scoring and ranking.

A rules engine can apply dynamic unit normalization rules that convert incoming measurements to platform-standard units while recording versioned conversion factors. In one example, the system can establish semantic consistency by mapping units and terminologies and can provide downstream compatibility by emitting types and ranges required by scoring and visualization modules. More specifically, the system can normalize patient parameters using conditional logic, such as age- or sex-adjusted reference multipliers and cost conversions to a cost-per-dose basis.

The harmonizer can perform outlier detection algorithm selection that assigns per-field models such as Z-score thresholds, Tukey fences, and/or isolation forests. In one example, the pipeline can safeguard data reliability by tagging improbable physiologic values and anomalous financial values with severity levels that guide later reconciliation or user review.

The harmonizer can orchestrate a missing-data resolution workflow that prioritizes automated retrieval before user intervention. In one example, the system can detect missing/conflicting data and request update by querying EMR, Laboratory Information Systems (LIS), pharmacy benefit manager (PBM), and pharmacy connectors, and subsequently issuing asynchronous prompts to clinicians and/or patients when automated retrieval fails. Then, the pipeline can re-validate newly obtained values through the same cleaning and normalization rules to enable completeness before scoring.

The harmonizer can ingest a current drug knowledge dataset and a patient-specific data package as inputs to a unified preprocessing pipeline. In one example, the system can align dosing, contraindications, resistance metrics, availability, and cost fields with patient demographics, organ function metrics, active medications, allergies, and coverage attributes.

The data acquisition layer 1516 can output a harmonized clinical and drug dataset that fuses normalized patient parameters with standardized drug attributes. In one example, the pipeline can emit a schema-conformant object that preserves ontology mappings, unit-normalized values, and validation provenance to enable downstream suitability scoring without additional transformation.

### Method Step—Suitability Scoring 1806

The method 1800 can include a suitability scoring step 1806. For example, the analysis engine 1532 executes suitability scoring by consuming the harmonized clinical and drug dataset and generating a medication suitability score list annotated with dosing guidance. In one example, the analysis engine 1532 incorporates outputs of the dose calculation engine 1534, the interaction and contraindication checker 1536, and the efficacy and resistance evaluator 1540 to align pharmacology, safety, and context-aware effectiveness for each candidate medication. Additionally, the analysis engine 1532 writes traceable factor contributions for each score to support downstream ranking and optimization. Therefore, the suitability scoring step reduces manual synthesis effort and enables physicians to integrate multi-source clinical evidence within prescribing time constraints.

The analysis engine 1532 subscribes to normalized real-time data update from the real-time resistance data connector 1706 and the environmental information collector 1522 to maintain synchronized suitability scores. In one example, the analysis engine 1532 triggers recalculation on threshold change to refresh the medication suitability score list using the updated harmonized dataset. Additionally, the integration and API layer 1552 propagates versioned updates to the presentation layer 1544 during a prescribing session and/or over a treatment course. Therefore, the real-time responsiveness addresses the absence of point-of-prescribing susceptibility data and mitigates outdated recommendations.

The analysis engine 1532 incorporates preliminary cost and coverage factors within compute composite suitability score using patient-specific cost and coverage data when available. In one example, the analysis engine 1532 normalizes cost, formulary status, and availability signals and applies configurable weighting coefficients to reflect institutional policy. Additionally or alternatively, the analysis engine 1532 defers fine-grained financial adjustments to the cost and coverage analyzer 1538 during ranking and optimization while retaining affordability sensitivity within suitability scoring. Therefore, the incorporation of cost metrics addresses the inability to compare effectiveness with insurance coverage, real-world prices/cost, and availability (e.g., in circumstances where medications are backordered, sold out, or in critical shortage).

The analysis engine 1532 computes a single quantitative metric per medication by aggregating efficacy, safety, resistance, organ-adjustability, and preliminary cost factors into a composite suitability score. In one example, the analysis engine 1532 applies a mathematical function (e.g., a weighted sum or multi-criteria decision analysis) with user-configurable coefficients and normalization across comparable ranges. Additionally, the analysis engine 1532 supports rule-based logic and/or learned models to produce reproducible scores suitable for downstream rank ordering. Therefore, the quantitative metric enables rapid comparison across options and supports reproducible, evidence-weighted decision-making.

The dose calculation engine 1534 calculates organ-adjusted dosage range by referencing renal and hepatic function, other blood tests that could affect drug dosing (such as coagulation functions), demographic measures, labeled guidance, and the like to output drug-specific organ-adjusted dose guidance. More specifically, the interaction and contraindication checker 1536 evaluates drug-patient contraindications and interactions using the harmonized clinical and drug dataset to produce a drug-patient interaction risk profile that gates or modifies dosing feasibility. In particular, the analysis engine 1532 computes composite suitability score by combining efficacy, resistance, safety, organ-adjusted dosing feasibility, and preliminary cost into a normalized score for each candidate. Therefore, the combined calculations address dose personalization across organ impairment and reduce adverse interaction risk during medication selection.

The analysis engine 1532 consumes a harmonized clinical and drug dataset and an updated harmonized dataset as versioned inputs to initialize suitability scoring and to refresh scores upon data change. In one example, the analysis engine 1532 validates schema conformance, aligns ontology terms, and enforces freshness windows (e.g., between 5 and 120 minutes) before the analysis engine 1532 passes patient parameters and drug attributes to the dose calculation engine 1534 and the interaction and contraindication checker 1536. Additionally, the analysis engine 1532 weights context signals from real-time resistance and epidemiological feeds within efficacy and/or feasibility calculations only when the updated harmonized dataset satisfies predefined completeness thresholds. Therefore, the structured, versioned inputs enable synchronized, up-to-date scoring that integrates multi-source clinical evidence and addresses lack of real-time susceptibility data.

The analysis engine 1532 generates a medication suitability score list as an output that enumerates candidate medications with a composite suitability score, per-factor subscores, and drug-specific organ-adjusted dose guidance. In one example, the analysis engine 1532 includes gating indicators for contraindications and interactions, rationale features with factor contributions, and provenance metadata such as data timestamps, rule set or model version, and calculation configuration, all exposed via an API and/or persisted for downstream ranking and optimization. Additionally, the analysis engine 1532 supports incremental recomputation to append delta updates when an updated harmonized dataset arrives, and supports a deterministic rule-based weighted sum scoring variant to enable reproducible results across sessions. Therefore, the structured list enables rapid comparison under physician time constraints while reducing adverse interaction risk through transparent gating and dosing feasibility annotations.

### Suitability Scoring 1806—Calculate Organ-Adjusted Dosage Range 1902

The method 1800, e.g., the suitability scoring step 1806, can include a calculate an organ-adjusted dosage range step 1902, e.g., for each candidate medication by combining renal metrics (e.g., eGFR or creatinine clearance), hepatic metrics (e.g., Child-Pugh category or transaminase levels), and body size metrics (e.g., body weight or body-surface area). More specifically, the calculation can reference labeled dosing instructions and/or peer-reviewed pharmacokinetic models to produce initial, maintenance, and maximum dose values and/or dosing intervals (e.g., every 12-48 hours) that are scaled according to organ impairment categories. In particular, the calculation can apply linear and/or non-linear transformations to dose and/or interval based on parameter thresholds (e.g., eGFR bands spanning 15-90 mL/min/1.73 m²) and can adjust multiple parameters concurrently. Additionally or alternatively, the calculation can flag medications with no safe dose due to contraindications or missing data and can annotate each computed value with a source citation and a computation method for downstream traceability.

The calculation can receive a harmonized clinical and drug dataset as input that aggregates normalized patient parameters and standardized drug attributes. More specifically, the dataset can provide contemporaneous laboratory values, demographics, comorbidities, and current therapies alongside dosing rules stratified by organ function, availability constraints, and packaging units. In particular, the calculation can consume values expressed in unified units and mapped vocabularies, which can enable deterministic rule matching and model selection. Thus, the calculation can operate without additional preprocessing and can leverage temporally aligned values to avoid stale guidance.

The dose calculation engine 1534 can execute the calculation to generate drug-specific organ-adjusted dose guidance as an output data object. More specifically, the engine can emit for each drug a recommended starting dose, a maintenance dose, and a maximum allowable dose, each optionally paired with an adjusted interval and route (e.g., dose 0.5-1.0 mg/kg every 24-48 hours), and each annotated with the applied rule or model and the source reference. In particular, the engine can embed warnings for organ-related limitations and/or comorbidity-dependent adjustments and can record the parameter values and thresholds used during computation. Thus, the output can feed subsequent composite suitability scoring and presentation while preserving calculation transparency for clinician review.

### Suitability Scoring 1806—Evaluate Drug-Patient Contraindications and Interactions 1904

The method 1800, e.g., the suitability scoring step 1806, can include an evaluating drug-patient contraindications and interactions step 1904. For example, the analysis engine 1532 can execute the evaluate drug-patient contraindications and interactions step by invoking the interaction and contraindication checker 1536 over a candidate medication set and a harmonized clinical and drug dataset. More specifically, the interaction and contraindication checker 1536 can traverse contraindication rules and interaction graphs, cross-reference current medications, allergies, comorbidities, and laboratory-derived renal and hepatic function, and assign graded severities to identified drug-drug and drug-condition risks. In one example, the interaction and contraindication checker 1536 can simulate pharmacokinetic and pharmacodynamic behavior under organ-adjusted parameters and can apply user-configurable alert thresholds to generate actionable findings. Therefore, the evaluation step can reduce the risk of adverse interactions while offsetting physician time constraints through automated, patient-specific screening.

The analysis engine 1532 can consume a harmonized clinical and drug dataset as an input to the evaluate drug-patient contraindications and interactions step. More specifically, the harmonized clinical and drug dataset can supply normalized patient parameters, standardized drug attributes, contemporaneous contraindication tables, and context such as real-time resistance data and formulary constraints aligned to a common ontology. In one example, the harmonized clinical and drug dataset can maintain temporally versioned entries so the interaction and contraindication checker 1536 evaluates candidate drugs against contemporaneous laboratory values and current labels. Therefore, the input dataset can provide unified, up-to-date evidence that addresses fragmented sources and supports accurate contraindication screening.

The interaction and contraindication checker 1536 can generate a drug-patient interaction risk profile as an output for each candidate medication. More specifically, the drug-patient interaction risk profile can enumerate contraindication flags, drug-drug and drug-condition interactions, severity and likelihood scores, and recommended alternatives and/or monitoring protocols, with links to provenance in the harmonized clinical and drug dataset. In one example, the drug-patient interaction risk profile can include cumulative risk indices and machine-readable structures consumed by the compute composite suitability score step. Therefore, the generated profile can enable downstream ranking and safe selection by quantifying risk in a structured, traceable artifact.

### Suitability Scoring 1806—Compute Composite Suitability Score 1906

The method 1800, e.g., the suitability scoring step 1806, can include a compute a composite suitability score step 1906. For example, the analysis engine 1532 can compute a composite suitability score for each candidate medication by aggregating weighted factors derived from the harmonized clinical and drug dataset. In one example, the analysis engine 1532 assigns configurable coefficients (e.g., between 0.05 and 0.6 per factor with a normalized total near 1.0) and combines efficacy, safety, resistance likelihood, organ-adjusted dosing feasibility, and preliminary cost impact via a scoring routine such as a weighted sum or a multi-criteria decision analysis. More specifically, the analysis engine 1532 can normalize heterogeneous factor ranges (e.g., map raw metrics to a common scale between 0 and 1) and apply a deterministic rule-based weighted sum scoring variant to produce a single numerical score per medication. Thus, the analysis engine 1532 fuses multi-dimensional clinical and operational evidence into a concise quantitative signal, which reduces physician time burden and supports decisions that reflect current efficacy, safety, resistance, dosing feasibility, and preliminary cost.

The analysis engine 1532 can receive the drug-patient interaction risk profile, the drug-specific organ-adjusted dose guidance, and the harmonized clinical and drug dataset as inputs to the composite suitability computation. More specifically, the analysis engine 1532 can attenuate the composite score according to interaction severity and contraindication flags from the drug-patient interaction risk profile, and can cap or boost the score according to feasibility bands from the drug-specific organ-adjusted dose guidance derived from renal and hepatic function parameters. In one example, the analysis engine 1532 can source contemporaneous efficacy and resistance features from the harmonized clinical and drug dataset, including real-time antibiogram signals, and incorporate those features as efficacy and resistance sub-scores before aggregation. Thus, the analysis engine 1532 directly mitigates adverse interaction risk, incorporates real-time resistance intelligence, and operationalizes patient-specific dose adjustment constraints within a unified computation.

The analysis engine 1532 can output a medication suitability score list that enumerates each candidate medication with a composite suitability score and optional per-factor sub-scores for downstream ranking and optimization. In one example, the analysis engine 1532 can attach calculation metadata and coefficient settings to each entry to enable traceability and to support rapid re-computation when the updated harmonized dataset becomes available. Additionally, the analysis engine 1532 can refresh the medication suitability score list upon a threshold-triggered recalculation to reflect evolving laboratory values, resistance feeds, and formulary or cost updates. Thus, the analysis engine 1532 delivers a structured, up-to-date comparison set that streamlines clinician review and integrates dynamic clinical and pharmaceutical data without manual synthesis.

### Method Step—Ranking and Optimization 1808

The method 1800 can include a ranking and optimization step 1808. For example, the multi-criteria ranking engine 1542 can execute the ranking and optimization step by consuming a medication suitability score list and producing a ranked medication recommendation set, which can enable or permit healthcare providers to confirm or double check a prescription includes the correct medication as well as the correct dose. More specifically, the multi-criteria ranking engine 1542 can apply configurable optimization logic across efficacy, safety, resistance, availability, and economic sub-scores to synthesize an ordered set. In one example, the analysis engine 1532 can coordinate downstream adjustments and selection to finalize an actionable recommendation object. Thus, the ranking and optimization step addresses physician time constraints by automating multi-factor synthesis into a concise, patient-specific ordering.

The multi-criteria ranking engine 1542 can define a weighting schema by loading a policy-driven matrix and assigning numeric weights to efficacy, safety, organ-adjusted dosing feasibility, resistance, availability, and financial factors. More specifically, the multi-criteria ranking engine 1542 can execute a tie-breaking protocol that orders candidates by secondary metrics (e.g., clinical efficacy, stewardship category, copay) when aggregate scores match. In one example, the analysis engine 1532 can generate a dose-adjusted candidate set by merging candidates with dose calculation engine 1534 outputs and by deprioritizing candidates without a safe dose. Thus, the weighting, tie-breaks, and dose gating address dose-adjustment complexity and prevent or reduce unusable or clinically inferior options from propagating into the ranked results.

The analysis engine 1532 can adapt to real-time data changes by subscribing to normalized real-time data update and by monitoring external clinical and pharmaceutical data streams. More specifically, the analysis engine 1532 can trigger recalculation on threshold change to re-rank candidates when resistance, pricing, availability, or patient parameters shift beyond configured bounds. In one example, the caching store 1530 can version intermediate inputs so that updated harmonized datasets drive deterministic re-computation. Thus, adaptive re-ranking addresses absence of real-time susceptibility data in prescribing workflows by reflecting current microbiology and market conditions in near real time.

The cost and coverage analyzer 1538 can reduce financial burden by ingesting patient-specific cost and coverage data and by computing projected out-of-pocket cost and institutional acquisition cost for each candidate. More specifically, the cost and coverage analyzer 1538 can surface formulary tier, prior authorization, and cash-pay alternatives to inform rank adjustments toward lower total cost of care. In one example, the summary dashboard 1546 can present projected savings relative to baseline regimens to support value-based selection. Thus, integrated financial analysis addresses absent comparison between effectiveness and coverage by steering recommendations toward effective, but also affordable, accessible therapies. The cost and coverage analyzer 1538 can also assist with prior authorization by making a record of medical need and documenting and transmitting the record to accelerate the care of medical patients.

The multi-criteria ranking engine 1542 can rank drugs by composite score by aggregating weighted sub-scores from the medication suitability score list into an initial ranked medication list. More specifically, the cost and coverage analyzer 1538 can apply cost and insurance adjustment to modify the initial ordering using patient-specific cost and coverage data and real-time availability. Subsequently, the multi-criteria ranking engine 1542 can select top medication set to emit a primary recommendation and one or more policy-diverse alternatives. Thus, staged scoring, financial adjustment, and final selection address integration of efficacy, safety, resistance, and access constraints into a single clinician-ready ordering.

The multi-criteria ranking engine 1542 can receive the medication suitability score list as an input that encodes per-drug sub-scores and organ-adjusted dosing guidance for a specific patient. More specifically, the multi-criteria ranking engine 1542 can execute the ranking and optimization step to transform the input into a ranked medication recommendation set while preserving traceable factor contributions. In one example, the presentation layer 1544 can consume the ranked medication recommendation set to render rationale and dosing context for clinician review. Thus, structured scoring input and ordered output address risk of adverse interactions and dose errors by carrying forward patient-specific constraints into the final recommendations.

### Ranking and Optimization 1808—Rank Drugs by Composite Score 2002

The method 1800, e.g., the ranking and optimization step 1808, can include a ranking drugs by composite score step 2002. For example, the multi-criteria ranking engine 1542 can execute the rank drugs by composite score step by receiving a medication suitability score list and sorting candidate medications in descending order of composite score to produce an initial ranked medication list. More specifically, the multi-criteria ranking engine 1542 can apply a tiebreaker protocol that first compares clinical efficacy sub-scores and subsequently compares safety sub-scores when composite scores match. In one example, the multi-criteria ranking engine 1542 can apply a deterministic rule-based weighted-sum model and/or a learned ranking model trained on historical outcomes to order candidates. Alternatively, the multi-criteria ranking engine 1542 can incorporate guideline-derived and/or provider-defined weights and can constrain ordering according to institution-specific formularies.

The multi-criteria ranking engine 1542 can ingest the medication suitability score list as an input artifact and can emit the initial ranked medication list as an output of the rank drugs by composite score step. More specifically, the multi-criteria ranking engine 1542 can map per-factor sub-scores within the medication suitability score list to a configurable weighting scheme and can compute ordering according to a selected multi-attribute decision technique. In one example, the multi-criteria ranking engine 1542 can expose parameters for per-indication re-weighting and/or cohort-level defaults to support consistent institutional practice. Thus, the multi-criteria ranking engine 1542 can generate a patient-specific ordering that serves downstream cost and coverage adjustment without embedding financial modifiers at this stage.

### Ranking and Optimization 1808—Apply Cost and Insurance Adjustment 2004

The method 1800, e.g., the ranking and optimization step 1808, can include an applying cost and insurance adjustment step 2004. For example, the cost and coverage analyzer 1538 can apply cost and insurance adjustment by re-scoring candidate medications according to real-time pricing and insurance constraints for the patient. In one example, the cost and coverage analyzer 1538 executes the apply cost and insurance adjustment step and updates weights for out-of-pocket exposure, formulary tiering, prior-authorization status, and pharmacy-specific acquisition cost, and can repeat the apply cost and insurance adjustment step when pricing or coverage feeds change. Additionally, the cost and coverage analyzer 1538 can incorporate projected administration and monitoring expenses to refine the re-scoring. Therefore, the cost and coverage analyzer 1538 addresses the inability to integrate up-to-date cost and coverage information and enables automated comparison across covered and out-of-network options.

The analysis engine 1532 can receive the initial ranked medication list as an input that reflects clinical suitability prior to financial considerations. More specifically, the analysis engine 1532 can also receive patient-specific cost and coverage data as an input that includes formulary placement, prior-authorization requirements, coinsurance or copay projections, contract prices, cash-pay alternatives, and out-of-network pricing for each candidate medication. In one example, the analysis engine 1532 aligns these inputs by medication identifier and coverage plan metadata to prepare re-scoring. Thus, the input pairing reduces physician time spent gathering fragmented financial data and prepares a reliable basis for downstream adjustment.

The cost and coverage analyzer 1538 can output a cost-adjusted ranked medication list that orders medications by a composite reflecting clinical suitability and patient-specific financial impact. In one example, the cost and coverage analyzer 1538 executes the adjustment and annotates each entry with coverage tier, prior-authorization flag, estimated out-of-pocket burden, and contract or cash pricing, and can compute projected per-episode or weekly savings relative to a baseline regimen. Alternatively, the cost and coverage analyzer 1538 can filter or re-sort entries according to user-defined affordability thresholds to support selection. Therefore, the produced list enables cost-informed prescribing while maintaining clinical appropriateness, addressing coverage comparison gaps and reducing manual review under time constraints.

### Ranking and Optimization 1808—Select Top Medication Set 2006

The method 1800, e.g., the ranking and optimization step 1808, can include a selecting a top medication set step 2006. For example, the multi-criteria ranking engine 1542 can execute the select top medication set step by sampling the highest scoring entries from a cost-adjusted ranked medication list and assembling a primary recommendation with one or more secondary alternatives. In one example, the multi-criteria ranking engine 1542 can select k options (e.g., between 2 and 7) using constraint-aware selection that enforces safety filters, coverage thresholds, and mechanism-of-action diversity. More specifically, the multi-criteria ranking engine 1542 can apply tie-breaking rules based on predicted efficacy variance, prior-authorization burden, and availability latency to finalize ordering. Then, the multi-criteria ranking engine 1542 can package the ordered options as a ranked medication recommendation set configured for downstream presentation and/or order placement.

The multi-criteria ranking engine 1542 can receive a cost-adjusted ranked medication list as input and can transform selected entries into an output ranked medication recommendation set. In one example, the multi-criteria ranking engine 1542 can evaluate each candidate using configurable weights and constraints, and can emit a structured object that enumerates the primary option and alternative options with dosing ranges, coverage annotations, expected out-of-pocket estimates, and interaction warnings. Additionally, the multi-criteria ranking engine 1542 can support event-based re-execution when patient variables or market variables change, thereby regenerating the ranked medication recommendation set. Thus, the multi-criteria ranking engine 1542 can provide deterministic or learned selection behavior while maintaining traceability from the cost-adjusted ranked medication list to the ranked medication recommendation set.

### Method Step—Recommendation Delivery 1810

The method 1800 can include a recommendation delivery step 1810. For example, the presentation layer 1544 can execute recommendation delivery by rendering the ranked medication recommendation set with patient-specific dosage guidance, efficacy metrics, safety annotations, availability, and cost data in a single interface. In one example, the presentation layer 1544 can generate explanations and provenance indicators that reference the data sources and variable weightings applied by the analysis engine 1532. Additionally, the presentation layer 1544 can support EMR order initiation and/or export so that a selected recommendation transitions to a final selected medication order without duplicate entry. Further, the presentation layer 1544 can refresh the rendered view in near real time when updated harmonized dataset inputs modify the ranking or dosing.

The presentation layer 1544 can apply confidence threshold highlighting by mapping composite suitability score ranges (e.g., ≥80-90% green, 65-85% yellow, <65-75% red) to row-level color and/or icon cues. In one example, the presentation layer 1544 can retrieve threshold values from an institution settings service at run time and can execute the highlighting logic on the client to achieve sub-100 ms updates. More specifically, the presentation layer 1544 can recalculate visual states when a user sorts or filters the ranked list, avoiding additional server requests. Thus, the interface can convey score confidence without delaying clinician interaction.

The presentation layer 1544 can juxtapose efficacy, organ-adjusted dosing ranges, contraindication flags, availability, and cost in a single viewport to facilitate rapid comparative assessment. In one example, the presentation layer 1544 can provide columnar tables, pictograms, and compact tooltips that summarize justification details on demand. Additionally, the presentation layer 1544 can integrate seamlessly with clinical workflow by allowing one-click progression from recommendation review to EMR order composition. Therefore, the clinician can identify a preferred option within seconds while maintaining continuity of care tasks.

The presentation layer 1544 can reduce prescription errors by embedding dose range guardrails, interaction alerts from the interaction and contraindication checker 1536, and contraindication warnings linked to patient allergies and organ function. In one example, the presentation layer 1544 can block order progression or require acknowledgment when high-risk conditions are detected. Additionally, the presentation layer 1544 can surface organ-adjusted dose guidance alongside unit calculators to prevent or reduce unit or frequency mismatch. Thus, the interface can intercept unsafe selections before order submission.

The presentation layer 1544 can allow clinician override and feedback capture by enabling acceptance, modification, or rejection of a recommendation with structured and/or free-text rationale that produces a clinician override and feedback record. In one example, the summary dashboard 1546 can generate visual summary interface elements for selection, sorting, and filtering, while a justification panel can provide justification and traceability metadata including data provenance, algorithm versions, and weighting factors. Additionally, the presentation layer 1544 can prompt optional outcome entry after therapy to support learning loops and audit trails. Further, the presentation layer 1544 can route accepted selections to EMR order entry to produce a final selected medication order.

The recommendation delivery step can consume the ranked medication recommendation set as an input and can render the ordered candidates with associated dosing, efficacy, safety, availability, and cost annotations. In one example, the presentation layer 1544 can stream incremental updates to the same set when the analysis engine 1532 re-ranks options based on updated harmonized clinical and drug dataset inputs. Additionally, the presentation layer 1544 can expose programmatic endpoints to deliver the ranked medication recommendation set to third-party systems. Therefore, downstream systems can display or action the same prioritized list without re-computation.

### Recommendation Delivery 1810—Generate Visual Summary Interface 2102

The method 1800, e.g., the recommendation delivery step 1810, can include a generate visual summary interface step 2102. For example, the recommendation delivery subsystem can execute a generate visual summary interface step that renders a patient-specific, structured display of candidate medications. In one example, the step can render a table and/or pictogram arrangement that lists, for each candidate, a drug name, a personalized dose based on renal and hepatic function and/or weight, an efficacy metric derived from real-time epidemiological data, a cost estimate based on coverage and pricing sources, and alert indicators for interactions and contraindications. Additionally, the step can enable sorting, filtering, and ranking by efficacy, cost, and/or safety, and can regenerate the display in response to updated patient data or drug information. Alternatively, the step can export the display to an EMR module and/or a print artifact for provider review.

More specifically, the generate visual summary interface step can ingest a ranked medication recommendation set as an input to populate ordered rows and associated dosing, efficacy, safety, and cost attributes. In one example, the step can also ingest a traceability metadata bundle as an input to surface rationale indicators and/or drill-down links that expose timestamps, data sources, algorithm versions, and intermediate scoring values. Then, the step can map recommendation ordering to display prominence and can annotate each medication with interaction warnings and formulary status derived from the input structures.

Further, the summary dashboard 1546 can execute the generate visual summary interface step to output a visual summary interface view that presents the ordered medication options with interactive controls. In one example, the summary dashboard 1546 can support drill-down to detailed efficacy evidence, packaging depictions, availability data, and dosing calculators while maintaining alert iconography for allergies and contraindications. Additionally, the summary dashboard 1546 can refresh the view in real time based on updated clinical or pricing feeds and can persist user-selected filters for workflow continuity. Thus, the summary dashboard 1546 can deliver the visual summary interface view for clinician action within the presentation layer 1544.

### Recommendation Delivery 1810—Allow Clinician Override and Feedback Capture 2104

The method 1800, e.g., the recommendation delivery step 1810, can include an allow clinician override and feedback capture step 2104. For example, the packaging display module 1550 can provide a step that enables a clinician to accept, modify, or reject a proposed medication and dosing regimen. In one example, the presentation layer 1544 can display patient-specific variables, contraindication and interaction warnings, and economic parameters, and can accept clinician-entered alternative drugs, dose adjustments, and/or rationale annotations. Additionally or alternatively, the presentation layer 1544 can prompt entry of structured and/or free-text outcome observations after therapy initiation and can associate the observations with the corresponding patient encounter and recommendation instance. In one embodiment, the presentation layer 1544 can record an audit trail of actions and can queue captured inputs for downstream analytics and quality processes.

The presentation layer 1544 can receive a ranked medication recommendation set as an input to populate selectable options with dosing guidance, predicted efficacy, safety annotations, and financial data. More specifically, the presentation layer 1544 can ingest a traceability metadata bundle as an input to expose algorithm versioning, weighting coefficients, timestamps, and intermediate scoring artifacts during an override workflow. Additionally, the presentation layer 1544 can render a visual summary interface view as an input-driven interactive surface that supports filtering, drill-down, and confirmation of a revised selection. Thus, the presentation layer 1544 can use the three inputs to contextualize clinician actions and to enable transparent justification for any deviation.

The presentation layer 1544 can execute the allow clinician override and feedback capture step to produce a clinician override and feedback record as an output and a final selected medication order as an output. In one embodiment, the presentation layer 1544 can structure the record with coded override reasons, modification types, outcome fields, free-text notes, timestamps, user identifiers, and links to the source recommendation and traceability metadata. Additionally, the presentation layer 1544 can generate the final selected medication order with drug, dose, route, frequency, and monitoring instructions formatted for EMR and/or e-prescribing submission. In one example, the presentation layer 1544 can persist both outputs to a repository for stewardship reporting and continuous model refinement.

### Method Step—Continuous Data Refresh 1812

The method 1800 can include a continuous data refresh 1812. For example, the data acquisition layer 1516 can execute a continuous data refresh step that, after generating an initial recommendation, subscribes and/or polls designated feeds for updates to clinical, efficacy, availability, and cost parameters. In one example, the medicine recommendation platform 1500 and/or the method 1800 can initiate background re-ingestion and harmonization upon detection of updates and can notify the presentation layer 1544 of an updated recommendation state. Additionally or alternatively, the analysis engine 1532 can schedule periodic refresh cycles and can suppress redundant runs within a configurable time window. Therefore, the continuous refresh capability addresses lack of real-time data and physician time constraints by maintaining current inputs without manual review.

More specifically, a rule engine of the analysis engine 1532 can implement a significant-change threshold setter that defines materiality for each monitored parameter using absolute and/or relative thresholds stored in a version-controlled policy. In one example, the multi-criteria ranking engine 1542 can perform incremental score re-computation by consulting a dependency graph to identify only candidate medications affected by the triggered parameters. Additionally or alternatively, the system can log policy versions and applied triggers for audit. Thus, the thresholding and incremental re-computation reduce recalculation load while ensuring timely incorporation of resistance, efficacy, and cost changes.

In particular, the real-time resistance data connector 1706 and the environmental information collector 1522 can monitor real-time data feeds, e.g., by subscribing to HL7 and/or FHIR event streams and by polling external clinical and pharmaceutical data streams at configurable intervals (e.g., between 1 and 15 minutes). The medicine recommendation platform 1500 and/or the method 1800 can improve and have real-time medication recommendations, which is currently not available or possible for a provider to do, especially in an urgent, time-sensitive situation. For example, there can be over 50 unique variables that should be factored in for each medication that is prescribed, however, since providers do not have adequate access to the required information or time to consider the information, the provider may only factor in a couple, which can result in errors that can lead to patient harm, death, complication, ineffectiveness, higher costs, and the like. In one example, the connectors can normalize each message into a normalized real-time data update and can forward the update to the analysis engine 1532 to trigger recalculation on threshold change. Additionally, the connectors can implement validation, retry, and adaptive polling frequency based on source reliability and clinical urgency. Therefore, continuous monitoring supplies current resistance, availability, and coverage data that support safe selection and reduce the risk of outdated contraindication or cost assessments.

Further, the data acquisition layer 1516 can generate an updated harmonized dataset as an output when a threshold-triggered refresh completes, with contents that include time-stamped patient parameters and current drug attributes mapped to a standardized schema. In one example, the analysis engine 1532 can consume the updated harmonized dataset within suitability scoring to recalculate dose guidance, interaction risk, and composite scores. Additionally or alternatively, the platform can version the dataset for traceability and rollback. Thus, the updated harmonized dataset enables downstream ranking operates on current, unified inputs, supporting dose adjustment, real-time resistance use, and up-to-date cost and coverage integration.

### Continuous Data Refresh 1812—Monitor Real-Time Data Feeds 2202

The method 1800, e.g., the continuous data refresh 1812 step, can include a monitor real-time data feeds step 2202. For example, the monitor real-time data feeds step can establish persistent subscriptions and/or periodic polling to HL7 and FHIR endpoints at a configurable cadence (e.g., between 1 and 15 minutes) to acquire near real-time laboratory, inventory, pricing, and coverage events. More specifically, the monitor real-time data feeds step can parse inbound messages, validate integrity, and normalize fields into an internal schema to generate a normalized real-time data update while recording provenance for audit logging. In one example, the monitor real-time data feeds step can adapt polling frequency based on source reliability and clinical urgency and can queue retries on transient failures to sustain continuity. Thus, the monitor real-time data feeds step reduces latency to current susceptibility, availability, and cost information, addressing lack of real-time resistance data and reducing physician time spent on manual updates.

The external clinical and pharmaceutical data streams can supply input records from laboratory information systems, pharmacy inventory systems, drug price databases, and insurance benefit APIs using HL7/FHIR and/or delimited or proprietary formats, and the monitor real-time data feeds step can emit the normalized real-time data update as output. In one example, the real-time resistance data connector 1706 can execute acquisition of regional antibiogram updates and machine-readable culture results, extract MIC values and susceptibility percentages, and contribute those values to the normalized real-time data update alongside inventory, pricing, and coverage fields. Additionally, the monitor real-time data feeds step can timestamp and version each element to support threshold comparisons and downstream recalculation. Therefore, the external clinical and pharmaceutical data streams enable up-to-date efficacy, availability, and coverage inputs, mitigating the inability to integrate dynamic drug information into prescribing decisions while supporting traceable decision support.

### Continuous Data Refresh 1812—Trigger Recalculation on Threshold Change 2204

The method 1800, e.g., the continuous data refresh 1812 step, can include a trigger recalculation on threshold change step 2204. For example, the analysis engine 1532 can execute trigger recalculation on threshold change, e.g., in response to a threshold change, by evaluating parameter deltas against configurable thresholds for efficacy, resistance, availability, and/or cost within a defined suppression window. More specifically, the analysis engine 1532 can initiate a full or partial rerun of suitability scoring, including the dose calculation engine 1534, the interaction and contraindication checker 1536, the efficacy and resistance evaluator 1540, and the cost and coverage analyzer 1538, followed by ranking and optimization when a threshold is met or exceeded. Additionally, the analysis engine 1532 can log the triggering event with time-stamped inputs and emit updated recommendation artifacts for presentation, while optionally batching near-simultaneous updates to conserve compute. Thus, the automated and configurable rerun addresses lack of real-time susceptibility and cost integration, reduces physician time burden, and mitigates interaction risk by revalidating scores when material changes occur.

A normalized real-time data update can serve as an input to trigger recalculation on threshold change by providing time-stamped, schema-mapped values suitable for direct comparison to previously stored values. More specifically, the analysis engine 1532 can consume the normalized real-time data update and produce an updated harmonized dataset as an output, which aggregates reconciled patient parameters and current drug attributes for downstream scoring and ranking. Additionally, the analysis engine 1532 can version the updated harmonized dataset to enable auditability and to propagate only materially changed features into subsequent calculations. Thus, the structured input and harmonized output enable timely dose adjustment, real-time resistance incorporation, and cost-aware ranking without manual review.

The cost of medicines, such as antibiotics, can be extremely high. Many patients cannot afford the cost of prescribed medicines due to numerous factors, including the prescribed medications falling out of their insurance network or not being covered by insurance at all. As a result, a patient's illness or disease can worsen and the patient's health can suffer. Thus, by analyzing cost and patient insurance coverage, the medicine recommendation platform 1500 and/or the method 1800 can assist a provider in recommending and prescribing an affordable medicine for a patient and more patients will be able to afford medication they are prescribed. In this way, the medicine recommendation platform 1500 and/or the method 1800 solves a long felt need in the art. Likewise, medication prescription errors cause harm to patients. For example, 16% of all readmissions are medication-related. Thus, not only is the patient harmed, but the medication that was prescribed in error and used was also wasted. Since the medicine recommendation platform 1500 and/or the method 1800 can assist a provider in recommending and prescribing a medicine for a patient, accounting for factors such as other diseases and medications used by the patient that can be easily overlooked during the traditional prescription process, medication prescription errors, and thus medication-related readmissions, can be reduced. In this way, the medicine recommendation platform 1500 and/or the method 1800 solves a long-felt need in the art.

Table 1 below summarizes antibiotics categorized by cost rank, cost symbol, and estimated daily cost range in typical hospital contract pricing (with dosing assumptions for a 70 kg adult). As seen, the daily costs for some antibiotics are above $920 per day. The medicine recommendation platform 1500 can be constrained to recommend the top-tier antibiotics (Cefiderocol, Ceftolozane-tazo, Meropenem-vaborbactam) for multidrug-resistant infections, especially since alternatives like Daptomycin offer equivalent efficacy for MRSA and are significantly less resource-intensive to administer and monitor.

TABLE 1
Top 10 Costliest IV Antibiotics (Per Day,
In-Patient, Including Admin Costs).

| | | | Cost |
|---|---|---|---|
| Rank | Antibiotic | Aprox. Cost/Day | Symbol |
| 1 | Cefiderocol | ~$920+ | $$$$ |
| 2 | Ceftolozane-tazobactam | ~$800-1200 | $$$$ |
| 3 | Meropenem-vaborbactam | ~$800+ | $$$$ |
| 4 | Colistin (CBA) | ~$200-500 per day | $$$ |
| 5 | Tigecycline | ~$200-500 per day | $$$ |
| 6 | Eravacycline | ~$200-500 per day | $$$ |
| 7 | Daptomycin | ~$100-200 per day | $$ |
| 8 | Cefoxitin | ~$100-200 per day | $$ |
| 9 | Oxacillin (continuous infusion) | ~$100-200 per day | $$ |
| 10 | Piperacillin-tazobactam | ~$50-100 per day | $ |

Table 2 below summarizes a weekly cost for the top five most expensive antibiotics of Table 1, an alternative weekly costs if an alternative drug were used, e.g., Daptomycin instead of Tigecycline, and the savings for the patient. The Total Weekly Costs includes an estimated nursing cost (approximately 10.5 min×\$50/hr=\$8.75) and an estimated pharmacy cost (approximately 7 min×\$60/hr=\$7.00), in addition to the cost of the medicine.

TABLE 2
Weekly Cost Rankings & Savings (Per Patient).

| | | Expensive | Alternative | |
|---|---|---|---|---|
| Expensive | Alternative | Weekly | Weekly | |
| Drug | Durg | Cost | Cost | Savings |
| Cefiderocol | Cefepime | ~$9,906.75 | ~$855.75 | ~$9,051.00 |
| Meropenem- | Meropenem | ~$9,528.75 | ~$960.75 | ~$8,568.00 |
| Vaborbactam | | | | |
| Ceftolozane- | Piperacillin- | ~$7,323.75 | ~$1,170.75 | ~$6,153.00 |
| Tazobactam | Tazo | | | |
| Colistin | Polymyxin B | ~$2,780.82 | ~$1,590.75 | ~$1,190.07 |
| Tigecycline | Doxycycline | ~$2,670.50 | ~$360.50 | ~$2,310.00 |

Table 3 below summarizes the annual impact according to hospital size. Even small hospitals can save over $7 million annually by optimizing antibiotic selection, while large hospitals may reduce their pharmacy and nursing budgets by over $70 million per year. These savings can be reinvested into staffing, equipment, or expanded patient care services.

TABLE 3
Annual Budget Impact by Hospital Size.

| Hospital Size | Weekly Savings | Annual Savings |
|---|---|---|
| Small | ~$136,360.35 | ~$7,090,738.20 |
| Medium | ~$545,441.40 | ~$28,362,952.80 |
| Large | ~$1,363,603.50 | ~$70,907,382.00 |

### Healthcare Team Communication System

Communication between a healthcare provider, such as a physician or nurse, and a pharmacist is limited. This is due to many factors, including limited time for direct communication between pharmacists and providers. Thus, there is a lack of visibility of prescription changes made by one party to the other, e.g., any changes to a patient's prescription made by either party, e.g., a pharmacist or a provider, is not readily apparent to the other party. Further, there is often an absence of communicated reasoning for modifications, e.g., the reasoning for and supporting the change is not apparent because it is typically not communicated to the other party at all. Thus, a time-consuming manual review of patient records is traditionally required to determine the change rationale whenever one party (or “team member”) changes a prescription, e.g., the other party has to review all information on file and all patient records to try to discern why the change was made. This is time consuming and not time efficient since trying to work backwards to discern the reasoning behind a change can take hours for a single patient or prescription modification.

The system described herein is a multi or two-sided software platform permitting pharmacists and healthcare providers (e.g., provider-side users, which can include providers such as physicians, nurses, staff, hospital administrators, and the like) to share real-time patient records, lab results, and prescription changes. For example, a pharmacist and the pharmacist's team members can access one side, and provider and the provider's team members (e.g., nurse, physician, staff) can access the other side. Changes made on either side are visible to the other. Further, the patient's medical records and history are accessible to both sides and are updated in real time. For example, new lab test results can be retrieved or received by the platform and accessed on the platform by either party. Each side of the platform can be tailored to the respective team member. For example, the pharmacist and the pharmacist's team members side of the platform can include a different layout than the provider and the provider's team members side of the platform. Additionally, hospital administrators may view activity on the platform, such as a history ledger of a patient's medical history, including real-time patient records, lab results, prescription changes, and the like.

The platform can include notifications and alerts, a messaging system, and/or a shared journal to capture a holistic view of the patient's records and the reasoning behind modifications. When one party or team member makes a change to the patient's prescription, file, or records, the other party can be notified. For example, if lab results for a patient are received and they indicate a change to the prescription is needed or desired, such as decreased kidney function, the team member (provider or pharmacist) can input the change. Not only would the change be readily apparent to the other party, but the system can be programmed so the party that does not make the change can be notified, e.g., receive an alert, of the change. The party that made the change can also be notified when the other party sees and/or acknowledges the change. Thus, changes made by either party are readily visible by the other and can be acknowledged, reducing or eliminating the need for manual record review.

Traditionally, if a member of the team makes a mistake during the process, that error can travel downstream and reach the patient with the undesired negative consequence of ineffectiveness, harm or even death. For example, once a medication is administered it cannot be taken back and it is too late to prevent and in many cases reduce harm. The pharmacist-provider communication platform can assist the prescriber, pharmacist, nurse, and the like to work together in their various roles to increase the likelihood (as compared to traditional communication means) that the appropriate medication in the correct amount of medication is administered to the patient. Also, typically the pharmacist's duty is pharmacovigilance and the pharmacist can continue to track the patient after a prescription is given from a prescriber. The system described herein can assist the pharmacist with this role by enabling more accurate data, which enables the pharmacist to more efficiently intervene if there are changes. For example, prescribers will often write medication orders with a sliding scale depending on a patient's blood test results (e.g., insulin based on blood sugar test results or blood thinners based on coagulation levels). Pharmacists track these labs and tests and then routinely communicate with the patient to adjust the prescribed dose.

As previously mentioned, the platform can include a message system, shared journal, or the like to facilitate further communication between the parties. For example, with a shared journal that each party can update, the party making a change can include a note regarding the reason why, such as “kidney functioning low-decreasing dose”. In another example, if the party that doesn't make the change is confused about why the change was made, that party can message the other party requesting an explanation. In other examples, e.g., when information or explanation is lacking, the system can include or can use a LLM to cross reference the audit trail in the EMR and cross reference records from a similar time, and pull and present the relevant information from that drug and the patient's condition at that time, which can save the user time. For example, the system can chronologically track and record journal entries and/or messages, real-time labs, medicines given and prescribed, and the like. So, if a team member changes a prescription due to newly received lab results, that person can leave an entry with a quick note to explain why. In this way, the other party can easily see new lab results were received, then see the prescription changed, and then know why it changed due to the note left by the other party, all without having to sort through all the patient's records and discern the reason. This solution streamlines collaboration, reduces time spent deciphering prescription adjustments, and enhances patient safety. Also, the system can employ LLM to process the EMR for certain dates and provide quick summaries as to why the provider changed the medication dose at a certain date by scouring the medical record chronologically to evaluate and determine the reason why the prescriber changed the dose, e.g., in response to a lab result. The summary can reduce the time typically spent by pharmacists and other prescribers to review the medical record, which may include information scattered throughout sources, to determine why a medication dose was modified.

For example, the summary tool can be further helpful in cases where the change is made by a less experienced or newer provider since the system can track and present to a user who made the modification, what the modification was, where the modification was made, when the modification was made (e.g., time and date as well as on a chronological timeline of the medical history of the patient, including lab results and other events), and why the modification was made (e.g., by direct user input explaining the modification or by the system evaluating the medical record and determining the reason). For example, a message thread of the shared journal can include a summary including one or more of: who made the prescription change, what the prescription change was, where the prescription change was made, when the prescription change was made, or why the prescription change was made. Thus, a clarified summary of the patient record is available, which can enable a provider, prescriber, pharmacist, or the like to proceed more correctly, as compared to proceeding without knowledge of who made a modification, what the modification was, when and where the modification was made, or why the modification was made. In this way, an error, e.g., made by a less experienced provider, can more easily be identified. For example, the system can provider the pharmacists (or other team member) the ability for pharmacists to not only review what the prescriber is doing or has done (e.g., making modifications), but also the ability to communicate with the prescriber (or other team member) if there is a development or potential error identified that could impact the safety of the patient. Thus, by providing the pharmacist (or other team member) with the ability to track changes over time and determine why the modification was made, for example who changed what medication and on what date and why, can reduce or prevent mistakes and repeat mistakes. For example, if a hypertensive medication was canceled, the system can enable to provider to know that it was canceled by the chief cardiologist because it was causing kidney failure, as opposed to canceled by an intern because they thought the patient had a minor rash.

As discussed herein, medication prescription errors cause harm to patients. For example, 16% of all readmissions are medication-related. Thus, not only is the patient harmed, but the medication that was prescribed in error and used was also wasted. Since the pharmacy-provider communication platform 2300 can assist the healthcare team in identifying changes to prescriptions and potential errors, medication prescription errors, and thus medication-related readmissions, can be reduced. In this way, the pharmacy-provider communication platform 2300 solves a long-felt need in the art.

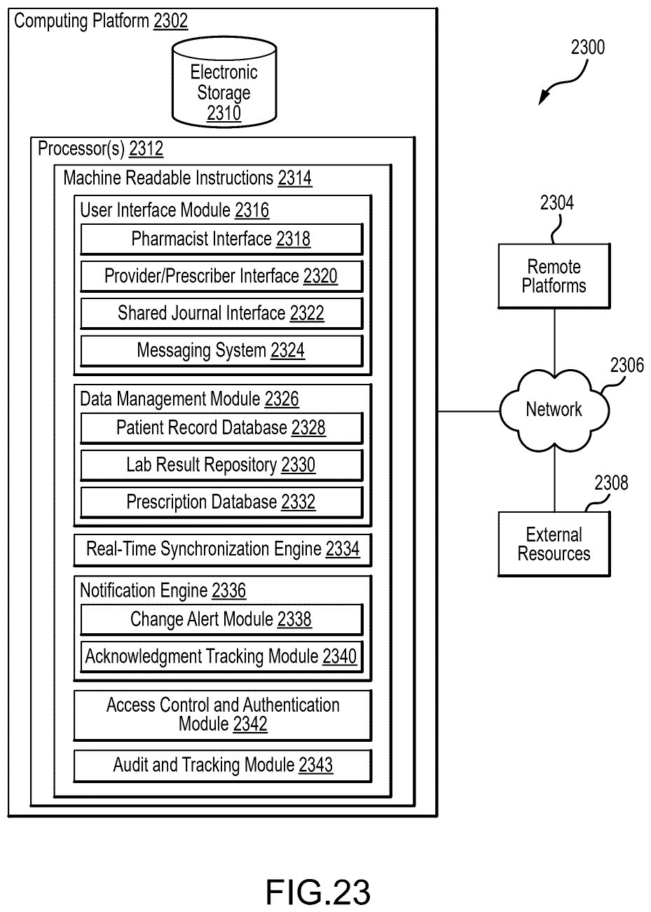

FIG. 23 illustrates a platform or system 2300 configured for enabling communication across a healthcare team, including between a pharmacy team and a provider team. The platform 2300 can be referred to herein as a pharmacy-provider communication platform 2300. In some implementations, the platform 2300 can include one or more servers or computing platform(s) 2302. Server(s) 2302 may be configured to communicate with one or more remote, e.g., client, computing platforms 2304 according to a client/server architecture and/or other architectures. Computing or remote platform(s) 2304 may be configured to communicate with other computing platforms via server(s) 2302 and/or according to a peer-to-peer architecture and/or other architectures. Users may access platform 2300 via client computing platform(s) 2304. In some examples, pharmacy-provider communication platform 2300 can be an agent, e.g., a system that can interact with various other system items. For example, pharmacy-provider communication platform 2300 can be accessible on an application and/or website.

Server(s) 2302 can include electronic storage 2310, one or more processors 2312, and/or other components. Server(s) 2302 can include communication lines, or ports to enable the exchange of information with a network and/or other computing platforms. Illustration of server(s) 2302 in FIG. 23 is not intended to be limiting. Server(s) 2302 can include a plurality of hardware, software, and/or firmware components operating together to provide the functionality attributed herein to server(s) 2302. For example, server(s) 2302 may be implemented by a cloud of computing platforms operating together as server(s) 2302.

Electronic storage 2310 may comprise non-transitory storage media that electronically stores information. The electronic storage media of electronic storage 2310 can include one or both of system storage that is provided integrally (i.e., substantially non-removable) with server(s) 2302 and/or removable storage that is removably connectable to server(s) 2302 via, for example, a port (e.g., a USB port, a firewire port, etc.) or a drive (e.g., a disk drive, etc.). Electronic storage 2310 can include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 2310 can include one or more virtual storage resources (e.g., cloud storage, a virtual private network, and/or other virtual storage resources). Electronic storage 2310 may store software algorithms, information determined by processor(s) 2312, information received from server(s) 2302, information received from client computing platform(s) 2302, and/or other information that enables server(s) 2302 to function as described herein.

In some examples, server(s) 2302, client computing platform(s) 2304, and/or external resources 2308 may be operatively linked via one or more electronic communication links. For example, such electronic communication links may be established, at least in part, via a network such as the Internet and/or other computing networks. It will be appreciated that this is not intended to be limiting, and that the scope of this disclosure includes implementations in which server(s) 2302, client computing platform(s) 2304, and/or external resources 2308 may be operatively linked via some other communication media.

External resources 2308 can include sources of information outside of platform 2300, external entities participating with platform 2300, and/or other resources. In some implementations, some or all of the functionality attributed herein to external resources 2308 may be provided by resources included in or otherwise in communication with the platform 2300, and vice versa.

Server(s) 2302 may be configured by machine-readable instructions 2314. Machine-readable instructions 2314 can include one or more instruction modules. The instruction modules can include computer program modules. The instruction modules can include one or more of a user interface module 2316, which can include one or more of a pharmacist interface 2318, provider interface 2320, shared journal interface 2322, or messaging system 2324; a data management module 2326, which can include one or more of a patient record database 2328, lab result repository 2330, or prescription database 2332; an real-time synchronization engine 2334; a notification engine 2336, which can include one or more of a change alert module 2338 or an acknowledgment tracking module 2340; an access control and authentication module 2342; or an audit and tracking module 2343.

Processor(s) 2312 may be configured to provide information processing capabilities in server(s) 2302. As such, processor(s) 2312 can include one or more of a digital processor, an analog processor, a digital circuit designed to process information, an analog circuit designed to process information, a state machine, and/or other mechanisms for electronically processing information. Although processor(s) 2312 is shown in FIG. 23 as a single entity, this is for illustrative purposes only. In some implementations, processor(s) 2312 can include a plurality of processing units. These processing units may be physically located within the same device, or processor(s) 2312 may represent processing functionality of a plurality of devices operating in coordination. Processor(s) 2312 may be configured to execute modules 2316, 2326, 2334, 2336, 2342, and/or 2343, and/or other modules, such as the sub-modules, e.g., 2318, 2320, 2322, 2324, 2328, 2330, 2332, 2338, and/or 2340. Processor(s) 2312 may be configured to execute modules 2316, 2326, 2334, 2336, 2342, and/or 2343, and/or other modules, such as the sub-modules, by software; hardware; firmware; some combination of software, hardware, and/or firmware; and/or other mechanisms for configuring processing capabilities on processor(s) 2312. As used herein, the term “module” may refer to any component or set of components that perform the functionality attributed to the module. This can include one or more physical processors during execution of processor readable instructions, the processor readable instructions, circuitry, hardware, storage media, or any other components.

Although modules 2316, 2326, 2334, 2336, 2342, and/or 2343, and/or other modules, such as the sub-modules, are illustrated in FIG. 23 as being implemented within a single processing unit, in implementations in which processor(s) 2312 includes multiple processing units, one or more of modules 2316, 2326, 2334, 2336, 2342, and/or 2343, and/or other modules, such as the sub-modules, may be implemented remotely from the other modules. The description of the functionality provided by the different modules 2316, 2326, 2334, 2336, 2342, and/or 2343, and/or other modules, such as the sub-modules, described herein is for illustrative purposes, and is not intended to be limiting, as any of modules 2316, 2326, 2334, 2336, 2342, and/or 2343, and/or other modules, such as the sub-modules, may provide more or less functionality than is described. For example, one or more of modules 2316, 2326, 2334, 2336, 2342, and/or 2343, and/or other modules, such as the sub-modules, may be eliminated, and some or all of its functionality may be provided by other ones of modules 2316, 2326, 2334, 2336, 2342, and/or 2343, and/or other modules, such as the sub-modules. As another example, processor(s) 2312 may be configured to execute one or more additional modules that may perform some or all of the functionality attributed below to one of modules 2316, 2326, 2334, 2336, 2342, and/or 2343, and/or other modules, such as the sub-modules.

### Pharmacy-Provider Communication Platform 2300

The pharmacy-provider communication platform 2300 can include and/or orchestrate a user interface module 2316, a data management module 2326, an real-time synchronization engine 2334, a notification engine 2336, an access control and authentication module 2342, and an audit and tracking module 2343 (and the sub-modules of each module 2316, 2326, 2334, 2336, 2342, and/or 2343) to facilitate and enable streamlined communication between members of a healthcare team. The pharmacy-provider communication platform 2300 can operate as a two-sided, network-connected system that synchronizes prescription records, patient history, and laboratory results across interfaces and application programming interfaces. In one example, the platform 2300 can propagate event deltas via a publish/subscribe stream and persist all transactions with user, timestamp, and context metadata in an auditable ledger. Additionally, the platform 2300 can adopt a modular architecture that integrates a messaging system and mobile access, e.g., without disrupting core synchronization pathways. Therefore, the platform 2300 reduces time spent coordinating by phone or personally researching a patient's file and resolves lack of visibility by presenting synchronized, authoritative data to both parties in real time (as used herein, “real time” can include a reasonable delay in transmission, such as greater than or equal to about 0 seconds and less than or equal to about 1 day, e.g., greater than or equal to about 0.1 seconds and less than or equal to about 30 minutes, e.g., greater than or equal to about 0.1 seconds and less than or equal to about 5 minutes, e.g., greater than or equal to about 0.1 seconds and less than or equal to about 1 minute).

The platform 2300 can embed a structured rationale annotation field within each modification record, where users can enter coded or free-text explanations. More specifically, the field can capture author identity, timestamps, and reference identifiers that link to laboratory results, guideline citations, and supporting records for deterministic parsing. In one example, the data management module can store the field alongside the modification payload to enable downstream analytics and inline rendering. Therefore, the field addresses absent reasoning and reduces manual review by coupling each change with machine-readable justification.

The platform 2300 can implement a unified patient data schema that normalizes prescriptions, laboratory observations, demographics, and clinical annotations as versioned JSON and/or XML resources with strict type validation. In one example, the schema can maintain referential integrity across prescriptions, laboratory results, and patient identifiers to prevent or reduce semantic drift across modules and external exchanges. Additionally, the schema can enable deterministic validation and forward-compatible extension when new clinical attributes are introduced. Therefore, the schema sustains consistent views for both parties and avoids divergence that contributes to visibility gaps.

The notification engine 2336 can execute automated change alert generation by evaluating rule sets, constructing multi-channel payloads, and dispatching alerts with deep links to the relevant patient context. In one example, the real-time synchronization engine 2334 can drive real-time bidirectional data propagation so that subscribed clients receive updates within sub-second latency under typical conditions. Additionally, the acknowledgment tracking module 2340 can record acknowledgment tracking and feedback by logging view events, generating originator confirmations, and escalating when thresholds expire. Therefore, the combined functions surface changes readily and document recipient awareness, which shortens coordination delays and resolves blind spots. Thus, the notification engine 2336 (and other modules described herein) can enable and assist a provider to conduct thorough medication reviews and MTM, provide bedside recommendations and assistance during rounds, lead safety initiatives—reconciliation, ADE prevention, execute targeted clinical interventions with high acceptance, align therapies with value-based care goals, advance antimicrobial stewardship and formularies, work in team-based clinics to improve chronic care, enhance community health efforts, track interventions, drive quality improvements, and the like.

The user interface module 2316 can execute clinical rationale capture and display by receiving and/or prompting for rationale at prescription change times and receiving and/or rendering explanations inline within change views and journals. In one example, the messaging system 2324 can provide secure asynchronous messaging that supports threaded discussions, attachments, and deep-link references to underlying data objects. Additionally, the shared journal interface 2322 can index rationale and message history chronologically to support targeted filtering and search. Therefore, the interface module 2316 presents context alongside changes, reducing the need for retrospective chart review and clarifying decision drivers.

The user interface module 2316 can render mirrored pharmacist and provider experiences (e.g., data similar to or the same as data shown on one interface can be presented on the other, e.g., the interface displays may appear different for different users, but can present information similar to or the same as information presented on the other interface(s), e.g., confidential information that can legally be viewed by one team member, such as the provider, can be hidden from other team members who are not authorized to view the information, such as pharmacy staff) while coordinating with the data management module 2326, the real-time synchronization engine 2334, the notification engine 2336, and the access control and authentication module 2342. In one example, the user interface module 2316 can present dashboards, change summaries, and shared journals that update according to synchronization events and role-based access rules. Additionally, the interfaces 2318, 2320 can surface notifications and acknowledgment states inline to streamline review and response workflows. Therefore, the module 2316 exposes current, role-appropriate information that reduces navigation effort and accelerates comprehension of recent changes.

The pharmacy-provider communication platform 2300 can execute a real-time change notification and acknowledgment method 2400 to synchronize data, generate prescription change notifications, present modification details, and log acknowledgments within an auditable chronology. In one example, the method 2400 can compose and deliver notification packages, render change summaries with access to supporting records, and update status logs upon capture of acknowledgment signal. Additionally, the method 2400 can maintain a time-ordered record of notifications, clarifications, and confirmations for compliance reporting. Therefore, the method 2400 can establish reliable, documented handoffs that improve transparency without requiring time-consuming direct communication or personal research.

### User Interface Module 2316

The user interface module 2316 can generate and render interactive graphical user interfaces that present unified patient record data, structured prescription modification entry data, and prescription change notification package content in real time. In one example, the user interface module 2316 can execute present modification details to a counterparty and render change summary while synchronizing with a real-time synchronization engine 2334 and receiving alerts from a notification engine 2336. Additionally, the user interface module 2316 can operate as a web client and/or mobile application interface within a containerized microservice deployment, and a change summary interface active configuration can dynamically configure views and controls. Therefore, the user interface module 2316 can reduce lack of visibility and limited time for direct communication by consolidating current data and change context in a single, continuously synchronized workspace.

A chronological change timeline widget can visualize event chronology for rapid clinical review by aggregating prescription laboratory result arrivals, acknowledgments, and journal entries into a scrollable, filterable sequence. In one example, the widget can request events via a paginated API from a data management module 2326 and/or an audit and tracking module 2343, render category iconography, and hyperlink each node to the originating data object for drill-down. Additionally, the widget can adapt time-axis granularity across hourly-to-weekly ranges and can surface reasoning by linking to shared journal interface 2322 entries associated with each event. Thus, the widget can eliminate time-consuming manual review and increase visibility into decision sequences by exposing an ordered, data-linked chronology.

More specifically, the user interface module 2316 can facilitate bidirectional messaging and journal entry by embedding rich-text editors and attachment components that post messages to a messaging system 2324 and append entries to a shared journal interface 2322. In one example, the editors can tag author identity and timestamps, and the audit and tracking module 2343 can generate cryptographically timestamped event record that feed a compliance report pipeline. Additionally or alternatively, an acknowledgment tracking module 2340 can link message exchanges to counterparty acknowledgment data to maintain stateful threads around prescription changes. Therefore, the module can address absence of communicated reasoning and limited time by capturing concise, attributable explanations inline with the clinical record. In cases where a reason is missing, the system can send a request message to the physician or other team member who made the change and request that they give an explanation for the modification, e.g., to the pharmacist and/or other healthcare team members so they are enabled to understand the reason and logic for the change.

In one example, the user interface module 2316 can render role-specific graphical user interfaces by querying an access control and authentication module 2342 for permission tokens and mapping permissions to interface fragments. Additionally, the user interface module 2316 can conditionally expose change summary interface active views, messaging tools, and supporting record links according to pharmacist and/or provider roles while maintaining a unified code base. Further, the user interface module 2316 can propagate the same role logic to the mobile application interface to maintain consistent capability across endpoints. Therefore, the module can reduce cognitive load and protect sensitive data while preserving visibility of relevant prescription changes for rapid action.

In particular, the user interface module 2316 can include a pharmacist interface 2318, a provider interface 2320, and a shared journal interface 2322 arranged in a hierarchy to coordinate role-specific workflows and shared documentation. In one example, the pharmacist interface 2318 can present controls to initiate prescription modification, annotate reasoning as journal entries, and review change alerts while an acknowledgment tracking module 2340 updates a status log. Additionally, the user interface module 2316 can expose a configurable prompt for pharmacist-side templates and/or required fields to standardize rationale capture and link entries to structured prescription modification entry objects. Thus, the pharmacist interface 2318 can increase visibility of modifications and convey reasoning efficiently, reducing coordination time between pharmacists and providers.

### User Interface Module 2316—Pharmacist Interface 2318

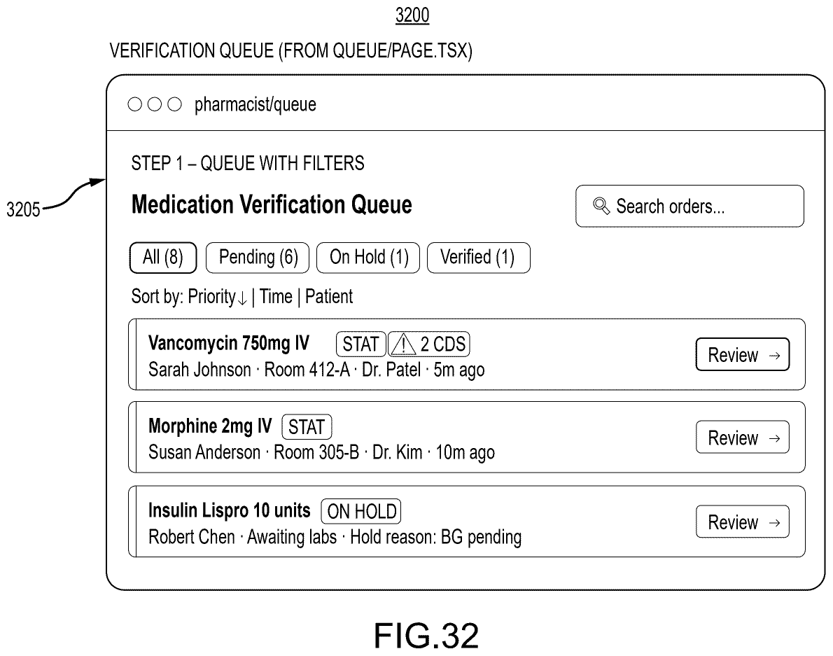

The pharmacist interface 2318 can present pharmacist-specific workflows that allow a pharmacist user to initiate prescription modification, review provider-initiated changes, and log rationale data within the user interface module 2316 of the pharmacy-provider communication platform 2300. In some examples, the pharmacist interface 2318 can present a GUI 3200 as depicted in FIGS. 32-38 among others. For example, as depicted in FIGS. 32-38 among others, the GUI 3200 can present a verification queue. The user interface module 2316, such as a presentation layer of the user interface module 2316, which can be similar to or the same as the presentation layer 1544, can present a medication queue in a first box or screen 3205 as depicted in FIG. 32, which can include a summary of medications that require verification, including total number or all, pending, on hold, and verified medications. The first screen 3205 can include a sorting feature that can order the list of medications based on time, patient, priority, and the like as well as a search feature in which a pharmacist can search for verifications based on any of the information linked to the verification, e.g., patient name or drug. The first screen 3205 can include a list of medications that require verification, including the drug name, dose, administration route, timing (e.g., STAT, ON HOLD, and the like), warnings (e.g., drug interactions or contraindications), patient name, room number, prescriber name, time elapsed since ordered, notes (e.g., verification is on hold due to awaiting labs), and a selection button (e.g., “review” or “select”) that can advance the pharmacist to the next step in the verification flow.

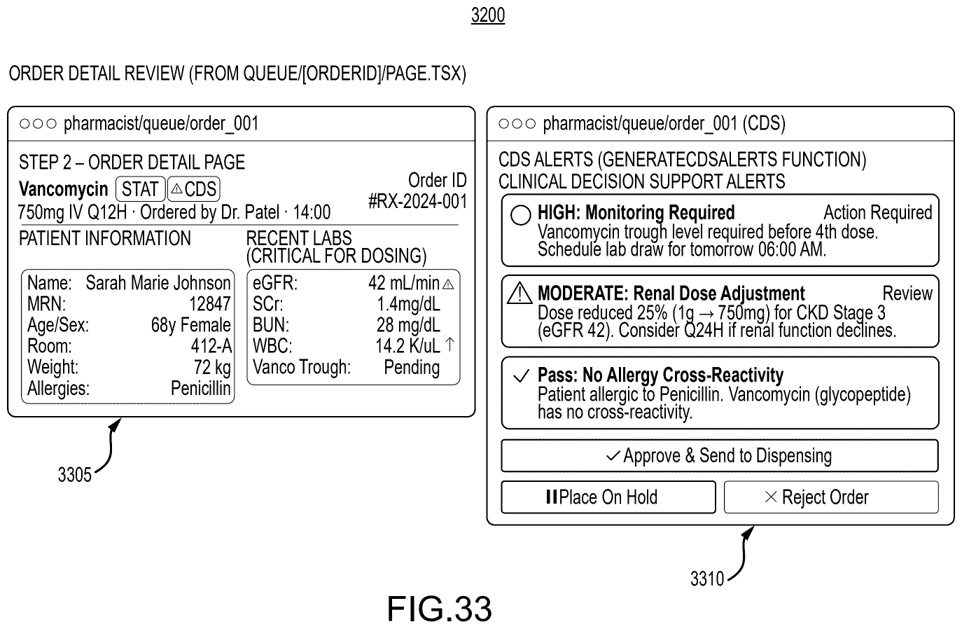

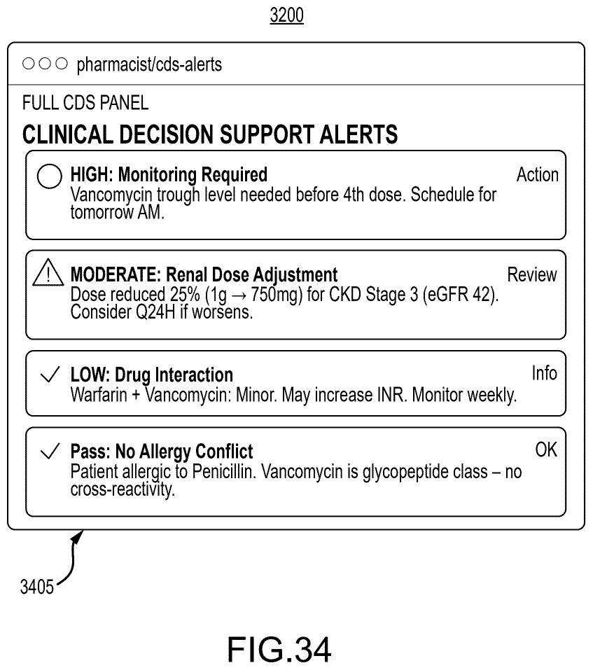

The user interface module 2316 can present an order detail page in a second box or screen 3305 as depicted in FIG. 33, which can include a name of the drug selected at screen 3205, dose, administration route, prescriber name, timing (e.g., STAT, ON HOLD, and the like), warnings (e.g., drug interactions or contraindications), order ID, patient name, patient identifier number, patient age, patient sex, patient room, patient weight, patient allergies, patient lab results, and the like. In some examples, a pharmacist can select a link to a clinical decision support (CDS) alert screen 3310 as depicted in FIG. 33, which can include a summary of alerts (e.g., monitoring required alerts, renal dose adjustment alerts, and the like), drug interactions, allergy warnings, and the like. The CDS alert screen 3310 can include options for a pharmacist to approve and send the medication to dispensing, place on hold, or reject the order. In some examples, the pharmacist can enter notes regarding reasoning for the decision, e.g., order rejected due to kidney functioning from lab results received after medication ordered. FIG. 34 depicts another example of a CDS alert screen 3405. In this example, the CDS alert screen 3405 can include additional information, such as information that may not be flagged, e.g., if there is a low risk of any drug interaction.

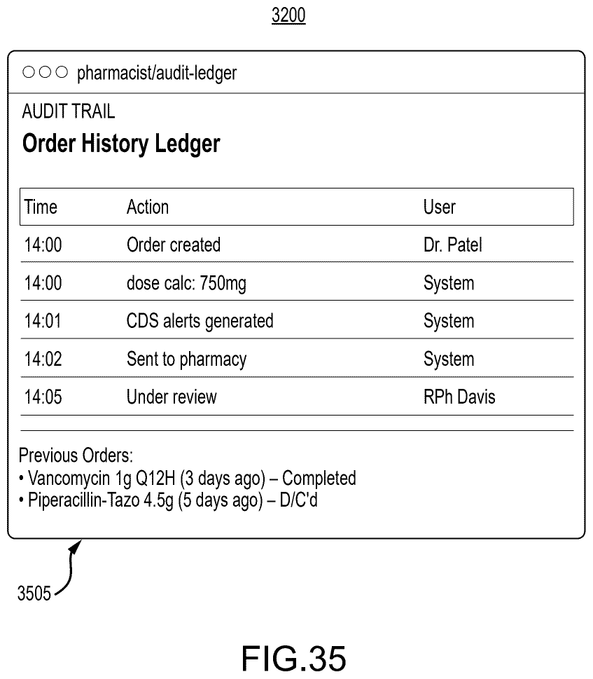

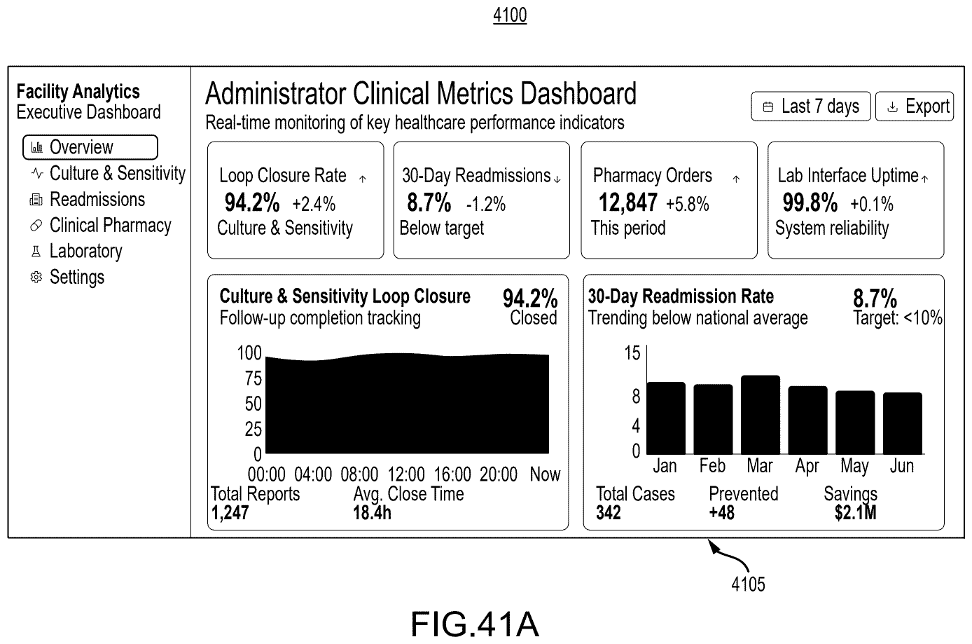

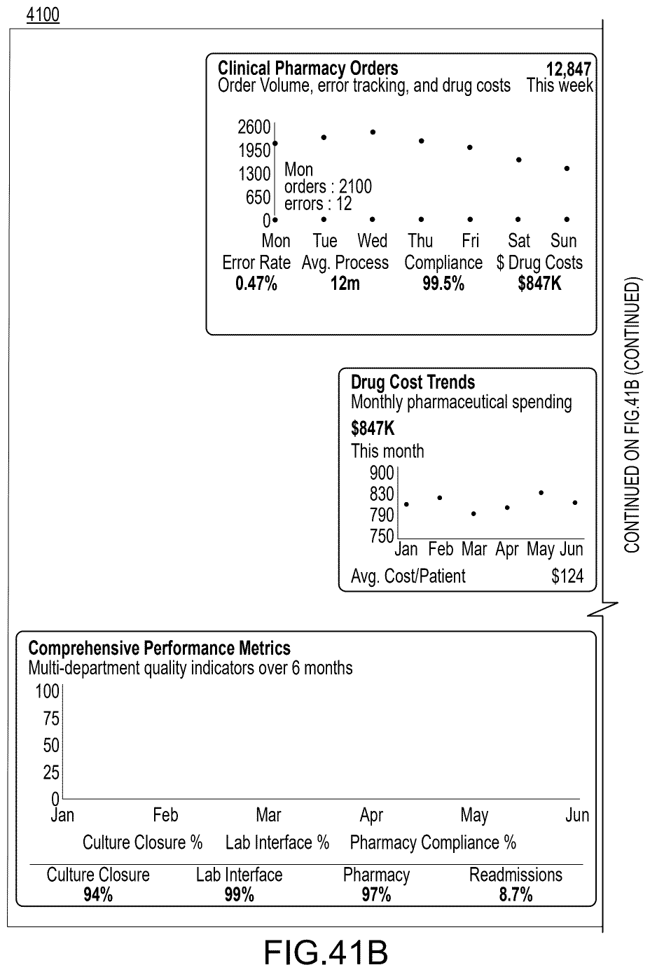

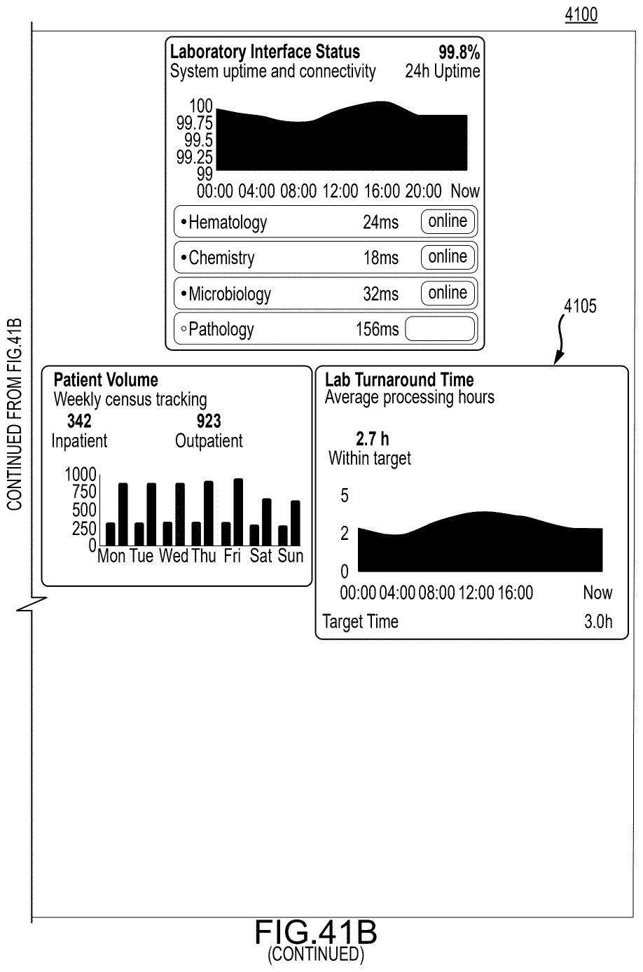

The user interface module 2316 can present an audit trail or order history ledger screen 3505 as depicted in FIG. 35, and described further herein. For example, any team member, including hospital administrators, can view a complete history ledger for a patients file. For example, FIG. 35 depicts the GUI 3200 of the pharmacist interface 2318 displaying the history ledger screen 3505, however, the GUI 2500 and/or the GUI 2900 of the provider interface 2320 can also present the history ledger screen 3505. Additionally, in some examples, the provider interface 2320 can include a GUI 4100 for other members of the provider team, such as hospital administrators, as depicted in FIGS. 41A and 41B among others. The GUI 4100 can also present the history ledger screen 3505. The history ledger screen 3505, which can be generated by the data management module 2326 as described herein, can include a tabular layout including a date and/or time section; an action section, e.g., order created, lab results received, alerts generated, sent to pharmacy, under review, and the like; a user section, e.g., which can be used to identify which user generated the respective action; and the like. The history ledger screen 3505 can include a summary of previous orders, e.g., for the patient, drug, prescriber, and the like.

The user interface module 2316, e.g., the GUI 4100 of the provider interface 2320, can present a clinical metrics dashboard 4105 as depicted in FIGS. 41A and 41B. The analysis engine 1532 can leverage data stored in the data storage layer 1524 and the real-time synchronization engine 2334 to compile and calculate performance indicators, e.g., in real-time, which can provide team members, such as hospital administrators with the ability to monitor the performance indicators in real-time. As used herein, “real-time” can be based on a sync schedule, e.g., of the real-time synchronization engine 2334, which can be a range of times including every millisecond, every five minutes, every couple of hours, every day, every week, every month, and the like. The clinical metrics dashboard 4105 can include filter time frames, such as data from the last 7 days, the last quarter, the last year, and the like. The clinical metrics dashboard 4105 can include options to download or export the data.

The clinical metrics dashboard 4105 can include a loop closure rate, e.g., the percentage of follow-up actions that are completed after an action, finding, or referral, which can be a helpful metric to assess the effectiveness of follow-up processes and increase patient safety. For example, hospitals that consistently achieve high closure rates are likely to have better patient outcomes and reduced risks of patient harm. Additionally, the clinical metrics dashboard 4105 can include a percent increase or decrease in the loop closure rate, which can be based on a default timeframe or a programmed timeframe, such as monthly, quarterly, yearly, and the like.

The clinical metrics dashboard 4105 can include a readmission rate, e.g., the percentage of patients readmitted to a hospital within a specific time frame (such as 30 days, as depicted in FIG. 41A, however, additional time frames are within the scope of this disclosure, e.g., including standard windows used in health services research) after being discharged. The readmission rate metric can serve as a measure of healthcare quality and continuity of patient care, e.g., indicating how well a hospital manages patient outcomes. For example, a readmission occurs when a patient who had been discharged is admitted again within a specified time interval, e.g., due to a drug interaction between two prescribed medications, contraindication, allergies, and the like. Additionally, the clinical metrics dashboard 4105 can include a percent increase or decrease in the readmission rate, which can be based on a default timeframe or a programmed timeframe, such as monthly, quarterly, yearly, and the like.

The clinical metrics dashboard 4105 can include a number of pharmacy orders within a specific time frame (such as a week or period, as depicted in FIG. 41A, however, additional time frames are within the scope of this disclosure, e.g., including standard windows used in health services research), e.g., the written directions by a prescribing practitioner for a specific medication to be administered to an individual. The pharmacy orders metric can serve as a way to measure hospital revenue, business, and the like. Additionally, the clinical metrics dashboard 4105 can include a percent increase or decrease in the pharmacy orders, which can be based on a default timeframe or a programmed timeframe, such as monthly, quarterly, yearly, and the like.

The clinical metrics dashboard 4105 can include a lab interface uptime, e.g., the percentage of time that the laboratory information system (LIS) is operational and functioning correctly. The lab interface uptime metric assist in increasing or evaluating operational efficiency, patient care quality, laboratory throughput, and overall financial health. For example, a high uptime directly influences these factors, as delays can lead to missed diagnoses and increased operational costs, so organizations that manage and prioritize uptime can leverage business intelligence to enhance their reporting dashboard and improve management reporting. Additionally, the clinical metrics dashboard 4105 can include a percent increase or decrease in the lab interface uptime, which can be based on a default timeframe or a programmed timeframe, such as monthly, quarterly, yearly, and the like.

The clinical metrics dashboard 4105 can include a culture and sensitivity loop closure analysis, e.g., in the form of a chart, table, graph, visual, or the like, which can be an expandable display or new window derived from a link in the loop closure rate box and/or a side bar tab of the clinical metrics dashboard 4105. The culture and sensitivity loop closure analysis can include analysis of the process by which healthcare providers collect and analyze samples from patients to identify the specific microorganisms causing an infection and determine the most effective treatment. This process can increase the accuracy with which diagnosis and appropriate treatment is delivered, e.g., compared to processes where this is not performed, thereby improving patient outcomes. The analysis can include follow-up completion tracking data over a period of time, e.g., a 24 hour period, showing the percentage of total reports closed with an average close time.

The clinical metrics dashboard 4105 can include a readmission rate analysis, e.g., in the form of a chart, table, graph, visual, or the like, which can be an expandable display or new window derived from a link in the readmission rate box and/or a side bar tab of the clinical metrics dashboard 4105. The readmission rate analysis can include analysis of the number of readmissions in specified time increments, such as monthly; a number of total cases (e.g., dependent upon a specific starting point, such as the beginning of a new year, quarter, period, and the like); a total percentage of readmissions, including an indication of whether the rate is above, below, or at target and by how much as well as an indication of the rate compared to national, local, state, and the like averages; a number of prevented readmission cases (e.g., based on the system flagging or warning a commonly prescribed medication due to a patient specific interaction, contraindication, allergy, or the like); an estimated cost savings (e.g., which can be based on one or more of the number of prevented cases, the data compared to national averages, the data compared to historical averages for the specific hospital, and the like); and the like.

The clinical metrics dashboard 4105 can include a number of pharmacy orders analysis, e.g., in the form of a chart, table, graph, visual, or the like, which can be an expandable display or new window derived from a link in the number of pharmacy orders box and/or a side bar tab of the clinical metrics dashboard 4105. The number of pharmacy orders analysis can include a breakdown of order volume over specified time periods, such as days, as well as a total volume over a specified time period, such as a week, (other breakdown and total volume time frames are within the scope of this disclosure, e.g., including standard windows used in health services research). The number of pharmacy orders analysis can include error tracking, e.g., an error rate calculated based on the number of errors compared to the number of orders; average processing time; a compliance rate; total drug costs; and the like.

The clinical metrics dashboard 4105 can include a lab interface uptime or status analysis, e.g., in the form of a chart, table, graph, visual, or the like, which can be an expandable display or new window derived from a link in the lab interface uptime box and/or a side bar tab of the clinical metrics dashboard 4105. The lab interface uptime analysis can include an operational and functioning connectivity of labs over a period of time, which can be a default or programmed period. The lab interface uptime analysis can include a tabular view of specific laboratories broken down and their current connectivity status, such as online, degraded, or the like. Additional icons can be used to indicate the status, such as green, yellow, or red bubbles.

The clinical metrics dashboard 4105 can include additional metrics, which can be summarized or mentioned in previously described boxes. For example, the total drug costs provided in the number of pharmacy orders box and/or analysis can include a link or expandable menu, which can lead to another detailed box breaking down the total drug costs. In other examples, the detailed box breaking down the total drug costs can be a stand alone box. The breaking down of the total drug costs box can include an analysis of pharmaceutical spending, e.g., based on a default or programmed time period, such as monthly, which can include a total amount spent for the specified time period, along with a chart, table, graph, visual, or the like depicting the total cost over sequential time periods, such as monthly. The breaking down of the total drug costs box can include an average cost per patient based on the total number of patients and the total costs over the same time period.

The clinical metrics dashboard 4105 can include additional metrics, such as a patient volume analysis or census tracking analysis, which can include a number of inpatients and a number of outpatients (e.g., over a time period). The number of inpatients and a number of outpatients can also be depicted in a chart, table, graph, visual, or the like. For example, as depicted in FIG. 41B, the number of inpatients and a number of outpatients are depicted in a bar graph for each say of a week and the total numbers for the week are summarized at the top of the box. Other additional metrics can include a lab turnaround time, which can include an average time for processing a lab, e.g., in a specified unit of time, such as hours. The lab turnaround time box can include a target time and a note of whether the turnaround time is within target or not. The lab turnaround time can also be depicted in a chart, table, graph, visual, or the like, as depicted in FIG. 41B. Other additional metrics can include a comprehensive performance metrics box, which can include a summary of multi-department quality indicators over a period of time, such as a day, a week, a month, 6 months, a year, and the like. For example, the comprehensive performance metrics box can include percentages or numbers from any of the previously discussed boxes. In some examples, the comprehensive performance metrics (e.g., the percentages) can be depicted in a chart, table, graph, visual, or the like, e.g., over a period of time. In this way, a team member can view each of the statistics side by side, which can help a team member in identifying trends or inefficiencies.

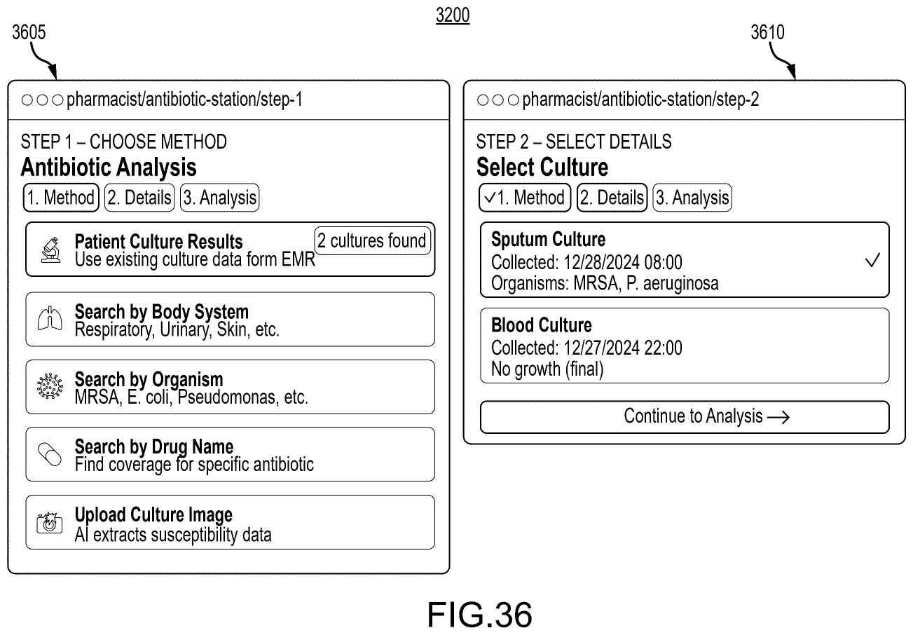

The systems described herein, e.g., the presentation layer 1544 of the medicine recommendation platform 1500, the user interface module 2316 of the pharmacy-provider communication platform 2300 (including the sub interfaces and systems), and the like, can present an antibiotic flow, e.g., can present one or more screens 3605, 3610, 3705, 3710, 3805, 3810 depicted in FIGS. 36-38. For example, one or more screens 3605, 3610, 3705, 3710, 3805, 3810 of the antibiotic flow depicted in FIGS. 36-38 can be presented by the GUI 3200 of the pharmacist interface 2318, e.g., such that the antibiotic flow is part of the verification process the pharmacist performs prior to verifying an order. In other examples, the GUI 2500 and/or the GUI 2900 of the provider interface 2320 can present one or more screens 3605, 3610, 3705, 3710, 3805, 3810 of the antibiotic flow depicted in FIGS. 36-38. In this example, a provider can review and verify antibiotic information, such as susceptibility, prior to submitting an order or prescribing a medication. Similarly, in this example, a nurse or medication administrator can review and verify antibiotic information, such as susceptibility, prior to administering the drug to the patient. In this way, the systems described herein can assist the healthcare team in prescribing and/or administering drugs that are more effective, e.g., compared to traditional ways that do not routinely or regularly review antibiotic information, such as susceptibility.

As depicted in FIG. 36, an antibiotic analysis in a first box or screen 3605 can be presented. The first screen 3605 can include a summary of the analysis steps, e.g., method, details, analysis (with the current step highlighted or otherwise flagged); options for methods to review or analyze a certain drug or medication, e.g., an antibiotic, such as culture results (e.g., captured from an EMR), a body system, an organism, a drug or medication name, an uploaded culture image, and the like; and the like. The first screen 3605 can include notifications, such as 2 cultures found. A user can select one or more of the options to continue to review. In the example depicted in FIG. 36, the Patient Culture Results is selected. In response to selection of an option, a details second box or screen 3610 can be presented. The second screen 3610 can include a summary of the analysis steps, e.g., method, details, analysis (with the current step highlighted or otherwise flagged and previous steps marked as completed or reviewed). In this example, the second screen 3610 can include a list of cultures, e.g., patient specific cultures, with culture specific information, such as name, date and time collected, whether organisms are detected, and if so, a list of the organism(s), and the like. The user can select a box to continue the flow, e.g., to continue to the analysis step of the antibiotic flow.

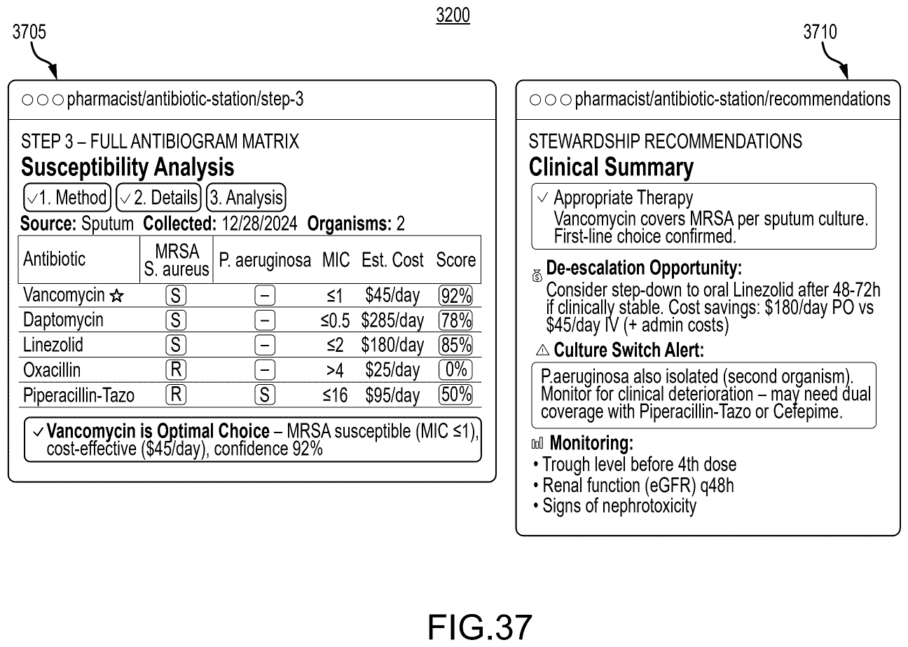

As depicted in FIG. 37, a susceptibility analysis in a third box or screen 3705 can be presented. The third screen 3705 can include a summary of the analysis steps, e.g., method, details, analysis (with the current step highlighted or otherwise flagged); a summary of the culture's source, collection date, and number of organisms found; a summary chart, e.g., a tabular chart, of potential drugs, such as antibiotics, including an identifier of the drug with respect to the organisms (e.g., “S” for if the respective organism is susceptible to the respective antibiotic, “R” for if the respective organism is resistant to the respective antibiotic, “-” if there is no data or not applicable, and the like), the Minimum Inhibitory Concentration (“MIC”), the estimated cost (which can be patient specific based on insurance information), and a score (e.g., a confidence score which can factor and weigh one or more of the susceptibility of the organism, the effectiveness of the drug or antibiotic, the cost, and the like, e.g., a the weight given to each factor can be default or can be programmed by the user); a recommendation, which can include the name of the recommended drug and reasoning, e.g., information in the summary chart for the recommended drug. In some examples, the recommended drug can be marked or flagged in the summary chart, e.g., bolded, with an icon such as a star, and the like. A recommendation page or clinical summary of a fourth box or screen 3710 can be presented. For example, a user can select the recommendation or other link to review additional details regarding the recommended drug, and in response to the selection the fourth screen 3710 can be presented. The fourth screen 3710 can include a summary of the therapy and why the recommendation is appropriate, which can include that the recommended drug covers the organism identified in the culture; a de-escalation opportunity summary, which can include a recommendation to de-escalate the antibiotic, e.g., after a period of time if clinically stable, which can reduce or inhibit harm to a patient's kidney function (which can be caused by antibiotics with higher strength than the de-escalated recommendation for example) and increase the efficiency of antibiotics overtime since bacteria and organisms can grow resistant to an antibiotic overtime; additional alerts, such as a culture switch alert which can include a recommendation for another drug or antibiotic based on other organisms in the culture; a monitoring summary, which can summarize patient data, such as trough level before a specific dose, renal function, signs of nephrotoxicity, and the like; and the like.

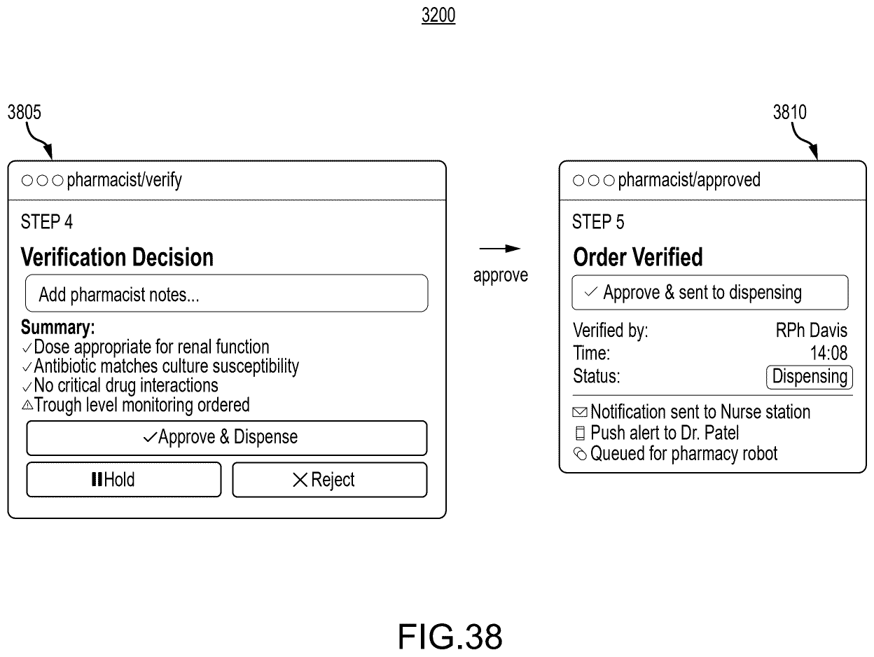

As depicted in FIG. 38, a verification decision of a fifth box or screen 3805 can be presented. The fifth screen 3805 can include an option for the team member, e.g., the prescriber, nurse, pharmacist, and the like, to add notes to the decision. In the example depicted in FIG. 38, a pharmacist is provided the opportunity to add notes, however, a prescriber or nurse can be given presented with the fifth screen 3805 on the provider interface 2320 prior to confirming a decision, such as ordering a medication or administering a medication. The notes input by team members can be viewable to other team members, e.g., on the shared journal interface 2322 described herein. The fifth screen 3805 can include a summary of the decision or recommendation, such as the dose compared to the patient's renal function, the antibiotic compared to the culture, a confirmation that there are none or a low risk of drug interactions or contraindications, cautionary warnings (e.g., trough level monitoring ordered), and the like. The fifth screen 3805 can include icons the team member can select to complete the flow, such as an approval icon; pause the flow, such as a hold or pause icon; cancel or stop the flow, such as a reject or cancel icon; and the like. A verification summary of a sixth box or screen 3810 can be presented. The sixth screen 3810 can include a confirmation of the team members selection at the fifth screen 3805, e.g., verification, hold, cancelation, or the like. In the example depicted in FIG. 38, the fifth screen 3805 includes an order verified summary, which can include the team member the order was verified by, the time of approval, and the status of the order. The fifth screen 3805 can include a summary of actions taken by the platform 2300, e.g., notifications sent to other team members, such as a nurse or the prescriber.

The user interface module 2316, e.g., a presentation layer, can present one or more of the screens 3205, 3305, 3310, 3405, 3505, 3605, 3610, 3705, 3710, 3805, 3810, and in some examples may not present one or more of the screens 3205, 3305, 3310, 3405, 3505, 3605, 3610, 3705, 3710, 3805, 3810, e.g., which can be dependent on user preference selections.

In one example, the pharmacist interface 2318 integrates with the shared journal interface 2322 and the notification engine 2336 to render change summaries, receive acknowledgment data, and trigger bi-directional clarification messages. Additionally, the pharmacist interface 2318 can invoke the Medication Recommendation Platform discussed herein and query unified patient record data to guide dose adjustments and medication substitutions. Therefore, the pharmacist interface 2318 reduces communication latency and improves mutual visibility of prescription changes, addressing limited time for direct communication and lack of visibility between parties. For example, the pharmacist interface 2318 can assist or enable a pharmacist to conduct thorough medication reviews and medication therapy management (MTM); lead safety initiatives, such as reconciliation, adverse drug event (ADE) prevention; execute targeted clinical interventions with high acceptance; align therapies with value-based care goals; advance antimicrobial stewardship and formularies; establish collaborative practice agreements; work in team-based clinics to improve chronic care; track interventions and drive quality improvements, and the like.

A chronological activity timeline can aggregate prescription modifications, laboratory-result arrivals, and journal entries into a client-rendered, time-ordered view that links each event to the corresponding database record. In one example, the pharmacist interface 2318 can capture and commit prescription modifications by packaging edited fields, structured rationale, and a digital signature into a transaction that the prescription database 2332 confirms before further edits proceed. Additionally, the pharmacist interface 2318 can generate acknowledgment events by recording view times and explicit confirmations and by forwarding the timestamps and identifiers to the acknowledgment tracking module 2340. Thus, the chronological activity timeline provides immediate context and traceability for changes, solving visibility gaps and reducing time spent locating dispersed events.

Generally, configurable alert threshold settings can allow a pharmacist user to set per-user bounds, frequency limits, and quiet periods that the notification engine 2336 applies when elevating events to high-priority alerts across devices via the data management module 2326. In one example, a role-adaptive layout engine can assemble interface widgets and workflows according to a role configuration retrieved at authentication, thereby presenting dose-adjustment controls, interaction panels, and audit viewers that match pharmacist duties. Additionally, client-side rendering can enforce access boundaries while avoiding duplicated backend services. Therefore, configurable alert threshold settings and a role-adaptive layout engine reduce alert fatigue and streamline task focus, addressing limited time for communication without obscuring clinically significant changes.

A structured rationale entry panel can embed selectable clinical factors and a free-text field within every prescription-modification dialog to capture pharmacist reasoning at the point of action. In one example, the pharmacist interface 2318 can tag each rationale element with unique identifiers and store the identifiers alongside the modified prescription record for analytics and audit queries. Additionally, the pharmacist interface 2318 can display read-only rationale when a provider-originated change is opened from the timeline. Therefore, the structured rationale entry panel supplies communicated reasoning and eliminates downstream manual record mining, addressing the absence of communicated rationale and time-consuming manual review.

The pharmacist interface 2318 can display synchronized patient data by querying the data management module 2326 for current prescription, laboratory, and demographic records and by subscribing to update events from the real-time synchronization engine 2334. In one example, incremental Document Object Model (DOM) patching can update only affected widgets when provider-side changes arrive, avoiding full-page reloads while maintaining consistency with the unified patient record data. Additionally, the pharmacist interface 2318 can surface supporting records from the shared journal interface 2322 when a change summary is active. Thus, the display of synchronized patient data prevents or reduces stale views and reduces manual reconciliation effort, addressing visibility gaps and time-consuming review of patient records.

### User Interface Module 2316—Provider/Prescriber Interface 2320

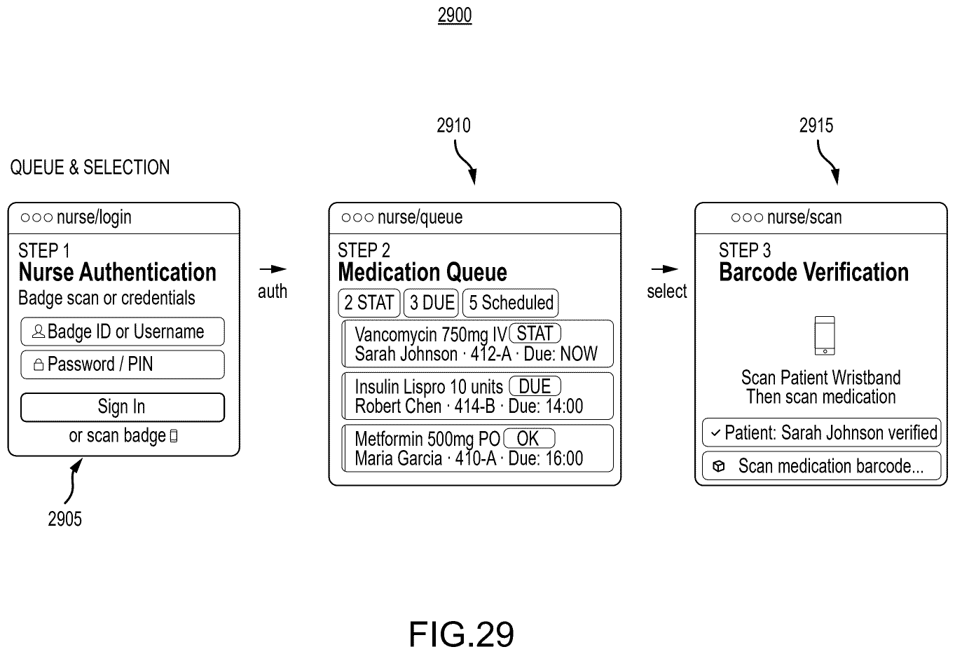

The provider interface 2320 can also be referred to herein as a prescriber interface. The provider interface 2320 can present unified patient record data and prescription change notification package within a single workspace to support present modification details to counterparty and render change summary. In some examples, the provider interface 2320 can present the GUI 2500 of FIGS. 25-28, as described herein, e.g., which respect to the medicine recommendation platform 1500 and/or the method 1800. In some examples, the provider interface 2320 can include a GUI 2900 for other members of the provider team, such as nurses or the provider administering a medication, as depicted in FIGS. 29-31 and 39-40 among others. For example, as depicted in FIG. 29 among others, the GUI 2900 can present a queue and selection flow. The user interface module 2316, such as a presentation layer of the user interface module 2316, which can be similar to or the same as the presentation layer 1544, presents an authentication login in a first box or screen 2905, which can include an ID or username input and a password or PIN input; a medication queue in a second box or screen 2910, which can include a summary of all medications due immediately or STAT, due, scheduled, administered, and the like, as well as a list of medications, including the dose administration route, patient, room number, and time for administration; and a verification in a third box or screen 2915, which can include a barcode scanner to scan a patient identifier, such as a wrist band, a patient identifier input box, or the like.

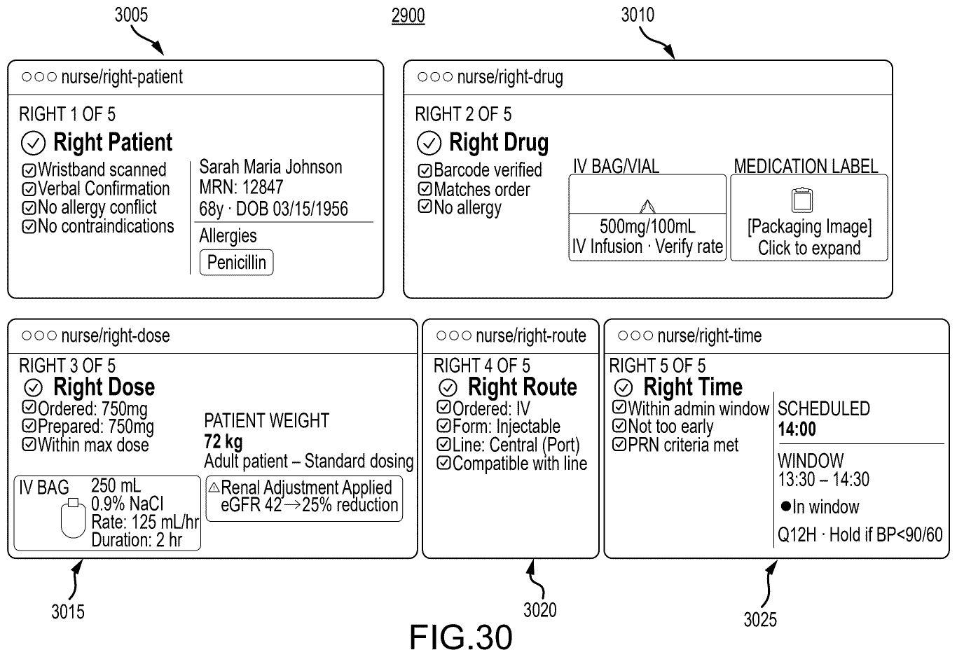

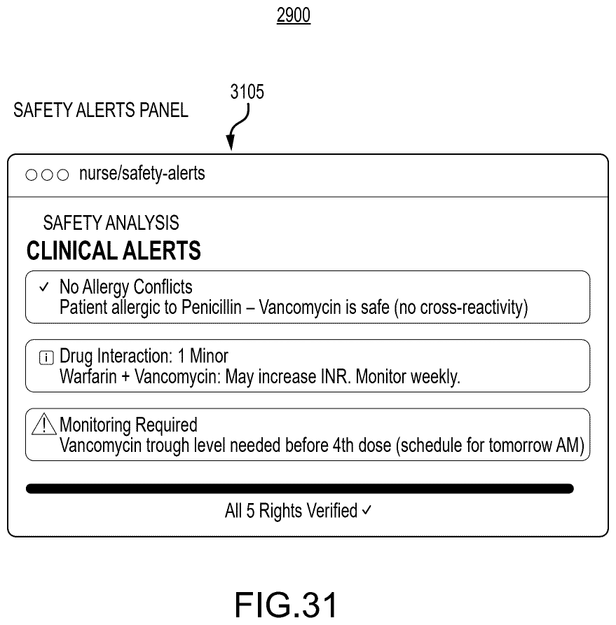

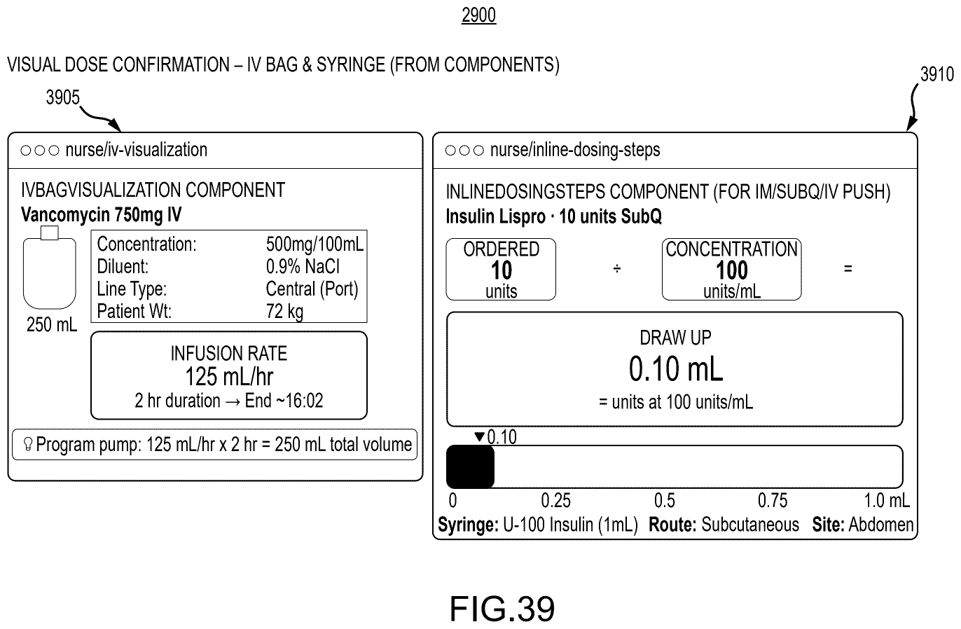

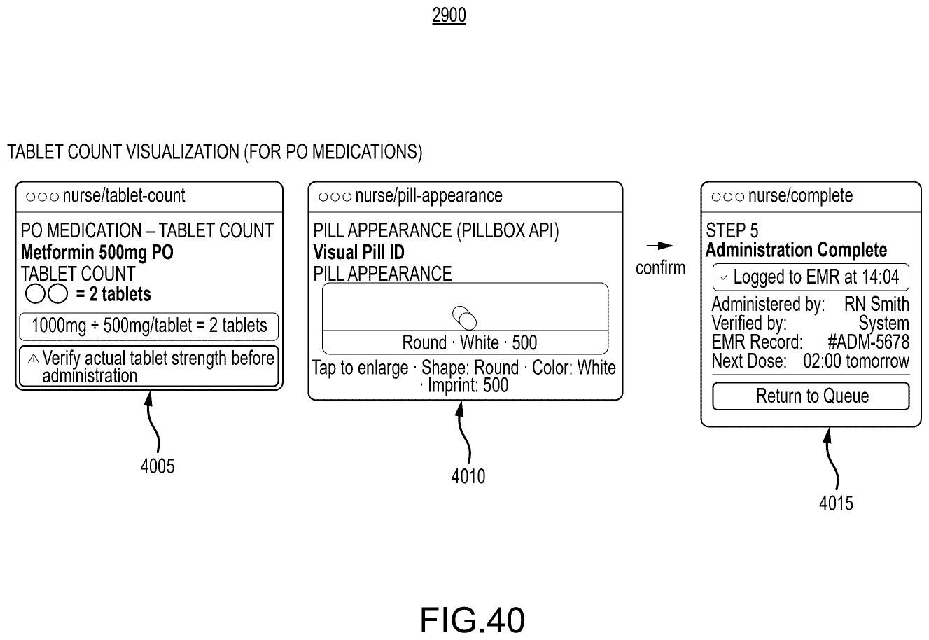

For example, as depicted in FIG. 30 among others, the GUI 2900 can present a verification flow. The user interface module 2316, such as a presentation layer of the user interface module 2316, which can be similar to or the same as the presentation layer 1544, presents a first verification in a first box or screen 3005, which can include a right patient verification checklist including identifier scanned, verbal confirmation, allergy conflicts, contraindications check, patient name, identifier number, age, allergies, and the like; a second verification in a second box or screen 3010, which can include a right drug verification checklist including barcode verification, order matching, allergy confirmation, administration route, dose, medication label, images of the drug to be administered (e.g., as further described with respect to FIGS. 39-40); and the like; a third verification in a third box or screen 3015, which can include a right dose verification checklist including a ordered dose, a prepared dose, within a max dose, type of drug and administration route, depiction of drug to be administered (e.g., as further described with respect to FIGS. 39-40), patient information such as weight and age, adjustment applies such as renal adjustments for kidney functions, and the like; a fourth verification in a fourth box or screen 3020, which can include a right route verification checklist including a list of the ordered administration route (e.g., IV, oral, and the like), form, line, compatibility with line, and the like; and a fifth verification in a fifth box or screen 3025, which can include a right time verification checklist including a list confirming the drug is administered within the admin window, not too early or late, PRN criteria met, time administration is scheduled for, the admin window, notes such as hold information, and the like. In another example, as depicted in FIG. 31 among others, the GUI 2900 can present a safety alert panel 3105, which can be included in the queue and selection flow, the verification flow, and/or another flow. The safety alert panel 3105 can include a list of clinical alerts and confirmations, including an analysis of whether allergy conflicts are present, an analysis of whether drug interactions are present, and a caution in the case of required monitoring.

As depicted in FIGS. 39 and 40 among others, the GUI 2900 can present a visualization flow or diagram, e.g., according to or with respect to the prescribed, ordered, verified, or the like medication or drug. For example, the GUI 2900 can present a visualization diagram of an IV pump interface in cases where the drug is administrated intravenously or subcutaneously (as depicted in FIG. 39), of a pill in cases where the drug is administered orally (as depicted in FIG. 39), of a syringe in cases where the drug is administered intravenously or subcutaneously (as depicted in FIG. 14), and the like.

For example, as depicted in FIG. 39, in cases where a drug is to be administered intravenously via an IV, the user interface module 2316, such as a presentation layer of the user interface module 2316, which can be similar to or the same as the presentation layer 1544, can present an IV pump visualization on a screen 3905. The first screen 3905 can include the name of the prescribed medication; the dose; the administration route (e.g., “IV”); an icon of the administration route (e.g., an IV bag and/or the size of the IV bag); a summary of information related to the drug to be administered, such as the concentration, the diluent, the line type, the patient weight, and the like; the infusion rate; the time frame, including the duration and the end time; a summary of the programming instructions, including the infusion rate, the duration of infusion, and the total volume of liquid (e.g., drug and diluent) to be infused; and the like. Further, in cases where a drug is to be administered subcutaneously via a syringe, the user interface module 2316 can present a syringe visualization on a screen 3910 (and/or the screen of interface 1400). The screen 3910 can include a drug name to be administered, the number of units ordered, related to (e.g., divided by) the concentration of the drug and the total amount of the drug to draw up (e.g., in the units used on the syringe with any conversion factors or notes). The screen 3910 can include a visualization of what the drug amount should look like in the syringe. For example, and depicted in FIG. 39, a bar representing a 1 mL syringe is displayed with a representation of the drug filled to the 0.10 mL mark, which aligns with the total amount of the drug to be drawn. In this example, incremental volumetric text is depicted. In other examples, volumetric marking can be included, e.g., within the syringe depiction or along the line with the volumetric text. The screen 3910 can include a summary of the syringe to use (e.g., the size), the administration route, the administration site (e.g., abdomen), and the like.

In other examples, as depicted in FIG. 40, in cases where a drug is to be administered orally via pill, the user interface module 2316, such as a presentation layer of the user interface module 2316, which can be similar to or the same as the presentation layer 1544, can present a dose visualization on a first screen 4005. The first screen 4005 can include the name of the drug; the dose of the drug per tablet (e.g., 500 mg); the administration route (e.g., medication orally (by mouth) “PO”); the total tablet or pill count, which can include icons to represent the number of tablets and/or written text of the number of tablets to administer; the breakdown of the dose to be administered (e.g., 1000 mg of drug divided by the dose per table (500 mg) equals 2 tablets to administer; any notes or warnings, such as a reminder for the medicine administrator to verify the strength of the tablet prior to administration; and the like. The user can select a link or proceed to another page that can present a visualization of the actual pill to be administered (e.g., as opposed to an icon of a generic pill) on a second screen 4010. The second screen 4010 can include a visual pill ID; a picture of the appearance of the pill that is to be administered in real time; a description of the pill, such as round, white, any imprinted markings on the pull, such as the dose per pill, and the like; and the like. The drug administrator can enlarge the image to further confirm the drug that is to be administered to the patient aligns with the visualization of the pill presented on the user interface module 2316. In this way, administration errors can be reduced since the visualization can serve as a confirmation that the drug and the dose of the drug to be administered is correct. The user can select a link or proceed to another page, e.g., after administering the drug to the patient in real time, that can present a completion confirmation on a third screen 4015. The third screen 4015 can include a note that the administration is complete; confirmation the administration is logged to the patient's EMR, which can include a time stamp; name of the team member administering the drug; the system that verified the dose and the drug; the EMR record locator or number; next steps, such as the next touch point with the patient, which can include the time, date, name, dose, and the like of the next drug to be administered; a completion button, which can take the team member back to the administration queue (e.g., of the screen 2910).

Additionally, the user interface module 2316 and/or the presentation layer 1544 can present any of the screens 2505, 2510, 2515, 2605, 2705, 2710, 2805, 2810, 2905, 2910, 2915, 3005, 3010, 3015, 3020, 3025, 3105, 3205, 3305, 3310, 3405, 3505, 3605, 3610, 3705, 3710, 3805, 3810, 3905, 3910, 4005, 4010, 4015, 4105 described herein with respect to FIGS. 25-41B on any of the GUIs 2500, 2900, 3200, 4100. Further, any system described herein, including the system 100, can present any of the screens 2505, 2510, 2515, 2605, 2705, 2710, 2805, 2810, 2905, 2910, 2915, 3005, 3010, 3015, 3020, 3025, 3105, 3205, 3305, 3310, 3405, 3505, 3605, 3610, 3705, 3710, 3805, 3810, 3905, 3910, 4005, 4010, 4015, 4105 described herein with respect to FIGS. 25-41B. Additionally, the user interface module 2316 and/or the presentation layer 1544 can present any of the screens 300-a-d, 401, 401a, 411, 421, 402, 413-415, 420, the screens of devices or interfaces 740, 940, 1005, 1140, 1399, 1400 depicted in FIGS. 3A-14. In any example, one or more of the screens 300-a-d, 401, 401a, 411, 421, 402, 413-415, 420, the screens of devices or interfaces 740, 940, 1005, 1140, 1399, 1400, 2505, 2510, 2515, 2605, 2705, 2710, 2805, 2810, 2905, 2910, 2915, 3005, 3010, 3015, 3020, 3025, 3105, 3205, 3305, 3310, 3405, 3505, 3605, 3610, 3705, 3710, 3805, 3810, 3905, 3910, 4005, 4010, 4015, 4105 may not be presented or included.

The provider interface 2320 can operate as a web portal and/or an EMR (which may be referred to herein as an electronic health record (EHR))-embedded module with access control and authentication module 2342 enforcement of role-based permissions. In one example, the provider interface 2320 can receive configuration from a change summary interface active state to emphasize change-focused workflows and to expose direct access to supporting records via linked views. Alternatively, the provider interface 2320 can expose controls to initiate, modify, and/or approve prescription orders, while a containerized microservice deployment can deliver the provider interface 2320 as a horizontally scalable frontend service.

A contextual data presentation panel can collocate laboratory values, active medications, and recent clinical events adjacent to prescribing controls, with time-stamped headers and status indicators that prioritize newly arrived data. In one example, the panel can attach hoverable provenance identifiers that link each datum to an originating source record and can reflow responsively across desktop and tablet displays. More specifically, an embedded rationale viewer can render explanatory notes, shared journal excerpts, and decision-support annotations sourced from the data management module 2326 via parameterized queries, with hyperlinks to laboratory results and shared journal interface 2322 entries. In this example, the embedded rationale viewer can preserve scroll position across sessions to streamline multi-stage review and can interoperate with a change summary interface active configuration to provide access to supporting records without context switching.

The provider interface 2320 can expose or present messaging system 2324 and/or shared journal interface 2322 widgets to facilitate bi-directional clarification messaging that remains bound to an active patient context. In one example, the messaging components can cryptographically sign each entry, forward read receipts and acknowledgment data to an acknowledgment tracking module 2340, and display reception indicators inline with the conversation. More specifically, the provider interface 2320 can capture acceptance, rejection, and/or clarification requests for each prescription modification and relay the status to the audit and tracking module 2343, which can timestamp events and generate an auditable change and acknowledgment record. Alternatively, the provider interface 2320 can trigger re-escalation of unattended critical alerts after a preset interval via the notification engine 2336 and can present a sortable acknowledgment table to summarize outstanding and completed actions for reporting.

### User Interface Module 2316—Shared Journal Interface 2322

The shared journal interface 2322 can operate as a bidirectional documentation component of the user interface module 2316 that both the pharmacist interface 2318 and the provider interface 2320 render concurrently. In one example, the shared journal interface 2322 can accept free-text and/or structured entries, execute provide access to supporting records, and execute append response to chronological journal while subscribing to a channel of the real-time synchronization engine 2334 for immediate cross-party visibility. Additionally, the shared journal interface 2322 can interoperate with the data management module 2326 and the integration gateway using healthcare data standards (e.g., HL7, FHIR) to resolve referenced records while the audit and tracking module 2343 records journal mutations. Therefore, the shared journal interface 2322 reduces lack of visibility of prescription changes and mitigates limited time for direct communication by creating a continuously synchronized, shared record.

More specifically, the shared journal interface 2322 can include a bidirectional chronicle panel that renders pharmacist-side and provider-side entries as discrete, expandable cards in strict chronological order with optional pinning of recent activity. In one embodiment, the shared journal interface 2322 can include a structured entry template library rendered as validated dynamic forms (e.g., JSON-schema-driven) to promote consistent capture of change context. Additionally, the shared journal interface 2322 can facilitate targeted retrieval of historical notes by executing full-text and faceted search across time range, author, event type, and patient filters. Thus, the bidirectional chronicle panel decreases time-consuming manual review by surfacing only relevant entries while maintaining a single temporal narrative accessible to both parties.

In particular, the shared journal interface 2322 can include event reference hyperlinking that attaches resolvable identifiers generated by the data management module 2326 to each journal card. In one example, the shared journal interface 2322 can enable contextual drill-down to source data by opening deep links to laboratory results, prescription orders, and/or patient chart sections in a dedicated pane while preserving journal focus. Additionally or alternatively, the shared journal interface 2322 can persist rationale for prescription changes by storing explanatory text and/or structured fields bound to the corresponding clinical event identifier. Therefore, event-linked navigation and persisted rationale eliminate absence of communicated reasoning and reduce manual chart searches.

Further, the shared journal interface 2322 can include automatic time-stamp metadata by querying a trusted time service (e.g., Network Time Protocol (NTP)) to embed immutable Coordinated Universal Time (UTC) values for each entry while rendering localized times on clients. In one example, the shared journal interface 2322 can delegate cryptographic sealing and ordering to the audit and tracking module 2343 to support reconstruction of event sequences across sites. Additionally, the shared journal interface 2322 can enforce access permissions at entry level by consulting the access control and authentication module 2342 during render and edit operations to restrict sensitive content. Therefore, immutable timing and entry-level authorization create an auditable, compliant record that sustains clear visibility of actions without unauthorized disclosure.

### User Interface Module 2316—Messaging System 2324

The messaging system 2324 can operate within the user interface module 2316 of the pharmacy-provider communication platform 2300 and present one-to-one and/or group threads linked to a patient record, a prescription, and/or a clinical event. More specifically, the messaging system 2324 can execute facilitate bi-directional clarification messaging and send follow-up query, while allowing attachment of laboratory results, prescription documents, and explanatory notes to each message. Additionally, the messaging system 2324 can integrate with the shared journal interface 2322 to append response to chronological journal and with the audit and tracking module 2343 to timestamp events and maintain an auditable chronological record. In one example, the messaging system 2324 can enforce access via the access control and authentication module 2342, synchronize message state through the real-time synchronization engine 2334, and deploy as a containerized microservice to scale across teams.

The messaging system 2324 can implement asynchronous secure communication by persisting queued messages in a server datastore and delivering messages during a next synchronization cycle to provide eventual delivery under intermittent connectivity. In one example, the messaging system 2324 can provide delivery and read receipt generation by updating message-header state flags when a recipient client acknowledges packet receipt and when a message pane gains focus, and the user interface module 2316 can render corresponding indicators and timestamps. Additionally, the messaging system 2324 can perform notification badge emission by publishing event objects to the notification engine 2336 upon creation of a new message, mention, and/or file attachment, and the notification engine 2336 can emit in-app badges and push alerts to draw attention to pending discussions. Alternatively, the messaging system 2324 can encrypt message payloads end-to-end and maintain signature metadata to protect confidentiality and integrity across networks.

### Data Management Module 2326

A data management module 2326 can persist, retrieve, and synchronize structured clinical data across the pharmacy-provider communication platform 2300. In one example, the data management module 2326 executes synchronize patient data and merge data into patient record to maintain an authoritative record with sub-second replication latency (e.g., between 50 and 900 ms). Additionally, the data management module 2326 can expose application programming interfaces for create, read, update, and delete operations with transactional consistency and conflict resolution under concurrent updates. Alternatively, the data management module 2326 can operate within a containerized microservice deployment to scale storage and throughput elastically according to workload.

The data management module 2326 can incorporate a unified clinical data schema that normalizes prescription entities, laboratory observations, and patient identifiers under shared keys and enumerations. More specifically, the data management module 2326 can persist structured clinical data by validating relationships, applying default values, and writing version metadata during atomic commits to durable storage. In one example, the data management module 2326 can embed standardized vocabularies (e.g., medication codes and LOINC identifiers) to enable deterministic synchronization and cross-source consistency. Thus, the data management module 2326 can maintain semantically consistent records suitable for downstream decision support and notification workflows.

The data management module 2326 can audit and trace data lineage by writing every mutation to an immutable transaction ledger linked to user identity metadata. More specifically, the data management module 2326 can retrieve contextual patient snapshots by assembling latest or historical versions of prescriptions, laboratory values, and demographics within bounded response times (e.g., between 5 and 200 ms under indexed queries). In one example, the data management module 2326 can expose query parameters to filter by time windows, actors, and rationale references to reconstruct clinical decision pathways. Therefore, the data management module 2326 can provide verifiable provenance for compliance reporting and retrospective review. For examples, hospital administrators can access the transaction ledger to review and report, track metrics, and the like.

The data management module 2326 can include a patient record database 2328, a lab result repository 2330, and a prescription database 2332. In one example, the patient record database 2328 can maintain longitudinal patient identifiers, demographics, diagnoses, allergies, and synchronized chart elements with role-based access and versioning. Additionally, the lab result repository 2330 can ingest structured laboratory results via standardized protocols (e.g., HL7, FHIR) and publish change events for downstream components. Further, the prescription database 2332 can store active and historical orders with modification metadata and can trigger downstream alerts when entries change.

### Data Management Module 2326—Patient Record Database 2328

The patient record database 2328 can store structured electronic health records for each patient, including demographic fields, diagnoses, allergies, laboratory results, and historical and current prescriptions. In one example, the data management module 2326 updates the patient record database 2328 in real time using the real-time synchronization engine 2334, while the integration gateway exchanges records via standardized protocols (e.g., FHIR and/or HL7). Additionally, the access control and authentication module 2342 can authorize pharmacist interface 2318 and provider interface 2320 requests, and the audit and tracking module 2343 can record read/write events with timestamps and user identifiers. Alternatively, a containerized microservice deployment can host the patient record database 2328 as a horizontally scalable service that supports low-latency retrieval of unified patient record data, which can be accessed from multiple EMR (similarly to or the same as described herein with respect to the data acquisition layer 1516).

The patient record database 2328 can include a structured multi-index schema that cross-references each clinical element by a globally unique patient identifier and a clinical data-class identifier. In one example, the schema can further index records by an event-time and/or encounter identifier to accelerate compound queries from the pharmacist interface 2318, provider interface 2320, and external FHIR consumers, using clustered B-trees and/or hashed shard keys to sustain millisecond-level retrieval under high concurrency.

The patient record database 2328 can include a versioned snapshot layer that captures immutable, copy-on-write snapshots after clinically meaningful events such as medication reconciliation. In one example, the versioned snapshot layer can reconstruct historical states with clinical rationale by correlating point-in-time prescription states with contemporaneous laboratory values from the lab result repository 2330 and explanatory entries from the shared journal interface 2322 and/or structured prescription modification entry, all presented without impacting live transactional workloads.

The patient record database 2328 can persist longitudinal electronic health records by continuously appending and maintaining complete clinical histories for each patient. In one example, the data management module 2326 can generate unified patient record data from the patient record database 2328 for downstream use by the real-time change notification and acknowledgment method, thereby providing a single authoritative source across pharmacy-provider workflows.

### Data Management Module 2326—Lab Result Repository 2330

The lab result repository 2330 can receive, store, and serve structured laboratory observations associated with a patient record managed by the data management module 2326. In one example, the lab result repository 2330 ingests payloads via HL7 messages, application programming interfaces, and/or other electronic data interchange formats, and the lab result repository 2330 persists queryable entries with versioning to support longitudinal review. Additionally, the lab result repository 2330 can enforce role-based access via the access control and authentication module 2342 and can expose references to stored observations to the shared journal interface 2322 to provide access to supporting records. Further, the lab result repository 2330 can integrate with external electronic health record systems through the integration gateway to maintain data consistency for unified patient record data used across the pharmacy-provider communication platform 2300.

The lab result repository 2330 can implement a multi-dimensional result indexing schema that keys each observation by patient identifier, collection timestamp, ordering facility identifier, and a standardized measurement code (e.g., LOINC), optionally using a composite primary key or a clustered index to enable deterministic retrieval of recent and historical values. In one example, the lab result repository 2330 stores units and reference ranges alongside the indexed fields to keep identical analytes collected at different times and locations discrete for trend analysis by the user interface module 2316. Additionally or alternatively, the lab result repository 2330 can execute an event trigger on result receipt that, after schema validation and commit, emits an event to the real-time synchronization engine 2334 containing a patient identifier, an analyte code, a criticality indicator, and a reference to the stored observation. Then, downstream components such as the notification engine 2336 can subscribe to the event stream to drive alerts and dosing recommendations within a time window (e.g., within several seconds) following laboratory release.

### Data Management Module 2326—Prescription Database 2332

The prescription database 2332 can store structured medication orders for a patient and can expose query interfaces to read current and prior versions of each order. More specifically, the prescription database 2332 can assign an immutable identifier to each prescription and can append revision rows that reference the identifier to maintain a continuous timeline. Additionally, the prescription database 2332 can reference authoring party metadata and timestamps for each revision to support downstream generation of a structured prescription modification entry by the data management module 2326 and/or the prescription database 2332. Thus, the prescription database 2332 can provide durable, low-latency retrieval of active views and historical sequences for use by the user interface module 2316 and the audit and tracking module 2343.

The prescription database 2332 can expose a real-time change feed that publishes a structured event upon each insert, update, and/or delete that affects prescription records. More specifically, the prescription database 2332 can emit modification events that encode primary keys, changed fields, and before-and-after values and can deliver the events to the real-time synchronization engine 2334 and/or the notification engine 2336 via a message broker. In particular, the prescription database 2332 can implement change data capture using write-ahead log hooks and/or triggers to achieve sub-second end-to-end event availability while maintaining exactly-once serialization semantics across transactions. Additionally or alternatively, the prescription database 2332 can persist active and historical orders by writing new history rows rather than overwriting prior data, thereby enabling the real-time change feed to reference both the latest state and a complete substitution lineage in each event. Enablement of batching the orders can reduce the likelihood that the prescriber would have to cancel orders that were just ordered or otherwise processed. Thus, the batch order feature reduces the amount of potentially time consuming steps.

### Real-Time Synchronization Engine 2334

The real-time synchronization engine 2334 can detect, process, and propagate insertions, updates, and deletions originating from pharmacist and/or provider interface 2320s. For example, the real-time synchronization engine 2334 can use an event-driven publish/subscribe protocol. The real-time synchronization engine 2334 can utilize a message broker and/or a streaming framework to deliver updates with sub-second end-to-end latency (e.g., between 50 and 800 milliseconds) while maintaining eventual consistency across user interface module 2316 clients and mobile application interface clients. In one example, the real-time synchronization engine 2334 can apply selective synchronization based on access control and authentication module 2342 permissions and subscription filters to limit propagation to relevant patient record database 2328, prescription database 2332, and lab result repository 2330 fields. Thus, the real-time synchronization engine 2334 provides immediate cross-party visibility of prescription and record changes, which reduces missed updates and shortens coordination time.

The real-time synchronization engine 2334 can include a conflict-resolution rule engine to evaluate concurrent writes that target overlapping entities. More specifically, the conflict-resolution rule engine can execute concurrent edit reconciliation by merging non-overlapping fields, applying timestamp and role-precedence rules, and arbitrating with domain rules that consider clinical severity codes and data freshness. In one example, the real-time synchronization engine 2334 can commit the reconciled state atomically to the data management module 2326 while persisting original deltas to an audit ledger for traceability. Thus, the conflict-resolution rule engine prevents divergence of shared records and reduces manual follow-up needed to resolve inconsistencies. The pharmacy-provider communication platform 2300 can create timestamps, e.g., for each event related to the prescription change, the prescription change notification package, or an acknowledgment signal to produce a chronological timestamped event record, and can also, e.g., the real-time synchronization engine 2334 can also, scour FHIR data from various EMR sources and centralize and organize this information chronologically to add clarity to the patient's history and make this information accessible. Thus, the negative consequences of siloing data, including different patient data, can be reduced or overcome by providing providers with access to critical and actionable health data for a patient.

The real-time synchronization engine 2334 can incorporate a durable audit trail logger that records each synchronization event in an append-only chain secured with cryptographic hashes and persisted to a write-once tier. More specifically, the durable audit trail logger can operate inline with real-time bidirectional data propagation so that every emitted update and every reconciliation decision is captured with a millisecond-resolution timestamp and origin metadata. In one example, the durable audit trail logger can survive message replay and upstream process restarts to preserve ordering and provenance guarantees. Thus, the durable audit trail logger establishes tamper-evident visibility into change history, which supports compliant collaboration and avoids retrospective, time-consuming record reconstruction.

The real-time synchronization engine 2334 can employ an event-driven change detector that subscribes to data management module 2326 commit hooks and registers Create, Read, Update, and Delete (CRUD) events in a non-blocking queue. More specifically, the event-driven change detector can generate an event payload for the notification engine 2336 that encodes change type, affected patient, modified fields, and a rationale reference to supporting records and/or the Medication Recommendation Platform outputs. In one example, the event-driven change detector can batch or coalesce events over a short window (e.g., between 10 and 250 milliseconds) to avoid redundant alerts while preserving ordering semantics. Thus, the event-driven change detector supplies structured, rationale-linked updates that eliminate separate manual review to understand why a modification occurred.

### Notification Engine 2336

The notification engine 2336 can monitor event buses and database change streams of the pharmacy-provider communication platform 2300 to detect prescription modifications, new laboratory results, and unread journal entries. In one example, the notification engine 2336 executes notify counterparty of modification and deliver notification using unified patient record data and a structured prescription modification entry to compose channel-specific payloads for in-application, email, short messaging service (SMS), and/or push delivery. Additionally, the notification engine 2336 can operate as a containerized microservice that scales horizontally and enforces recipient routing and prioritization based on clinical severity and organizational policy. Therefore, the notification engine 2336 provides timely visibility of prescription changes and related rationale, addressing limited direct communication time and lack of visibility.

More specifically, a duplicate suppression buffer can store hash digests of recently generated alert payloads in a circular, time-indexed structure residing in volatile memory. In one embodiment, the notification engine 2336 performs an O(1) lookup prior to dispatch and applies redundant alert suppression within a configurable time window, optionally coalescing payloads when near-duplicates are detected. Additionally, a high-resolution timer can prune expired entries to bound memory usage without external dependencies. Thus, the duplicate suppression buffer reduces alert fatigue and cognitive overload, preserving attention for clinically meaningful changes.

In one example, a persistent notification ledger can record an append-only sequence of creation, delivery attempts, suppressions, and acknowledgment events, each cryptographically chained with a secure hash algorithm, e.g., with a digest size of 256 bits (SHA-256 digest), and a monotonic sequence number. Additionally, the notification engine 2336 can serialize event payloads in a compact schema and flush records to durable storage at configurable intervals, optionally replicating to off-site archives for disaster recovery. Further, the audit and tracking module 2343 can query the ledger to generate compliance audit logging and downstream reports. Therefore, the persistent notification ledger provides tamper-evident provenance that supports regulatory audits and resolves disputes about communicated reasoning and timing.

Additionally or alternatively, the notification engine 2336 can expose REST endpoints and publish FHIR-compliant resources (and international equivalents) to interoperate with external EMRs or EHRs and decision support tools. In one embodiment, the notification engine 2336 executes real-time event monitoring with sub-second latency against platform streams and applies an Event Subscription Registry to filter clinically relevant events. Further, the integration gateway can mediate authentication and protocol adaptation to external systems while preserving message schemas. Thus, cross-system interoperability propagates prescription changes and context beyond a single platform, improving visibility across care settings.

In one embodiment, the notification engine 2336 can execute escalation on non-acknowledgment according to a configurable escalation matrix that targets secondary recipients when an acknowledgment does not arrive within a threshold. Additionally, the notification engine 2336 can apply user preference personalization by retrieving quiet hours, channel ranking, and preferred windows to sequence retries and format content. Further, the notification engine 2336 can continue escalation iteratively until an acknowledgment tracking record is stored. Therefore, the escalation logic reduces missed alerts and accelerates response when direct communication time is limited.

In particular, the change alert module 2338 can compose a prescription change notification package containing modified data elements, actor identity and role, timestamps, and linked reasoning extracted from structured entries and/or journal context. Additionally, the acknowledgment tracking module 2340 can detect, timestamp, and relay recipient acknowledgment events to the audit and tracking module 2343 while updating a status log for real-time dashboards. Further, the notification engine 2336 can coordinate these hierarchical components to route messages via the messaging system 2324 and mobile application interface according to channel policies. Thus, the coordinated modules present concise change summaries with accessible reasoning and confirmations, reducing manual record review and improving shared understanding.

### Notification Engine 2336—Change Alert Module 2338

The change alert module 2338 can operate as a subcomponent of the notification engine 2336 and can compose a prescription change notification package that includes modified data elements, an identity and role of a change initiator, a timestamp, and contextual reasoning. The change alert module 2338 can ingest unified patient record data and a structured prescription modification entry to format a structured alert payload and to prepare channel-specific renderings for in-application, email, SMS, and push delivery. In one example, the change alert module 2338 can queue alerts for batch delivery and/or escalation according to organizational policy and can expose configuration to a containerized microservice deployment. Thus, the change alert module 2338 can provide a consistent alert artifact that downstream components can deliver and audit.

The change alert module 2338 can listen for acknowledgment callbacks from the user interface module 2316 and/or the mobile application interface and can correlate the callbacks to an originating alert record. More specifically, the change alert module 2338 can stamp the alert record with an acknowledging user identifier and a timestamp and can emit an acknowledgment event to the acknowledgment tracking module 2340 and the audit and tracking module 2343. In one example, the change alert module 2338 can package acknowledgment metadata as counterparty acknowledgment data for downstream persistence and reporting. Thus, the change alert module 2338 can maintain closed-loop confirmation of delivery and review.

The change alert module 2338 can generate an immutable audit entry for each alert state transition, including creation, dispatch, escalation, and acknowledgment. More specifically, the change alert module 2338 can format the audit entry per an ingest specification of the audit and tracking module 2343 and can include a cryptographic hash of the alert payload and a sequence index. In one example, the change alert module 2338 can request a cryptographically timestamped event record and can attach a reference to support end-to-end verification. Thus, the change alert module 2338 can provide a verifiable chain suitable for compliance review.

The change alert module 2338 can synthesize an alert object in response to a qualifying change event sourced from the Medication Recommendation Platform and/or data management module 2326. More specifically, the change alert module 2338 can populate live fields and can complete generation within a time budget (e.g., between 50 and 100 milliseconds) to support near real-time clinical awareness. Additionally or alternatively, the change alert module 2338 can execute recipient preference routing by referencing a profile of a recipient from the access control and authentication module 2342 to honor quiet hours, registered device tokens, and escalation rules. Thus, the change alert module 2338 can tailor delivery pathways to reduce alert fatigue while preserving timely acknowledgment.

In cases where alerts are generated or received at an inopportune time, e.g., during an emergency, the alert has the potential to create “alarm fatigue”, which can result in the receiving team member ignoring the alert. The change alert module 2338 can generate alerts in an appropriate place (e.g., side bar of screen that does not prevent other actions on the screen from being taken) and at an appropriate time (e.g., can monitor screen to determine whether an emergency is taking place). In this way, the user is inhibited from instinctively dismissing the alert without readying or acknowledging the alert, e.g., since the alert will not interrupt a critical action the team member was performing. Additionally, the change alert module 2338 can be or can include an alert folder and can store alerts in the alert folder. The user can access the alert folder at convenient times for each particular user to confirm the alert is properly address, and then can dismiss the alert or otherwise move the alert to storage. Further, the change alert module 2338 can present, e.g., via other modules described herein, the alerts together. Thus, the alerts can be presented so that information is provided to end users in a coherent way, e.g., in a way a user can compare, confirm, and use the alert. Further, the alerts can be traceable and can be accessed at different times, e.g., when a user is preparing an order.

### Notification Engine 2336—Acknowledgment Tracking Module 2340

The acknowledgment tracking module 2340 can monitor, record, and manage recipient interactions with a prescription change notification package within the notification engine 2336. In one example, the acknowledgment tracking module 2340 can operate as a containerized microservice that exposes event ingestion and status update APIs to a user interface module 2316 and a mobile application interface while persisting state in a durable store with acknowledgment processing latency (e.g., between 10 and 200 ms). Additionally, the acknowledgment tracking module 2340 can forward acknowledgment artifacts to the audit and tracking module 2343 for aggregation and compliance reporting and can receive configuration from an access control and authentication module 2342 to enforce role-based handling. Therefore, the acknowledgment tracking module 2340 can maintain end-to-end visibility across pharmacist interface 2318, provider interface 2320, and shared journal interface 2322 clients without duplicative alerts.

More specifically, the acknowledgment tracking module 2340 can detect user acknowledgment events by consuming low-level interactions from the user interface module 2316 and/or the mobile application interface, including clicks, taps, or explicit acknowledge and dismiss commands. In one example, the acknowledgment tracking module 2340 can interpret interactions according to configurable heuristics and produce an acknowledgment signal that includes an alert identifier, identity of a recipient, device metadata, and a detection timestamp. Additionally, the acknowledgment tracking module 2340 can validate permissions for the identity of the recipient before accepting the acknowledgment signal and can emit a status update to a status log for downstream consumers.

Additionally, the acknowledgment tracking module 2340 can maintain an escalation timer queue that schedules follow-up actions for unacknowledged alerts according to policy-defined thresholds (e.g., between 1 and 120 minutes). In one example, the escalation timer queue can run as a distributed, replicated priority structure that preserves timer granularity with sub-second jitter and automatic failover. Further, the acknowledgment tracking module 2340 can enforce escalation workflows by resending alerts, notifying supervisory roles, and/or invoking out-of-band channels via a messaging system 2324 based on an escalation policy pointer associated with each timer entry. Then, the acknowledgment tracking module 2340 can cancel or reschedule timers upon receipt of a valid acknowledgment signal.

In particular, the acknowledgment tracking module 2340 can generate immutable acknowledgment records that bind an alert identifier with identity of a recipient, organizational role, device fingerprint, and a cryptographic timestamp packaged as schematized JSON or a FHIR AuditEvent resource. In one example, the acknowledgment tracking module 2340 can assign a sequential ledger number and submit the record to the audit and tracking module 2343 for storage and for creation of a cryptographically timestamped event record used in a compliance report. Additionally or alternatively, the acknowledgment tracking module 2340 can update notification engine 2336 status in real time to suppress redundant alerts and to synchronize acknowledged state across pharmacist interface 2318, provider interface 2320, and shared journal interface 2322 clients. Thus, the acknowledgment tracking module 2340 can preserve an auditable chain of custody while reducing alert fatigue.

### Access Control and Authentication Module 2342

The access control and authentication module 2342 can authenticate users and enforce authorization across the pharmacy-provider communication platform 2300 by applying multi-factor authentication and role-based access control. More specifically, the access control and authentication module 2342 can isolate pharmacist-only and provider-only actions across the user interface module 2316, the shared journal interface 2322, and/or the messaging system 2324 by filtering requests according to an authenticated role. In one example, the access control and authentication module 2342 can federate credentials through the integration gateway to an external identity provider while co-locating token verification and session middleware to reduce authorization latency under a containerized microservice deployment. Additionally, the access control and authentication module 2342 can log authentication attempts, access events, and permission changes to the audit and tracking module 2343 to support subsequent timestamp events and compliance report generation.

A role-specific permission matrix can store an encrypted mapping of a user identifier to at least one role descriptor and a set of discrete permission flags within a data store managed by the access control and authentication module 2342. More specifically, the access control and authentication module 2342 can construct an authorization context after authentication and can enforce role-based authorization by evaluating each API request against the permission flags before allowing access to protected resources. In one example, the role-specific permission matrix can apply column-level constraints to prevent or reduce mutually exclusive assignments and can support context modifiers based on time windows and/or device security posture. Additionally, the access control and authentication module 2342 can cache permission sets for an exemplary interval (e.g., between 30 and 300 seconds) and can refresh the cache upon receipt of a revocation event synchronized via the integration gateway.

Action attribution tagging can append an authenticated user identifier and a high-precision timestamp to every data-modifying request that the access control and authentication module 2342 authorizes. More specifically, the access control and authentication module 2342 can propagate the attribution through the shared journal interface 2322, the acknowledgment tracking module 2340, and the notification engine 2336 so downstream services embed the attribution in a structured prescription modification entry and a prescription change notification package. Additionally, the access control and authentication module 2342 can forward the attribution and a session identifier to the audit and tracking module 2343 to timestamp events and produce a cryptographically timestamped event record for later compliance report generation. Alternatively, the access control and authentication module 2342 can include device metadata and/or location claims within the attribution to maintain traceability for updates within a clarification message thread.

### Real-Time Change Notification and Acknowledgment Method 2400

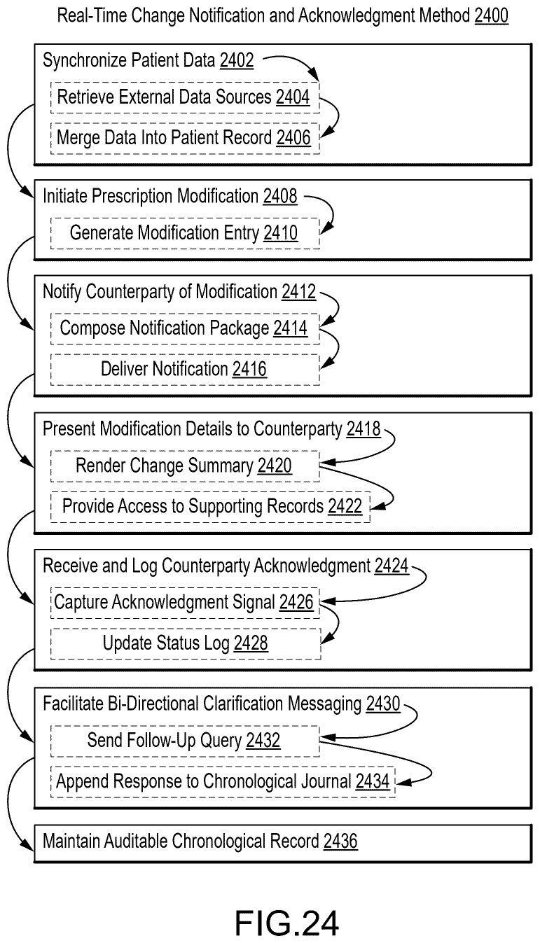

FIG. 24 depicts a method 2400, which can be referred to herein as a real-time change notification and acknowledgment method 2400. The pharmacy-provider communication platform 2300 can execute the real-time change notification and acknowledgment method 2400 that sequences data synchronization, modification capture, notification delivery, counterparty presentation, acknowledgment logging, clarification messaging, and audit logging. In one example, the platform 2300 and/or the method 2400 can generate a prescription change notification package and can persist an auditable change and acknowledgment record while the acknowledgment tracking module 2340 logs counterparty acknowledgment in real time. More specifically, the notification engine 2336 can deliver alerts across configured modalities while the user interface module 2316 renders modification details and supporting records to both pharmacist-side and provider-side users. Thus, the method 2400 can reduce latency and visibility gaps for prescription changes and can establish an auditable trail suitable for compliance and workflow coordination.

The notification engine 2336 can ensure or enable or assist in immediate counterparty awareness by emitting an event to the opposing party whenever a structured prescription modification entry is created. In one example, the acknowledgment tracking module 2340 can support escalation for unacknowledged changes by monitoring acknowledgment state within a policy-defined interval and by re-notifying via alternate channels and/or roles. More specifically, the change alert module 2338 can prioritize clinically significant modifications and can route alerts to mobile application interface endpoints and web endpoints in parallel. Therefore, the platform 2300 and/or the method 2400 can address limited time for direct communication and lack of visibility by guaranteeing prompt delivery and by automating acknowledgment follow-up.

The user interface module 2316 can provide transparent change rationale by rendering a comparative change summary with inline reasoning and by linking to supporting clinical artifacts. In one example, the platform 2300 and/or the method 2400 can reduce manual chart review burden by embedding laboratory results and journal context alongside pre- and post-modification parameters and by enabling single-click access to retrieved supporting record data. Additionally, the Medication Recommendation Platform 1500 can contribute structured rationale derived from unified patient record data when available to complement free-text notes. Thus, the counterparty can rapidly evaluate appropriateness without navigating disparate systems, addressing absent reasoning and time-consuming review.

The data management module 2326 can synchronize patient data by retrieving external data sources via the integration gateway and by merging data into a longitudinal patient record. In one example, a user of the pharmacist interface 2318 can initiate prescription modification, e.g., after the Medication Recommendation Platform 1500 evaluates clinical data for change need and the prescription database 2332 generates a structured modification entry. Additionally, the notification engine 2336 can notify the counterparty of modification by composing and delivering a notification package, and the user interface module 2316 can present modification details to the counterparty by rendering a change summary and by providing access to supporting records. Then, the acknowledgment tracking module 2340 can receive and log counterparty acknowledgment, the messaging system 2324 can facilitate bi-directional clarification messaging by sending follow-up queries and appending responses to a shared journal, and the audit and tracking module 2343 can maintain an auditable chronological record to enable end-to-end traceability.

Generally, unified patient record data can serve as input to the real-time change notification and acknowledgment method 2400 so that all downstream steps reference consistent context. In one example, the method 2400 can output a prescription change notification package for immediate delivery and can output an auditable change and acknowledgment record for durable compliance evidence. More specifically, the platform 2300 and/or the method 2400 can structure outputs with identifiers, timestamps, rationale fields, and delivery-acknowledgment metadata to support automated parsing and review. Therefore, consistent inputs and structured outputs can streamline communication while preserving accountability.

The pharmacy-provider communication platform 2300 and/or the method 2400 can execute the real-time change notification and acknowledgment method 2400 by coordinating the notification engine 2336, user interface module 2316, messaging system 2324, real-time synchronization engine 2334, and audit and tracking module 2343 (and or other modules of the platform 2300). In one example, a containerized microservice deployment can scale these components independently to meet variable alert volumes and acknowledgment workloads. Additionally, the access control and authentication module 2342 can gate method 2400 execution to authorized users across pharmacist and provider roles. Thus, the platform 2300 and/or the method 2400 can operationalize real-time collaboration with secure delivery, rapid acknowledgment capture, and comprehensive audit support.

### Method Step—Synchronize Patient Data 2402

The method 2400 can include a synchronize patient data step 2402. The data management module 2326 can execute the synchronize patient data step 2402. The synchronize patient data step 2402 can orchestrate retrieving external data sources and merging data into a patient record to produce unified patient record data. In one example, the data management module 2326 can trigger synchronization in real time, at scheduled intervals (e.g., between several seconds and several hours), and/or in response to external events such as newly posted laboratory results. More specifically, the data management module 2326 can exchange data via standardized interfaces, such as HL7 and/or FHIR APIs, and apply configurable cohort and data-type filters to constrain scope. In one embodiment, the data management module 2326 can complete the step by writing normalized updates into a longitudinal store so that subsequent modules operate on a synchronized view.

The data management module 2326 can harmonize records originating from EMRs or EHR, Laboratory Information Systems (LIS), and pharmacy systems into a unified internal schema to ensure or enable or assist in cross-system consistency. In one example, the data management module 2326 can maintain up-to-date patient record content by applying validation, deduplication, and conflict resolution rules with version tracking and provenance metadata. More specifically, the data management module 2326 can reconcile patient identity via deterministic and/or probabilistic matching and then align clinical terminology using standardized vocabularies. In one embodiment, the data management module 2326 can propagate accepted changes to all connected user interfaces with sub-second to multi-second latency to reduce stale views.

The integration gateway can retrieve external data sources by initiating connections to EMRs or EHR, LIS, and pharmacy endpoints at polling intervals (e.g., between 5 seconds and 24 hours) and/or upon receipt of push notifications. In one example, the integration gateway can issue parameterized queries and receive external clinical data payload objects in formats such as JSON, XML, HL7 v2, and/or FHIR bundles. More specifically, the integration gateway can normalize, validate, and deduplicate the received data before handing the data to the data management module 2326 for merge data into patient record. In one embodiment, the integration gateway can persist transient queues and retry failed transfers according to exponential backoff ranges to ensure or enable or assist in completeness.

The integration gateway can receive an external clinical data payload as an input comprising laboratory results, medication dispense events, diagnosis codes, allergy data, and/or encounter summaries. In one example, the integration gateway can secure the payload via authenticated channels and map the payload fields to the internal schema prior to storage. More specifically, the data management module 2326 can transform the payload into unified patient record data as an output by resolving identifiers, applying terminology mappings, and writing chronologically ordered entries. In one embodiment, the data management module 2326 can annotate each merged entry with source system, acquisition timestamp, and transformation metadata to support traceability.

The data management module 2326 can execute synchronize patient data by persisting, reconciling, and merging normalized objects into a longitudinal patient record. In one example, the data management module 2326 can enforce transactional consistency with ACID semantics and/or eventual consistency across distributed replicas while exposing APIs to downstream modules. More specifically, the data management module 2326 can emit unified patient record data to the Medication Recommendation Platform 1500, notification engine 2336, and user interface module without requiring additional format translation. In one embodiment, the data management module 2326 can support rollback of erroneous entries and maintain an audit trail to enable regulatory reporting and downstream compliance analytics.

### Synchronize Patient Data 2402—Retrieve External Data Sources 2404

The method 2400, e.g., the synchronize patient data step 2402, can include a retrieve external data sources step 2404. For example, the data management module 2326 can execute the retrieve external data sources step 2404 by establishing secure sessions to external EMRs or EHR, LIS, Health Information Exchange (HIE), and/or pharmacy systems according to a trigger policy (e.g., push events and/or polling intervals between 30 and 300 seconds). In one example, the integration gateway submits structured queries and receives structured objects formatted according to industry schemas (e.g., FHIR, HL7 v2, National Council for Prescription Drug Programs (NCPDP), JSON, and/or XML). More specifically, the integration gateway parses and normalizes received objects into an internal schema, maps terminologies, and performs validation and/or deduplication to prepare data for downstream synchronization. Thus, the integration gateway produces data suitable for subsequent merging while maintaining a consistent structure across disparate sources.

The real-time synchronization engine 2334 requests and ingests updated encounters, laboratory results, medication orders, and/or dispense events to acquire fresh clinical data. In one embodiment, the integration gateway maintains data integrity by enforcing schema conformance, applying deduplication rules, and tagging provenance and source timestamps for each element. Additionally, the real-time synchronization engine 2334 rejects or quarantines nonconforming elements according to configurable policies to prevent or reduce contamination of the shared record. Thus, the platform 2300 and/or the method 2400 reduces reliance on stale or conflicting information during clinical decision workflows.

The integration gateway generates an external clinical data payload as the output of the retrieval step, where the payload contains patient-specific clinical elements and associated provenance. In one example, the integration gateway encapsulates laboratory observations, medication history, diagnoses, allergies, and/or encounter summaries in transport formats compatible with the upstream systems (e.g., FHIR bundles, HL7 messages, and/or JSON documents). More specifically, the integration gateway includes metadata such as source system identifiers, received timestamps, and normalization status to support reconciliation during merging. Thus, the external clinical data payload arrives ready for the data management module 2326 to merge into the patient record in near real time.

Synchronize Patient Data 2402—Merge Data into Patient Record 2406

The method 2400, e.g., the synchronize patient data step 2402, can include a merge data into the patient record step 2406. For example, the data management module 2326 executes the merge data into the patient record step 2406. For example, the data management module 2326 can ingest heterogeneous clinical inputs, normalize structures and terminologies to a unified schema (e.g., HL7 FHIR or a proprietary model), and resolve patient identity using deterministic and/or probabilistic matching. More specifically, the data management module 2326 can map codes, units, and value sets, and then write normalized entries into the patient record database 2328 as chronologically ordered events with version identifiers and provenance annotations. In one example, the data management module 2326 applies encounter-aware matching using demographic fields and encounter metadata, and annotates merged entries with source system, received timestamps, and update actors for traceability. Thus, the data management module 2326 produces unified patient record data that downstream engines can query without additional translation.

The data management module 2326 enforces data integrity by validating schema conformance, performing de-duplication across redundant feeds, and maintaining referential relationships among patients, prescriptions, and laboratory results. More specifically, the data management module 2326 can execute transactional writes with rollback support, apply conflict resolution on concurrent updates using version vectors and/or timestamp ordering, and record immutable provenance logs. In one example, the audit and tracking module 2343 generates cryptographically timestamped records and verification hashes to detect tampering across a range of update intervals (e.g., between tens of milliseconds and several seconds). Thus, the platform 2300 and/or the method 2400 maintains logically consistent patient records that support reliable downstream clinical decisions.

The data management module 2326 reduces manual reconciliation by automatically consolidating external updates into a single, query-able timeline. More specifically, the real-time synchronization engine 2334 can propagate merged entries with sub-second latency to produce a unified view for pharmacists and providers, while the unified patient record data enables filtering by event type and time range. In one example, the platform 2300 and/or the method 2400 indexes merged entries by patient, encounter, code system, and event time to accelerate retrieval and eliminate repetitive cross-system review. Thus, the platform 2300 and/or the method 2400 provides a unified timeline that reduces human effort across routine review tasks.

The data management module 2326 executes the merge using an external clinical data payload as input and unified patient record data as output. More specifically, the data management module 2326 can parse payloads formatted according to HL7, FHIR, and/or NCPDP SCRIPT, validate required segments, and map fields to the internal schema prior to write operations. In one example, the platform 2300 and/or the method 2400 receives the external clinical data payload via secure APIs or message feeds, annotates entries with transport and source metadata, and commits merged results to the patient record database 2328 for immediate availability. Thus, the execution flow transforms incoming external content into synchronized unified patient record data suitable for subsequent method 2400 steps.

### Method Step—Initiate Prescription Modification 2408

The method 2400 can include an initiate prescription modification step 2408. The pharmacist interface 2318 executes the initiate prescription modification 2408 step by capturing a proposed change to a patient regimen, including medication identifier, modification type, and revised parameters. In one example, the platform 2300 and/or the method 2400 records a rationale as free-text and/or coded terminology and associates timestamps, author identity, and source of clinical indication. Additionally, the platform 2300 and/or the method 2400 validates completeness, prompts for missing rationale or supporting data, and formats a machine-readable order entry for downstream processing in real time. Therefore, the platform 2300 and/or the method 2400 reduces communication delays and lack of visibility by recording a precise, shareable modification at the moment of decision.

The platform 2300 and/or the method 2400 encodes the modification as a discrete event that the notification engine 2336 can consume to enable real-time notification workflow. In one example, the platform 2300 and/or the method 2400 enables accurate prescription capture by binding each changed attribute to explicit fields and by rejecting ambiguous entries. Additionally, the platform 2300 and/or the method 2400 stores versioned updates to preserve a clear change lineage for subsequent processing. Therefore, the platform 2300 and/or the method 2400 reduces latency and ambiguity, addressing limited communication time and downstream clarification cycles.

The platform 2300 and/or the method 2400 provides transparent clinical justification by persisting structured rationale codes and linked references to supporting records. In one example, the user interface module reduces cognitive load on clinicians by auto-populating rationale templates with recent laboratory values, vitals, and protocol citations. Additionally, the platform 2300 and/or the method 2400 allows role-appropriate editing to refine justification without duplicative data entry. Therefore, the platform 2300 and/or the method 2400 eliminates manual chart mining and absent reasoning by presenting clear, shared justification with minimal user effort.

The Medication Recommendation Platform 1500 evaluates clinical data for change need by comparing unified patient record data against guideline rules to generate a prescription adjustment recommendation. In one example, the prescription database 2332 generates a modification entry by merging the recommendation with current prescription context and recording proposed parameters for user confirmation. Additionally, the user can adjust thresholds or accept recommendations to finalize a structured entry. Therefore, the automated analysis and pre-population reduce time-consuming manual review while preserving clinician control.

The initiate prescription modification step 2408 ingests unified patient record data as an input and produces a structured prescription modification entry as an output. In one example, the pharmacist interface 2318 executes the step by presenting synchronized prescription, laboratory, and history data and then committing a machine-readable entry linked to the patient record. Additionally, the platform 2300 and/or the method 2400 timestamps and versions the entry to support downstream notification, acknowledgment, and auditing. Therefore, the synchronized input and structured output maintain accurate shared context, resolving lack of visibility between pharmacists and providers.

### Initiate Prescription Modification 2408—Generate Modification Entry 2410

The method 2400, e.g., the initiate prescription modification step 2408, can include a generate a modification entry step 2410. For example, the pharmacy-provider communication platform 2300 and/or the method 2400 can generate a modification entry by assembling updated prescription parameters and a rationale into a machine-readable object. In one example, the user interface module can require completion of one or more of medication name, dosage, frequency, route of administration, duration, or a rationale prior to finalizing generate modification entry. More specifically, the data management module 2326 can attach a timestamp, an identifier of the patient and the affected prescription, an identity and role of the initiating party, a source of triggering evidence, and a method of entry indicator during generate modification entry. Additionally, the platform 2300 and/or the method 2400 can pre-populate fields with recent laboratory values and/or clinical notes to reduce manual data entry during the generate modification entry 2410 step.

The user interface module 2316 can enforce field-level validation to assist in data completeness by verifying presence of all clinically relevant parameters and an explicit rationale. More specifically, the Medication Recommendation Platform 1500 can evaluate captured laboratory values, adverse-event indicators, and guideline references to preserve clinical context so downstream reviewers can interpret the change without additional record review. Additionally, the platform 2300 and/or the method 2400 can display inline prompts and/or selectable rationale templates that map to standardized clinical terminologies to maintain structured completeness. Thus, the platform 2300 and/or the method 2400 can reduce clarification cycles while maintaining consistent context for automated alerting and review.

The prescription database 2332 can receive a prescription adjustment recommendation and unified patient record data as inputs to parameterize generate modification entry. In one example, the Medication Recommendation Platform 1500 can supply recommended parameter changes and a clinical justification from the prescription adjustment recommendation, while the data management module 2326 can supply corroborating unified patient record data including identifiers, recent results, and prior orders. More specifically, the platform 2300 can reconcile conflicts between the recommendation and the unified patient record data by flagging discrepancies and suggesting normalized values before a prescription or change is finalized. Then, the platform 2300 and/or the method 2400 can use the reconciled content as the basis for the finalized entry.

The prescription database 2332 can execute generate modification entry and output a structured prescription modification entry that encodes parameters, rationale, timestamp, initiator identity, and references to supporting data. In one example, the data management module 2326 can assign version identifiers and maintain links to external records using standardized exchange formats (e.g., FHIR resources) for interoperability. Additionally or alternatively, the audit and tracking module 2343 can append audit metadata to the structured prescription modification entry to enable downstream notification and acknowledgment workflows. Thus, the platform 2300 and/or the method 2400 can produce a machine-readable artifact suitable for automated processing, display, and audit across pharmacy and provider interfaces 2320.

### Method Step—Notify Counterparty of Modification 2412

The method 2400 can include a notify counterparty of modification step 2412. The notification engine 2336 executes the notify counterparty of modification step 2412 by detecting a committed change event and initiating routing to a designated recipient endpoint. In one example, the notification engine 2336 triggers the notify counterparty of modification step 2412 upon receipt of a structured change signal and persists delivery state for audit. Thus, the notify counterparty of modification step 2412 reduces lack of visibility and reduces dependence on direct communication windows.

The notification engine 2336 targets immediate counterparty awareness by publishing alerts with a delivery latency constrained to a target window (e.g., between 100 milliseconds and 5 seconds, in some cases up to 1 day). More specifically, the notification engine 2336 facilitates rapid acknowledgment by embedding actionable controls that enable a recipient to confirm receipt and/or open a detailed view within a single interaction. Thus, real-time delivery and embedded response elements address limited time for direct communication and reduce uncoordinated therapy changes.

The change alert module 2338 executes compose notification package by aggregating change elements, rationale, and patient context into a standardized object formatted for interoperable parsing (e.g., JSON and/or FHIR). Additionally, the notification engine 2336 executes delivering a notification by selecting channels according to recipient preferences and/or urgency parameters and by dispatching the object with delivery receipts enabled. Thus, structured composition paired with adaptive delivery communicates reasoning alongside the change and avoids time-consuming manual record review.

The notify counterparty of modification step 2412 ingests a structured prescription modification entry as an input that encodes identifiers, parameter deltas, initiator role, a timestamp, and rationale codes and/or notes. In one example, the notify counterparty of modification step also ingests unified patient record data to reference supporting laboratory values and prior therapies without separate retrieval. Thus, standardized inputs expose change context in-line and reduce manual synthesis of disparate records.

The notification engine 2336 produces a prescription change notification package as an output that includes change fields, rationale content, recipient routing metadata, and acknowledgment hooks. More specifically, the notification engine 2336 records unique identifiers with delivery and read timestamps to enable downstream auditing and status propagation. Thus, the generated package provides immediate awareness, carries communicated reasoning, and supports compliance tracking without additional manual effort.

### Notify Counterparty of Modification 2412—Compose Notification Package 2414

The method 2400, e.g., the notify counterparty of modification step 2412, can include a compose a notification package step 2414. For example, the change alert module 2338 can compose a notification package by aggregating fields that describe a detected prescription modification and related clinical context. In one example, the change alert module 2338 assembles a machine-readable object (e.g., HL7 FHIR resource or JSON schema) that includes modified parameters, an initiator identity, a creation timestamp, and links and/or embedded references to supporting records. Additionally, the change alert module 2338 can attach a rationale note and delivery metadata, and can optionally apply a digital signature to enable authenticity and integrity verification. Alternatively, the notification engine 2336 can trigger the composition automatically on a qualifying event and/or expose a manual compose action through a user interface module.

The change alert module 2338 can embed a rationale and direct links to supporting laboratory results within the same structured object to facilitate rapid clinical decision-making. More specifically, the notification engine 2336 can surface context at the moment of receipt so that a counterparty renders an acknowledgment and/or judgment without querying external systems. Additionally, the platform 2300 and/or the method 2400 can precompute concise summaries from unified patient record data to reduce review time. Thus, the composed package can reduce navigation overhead and accelerate clinically safe responses.

The change alert module 2338 can accept a structured prescription modification entry and unified patient record data as inputs to the composition process. In one example, the change alert module 2338 maps fields of the structured prescription modification entry to normalized notification elements and enriches the payload by querying unified patient record data for recent laboratory values, relevant notes, and longitudinal medication history. Additionally, the change alert module 2338 can reconcile conflicting values using precedence rules derived from unified patient record data and can include references to underlying records for auditability. Alternatively, the platform 2300 and/or the method 2400 can defer large artifacts to retrievable links to maintain package size within a target limit (e.g., between 50 KB and 500 KB).

The change alert module 2338 can emit a prescription change notification package as the output of the composition operation. In one example, the package includes change specifics, initiator identity and role, timestamps for creation and/or last update, delivery and acknowledgment metadata, and pointers to supporting documentation. Additionally, the notification engine 2336 can register the package for downstream delivery and acknowledgment tracking while the audit and tracking module 2343 logs the composition event. Thus, the composed product can interoperate with provider and pharmacist interface 2318s for immediate rendering and subsequent auditing.

### Notify Counterparty of Modification 2412—Deliver Notification 2416

The method 2400, e.g., the notify counterparty of modification step 2412, can include a deliver notification step 2416. For example, the notification engine 2336 delivers a prescription change notification package to a designated counterparty according to channel policies of the pharmacy-provider communication platform 2300. In one example, the notification engine 2336 selects one or more channels (e.g., in-application alert, email, SMS, push) based on recipient preferences and/or an urgency indicator of the package, and the notification engine 2336 transmits the package in real time or near real time with a target latency (e.g., between 0.5 and 30 seconds). Additionally, the notification engine 2336 can apply delivery thresholds and deduplication windows (e.g., between 5 and 600 seconds) to suppress redundant alerts, and the notification engine 2336 can execute retries with exponential backoff when a channel endpoint of the recipient fails. Further, the notification engine 2336 can record delivery and open events as transport metadata and can expose actionable elements within the alert, such as an acknowledgment control and/or a deep link to a change summary rendered by the user interface module.

The notification engine 2336 prompts counterparty awareness by presenting a concise indicator of a prescription change together with a patient identifier and a timestamp of the change. More specifically, the notification engine 2336 can preserve clinical context by embedding structured deltas and a rationale reference within the alert and by providing links to supporting records rendered by the shared journal interface 2322, thereby enabling comprehension without leaving the notification surface. Additionally or alternatively, the notification engine 2336 can surface an acknowledgment control to elicit a rapid response from a device of the recipient and thereby reduce reliance on manual chart review.

The notification engine 2336 receives a prescription change notification package as an input and transmits the prescription change notification package to configured endpoints without material alteration of clinical content. In one example, the notification engine 2336 encapsulates the prescription change notification package within a channel-specific envelope while preserving identifiers, timestamps, and acknowledgment metadata for downstream logging. Additionally, the notification engine 2336 can map standardized fields of the prescription change notification package to display elements of the pharmacist interface 2318 and/or the provider interface 2320 to maintain interoperability during delivery.

### Method Step—Present Modification Details to Counterparty 2418

The method 2400 can include a present modification details to a counterparty step 2418. The user interface module 2316 can present modification details to a counterparty by rendering a comparative view that aligns original prescription parameters with revised prescription parameters upon interaction with a notification. In one example, the user interface module 2316 presents an inline rationale note adjacent to each changed parameter and optionally triggers the presentation automatically upon alert acknowledgment. Additionally, the user interface module 2316 can record a view event and an acknowledgment event for audit and workflow tracking, e.g., via the audit and tracking module 2343. Therefore, the user interface module 2316 addresses lack of visibility of prescription changes and absence of communicated reasoning by exposing structured differences and associated rationale at the moment of review.

The user interface module 2316 can reduce cognitive load by collating change details, rationale notes, and high-salience patient indicators within a single frame that emphasizes side-by-side comparison. In one example, the user interface module 2316 applies progressive disclosure via expandable panels and role-specific filters that prioritize fields by clinical relevance, date range, and modification type. Therefore, the user interface module 2316 reduces time-consuming manual review by eliminating cross-application navigation and by enabling rapid scanning of only the most relevant information.

The shared journal interface 2322 can provide access to supporting records by exposing linked or embedded viewers for laboratory results, chart notes, prescription histories, and guideline references in proximity to the change summary. In one example, the user interface module 2316 can render a change summary with visual indicators for each modified field and interactive elements that reveal previous and updated values, timestamps, and identities of initiating users, while the shared journal interface 2322 retrieves associated documents via secure links and presents the documents in a side panel or modal viewer. Therefore, the shared journal interface 2322 and the user interface module 2316 expedite understanding of clinical context without separate record searches, addressing limited time for direct communication between pharmacists and providers.

The user interface module 2316 can execute presentation by parsing a prescription change notification package and unified patient record data to populate the comparative view, inline rationale, and contextual links. In one example, the user interface module 2316 maps standardized fields (e.g., HL7 or FHIR resources) to interface components, applies delivery and acknowledgment metadata to drive stateful prompts, and synchronizes updates across pharmacist and provider endpoints. Therefore, the user interface module 2316 enables reliable, real-time display of consistent change information across roles, further reducing uncertainty and repeated clarification cycles.

### Present Modification Details to Counterparty 2418—Render Change Summary 2420

The method 2400, e.g., the present modification details to counterparty step 2418, can include a render change summary step 2420. For example, the user interface module 2316 can execute the render change summary step 2420 by generating a dedicated view that visually distinguishes modified prescription and/or patient record fields between two or more states. In one example, the user interface module 2316 highlights deltas of medication dosage, frequency, route, and formulation using color accents, underlining, and/or iconography, and attaches interactive elements that reveal previous and updated values, timestamps, and the identity of a change initiator upon hover or tap. Additionally, the user interface module 2316 can aggregate multiple modifications into a consolidated summary panel and trigger rendering automatically upon detection of a record update or upon user request, with configurable tracking of fields and visual encodings. Therefore, the render change summary step addresses lack of visibility of prescription changes and reduces time-consuming manual review of patient records.

The user interface module 2316 can support immediate comprehension of clinical impact by presenting only the delta and by annotating direction and magnitude of change adjacent to affected fields. Additionally or alternatively, the user interface module 2316 can reduce navigation overhead by collocating change details and contextual metadata within a single screen that reduces user transitions across tabs and windows. In one example, the user interface module 2316 positions acknowledgment and clarification controls proximal to the highlighted fields to reduce cognitive switching. Therefore, the configuration supports limited time for direct communication and reduces errors associated with overlooked subtle adjustments.

The user interface module 2316 can ingest the prescription change notification package as an input together with unified patient record data to populate the change summary view. In one example, the user interface module 2316 maps updated medication elements, a change initiator identity, timestamps, clinical rationale text and/or codes, and links to supporting documentation from the prescription change notification package, while retrieving longitudinal context and prior values from unified patient record data. Additionally, the user interface module 2316 can format the mapped content into field-level diffs with inline rationale disclosure and optional attachments accessible via expandable controls. Therefore, the combined inputs provide communicated reasoning for prescription modifications and reduce manual reconstruction of change rationale from disparate records.

Generally, execution of the render change summary step by the user interface module 2316 can result in a change summary interface active configuration that persists until dismissal or acknowledgment. In one example, the user interface module 2316 maintains real-time updates of newly received modifications and enables direct access to supporting records from the active view, while configuring pharmacist interface 2318, provider interface 2320, and shared journal interface 2322 layouts to emphasize the current patient and change type. Additionally or alternatively, the user interface module 2316 can present acknowledgment actions and comment entry points within the active configuration to streamline subsequent steps. Therefore, the active configuration improves shared visibility of changes and shortens the communication loop between pharmacists and providers.

### Present Modification Details to Counterparty 2418—Provide Access to Supporting Records 2422

The method 2400, e.g., the present modification details to counterparty step 2418, can include a provide access to supporting records step 2422. For example, the user interface module 2316, such as the shared journal interface 2322, can provide access to supporting records by rendering contextual links and/or an embedded viewer within the active communication thread. More specifically, the shared journal interface 2322 can retrieve or receive a supporting record and present metadata such as source, retrieval time, and a brief summary in a side panel or modal view. In one example, the shared journal interface 2322 can enable user annotations and links that associate specific excerpts of the retrieved supporting record with a journal entry or a message. Thus, the shared journal interface 2322 can maintain local context while exposing underlying materials relevant to a pending prescription change.

The user interface module 2316 can maintain workflow continuity by displaying supporting records inline without forcing navigation away from the change discussion. More specifically, the user interface module 2316 can preserve partially entered acknowledgment data and/or comments while a user expands and reviews a supporting viewer. In one example, the user interface module 2316 can synchronize the viewer state and the draft entry state so that dismissing the viewer returns the user to the precise location in the thread. Thus, the user interface module 2316 can reduce task switching latency and prevent or reduce loss of in-progress inputs.

The shared journal interface 2322 can parse a prescription change notification package and unified patient record data to locate references to supporting materials relevant to the displayed change. More specifically, the shared journal interface 2322 can resolve identifiers in the prescription change notification package and cross-reference fields in the unified patient record data, then retrieve a supporting record for immediate review. In one example, the shared journal interface 2322 can cache a subset of referenced objects for rapid reopening within the same session. Thus, the shared journal interface 2322 can transform structured change context into a directly accessible supporting document view.

The change summary interface active configuration can enable provide access to supporting records by exposing controls adjacent to each changed field. More specifically, the user interface module 2316 can surface a “view evidence” control that launches the shared journal interface 2322 to execute retrieval and rendering of a retrieved supporting record. In one example, the user interface module 2316 can keep the change summary visible while the shared journal interface 2322 overlays the supporting viewer for side-by-side review. Thus, the configuration can streamline evidence inspection during change review and acknowledgment.

### Method Step—Receive and Log Counterparty Acknowledgment 2424

The method 2400 can include a receive and log counterparty acknowledgment step 2424. The acknowledgment tracking module 2340 can receive and log counterparty acknowledgment by ingesting an acknowledgment payload associated with a specific prescription change event. The acknowledgment tracking module 2340 can generate a timestamp, associate an acknowledging user identifier, and bind the payload to a prescription change identifier and/or a clarification message thread. In one example, the acknowledgment tracking module 2340 can persist the payload and metadata in a persistent, query-able store and record an acknowledgment method 2400 code indicating a web interface, mobile application, and/or API source. Additionally, the acknowledgment tracking module 2340 can support multiple acknowledgment types, maintain sequence ordering for auditability, and expose authorized read access to the acknowledgment journal.

The notification engine 2336 can dispatch originator status update events after successful persistence of an acknowledgment payload. In one example, a message broker can publish an acknowledgment-received event containing an acknowledgment identifier, a counterparty identity, and an optional comment string to a channel subscribed by the originator client. Additionally, the acknowledgment tracking module 2340 can schema-validate acknowledgment payloads against a predefined schema that requires a prescription-change identifier, an acknowledging user identifier, and a timestamp, and optionally includes a comment field and an acknowledgment-method 2400 code. In one example, the schema-validation routine can reject malformed payloads to prevent or reduce ambiguous records from entering an audit log and to enable uniform downstream processing.

The acknowledgment tracking module 2340 can capture acknowledgment signal via explicit user interaction and/or passive viewing heuristics. In one example, the acknowledgment tracking module 2340 can detect a button press, checkbox selection, or menu action, and alternatively or additionally can infer a read receipt when a change summary remains active for a threshold duration (e.g., between 5 and 15 seconds). Subsequently, the acknowledgment tracking module 2340 can update status log by creating a chronological entry that transitions a related task state from pending to acknowledged and stores user identity, device metadata, and a generated timestamp. In one example, the acknowledgment tracking module 2340 can emit an internal event to trigger interface refresh and downstream workflow without client polling.

The acknowledgment tracking module 2340 can generate counterparty acknowledgment data as a structured, timestamped record linked to a prescription change identifier and an acknowledging user identifier. In one example, the counterparty acknowledgment data can encode a type code such as viewed, accepted, rejected, and/or requires clarification, along with an optional free-text or templated comment and an acknowledgment-method 2400 indicator. Additionally, the acknowledgment tracking module 2340 can store the counterparty acknowledgment data in a secure, tamper-evident store and provide query endpoints for authorized parties. In one example, the acknowledgment tracking module 2340 can transmit the counterparty acknowledgment data to an audit and tracking module 2343 for longitudinal retention and compliance reporting.

### Receive and Log Counterparty Acknowledgment 2424—Capture Acknowledgment Signal 2426

The method 2400, e.g., the receive and log counterparty acknowledgment step 2424, can include a capture an acknowledgment signal step 2426. For example, the acknowledgment tracking module 2340 can capture an acknowledgment signal in response to a delivered prescription change notification via explicit user interaction and/or passive display detection. More specifically, the user interface module 2316 can emit the acknowledgment signal when a pharmacist interface 2318 or provider interface 2320 renders an acknowledgment control and a recipient selects the control, and alternatively when the notification remains in a foreground view for a threshold duration (e.g., between 5 and 30 seconds). In one example, the acknowledgment tracking module 2340 can attach metadata including a recipient identifier, a notification identifier, a timestamp, and device/application context, and can persist the data in association with a corresponding structured prescription modification entry and prescription change notification package. Additionally, the acknowledgment tracking module 2340 can transmit the captured data through the access control and authentication module 2342 to the audit and tracking module 2343 for durable storage and downstream processing.

The pharmacy-provider communication platform 2300 and/or the method 2400 can confirm recipient awareness by binding each acknowledgment signal to a unique recipient identifier and a corresponding notification identifier. More specifically, the acknowledgment tracking module 2340 can require explicit user confirmation for selected categories of changes and/or accept passive acknowledgment according to configurable criteria to close the communication loop between pharmacy and provider teams. Additionally, the user interface module 2316 can render status indicators to reflect awareness state for the originating party.

The audit and tracking module 2343 can aggregate acknowledgment events to compute elapsed time between notification issuance and acknowledgment for latency analytics. More specifically, the audit and tracking module 2343 can derive distributions and benchmarks, while the real-time synchronization engine 2334 can provide high-resolution event ordering via timestamps to identify bottlenecks. Additionally or alternatively, the notification engine 2336 can apply the captured acknowledgment signal as a terminating condition to cancel pending reminders and to disable or de-queue escalation steps according to configurable thresholds.

The acknowledgment tracking module 2340 can generate the acknowledgment signal as a structured object comprising a recipient identifier, a notification identifier, a timestamp, device/application context, and an indicator of an explicit or passive modality. More specifically, the acknowledgment tracking module 2340 can serialize the object in a standardized interchange format such as JSON or XML and can transmit the object over an authenticated, encrypted channel to the audit and tracking module 2343. Additionally, the acknowledgment tracking module 2340 can output the acknowledgment signal for subsequent update status log processing and/or originator notification.

### Receive and Log Counterparty Acknowledgment 2424—Update Status Log 2428

The method 2400, e.g., the receive and log counterparty acknowledgment step 2424, can include an update status log step 2428. For example, the acknowledgment tracking module 2340 can update a persistent, chronological status log by appending an acknowledgment event that references a prescription change notification. In one example, the module 2340 records a recipient identifier, a notification identifier, a timestamp, and optional metadata such as device type, network address, and contextual notes, and the data store can comprise a database table and/or a distributed ledger. Subsequently, the user interface module 2316 can transition a status indicator from a pending state to an acknowledged state and trigger a real-time refresh and/or a notification to authorized viewers. Thus, the platform 2300 and/or the method 2400 increases visibility of prescription changes and reduces coordination time between parties.

The pharmacy-provider communication platform 2300 and/or the method 2400 can maintain synchronized workflow state by committing the acknowledgment event and the status-indicator transition as a single atomic transaction replicated by the real-time synchronization engine 2334 across the pharmacist interface 2318, the provider interface 2320, and the shared journal interface 2322. In one example, the audit and tracking module 2343 can record acknowledgment latency and related metadata to enable turnaround-time analysis, bottleneck detection, and service-level-agreement monitoring. Additionally or alternatively, the platform 2300 and/or the method 2400 can batch-process multiple acknowledgments while preserving event order to avoid race conditions across concurrent sessions. Therefore, the synchronized life-cycle view enables timely clinical decisions and reduces manual follow-up to confirm state.

The acknowledgment tracking module 2340 can receive an acknowledgment signal that includes a recipient identifier, a notification identifier, a timestamp, device and application metadata, and an acknowledgment modality indicator (e.g., explicit action or passive view exceeding a threshold duration, for example between 2 and 10 seconds). In one example, the module validates the acknowledgment signal, enriches the record with optional comment fields and/or geolocation subject to policy, and generates counterparty acknowledgment data that links to a prescription change record and an audit trail. Additionally, the module can forward the counterparty acknowledgment data to the audit and tracking module 2343 and optionally notify the notification engine 2336 to inform the originator. Thus, structured acknowledgment data with modality and comments conveys reasoning and status without requiring manual chart review.

### Method Step—Facilitate Bi-Directional Clarification Messaging 2430

The method 2400 can include a facilitate bi-directional clarification messaging step 2430. The messaging system 2324 can facilitate bi-directional clarification messaging by creating a communication thread directly associated with a structured prescription modification entry. More specifically, the messaging system 2324 can anchor each message to the relevant modification context and can support synchronous and/or asynchronous exchanges between pharmacist-side and provider-side users. Additionally, the notification engine 2336 can publish message alerts and the audit and tracking module 2343 can apply timestamps to preserve chronological order for later review. In one example, the shared journal interface 2322 can expose the thread within the user interface module 2316 to maintain continuity with related prescription change artifacts.

The pharmacy-provider communication platform 2300 and/or the method 2400 can accelerate cross-team understanding by providing a low-latency channel for requesting rationale, laboratory evidence, and guideline citations. More specifically, the access control and authentication module 2342 can limit information exposure by enforcing role-based visibility so that only authorized users view or participate in a thread. Additionally, the notification engine 2336 can surface high-priority prompts to reduce turnaround time from hours to minutes. In one example, the Medication Recommendation Platform 1500 can suggest clarifying prompts that reference guideline codes derived from unified patient record data.

The shared journal interface 2322 can append response to a chronological journal by writing each submitted reply as a discrete, time-stamped entry linked to the underlying modification. Additionally, the messaging system 2324 can send follow-up query entries and can associate delivered, read, and acknowledged states with the same journal context. In one example, the audit and tracking module 2343 can generate immutable metadata for user role, timestamps (e.g., according to a synchronized time source), and subsequent edits. Thus, authorized reviewers can reconstruct decision-making sequences without manual collation of disparate records.

The messaging system 2324 can ingest a structured prescription modification entry and unified patient record data as inputs to prepopulate message context, such as medication parameters, rationale codes, and links to supporting records. More specifically, the user interface module 2316 can render query templates conditioned on the modification type and on clinical signals derived from unified patient record data. Additionally, the integration gateway can resolve external references so that attached laboratory results or notes remain accessible from the message context. In one embodiment, the real-time synchronization engine 2334 can refresh displayed fields to reflect updates made during ongoing discussions.

The messaging system 2324 can execute the clarification message thread by instantiating, indexing, and persisting a bidirectional conversation bound to a single prescription change record. More specifically, the messaging system 2324 can produce the clarification message thread as an output that includes threaded replies, attachments, and status indicators. Additionally, the audit and tracking module 2343 can record creation, participation, closure, and export events for compliance. In one embodiment, the shared journal interface 2322 can display the active thread alongside the change summary to maintain context across communication and documentation workflows.

### Facilitate Bi-Directional Clarification Messaging 2430—Send Follow-Up Query 2432

The method 2400, e.g., the facilitate bi-directional clarification messaging step 2430, can include a send follow-up query step 2432. For example, the messaging system 2324 can execute the send follow-up query step 2432 by transmitting a clarification request between the pharmacist interface 2318 and the provider interface 2320 within a clarification context. In one example, the user interface module 2316 can present context-aware templates and/or a free-text composer, and the messaging system 2324 can deliver the submission through a secure channel while notifying intended recipients. Additionally, the audit and tracking module 2343 can log the query, associate the query with a patient record, and track state values (e.g., sent through responded). Alternatively, the notification engine 2336 can auto-trigger the step when predefined conditions are met, such as absence of an explanatory note within a prescription change notification package.

The pharmacy-provider communication platform 2300 and/or the method 2400 can clarify prescription rationale by enabling users to request concise reasoning for a prescription modification through guided prompts. More specifically, the user interface module 2316 can reduce manual chart review by presenting structured queries aligned to clinical context. Additionally, the platform 2300 and/or the method 2400 can accelerate therapy decisions by returning targeted explanations to the requesting party. Further, the platform 2300 and/or the method 2400 can help assist in patient safety by exposing misunderstandings and data omissions to both parties for timely correction.

The clarification message thread can provide input context to the send follow-up query step, and the messaging system 2324 can generate a follow-up query message as output. In one example, the messaging system 2324 can link the follow-up query message to the clarification message thread, record author identity and timestamp metadata, and support delivery confirmation and read acknowledgment. Additionally, the shared journal interface 2322 can present the clarification message thread alongside supporting records and can accept attachments referenced by the follow-up query message. Then, the audit and tracking module 2343 can propagate status changes to the maintain auditable chronological record step for downstream compliance reporting. Additionally, the prescribers can override a chance, e.g., in an emergency situation. For example, if a pharmacist is blocking a necessary prescription in an emergency where urgency needed, the prescriber can override the block to address the time-sensitive emergency.

### Facilitate Bi-Directional Clarification Messaging 2430—Append Response to Chronological Journal 2434

The method 2400, e.g., the facilitate bi-directional clarification messaging step 2430, can include an append a response to a chronological journal step 2434. For example, the user interface module 2316, e.g., the shared journal interface 2322, can append a response to a chronological journal by persisting a discrete entry that references a prescription change and/or a clarification event. In one example, the shared journal interface 2322 can attribute the entry to an authenticated user role and preserve insertion order within a patient-scoped stream. Additionally, the shared journal interface 2322 can support structured and/or free-text payloads with validation prior to commit. Therefore, the step increases visibility of prescription changes and preserves expressed rationale, reducing missed context between pharmacists and providers.

More specifically, the audit and tracking module 2343 can generate a synchronized timestamp by querying an NTP-aligned clock source and embedding a UTC value with sub-second precision into the entry prior to storage. In one example, the audit and tracking module 2343 can anchor the entry to a deterministic time base to enable consistent ordering across geographically distributed clients. Additionally, the audit and tracking module 2343 can retain the time source identifier for forensic verification. Therefore, the step eliminates ambiguity in event sequencing, supporting rapid, low-effort reconstruction instead of manual collation.

In one example, the data management module 2326 can resolve identifiers referenced by the response and link contextual clinical references to the entry as immutable pointers to unified patient record data and/or supporting documents. Additionally or alternatively, the access control and authentication module 2342 can attach role-based metadata at write time to encode the author role as an enumerated value. Further, the user interface module 2316 can surface these links to provide immediate clinical context with single-click traversal to labs, orders, or prior notes. Therefore, the step embeds rationale and evidence next to the dialogue, reducing manual chart review and misinterpretation.

Then, the notification engine 2336 can broadcast a real-time update by publishing a lightweight event to subscribed pharmacist interface 2318 and provider interface 2320 sessions for the active patient. In one example, the notification engine 2336 can include the entry identifier and a minimal diff to allow clients to fetch the full record on demand. Additionally, the audit and tracking module 2343 can persist the event to maintain immutable communication chronology using append-only semantics. Therefore, the step enables timely awareness under limited communication windows while preserving a durable timeline.

Additionally, the messaging system 2324 can consume a clarification message thread as an input context that binds the response to a specific prescription modification dialogue. In one example, the messaging system 2324 can also consume a follow-up query message as an input to correlate the response with the originating question and delivery status. Therefore, the step maintains continuity of the discussion so reviewers do not search disparate channels to infer relationships.

In one example, the shared journal interface 2322 can generate a follow-up response journal entry as an output object that stores structured explanation fields, role metadata, and the synchronized timestamp. Additionally, the shared journal interface 2322 can expose bidirectional navigation between the response and the linked query within the same patient context. Further, the shared journal interface 2322 can permit filters by role, time range, and event type for expedited retrieval. Therefore, the step delivers an auditable, readily accessible record of reasoning, improving visibility and minimizing time-consuming manual review.

### Method Step—Maintain Auditable Chronological Record 2436

The method 2400 can include a maintain an auditable chronological record step 2436. The audit and tracking module 2343 can maintain an auditable chronological record by appending an event object to a persistent, tamper-evident store for each action of the pharmacy-provider communication platform 2300. More specifically, the audit and tracking module 2343 can assign a unique identifier, associate user and patient context, and link the event to related records to support end-to-end traceability. In one example, the audit and tracking module 2343 can apply cryptographic hashing and/or digital signatures to each append operation and can restrict mutation to authorized system processes. Additionally, the audit and tracking module 2343 can support standardized export and query operations (e.g., HL7, FHIR) and can schedule periodic integrity verification over a configurable interval (e.g., between 5 minutes and 24 hours).

The audit and tracking module 2343 can enable chronological reconstruction by storing forward-linked entries that reference prior hashes and by enforcing monotonic sequence numbers. More specifically, the audit and tracking module 2343 can provide a replay function that iterates events in recorded order to reveal cause-and-effect relationships without external cross-referencing. In one example, the audit and tracking module 2343 can detect gaps or reordering and can raise an alert when sequence validation fails. Thus, authorized reviewers can reconstruct clinical workflows across prescription changes, notifications, acknowledgments, and messaging exchanges.

The audit and tracking module 2343 can timestamp events by generating a server-sourced timestamp and a digital signature for each event. More specifically, the audit and tracking module 2343 can obtain time from a synchronized source (e.g., NTP or a secure clock), bind the timestamp to user identity and event type, and sign the event using asymmetric cryptography (e.g., RSA or ECDSA). In one example, the audit and tracking module 2343 can persist a cryptographically timestamped event record and can include device and/or network metadata to enhance provenance. Thus, downstream validation can verify occurrence, identity, and precise time across distributed users.

The audit and tracking module 2343 can ingest inputs to the auditable chronological record that include a structured prescription modification entry, a prescription change notification package, counterparty acknowledgment data, and a clarification message thread. More specifically, the audit and tracking module 2343 can link the structured prescription modification entry to subsequent notifications and acknowledgments to preserve modification lineage. Additionally, the audit and tracking module 2343 can register delivery and read states from the prescription change notification package and can bind acknowledgment types and timestamps from the counterparty acknowledgment data. In one example, the audit and tracking module 2343 can thread queries and responses from the clarification message thread so that message exchanges appear inline with the associated prescription change context.

The audit and tracking module 2343 can generate an auditable change and acknowledgment record as an output that consolidates modification details, delivery events, acknowledgment actions, and related messages in a tamper-evident sequence. More specifically, the audit and tracking module 2343 can populate fields for patient and prescription identifiers, initiator identity and role, change parameters, delivery method 2400 and time, acknowledgment identity and time, and references to supporting artifacts. In one example, the audit and tracking module 2343 can expose retrieval and filtering by user, patient, event type, and time window and can export the record for external compliance workflows. Thus, authorized parties can review a complete, verifiable history for compliance, quality assurance, and dispute resolution.

### Medical Device Programming Instruction Display Platform.

Current IV pump programming approaches, such as manual input, result in mistakes occurring in approximately four out of five programming attempts, representing a significant patient safety concern in healthcare environments. Additionally, traditional IV pump administration systems are directed to automatically injecting the fluid without human interaction or invention during administration, but do not assist the provider in actually programming the pump prior to administration and thus still represent substantial challenges that contribute to widespread errors in medical treatment.

The high error rate in IV pump administration may stem from multiple underlying factors that complicate the programming process. Healthcare providers may be required to perform numerous complex calculations manually when programming IV pumps, including determining appropriate flow rates, calculating dosage amounts, and establishing treatment duration parameters. These calculations can involve multiple variables such as patient weight, medication concentration, desired dosage per unit time, and total treatment volume. Unit conversions can represent another source of programming errors in traditional IV pump systems. Healthcare providers may need to convert between different measurement units, such as converting between milligrams and micrograms, milliliters and liters, or hours and minutes. These conversions may be performed under time pressure in clinical settings, increasing the likelihood of calculation errors that can lead to incorrect pump programming.

Traditional IV pump administration systems also lack standardization across different pump manufacturers and models. Each pump brand or series has different programming interfaces, menu structures, and input requirements. Healthcare providers may need to learn and remember multiple programming procedures for different pump types, which may contribute to confusion and programming mistakes when switching between different pump models. Further, traditional IV pump administration systems, e.g., that replaces the provider during administration, is tailored to specific brands of pumps.

The complexity of traditional IV pump administration systems may be further compounded by the need to interpret and translate prescription information into pump-specific parameters. Healthcare providers still need to determine whether medications should be administered as bolus doses or continuous infusions, calculate appropriate dilution ratios, and establish safety parameters such as maximum flow rates and occlusion pressure limits, as well as flushing procedures. These determinations may require clinical judgment combined with mathematical calculations, creating additional opportunities for errors in the programming process.

The programming instruction display platform described herein can be similar to or the same as the pharmacy-provider communication platform 2300 and/or the medicine recommendation platform 1500, e.g., one or more layers or engines 1516, 1524, 1532, 1544, 1552 of the medicine recommendation platform 1500 and/or one or more modules or engines 2316, 2326, 2334, 2336, 2342, 2343 of the pharmacy-provider communication platform 2300 can implement the programming instruction display platform and the respective functions described herein. For example, the presentation layer 1544 and/or the user interface module 2316 can present the screens described herein to a team member, such as a nurse, e.g., via the provider interface 2320.

The programming instruction display platform addresses the challenges associated with manual programming of IV pumps as well as traditional IV pump administration systems by providing comprehensive guidance and verification capabilities for healthcare providers. The platform offers universal compatibility that enables the system to function with IV pumps from any manufacturer, brand, or series, reducing or eliminating the need for healthcare providers to learn multiple programming procedures for different pump models.

The programming instruction display platform can receive input, e.g., via the data acquisition layer 1516 and/or the data management module 2326, regarding pump specifications, including make, model, brand, series, and type information. Healthcare providers can select pump information from a provided list or submit images of the pump to identify the specific pump model. The platform can also receive patient information including patient-specific records, charts, measurements, prescriptions, disease information, and details regarding the liquid to be administered. For example, the platform can receive patient-specific demographic information, such as patient age, patient weight, patient height, patient gender, or patient ethnicity; patient-specific measurements, such as patient body surface area, patient vital signs, patient values from laboratory test, or patient organ function parameters; patient-specific disease information, such as patient condition severity, treatment protocols, clinical guidelines, or contraindications; and patient-specific prescription, such as medication name, concentration, dosing requirements, administration routes, treatment duration, or physician-specified parameters. Anthropometric measurements are not the only measurements the platform can receive or healthcare providers can rely on to make determinations. For example, the platform can receive other measurements that can be used to determine appropriate dosing, such as kidney function measurements, liver function measurements, coagulation function measurements, and the like, as well as other tests, labs, or measurements that can be used to further or better understand the patient condition.

The platform can calculate or retrieve appropriate treatment plans based on the received patient information and pump specifications, e.g., via the data storage layer 1524 and/or the analysis engine 1532. The treatment plan can include determinations regarding the correct fluid type, fluid amount, administration method, and whether the liquid should be administered as a bolus dose or metered out over time, and if so, the rate. The platform can perform the complex calculations and unit conversions that traditionally contribute to programming errors.

The programming instruction display platform can provide step-by-step visual instructions that guide healthcare providers through the pump programming process, e.g., via the presentation layer 1544 and/or the user interface module 2316. The visual instructions can include images showing what the IV pump display screen should appear like at each programming step, providing clear visual guidance that corresponds to the specific pump model being programmed. The step-by-step approach can reduce programming complexity by breaking down the process into manageable sequential tasks.

As depicted in FIG. 39, the platform can display a final verification screen 3905 that can show the completed pump face display matching the specific brand and containing all correct programming information, e.g., via the presentation layer 1544 and/or the user interface module 2316. The verification screen can enable healthcare providers to compare the actual programmed pump display with the expected display before patient treatment begins. The verification process can include displaying the appropriate drip rate, liquid to be dispensed, treatment duration, and other programming parameters specific to the treatment plan and pump model. In other examples, the platform can display the verification screen 3910, which can display a visualization of a drug to be administered subcutaneously, e.g., via a syringe, as described herein with respect to FIG. 39. In other examples, the platform can display a verification screen 4005, a medication visualization screen 4010, an administration confirmation screen 4015, and the like related to an oral medication, as depicted in and described herein with respect to FIG. 40. In other examples, the platform can display the screen of interface 1400 depicting the syringe with the dose of medicine to be administered.

The universal compatibility aspect of the programming instruction display platform can enable the system to generate appropriate visual instructions and verification displays for any IV pump type, regardless of manufacturer specifications. The platform can maintain databases of pump interface information for different manufacturers and models, allowing the system to provide accurate visual representations of pump displays across various pump types without being limited to specific pump models or manufacturers.

The programming instruction display platform can operate through multiple integrated input mechanisms that enable healthcare providers to specify pump information through various convenient methods, e.g., via the data acquisition layer 1516. A user selection interface can present healthcare providers with comprehensive lists of available pump manufacturers, models, brands, and series information organized in searchable or filterable formats. The selection interface can include dropdown menus, searchable databases, or categorized listings that allow healthcare providers to quickly locate and select the specific pump model being used for patient treatment.

Alternatively or additionally, the programming instruction display platform includes image submission capability and can enable healthcare providers to capture and submit one or more photographs of the IV pump, e.g., a model number of the IV pump, a display screen of the IV pump, or the like, and the programming instruction display platform can identify pump specifications automatically, e.g., via the data acquisition layer 1516 and/or the data storage layer 1524. For example, the image processing functionality can analyze submitted pump images to extract identifying information such as manufacturer logos, model numbers, display configurations, physical characteristics that correspond to specific pump types, or other identifiers to give more specific and relevant instruction. The image recognition system can compare submitted images against a database of pump images to determine the appropriate pump specifications and programming requirements.

Alternative input mechanisms can include barcode scanning capabilities, manual text entry fields, or voice recognition systems that allow healthcare providers to specify pump information through various interaction methods. The multiple input options can accommodate different clinical workflows and healthcare provider preferences while ensuring accurate pump identification regardless of the input method selected.

The platform can process comprehensive patient information through multiple data input channels that capture relevant clinical parameters for treatment planning. Patient-specific records can include demographic information, medical history, current medications, allergies, and previous treatment responses that influence IV therapy decisions. Electronic health record (EHR), which may be referred to herein as electronic medical record (EMR), integration can enable automatic retrieval of patient information from hospital information systems, reducing manual data entry requirements and minimizing transcription errors.

Clinical measurements or records can include patient demographic information, such as age, weight, height, gender, ethnicity; body surface area; vital signs; values from laboratory tests; and organ function parameters that affect medication dosing calculations. The platform can receive real-time measurement inputs, e.g., from a provider or a patient, or retrieve stored measurement data from connected monitoring devices or laboratory information systems. Prescription information can include medication names, concentrations, dosing requirements, administration routes, and physician-specified parameters that define the treatment objectives. Prescription information can be retrieved from respective FDA labels, drug manufacturer labels, direct user input, or the like.

Disease-specific information can influence treatment planning by providing context regarding patient condition severity, treatment protocols, and clinical guidelines that apply to specific medical conditions. The platform can incorporate disease-specific dosing algorithms, contraindication checks, e.g., as listed on FDA labels, and safety parameters that correspond to particular diagnoses or treatment indications. Liquid administration details can specify the type of medication or fluid to be administered, concentration requirements, diluent specifications, and compatibility considerations that affect pump programming parameters.

The platform can determine appropriate treatment plans, e.g., via the analysis engine 1532, through computational algorithms that calculate dosing parameters based on one or more of patient-specific variables, disease-specific information, medication-specific information, and clinical guidelines. Calculation engines can process patient weight, medication concentration, desired dose per unit time, and treatment duration to determine flow rates, total volumes, and administration schedules. The computational approach can incorporate pharmacokinetic models, dosing equations, and safety algorithms that account for patient-specific factors and medication characteristics.

Treatment plan retrieval systems can access pre-existing treatment protocols stored in databases or clinical decision support systems. The retrieval system can match patient characteristics and clinical conditions with established treatment protocols that have been validated, e.g., by the FDA, provider, or the like, for specific patient populations or medical conditions. Database-stored treatment plans can include standardized dosing regimens, institutional protocols, or evidence-based guidelines that provide appropriate treatment parameters for common clinical scenarios.

The platform can determine administration methods by analyzing one or more of medication properties, disease information, patient factors, and clinical requirements to specify whether liquid or medicine should be administered as immediate bolus doses or metered out over extended time periods. Bolus administration determination can consider factors such as medication onset requirements, patient hemodynamic stability, and clinical urgency that favor rapid medication delivery. Continuous infusion determination can account for medication half-life, therapeutic window requirements, and patient tolerance factors that favor gradual medication administration over time.

A practical clinical use case can demonstrate platform operation when a healthcare provider programs an IV pump for antibiotic administration to a pediatric patient. The healthcare provider can select the pump model from a dropdown menu showing “Brand X Model 200” or submit a photograph of the pump for automatic identification. Patient information input can include the child's weight of 25 kilograms, diagnosis of pneumonia, prescribed antibiotic of ceftriaxone 50 mg/kg/day, and administration schedule of every 12 hours.

The platform can calculate the appropriate dose as 1,250 mg per administration based on the patient weight and prescribed dosing regimen. The system can determine that the antibiotic should be administered as a continuous infusion over 30 minutes rather than as a bolus dose, based on medication safety guidelines and pediatric administration protocols, e.g., from the respective FDA label for ceftriaxone. The platform can generate step-by-step programming instructions showing the healthcare provider how to input the calculated flow rate, infusion duration, and medication volume into the specific pump model. For example, the program can display an array of images of the “Brand X Model 200” pump, each image displaying a chronological programming step, with the final image displaying what the pump should look like just prior to the start of drug administration. For example, the final verification screen can display the expected pump interface showing “Ceftriaxone 1,250 mg in 50 mL, Rate: 100 mL/hr, Duration: 30 minutes”, which enables the healthcare provider to confirm the real-time IV pump has been correctly programmed before initiating patient treatment.

Any of the features, components, and/or parts, including the arrangements and configurations thereof shown in and of the figures can be included, either alone or in any combination, in any of the other examples of devices, features, components, and parts shown in the other figures described herein. Similarly, any of the features, elements, or modules described herein with respect to one system or platform or method can be used with or by any of the other systems or platforms or methods described herein. For example, the pharmacy-provider communication platform 2300 and/or the method 2400 can present the comprehensive view of candidate drugs with sortable attributes for dose recommendations, expected efficacy, interaction risk indicators, and cost estimates prepared or presented by the summary dashboard 1546 via the user interface module 2316 (e.g., the comprehensive view can be presented on the pharmacist interface 2318, the provider interface 2320, the shared journal interface 2322, and/or the messaging system 2324). In this way, the entire healthcare team can be informed of the medication recommendation prepared by the medicine recommendation platform 1500, and likewise informed of potential medication complications, e.g., due to a patient's comorbidity, potential contraindications, and the like.

The word “herein” includes the descriptions provided throughout this specification, including the Field, Background, Summary, Brief Description of the Drawings, Detailed Description, Claims, and Abstract. As used herein, the word “exemplary” is used herein to mean “serving as an example, instance, or illustration.” Any embodiment described herein as “exemplary” is not necessarily to be construed as preferred or advantageous over other embodiments. As used herein, the terms “about” and “substantially” herein are to be construed as +/−10%, unless stated otherwise. As used herein, every range of values (such as of the form, “from about a to about b,” or, equivalently, “from approximately a to b,” or, equivalently, “from approximately a-b” or, equivalently, “greater than about a and less than about b”, for example) disclosed herein is to be understood to set forth every number and range encompassed within the broader range of values. As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

As used herein, the terms “first”, “second”, “third” etc. may be used to describe various elements, components, regions, layers and/or sections, these elements, components, regions, layers and/or sections and should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present disclosure.

As used herein, spatially relative terms, such as “beneath”, “below”, “lower”, “under”, “above”, “upper”, “over”, and the like, may be used herein for ease of description to describe one element or feature's relationship to another element(s) or feature(s) as illustrated in the figures. It will be understood that the spatially relative terms are intended to encompass different orientations of the device or system, e.g., in use or operation in addition to the orientation depicted in the figures. For example, if the device or system in the figures is turned over, elements described as “below” or “beneath” or “under” other elements or features would then be oriented “above” the other elements or features. Thus, the exemplary terms “below” and “under” can encompass both an orientation of above and below. The device or system may be otherwise oriented (rotated 90 degrees or at other orientations) and the spatially relative descriptors used herein are to be interpreted accordingly. In addition, it will also be understood that when an object or component is referred to as being “between” two objects or components, the object or component can be the only object or component between the two objects or components, or one or more additional objects or components may also be present.

The terminology used herein is for the purpose of describing particular embodiments only and is not intended to be limiting of the disclosure. As used herein, the singular forms “a,” “an,” and “the” are to be construed as open ended, e.g., meaning “at least one”, and as such are intended to include the plural forms as well, unless the context clearly indicates otherwise. It will be further understood that the terms “comprises” and/or “comprising,” when used in this specification, specify the presence of stated features, integers, steps, operations, elements, components, and/or groups but do not preclude the presence or addition of one or more other features, integers, steps, operations, elements, components, and/or groups thereof. As used herein, the term “and/or” includes any and all combinations of one or more of the associated listed items and may be abbreviated as “/”. For example, the term “and/or” when used in the context of a listing of entities, refers to the entities being present singly or in combination, and the phrase “A, B, C, and/or D” includes A, B, C, and D individually, but also includes any and all combinations and subcombinations of A, B, C, and D.

As used herein, when an element is referred to as being “on,” “connected,” “attached,” “mounted,” “coupled,” or “adjacent” another element, it can be directly on, connected, coupled, or adjacent to the other element, or intervening elements may be present. For example, as used herein, the term “adjacent” can mean next to, neighboring, or abutting and in contact, but does not necessarily mean in contact unless otherwise stated. In contrast, when an element is referred to as being “directly on,” “directly connected,” “directly coupled,” or “immediately adjacent” another element, there are no intervening elements present. As used herein, the phrases “connected,” “attached,” “mounted,” “coupled,” and the like can be used interchangeably. For example, these terms can be used herein to mean a removable coupling, a fixed coupling, a fixedly removable couple, a permanent coupling, and the like, unless explicitly stated otherwise.

It should be understood that logical operations may be performed in any order, unless explicitly claimed otherwise or a specific order is inherently necessitated by the claim language. Additionally, some of the operations described may be skipped or not included in described methodologies. For example, in methodologies directly or indirectly set forth herein, various steps and operations are described in one possible order of operation but those skilled in the art will recognize the steps and operation may be rearranged, replaced, or eliminated without necessarily departing from the spirit and scope of the present disclosure. It is intended that all matter contained in the description or shown in the accompanying drawings shall be interpreted as illustrative only and not limiting. Changes in detail or structure may be made without departing from the spirit of the present disclosure as defined in the appended claims.

Other examples and implementations are within the scope and spirit of the disclosure and appended claims. Thus, the foregoing descriptions of the specific examples described herein are presented for purposes of illustration and description. They are not targeted to be exhaustive or to limit the examples to the precise forms disclosed. It will be apparent to one of ordinary skill in the art that many modifications and variations are possible in view of the above teachings.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed:

1. A medicine recommendation platform comprising:
a data acquisition layer comprising a patient information collector and a drug information collector, each operable to ingest patient-specific clinical data and drug-specific data, respectively;
an analysis engine comprising a multi-criteria ranking engine configured to compute composite suitability score for candidate medications by weighting one or more of medication efficacy, medication safety, medication resistance, medication cost, or patient-specific coverage factors; and
a presentation layer comprising a summary dashboard configured to display a ranked medication recommendation set produced by the analysis engine.

2. The medicine recommendation platform of claim 1, further comprising:
a real-time sensitivity and resistance data connector communicatively linked to the drug information collector and configured to supply continuously updated drug efficacy information;
wherein the analysis engine further comprises an efficacy and resistance evaluator configured to incorporate the drug efficacy information.

3. The medicine recommendation platform of claim 1, wherein the analysis engine further comprises:
an interaction and contraindication checker configured to screen the candidate medications against one or more of patient allergies, comorbidities, or concomitant therapies.

4. The medicine recommendation platform of claim 3, wherein the interaction and contraindication checker is configured to assign severity grades to identified risks and to annotate the ranked medication recommendation set with risk indicators.

5. The medicine recommendation platform of claim 1, wherein the analysis engine further comprises:
a cost and coverage analyzer configured to retrieve patient-specific cost and formulary information.

6. The medicine recommendation platform of claim 1, further comprising:
a data-refresh subsystem comprising:
a monitoring module configured to monitor real-time data feeds; and
a recalculation trigger module configured to trigger score recalculation upon detecting parameter changes surpassing a configurable threshold.

7. The medicine recommendation platform of claim 1, wherein the data acquisition layer further comprises an EMR interface configured to retrieve structured patient data.

8. The medicine recommendation platform of claim 1, wherein the analysis engine further comprises a dose calculation engine configured to generate drug-specific organ-adjusted dose guidance based on organ function.

9. The medicine recommendation platform of claim 1, further comprising:
a pictogram generator and a packaging display module configured to render visual identifiers of at least one candidate medication in the ranked medication recommendation set.

10. A patient-specific medication recommendation method comprising:
acquiring, via a data acquisition step, data including patient-specific clinical parameters and contemporaneous drug information;
calculating patient-specific dosage ranges for at least one candidate medication;
evaluating contraindications and interaction risks for each candidate medication relative to a patient profile;
computing, with a multi-criteria ranking routine, a composite suitability score for each candidate medication that integrates one or more of medication efficacy, medication resistance, medication safety, medication cost, or patient-specific coverage factors; and
presenting a ranked medication recommendation set on a summary dashboard.

11. The patient-specific medication recommendation method of claim 10, further comprising:
harmonizing the acquired data to generate a harmonized clinical and drug dataset.

12. The patient-specific medication recommendation method of claim 10, wherein acquiring the patient-specific clinical parameters further comprises extracting:
patient insurance information.

13. The patient-specific medication recommendation method of claim 10, wherein acquiring the patient-specific clinical parameters further comprises extracting:
laboratory results including one or more of serum creatinine, liver enzymes, or a blood test.

14. The patient-specific medication recommendation method of claim 10, wherein calculating the patient-specific dosage ranges scale doses according to respective FDA-labels or manufacturer label prescribing information.

15. The patient-specific medication recommendation method of claim 10, wherein computing the composite suitability score applies user-configurable weighting coefficients to one or more of the medication efficacy, medication resistance, medication safety, medication cost, or patient-specific coverage factors.

16. The patient-specific medication recommendation method of claim 10, further comprising:
monitoring real-time clinical or pharmaceutical data feeds and, in response to detecting a threshold change in a monitored parameter, automatically recomputing the composite suitability scores and updating the presented ranked medication recommendation set.

17. The patient-specific medication recommendation method of claim 10, further comprising:
generating a visual summary interface view that displays, for each candidate medication, an efficacy percentage, a recommended dose, contraindication alerts, and an estimated patient out-of-pocket cost or institutional cost.

18. A non-transitory computer-readable storage medium storing instructions that, when executed by one or more processors, cause a system to perform the method of claim 10.

19. The non-transitory computer-readable storage medium of claim 18, wherein the instructions further cause the system to monitor real-time data feeds.

20. The non-transitory computer-readable storage medium of claim 18, wherein the instructions further cause the system to trigger automatic score recomputation in response to a susceptibility rate changing by at least a predefined percentage.
